(19) United States
(12) Patent Application Publication
(10) Pub. No.: US 2015/0372244 A1
(43) Pub. Date: Dec. 24, 2015

(54) ORGANIC LIGHT-EMITTING DEVICE

(71) Applicant: CANON KABUSHIKI KAISHA, Tokyo (JP)

(72) Inventors: Shigemoto Abe, Yokohama-shi (JP); Kengo Kishino, Tokyo (JP); Jun Kamatani, Tokyo (JP); Naoki Yamada, Inagi-shi (JP); Tetsuya Kosuge, Yokohama-shi (JP); Takayuki Horiuchi, Tokyo (JP); Yosuke Nishide, Kawasaki-shi (JP); Hirokazu Miyashita, Tokyo (JP); Akihito Saitoh, Gotemba-shi (JP)

(21) Appl. No.: 14/764,376

(22) PCT Filed: Feb. 4, 2014
(86) PCT No.: PCT/JP2014/052981

§ 371 Date: Jul. 29, 2015

(30) Foreign Application Priority Data
Feb. 6, 2013 (JP) 2013-021049

(51) Int. Cl. H01L 29/08; H01L 35/24; H05B 39/04; H05B 41/36

(52) U.S. Cl. CPC H01L 51/0085; H01L 51/0072; H01L 51/0052; H01L 51/0058; H01L 51/0067; H01L 51/0074; H01L 51/0073; H01L 51/0054; H01L 27/3248; G03G 15/04036; H05B 33/0896; H01L 51/5028

(57) ABSTRACT

Provided is an organic light-emitting device having high efficiency and improved driving durability performance. The organic light-emitting device includes a pair of electrodes and an organic compound layer placed between the pair of electrodes, in which the organic compound layer includes an iridium complex having a specific skeleton and a heterocycle-containing compound as a host.

### TECHNICAL FIELD

The present invention relates to an organic light-emitting device.

### BACKGROUND ART

An organic light-emitting device (organic electroluminescence device or organic EL device) is an electronic device including an anode and a cathode, and an organic compound layer placed between both the electrodes. A hole and an electron injected from the respective electrodes recombine in the organic compound layer to produce an exciton, and the organic light-emitting device emits light upon return of the exciton to its ground state. Recent development of the organic light-emitting devices is significant and the developed devices have, for example, the following features. The organic light-emitting devices can be driven at low voltages, emit light beams having various wavelengths, have high-speed responsivity, and can be reduced in thickness and weight.

Of the organic light-emitting devices, a phosphorescent device is a light-emitting device that includes a phosphorescent material in its organic compound layer for forming the organic light-emitting device and provides light emission derived from a triplet exciton of the material. By the way, the phosphorescent device has room for additional improvements in emission efficiency and durability lifetime, and there are demands for an improvement in emission quantum yield of the phosphorescent material and suppression of degradation of a molecular structure of a host molecule in an emission layer.

PTL 1 discloses Ir(pbiq)₃ shown below as an iridium complex having an arylbenzo[f]isoquinoline as a ligand (hereinafter referred to as biq-based Ir complex) known as a red phosphorescent material having a high emission quantum yield. In addition, PTL 1 discloses an organic light-emitting device whose emission layer contains Ir(pbiq)₃ shown below as a guest. By the way, high emission efficiency of the organic light-emitting device disclosed in PTL 1 largely depends on the high emission quantum yield of the biq-based Ir complex incorporated as the guest into the emission layer.

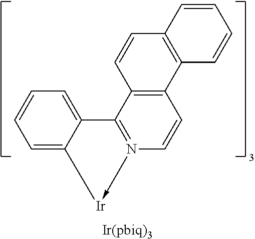

In addition, PTL 2 discloses an organic light-emitting device using, as a host for an emission layer, a benzo-fused thiophene or benzo-fused furan compound that is a heterocycle-containing compound.

### CITATION LIST

### Patent Literature

PTL 1: Japanese Patent Application Laid-Open No. 2009-114137
PTL 2: Japanese Patent Translation Publication No. 2010-535809

### Non Patent Literature

NPL 1: Tetrahedron, (2010), Vol. 66, p. 2111-2118
NPL 2: J. Am. Chem. Soc., (2001), Vol. 123, p. 4304-4312

### SUMMARY OF INVENTION

### Solution to Problem

Thus, the present invention provides an organic light-emitting device, including: a pair of electrodes; and an organic compound layer placed between the pair of electrodes, in which the organic compound layer includes an iridium complex represented by the following general formula [1] and a heterocycle-containing compound as a host:

Ir(L)ₘ(L′)ₙ [1]

in the formula [1], Ir represents iridium, L and L′ represent bidentate ligands different from each other, provided that L and L′ each represent a ligand containing at least one alkyl group, m represents 2, n represents 1, and a partial structure Ir(L)ₘ includes a partial structure represented by the following general formula [2]:

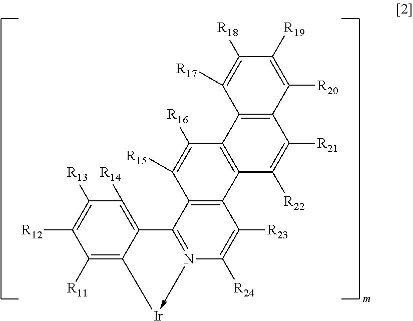

In the formula [2]: R₁₁ to R₁₄ each represent a hydrogen atom, a fluorine atom, a substituted or unsubstituted alkyl group, an alkoxy group, a substituted amino group, a substituted or unsubstituted aryl group, or a substituted or unsubstituted heterocyclic group, and may be identical to or different from one another, and R₁₅ to R₂₄ each represent a hydrogen atom, a fluorine atom, a substituted or unsubstituted alkyl group, an alkoxy group, or a substituted amino group, and may be identical to or different from one another; and a partial structure Ir(L′)ₙ includes a partial structure containing a monovalent bidentate ligand.

Further features of the present invention will become apparent from the following description of exemplary embodiments with reference to the attached drawings.

### BRIEF DESCRIPTION OF DRAWINGS

FIG. 1 is a schematic sectional view illustrating a display apparatus including an organic light-emitting device and a switching device connected to the organic light-emitting device.

### DESCRIPTION OF EMBODIMENTS

Preferred embodiments of the present invention will now be described in detail in accordance with the accompanying drawing.

In PTL 1, an iridium complex having an arylnaphtho[2,1-f]isoquinoline ligand has not been used as the guest to be incorporated into the emission layer. In addition, the luminescent color of the organic light-emitting device disclosed in PTL 2 is green and an organic light-emitting device whose luminescent color is red has not been disclosed.

The present invention has been accomplished to solve the problems, and an object of the present invention is to provide an organic light-emitting device having high efficiency and improved driving durability.

Hereinafter, the present invention is described in detail.

(1) Organic Light-Emitting Device

An organic light-emitting device of the present invention includes: a pair of electrodes; and an organic compound layer placed between the pair of electrodes. In addition, in the present invention, the organic compound layer includes an iridium complex represented by the following general formula [1] and a heterocycle-containing compound as a host.

Ir(L)ₘ(L′)ₙ [1]

It is to be noted that details about the iridium complex represented by the general formula [1] and the heterocycle-containing compound are described later.

The specific device construction of the organic light-emitting device of the present invention is, for example, a multilayer-type device construction obtained by sequentially stacking, on a substrate, electrode layers and an organic compound layer described in each of the following constructions (1) to (6). It is to be noted that in each of the device constructions, the organic compound layer necessarily includes an emission layer including a light-emitting material.

(1) Anode/emission layer/cathode

(2) Anode/hole transport layer/emission layer/electron transport layer/cathode

(3) Anode/hole transport layer/emission layer/electron transport layer/electron injection layer/cathode

(4) Anode/hole injection layer/hole transport layer/emission layer/electron transport layer/cathode

(5) Anode/hole injection layer/hole transport layer/emission layer/electron transport layer/electron injection layer/cathode

(6) Anode/hole transport layer/electron blocking layer/emission layer/hole blocking layer/electron transport layer/cathode

It is to be noted that those device construction examples are only very basic device constructions and the device construction of the organic light-emitting device of the present invention is not limited thereto.

For example, the following various layer constructions can each be adopted: an insulating layer, an adhesion layer, or an interference layer is provided at an interface between an electrode and the organic compound layer, the electron transport layer or the hole transport layer is formed of two layers having different ionization potentials, or the emission layer is formed of two layers including different light-emitting materials.

In the present invention, the aspect according to which light output from the emission layer is extracted (device form) may be the so-called bottom emission system in which the light is extracted from an electrode on a side closer to the substrate or may be the so-called top emission system in which the light is extracted from a side opposite to the substrate. In addition, a double-face extraction system in which the light is extracted from each of the side closer to the substrate and the side opposite to the substrate can be adopted.

Of the device constructions (1) to (6), the construction (6) is preferred because the construction includes both the electron blocking layer and the hole blocking layer. In other words, the construction (6) including the electron blocking layer and the hole blocking layer provides an organic light-emitting device that does not cause any carrier leakage and has high emission efficiency because both carriers, i.e., a hole and an electron can be trapped in the emission layer with reliability.

In the organic light-emitting device of the present invention, the iridium complex represented by the general formula [1] and the heterocycle-containing compound are preferably incorporated into the emission layer out of the organic compound layer. In this case, the emission layer includes at least the iridium complex represented by the general formula [1] and the heterocycle-containing compound. The applications of the compounds to be incorporated into the emission layer in this case vary depending on their content concentrations in the emission layer. Specifically, the compounds are classified into a main component and a sub-component depending on their content concentrations in the emission layer.

The compound serving as the main component is a compound having the largest weight ratio (content concentration) out of the group of compounds to be incorporated into the emission layer and is a compound also called a host. In addition, the host is a compound present as a matrix around the light-emitting material in the emission layer, and is a compound mainly responsible for the transport of a carrier to the light-emitting material and the donation of an excitation energy to the light-emitting material.

In addition, the compound serving as the sub-component is a compound except the main component and can be called a guest (dopant), a light emission assist material, or a charge injection material depending on a function of the compound. The guest as one kind of sub-component is a compound (light-emitting material) responsible for main light emission in the emission layer. The light emission assist material as one kind of sub-component is a compound that assists the light emission of the guest, and is a compound having a smaller weight ratio (content concentration) in the emission layer than that of the host. The light emission assist material is also called a second host by virtue of its function. In the present invention, the (light emission) assist material is preferably an iridium complex, provided that the iridium complex to be used as the (light emission) assist material is an iridium complex except the iridium complex represented by the general formula [1].

The concentration of the guest with respect to the host is 0.01 wt % or more and 50 wt % or less, preferably 0.1 wt % or more and 20 wt % or less with reference to the total amount of the constituent materials for the emission layer. The concentration of the guest is particularly preferably 10 wt % or less from the viewpoint of preventing concentration quenching.

In the present invention, the guest may be uniformly incorporated into the entirety of the layer in which the host serves as a matrix, or may be incorporated so as to have a concentration gradient. In addition, the guest may be partially incorporated into a specific region in the emission layer to make the layer a layer having a region free of the guest and formed only of the host.

In the present invention, the following aspect is preferred: both the iridium complex represented by the general formula [1] and the heterocycle-containing compound are incorporated as the guest and the host, respectively, into the emission layer. In this case, in addition to the iridium complex represented by the general formula [1], another phosphorescent material may be further incorporated into the emission layer for assisting the transfer of an exciton or a carrier.

In addition, a compound different from the heterocycle-containing compound may be further incorporated as the second host into the emission layer for assisting the transfer of the exciton or the carrier.

(2) Iridium Complex

Next, the iridium complex as one constituent material for the organic light-emitting device of the present invention is described. The iridium complex as one constituent material for the organic light-emitting device of the present invention is a compound represented by the following general formula [1]. It is to be noted that the iridium complex represented by the following general formula [1] emits red light.

Ir(L)ₘ(L′)ₙ [1]

In the formula [1], Ir represents iridium.

In the formula [1], L and L′ represent bidentate ligands different from each other. As described above, the two kinds of ligands (L and L′) of the iridium complex represented by the formula [1] are bidentate ligands different from each other, and hence the two kinds of ligands are in a relationship of different ligand species.

It is to be noted that one of L and L′ in the formula [1] represents a ligand having an alkyl group.

In the formula [1], m represents 2.

In the formula [1], n represents 1.

In the formula [1], a partial structure Ir(L)ₘ is specifically a partial structure represented by the following general formula [2].

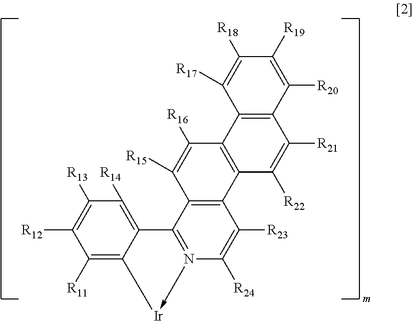

In the formula [2], R₁₁ to R₁₄ each represent a hydrogen atom, a fluorine atom, a substituted or unsubstituted alkyl group, an alkoxy group, a substituted amino group, a substituted or unsubstituted aryl group, or a substituted or unsubstituted heterocyclic group, and may be identical to or different from one another.

In the formula [2], R₁₅ to R₂₄ each represent a hydrogen atom, a fluorine atom, a substituted or unsubstituted alkyl group, an alkoxy group, or a substituted amino group, and may be identical to or different from one another.

The alkyl group represented by any one of R₁₁ to R₂₄ is preferably an alkyl group having 1 or more and 10 or less carbon atoms, more preferably an alkyl group having 1 or more and 6 or less carbon atoms. Specific examples of the alkyl group having 1 or more and 6 or less carbon atoms include a methyl group, an ethyl group, an n-propyl group, an i-propyl group, an n-butyl group, an i-butyl group, a sec-butyl group, a tert-butyl group, an n-pentyl group, an i-pentyl group, a tert-pentyl group, a neopentyl group, an n-hexyl group, and a cyclohexyl group. Of those alkyl groups, a methyl group or a tert-butyl group is preferred.

Specific examples of the alkoxy group represented by any one of R₁₁ to R₂₄ include a methoxy group, an ethoxy group, an i-propoxy group, an n-butoxy group, and a tert-butoxy group. Of those alkoxy groups, a methoxy group is preferred.

Specific examples of the substituted amino group represented by any one of R₁₁ to R₂₄ include an N-methylamino group, an N-ethylamino group, an N,N-dimethylamino group, an N,N-diethylamino group, an N-methyl-N-ethylamino group, an N-benzylamino group, an N-methyl-N-benzylamino group, an N,N-dibenzylamino group, an anilino group, an N,N-diphenylamino group, an N,N-dinaphthylamino group, an N,N-difluorenylamino group, an N-phenyl-N-tolylamino group, an N,N-ditolylamino group, an N-methyl-N-phenylamino group, an N,N-dianisoylamino group, an N-mesityl-N-phenylamino group, an N,N-dimesitylamino group, an N-phenyl-N-(4-tert-butylphenyl)amino group, and an N-phenyl-N-(4-trifluoromethylphenyl)amino group. Of those substituted amino groups, an N,N-dimethylamino group or an N,N-diphenylamino group is preferred.

Specific examples of the aryl group represented by any one of R₁₁ to R₁₄ include a phenyl group, a naphthyl group, a phenanthryl group, an anthryl group, a fluorenyl group, a biphenylenyl group, an acenaphthylenyl group, a chrysenyl group, a pyrenyl group, a triphenylenyl group, a picenyl group, a fluoranthenyl group, a perylenyl group, a naphthacenyl group, a biphenyl group, and a terphenyl group. Of those aryl groups, a phenyl group, a naphthyl group, a fluorenyl group, or a biphenyl group is preferred, and a phenyl group is more preferred.

Specific examples of the heterocyclic group represented by any one of R₁₁ to R₁₄ include a thienyl group, a pyrrolyl group, a pyrazinyl group, a pyridyl group, an indolyl group, a quinolyl group, an isoquinolyl group, a naphthyridinyl group, an acridinyl group, a phenanthrolinyl group, a carbazolyl group, a benzo[a]carbazolyl group, a benzo[b]carbazolyl group, a benzo[c]carbazolyl group, a phenazinyl group, a phenoxazinyl group, a phenothiazinyl group, a benzothiophenyl group, a dibenzothiophenyl group, a benzofuranyl group, a dibenzofuranyl group, an oxazolyl group, and an oxadiazolyl group.

A substituent that the alkyl group, the aryl group, and the heterocyclic group may each further have is not particularly limited. Examples thereof may include: alkyl groups such as a methyl group, an ethyl group, an n-propyl group, an i-propyl group, an n-butyl group, an i-butyl group, a sec-butyl group, a tert-butyl group, an n-pentyl group, an i-pentyl group, a tert-pentyl group, a neopentyl group, an n-hexyl group, and a cyclohexyl group; alkoxy groups such as a methoxy group, an ethoxy group, an i-propoxy group, an n-butoxy group, and a tert-butoxy group; substituted amino groups such as an N-methylamino group, an N-ethylamino group, an N,N-dimethylamino group, an N,N-diethylamino group, an N-methyl-N-ethylamino group, an N-benzylamino group, an N-methyl-N-benzylamino group, an N,N-dibenzylamino group, an anilino group, an N,N-diphenylamino group, an N,N-dinaphthylamino group, an N,N-difluorenylamino group, an N-phenyl-N-tolylamino group, an N,N-ditolylamino group, an N-methyl-N-phenylamino group, an N,N-dianisoylamino group, an N-mesityl-N-phenylamino group, an N,N-dimesitylamino group, an N-phenyl-N-(4-tert-butylphenyl)amino group, and an N-phenyl-N-(4-trifluoromethylphenyl)amino group; aryl groups such as a phenyl group, a naphthyl group, a phenanthryl group, an anthryl group, a fluorenyl group, a biphenylenyl group, an acenaphthylenyl group, a chrysenyl group, a pyrenyl group, a triphenylenyl group, a picenyl group, a fluoranthenyl group, a perylenyl group, a naphthacenyl group, a biphenyl group, and a terphenyl group; heterocyclic groups such as a thienyl group, a pyrrolyl group, a pyrazinyl group, a pyridyl group, an indolyl group, a quinolyl group, an isoquinolyl group, a naphthyridinyl group, an acridinyl group, a phenanthrolinyl group, a carbazolyl group, a benzo[a]carbazolyl group, a benzo[b]carbazolyl group, a benzo[c]carbazolyl group, a phenazinyl group, a phenoxazinyl group, a phenothiazinyl group, a benzothiophenyl group, a dibenzothiophenyl group, a benzofuranyl group, a dibenzofuranyl group, an oxazolyl group, and an oxadiazolyl group; a cyano group; and a trifluoromethyl group.

The substituent, which the alkyl group, the aryl group, and the heterocyclic group may each further have, is preferably a methyl group, a tert-butyl group, a methoxy group, an N,N-dimethylamino group, an N,N-diphenylamino group, a phenyl group, a naphthyl group, a fluorenyl group, or a biphenyl group. Of those, a methyl group, a tert-butyl group, or a phenyl group is particularly preferred.

It is understood from the foregoing that one of the ligands constituting the iridium complex represented by the formula [1] is a ligand using 1-phenylnaphtho[2,1-f]isoquinoline (niq) as a main skeleton as represented by the formula [2]. In addition, the niq-based iridium complex (Ir complex) serves as a ligand having an alkyl group particularly when the ligand L′ to be described later is free of any alkyl group.

Next, L′ is described. A partial structure Ir(L′)ₙ is a structure containing a monovalent bidentate ligand (L′). Examples of L′ may include acetylacetone, phenylpyridine, picolinic acid, an oxalate, and salen.

The partial structure Ir(L′)ₙ in the formula [1] is preferably a partial structure represented by any one of the following general formulae [3] to [5], more preferably a partial structure represented by the general formula [3].

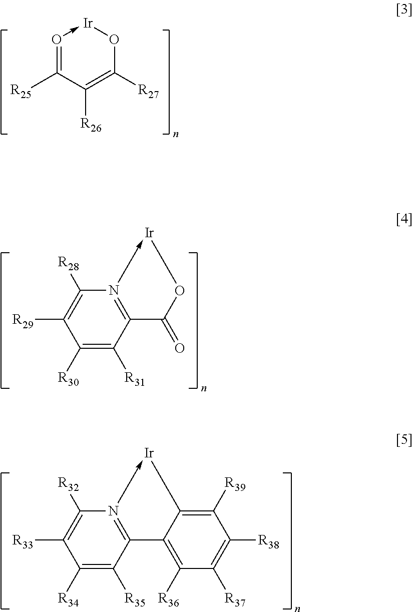

In formulae [3] to [5], R₂₅ to R₃₉ each represent a hydrogen atom, an alkyl group, an alkoxy group, a substituted amino group, a substituted or unsubstituted aryl group, or a substituted or unsubstituted heterocyclic group, and may be identical to or different from one another.

Specific examples of the alkyl group represented by any one of R₂₅ to R₃₉ are same as the specific examples of the alkyl group represented by any one of R₁₁ to R₂₄ in the formula [2]. The alkyl group is preferably an alkyl group having 1 or more and 10 or less carbon atoms, more preferably an alkyl group having 1 or more and 6 or less carbon atoms, still more preferably a methyl group or a tert-butyl group.

Specific examples of the alkoxy group represented by any one of R₂₅ to R₃₉ are the same as the specific examples of the alkoxy group represented by any one of R₁₁ to R₂₄ in the formula [2]. The alkoxy group is preferably a methoxy group.

Specific examples of the substituted amino group represented by any one of R₂₅ to R₃₉ are the same as the specific examples of the substituted amino group represented by any one of R₁₁ to R₂₄ in the formula [2]. The substituted amino group is preferably an N,N-dimethylamino group or an N,N-diphenylamino group.

Specific examples of the aryl group represented by any one of R₂₅ to R₃₉ are the same as the specific examples of the aryl group represented by any one of R₁₁ to R₁₄ in the formula [2]. The aryl group is preferably a phenyl group, a naphthyl group, a fluorenyl group, or a biphenyl group, more preferably a phenyl group.

Specific examples of the heterocyclic group represented by any one of R₂₅ to R₃₉ are the same as the specific examples of the heterocyclic group represented by any one of R₁₁ to R₁₄ in the formula [2].

A substituent, which the alkyl group and the heterocyclic group may each further have, is not particularly limited. Examples thereof may include: alkyl groups such as a methyl group, an ethyl group, an n-propyl group, an i-propyl group, an n-butyl group, an i-butyl group, a sec-butyl group, a tert-butyl group, an n-pentyl group, an i-pentyl group, a tert-pentyl group, a neopentyl group, an n-hexyl group, and a cyclohexyl group; alkoxy groups such as a methoxy group, an ethoxy group, an i-propoxy group, an n-butoxy group, and a tert-butoxy group; substituted amino groups such as an N-methylamino group, an N-ethylamino group, an N,N-dimethylamino group, an N,N-diethylamino group, an N-methyl-N-ethylamino group, an N-benzylamino group, an N-methyl-N-benzylamino group, an N,N-dibenzylamino group, an anilino group, an N,N-diphenylamino group, an N,N-dinaphthylamino group, an N,N-difluorenylamino group, an N-phenyl-N-tolylamino group, an N,N-ditolylamino group, an N-methyl-N-phenylamino group, an N,N-dianisoylamino group, an N-mesityl-N-phenylamino group, an N,N-dimesitylamino group, an N-phenyl-N-(4-tert-butylphenyl)amino group, and an N-phenyl-N-(4-trifluoromethylphenyl)amino group; aryl groups such as a phenyl group, a naphthyl group, a phenanthryl group, an anthryl group, a fluorenyl group, a biphenylenyl group, an acenaphthylenyl group, a chrysenyl group, a pyrenyl group, a triphenylenyl group, a picenyl group, a fluoranthenyl group, a perylenyl group, a naphthacenyl group, a biphenyl group, and a terphenyl group; heterocyclic groups such as a thienyl group, a pyrrolyl group, a pyrazinyl group, a pyridyl group, an indolyl group, a quinolyl group, an isoquinolyl group, a naphthyridinyl group, an acridinyl group, a phenanthrolinyl group, a carbazolyl group, a benzo[a]carbazolyl group, a benzo[b]carbazolyl group, a benzo[c]carbazolyl group, a phenazinyl group, a phenoxazinyl group, a phenothiazinyl group, a benzothiophenyl group, a dibenzothiophenyl group, a benzofuranyl group, a dibenzofuranyl group, an oxazolyl group, and an oxadiazolyl group; a cyano group; and a trifluoromethyl group.

The substituent, which the aryl group and the heterocyclic group may each further have, is preferably a methyl group, a tert-butyl group, a methoxy group, an N,N-dimethylamino group, an N,N-diphenylamino group, a phenyl group, a naphthyl group, a fluorenyl group, or a biphenyl group. Of those, a methyl group, a tert-butyl group, or a phenyl group is particularly preferred.

In the present invention, R₁₁ to R₂₄ in the general formula [2] each represent preferably a substituent selected from a hydrogen atom, a fluorine atom, and an alkyl group having 1 to 10 carbon atoms, more preferably a substituent selected from a hydrogen atom, a fluorine atom, a methyl group, and a tert-butyl group.

In the present invention, R₂₅ to R₃₉ represented in any one of the general formulae [3] to [5] each represent preferably a substituent selected from a hydrogen atom and an alkyl group having 1 to 10 carbon atoms, more preferably a substituent selected from a hydrogen atom, a methyl group, and a tert-butyl group.

In the present invention, at least one of R₁₁ to R₃₉ represents preferably an alkyl group having 1 to 10 carbon atoms, more preferably a methyl group or a tert-butyl group.

(Method of Synthesizing Iridium Complex)

Next, a method of synthesizing the iridium complex represented by the general formula [1] is described. The iridium complex represented by the general formula [1] is synthesized with reference to NPL 1 or 2, or the like through, for example, processes described in the following items (I) and (II):

(I) the synthesis of an organic compound serving as a ligand; and

(II) the synthesis of the organometallic complex.

Here, the process (I) is a method of synthesizing the organic compound serving as a ligand according to, for example, a synthesis route 1 or 2 shown below.

<Synthesis Route 1>

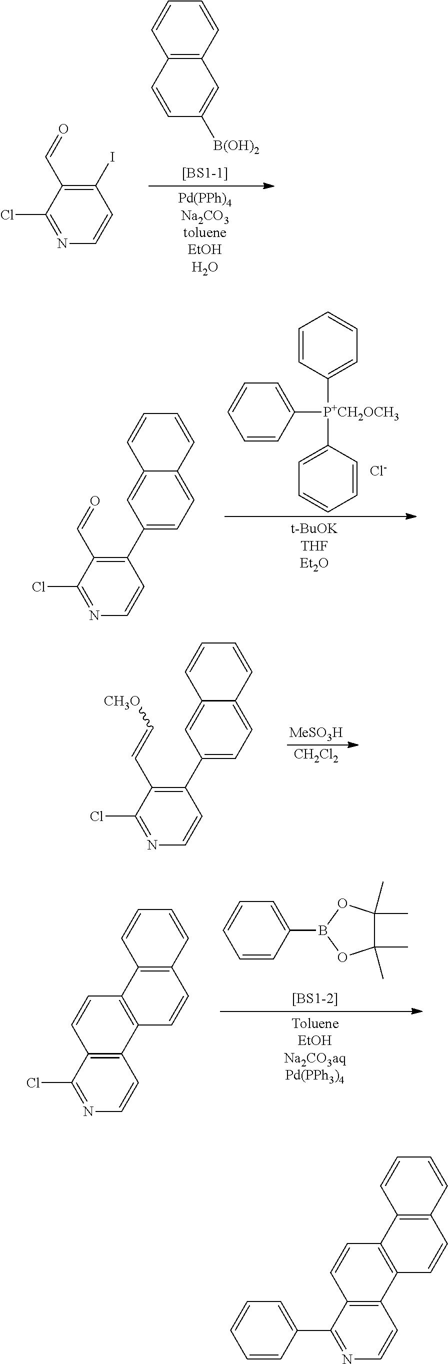

<Synthesis Route 2>

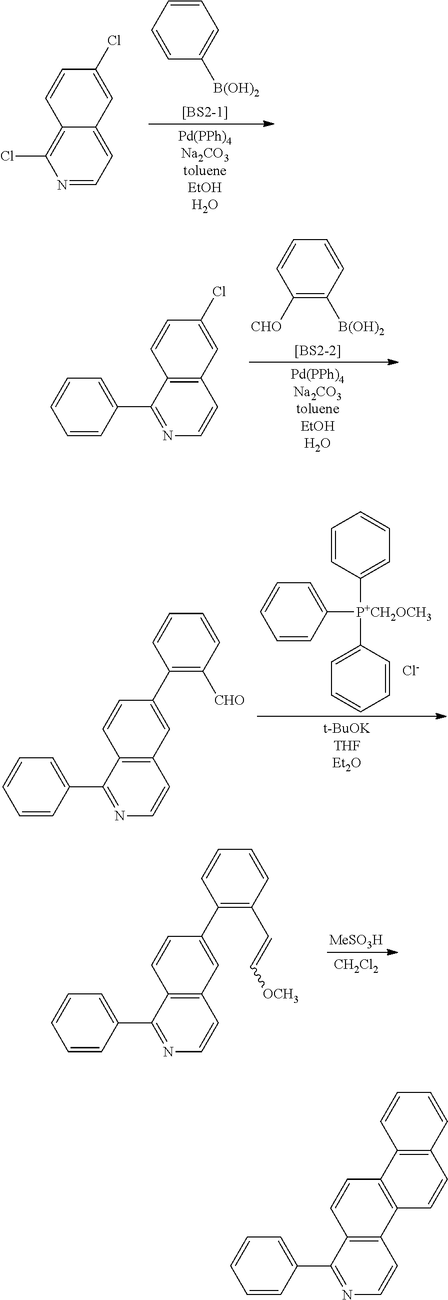

It is to be noted that a boronic acid compound to be coupled in each of the synthesis routes 1 and 2 is not limited to compounds (BS 1-1 to BS 2-2) represented in the synthesis routes 1 and 2. In the synthesis route 1, the target organic compound serving as a ligand can be synthesized by appropriately changing each of BS 1-1 and BS 1-2 as boronic acid compounds to another compound. In addition, in the synthesis route 2, the target organic compound serving as a ligand can be synthesized by appropriately changing each of BS 2-1 and BS 2-2 as boronic acid compounds to another compound.

Meanwhile, the process (II) is a method of synthesizing the iridium complex according to, for example, a synthesis route 3.

<Synthesis Route 3>

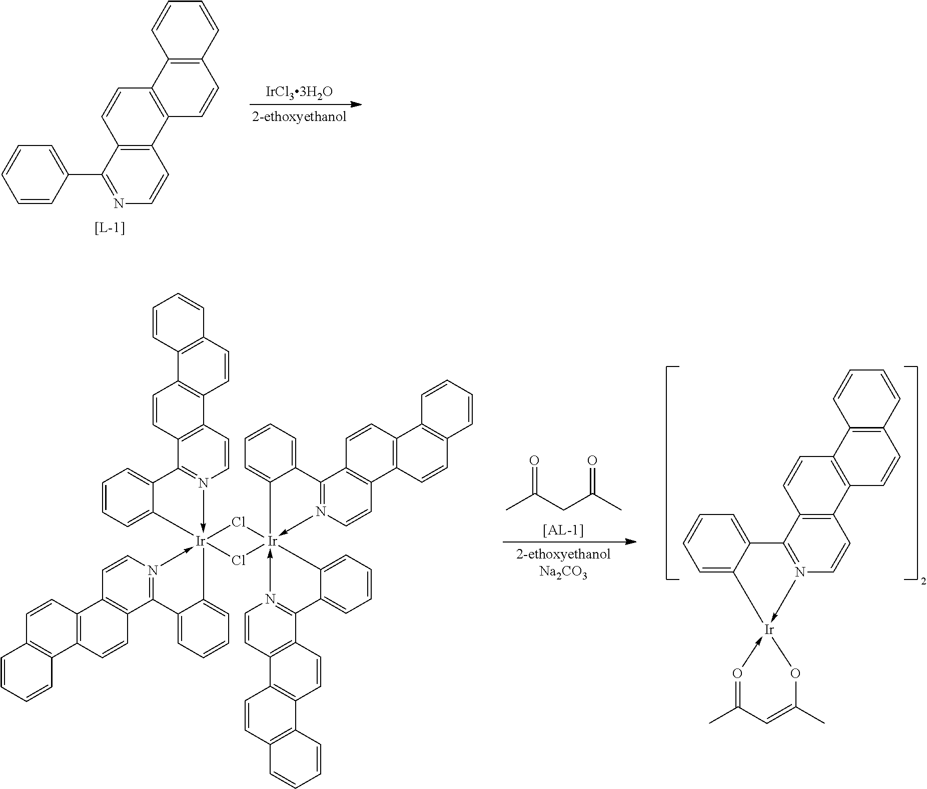

According to the synthesis route 3, an organometallic complex having two or more kinds of ligands (L and L′) can be synthesized. Here, in the synthesis route 3, the target complex can be synthesized by appropriately changing each of a luminous ligand (L−1) and an auxiliary ligand (AL-1) to another ligand. For example, AL-1 can be changed to a pyridylpyridine derivative. It is to be noted that in such case, a reaction condition upon introduction of the ligand is appropriately changed. Specifically, reagents (2-ethoxyethanol and sodium carbonate) described in the synthesis scheme have only to be changed to ethanol and silver trifluoromethanesulfonate.

In addition, when the iridium complex represented by the general formula [1] is used as a constituent material for an organic light-emitting device, sublimation purification is preferably performed as purification immediately before the use. The sublimation purification realizes an increase in purity of the organic compound because of its large purifying effect. However, when the molecular weight of the organic compound increases, the sublimation purification requires higher temperature, and at the time, for example, its thermal decomposition is liable to occur owing to the high temperature. Therefore, the molecular weight of the organic compound to be used as a constituent material for an organic light-emitting device is preferably 1,200 or less, more preferably 1,100 or less in order that the sublimation purification can be performed without any excessive heating.

(3) Heterocycle-Containing Compound

Next, the heterocycle-containing compound to be used as the host for the emission layer in the organic light-emitting device of the present invention is described. The heterocycle-containing compound in the organic light-emitting device of the present invention is a heteroaromatic compound containing a heteroatom such as a nitrogen, oxygen, or sulfur atom. The heterocycle-containing compound is preferably a compound represented by the following general formula [6] or [7].

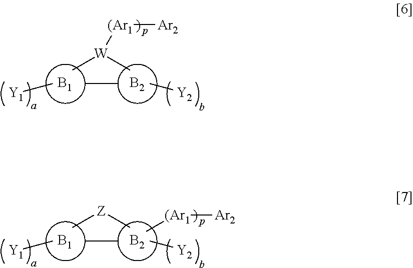

In the general formula [6], W represents a nitrogen atom. In the general formula [7], Z represents an oxygen atom or a sulfur atom.

In the general formulae [6] and [7], a ring B₁ and a ring B₂ each represent an aromatic ring selected from a benzene ring, a naphthalene ring, a phenanthrene ring, a triphenylene ring, and a chrysene ring. That is, the compound represented by the general formula [6] has a heterocycle formed of W (nitrogen atom), the ring B₁, and the ring B₂. In addition, the compound represented by the general formula [7] has a heterocycle formed of Z (oxygen atom or sulfur atom), the ring B₁, and the ring B₂. Here, in the general formulae [6] and [7], the ring B₁ and the ring B₂ may be identical to or different from each other.

It is to be noted that the ring B₁ and the ring B₂ may each further have any one of a group of substituents to be described later, that is, a substituent except Y₁, Y₂, and -(Ar₁)ₚ—Ar₂. Specific examples thereof include: an alkyl group having 1 to 4 carbon atoms selected from a methyl group, an ethyl group, an n-propyl group, an i-propyl group, an n-butyl group, an i-butyl group, a sec-butyl group, and a tert-butyl group; a halogen atom selected from fluorine, chlorine, bromine, and iodine atoms; alkoxy groups such as a methoxy group, an ethoxy group, an i-propoxy group, an n-butoxy group, and a tert-butoxy group; substituted amino groups such as an N-methylamino group, an N-ethylamino group, an N,N-dimethylamino group, an N,N-diethylamino group, an N-methyl-N-ethylamino group, an N-benzylamino group, an N-methyl-N-benzylamino group, an N,N-dibenzylamino group, an anilino group, an N,N-diphenylamino group, an N,N-dinaphthylamino group, an N,N-difluorenylamino group, an N-phenyl-N-tolylamino group, an N,N-ditolylamino group, an N-methyl-N-phenylamino group, an N,N-dianisoylamino group, an N-mesityl-N-phenylamino group, an N,N-dimesitylamino group, an N-phenyl-N-(4-tert-butylphenyl)amino group, and an N-phenyl-N-(4-trifluoromethylphenyl)amino group; aromatic hydrocarbon groups such as a phenyl group, a naphthyl group, a phenanthryl group, an anthryl group, a fluorenyl group, a biphenylenyl group, an acenaphthylenyl group, a chrysenyl group, a pyrenyl group, a triphenylenyl group, a picenyl group, a fluoranthenyl group, a perylenyl group, a naphthacenyl group, a biphenyl group, and a terphenyl group; heteroaromatic groups such as a thienyl group, a pyrrolyl group, a pyrazinyl group, a pyridyl group, an indolyl group, a quinolyl group, an isoquinolyl group, a naphthyridinyl group, an acridinyl group, a phenanthrolinyl group, a carbazolyl group, a benzo[a]carbazolyl group, a benzo[b]carbazolyl group, a benzo[c]carbazolyl group, a phenazinyl group, a phenoxazinyl group, a phenothiazinyl group, a benzothiophenyl group, a dibenzothiophenyl group, a benzofuranyl group, a dibenzofuranyl group, an oxazolyl group, and an oxadiazolyl group; a cyano group; and a trifluoromethyl group. Here, the alkyl group that substituent represented by the ring B₁ or the ring B₂ may further have includes one in which a hydrogen atom in the substituent is substituted with a fluorine atom.

Of those substituents listed above, a methyl group, a tert-butyl group, a methoxy group, an ethoxy group, a carbazolyl group, a dibenzothienyl group, a dibenzofuranyl group, a phenyl group, a naphthyl group, a fluorenyl group, or a biphenyl group is preferred. When the substituent, which the substituent represented by the ring B₁ or the ring B₂ may further have, is an aromatic hydrocarbon group, a phenyl group is particularly preferred.

In the general formulae [6] and [7], Y₁ and Y₂ each represent an alkyl group or an aromatic hydrocarbon group.

The alkyl group represented by Y₁ or Y₂ is preferably an alkyl group having 1 to 4 carbon atoms. Specific examples thereof include a methyl group, an ethyl group, an n-propyl group, an i-propyl group, an n-butyl group, an i-butyl group, a sec-butyl group, and a tert-butyl group. Of those alkyl groups, a methyl group or a tert-butyl group is preferred.

Specific examples of the aromatic hydrocarbon group represented by Y₁ or Y₂ include, but, of course, not limited to, a phenyl group, a naphthyl group, a phenanthryl group, an anthryl group, a fluorenyl group, a biphenylenyl group, an acenaphthylenyl group, a chrysenyl group, a pyrenyl group, a triphenylenyl group, a picenyl group, a fluoranthenyl group, a perylenyl group, a naphthacenyl group, a biphenyl group, and a terphenyl group. Of those aromatic hydrocarbon groups, a phenyl group, a naphthyl group, a fluorenyl group, or a biphenyl group is preferred, and a phenyl group is more preferred.

When any one of the substituents represented by Y₁ and Y₂ is an alkyl group having 1 to 4 carbon atoms or an aromatic hydrocarbon group, the corresponding substituent may further have any other substituent. Specific examples of the substituent that the substituent represented by Y₁ or Y₂ may further have include: alkyl groups having 1 to 4 carbon atoms such as a methyl group, an ethyl group, an n-propyl group, an i-propyl group, an n-butyl group, an i-butyl group, a sec-butyl group, and a tert-butyl group; a halogen atom selected from fluorine, chlorine, bromine, and iodine atoms; alkoxy groups such as a methoxy group, an ethoxy group, an i-propoxy group, an n-butoxy group, and a tert-butoxy group; substituted amino groups such as an N-methylamino group, an N-ethylamino group, an N,N-dimethylamino group, an N,N-diethylamino group, an N-methyl-N-ethylamino group, an N-benzylamino group, an N-methyl-N-benzylamino group, an N,N-dibenzylamino group, an anilino group, an N,N-diphenylamino group, an N,N-dinaphthylamino group, an N,N-difluorenylamino group, an N-phenyl-N-tolylamino group, an N,N-ditolylamino group, an N-methyl-N-phenylamino group, an N,N-dianisoylamino group, an N-mesityl-N-phenylamino group, an N,N-dimesitylamino group, an N-phenyl-N-(4-tert-butylphenyl)amino group, and an N-phenyl-N-(4-trifluoromethylphenyl)amino group; aromatic hydrocarbon groups such as a phenyl group, a naphthyl group, a phenanthryl group, an anthryl group, a fluorenyl group, a biphenylenyl group, an acenaphthylenyl group, a chrysenyl group, a pyrenyl group, a triphenylenyl group, a picenyl group, a fluoranthenyl group, a perylenyl group, a naphthacenyl group, a biphenyl group, and a terphenyl group; heteroaromatic groups such as a thienyl group, a pyrrolyl group, a pyrazinyl group, a pyridyl group, an indolyl group, a quinolyl group, an isoquinolyl group, a naphthyridinyl group, an acridinyl group, a phenanthrolinyl group, a carbazolyl group, a benzo[a]carbazolyl group, a benzo[b]carbazolyl group, a benzo[c]carbazolyl group, a phenazinyl group, a phenoxazinyl group, a phenothiazinyl group, a benzothiophenyl group, a dibenzothiophenyl group, a benzofuranyl group, a dibenzofuranyl group, an oxazolyl group, and an oxadiazolyl group; a cyano group; and a trifluoromethyl group. Of those substituents, a methyl group, a tert-butyl group, a phenyl group, a naphthyl group, a fluorenyl group, or a biphenyl group is preferred, and a phenyl group is more preferred.

In the general formulae [6] and [7], a represents an integer of 0 to 4, and when a represents 2 or more, multiple Y₁'s may be identical to or different from each other.

In the general formulae [6] and [7], b represents an integer of 0 to 4, provided that when the ring B₂ represents a benzene ring, b represents an integer of 0 to 3. When b represents 2 or more, multiple Y₂'s may be identical to or different from each other.

In the general formulae [6] and [7], Ar₁ represents a divalent aromatic hydrocarbon group. Specific examples of the divalent aromatic hydrocarbon group represented by Ar₁ include a phenylene group, a biphenylene group, a terphenylene group, a naphthalenediyl group, a phenanthrenediyl group, an anthracenediyl group, a benzo[a]anthracenediyl group, a fluorenediyl group, a benzo[a]fluorenediyl group, a benzo[b]fluorenediyl group, a benzo[c]fluorenediyl group, a dibenzo[a,c]fluorenediyl group, a dibenzo[b,h]fluorenediyl group, a dibenzo[c,g]fluorenediyl group, a biphenylenediyl group, an acenaphthylenediyl group, a chrysenediyl group, a benzo[b]chrysenediyl group, a pyrenediyl group, a benzo[e]pyrenediyl group, a triphenylenediyl group, a benzo[a]triphenylenediyl group, a benzo[b]triphenylenediyl group, a picenediyl group, a fluoranthenediyl group, a benzo[a]fluoranthenediyl group, a benzo[b]fluoranthenediyl group, a benzo[j]fluoranthenediyl group, a benzo[k]fluoranthenediyl group, a perylenediyl group, and a naphthacenediyl group. Of those, a substituent selected from a phenylene group, a biphenylene group, a terphenylene group, a naphthalenediyl group, a fluorenediyl group, a phenanthrenediyl group, a chrysenediyl group, and a triphenylenediyl group is preferred from the viewpoint of ease of sublimation purification.

It is to be noted that Ar₁ may further have a substituent. Specific examples thereof include: an alkyl group having 1 to 4 carbon atoms selected from a methyl group, an ethyl group, an n-propyl group, an i-propyl group, an n-butyl group, an i-butyl group, a sec-butyl group, and a tert-butyl group; a halogen atom selected from fluorine, chlorine, bromine, and iodine atoms; alkoxy groups such as a methoxy group, an ethoxy group, an i-propoxy group, an n-butoxy group, and a tert-butoxy group; substituted amino groups such as an N-methylamino group, an N-ethylamino group, an N,N-dimethylamino group, an N,N-diethylamino group, an N-methyl-N-ethylamino group, an N-benzylamino group, an N-methyl-N-benzylamino group, an N,N-dibenzylamino group, an anilino group, an N,N-diphenylamino group, an N,N-dinaphthylamino group, an N,N-difluorenylamino group, an N-phenyl-N-tolylamino group, an N,N-ditolylamino group, an N-methyl-N-phenylamino group, an N,N-dianisoylamino group, an N-mesityl-N-phenylamino group, an N,N-dimesitylamino group, an N-phenyl-N-(4-tert-butylphenyl)amino group, and an N-phenyl-N-(4-trifluoromethylphenyl)amino group; aromatic hydrocarbon groups such as a phenyl group, a naphthyl group, a phenanthryl group, an anthryl group, a fluorenyl group, a biphenylenyl group, an acenaphthylenyl group, a chrysenyl group, a pyrenyl group, a triphenylenyl group, a picenyl group, a fluoranthenyl group, a perylenyl group, a naphthacenyl group, a biphenyl group, and a terphenyl group; heteroaromatic groups such as a thienyl group, a pyrrolyl group, a pyrazinyl group, a pyridyl group, an indolyl group, a quinolyl group, an isoquinolyl group, a naphthyridinyl group, an acridinyl group, a phenanthrolinyl group, a carbazolyl group, a benzo[a]carbazolyl group, a benzo[b]carbazolyl group, a benzo[c]carbazolyl group, a phenazinyl group, a phenoxazinyl group, a phenothiazinyl group, a benzothiophenyl group, a dibenzothiophenyl group, a benzofuranyl group, a dibenzofuranyl group, an oxazolyl group, and an oxadiazolyl group; a cyano group; and a trifluoromethyl group. Here, the alkyl group that Ar₁ may further have includes one in which a hydrogen atom in the substituent is substituted with a fluorine atom.

Of those substituents listed above, a methyl group, a tert-butyl group, a methoxy group, an ethoxy group, a carbazolyl group, a dibenzothienyl group, a dibenzofuranyl group, a phenyl group, a naphthyl group, a fluorenyl group, or a biphenyl group is preferred. When the substituent, which the substituent represented by Ar₁ may further have, is an aromatic hydrocarbon group, a phenyl group is particularly preferred.

In the formulae [6] and [7], p represents an integer of 0 to 4. When p represents 2 or more, multiple Ar₁'s may be identical to or different from each other.

In the formulae [6] and [7], Ar₂ represents a substituted or unsubstituted monovalent aromatic hydrocarbon group. Specific examples thereof include a phenyl group, a naphthyl group, a phenanthryl group, an anthryl group, a benzo[a]anthryl group, a fluorenyl group, a benzo[a]fluorenyl group, a benzo[b]fluorenyl group, a benzo[c]fluorenyl group, a dibenzo[a,c]fluorenyl group, a dibenzo[b,h]fluorenyl group, a dibenzo[c,g]fluorenyl group, a biphenylenyl group, an acenaphthylenyl group, a chrysenyl group, a benzo[b]chrysenyl group, a pyrenyl group, a benzo[e]pyrenyl group, a triphenylenyl group, a benzo[a]triphenylenyl group, a benzo[b]triphenylenyl group, a picenyl group, a fluoranthenyl group, a benzo[a]fluoranthenyl group, a benzo[b]fluoranthenyl group, a benzo[j]fluoranthenyl group, a benzo[k]fluoranthenyl group, a perylenyl group, and a naphthacenyl group. Of those, a phenyl group, a biphenyl group, a terphenyl group, a naphthyl group, a fluorenyl group, a phenanthryl group, a chrysenyl group, or a triphenylenyl group is preferred from the viewpoint of ease of sublimation purification.

Specific examples of the substituent that the monovalent aromatic hydrocarbon group represented by Ar₂ may further have include: alkyl groups such as a methyl group, an ethyl group, an n-propyl group, an i-propyl group, an n-butyl group, an i-butyl group, a sec-butyl group, a tert-butyl group, an n-pentyl group, an i-pentyl group, a tert-pentyl group, a neopentyl group, an n-hexyl group, and a cyclohexyl group; a halogen atom selected from fluorine, chlorine, bromine, and iodine atoms; alkoxy groups such as a methoxy group, an ethoxy group, an i-propoxy group, an n-butoxy group, and a tert-butoxy group; substituted amino groups such as an N-methylamino group, an N-ethylamino group, an N,N-dimethylamino group, an N,N-diethylamino group, an N-methyl-N-ethylamino group, an N-benzylamino group, an N-methyl-N-benzylamino group, an N,N-dibenzylamino group, an anilino group, an N,N-diphenylamino group, an N,N-dinaphthylamino group, an N,N-difluorenylamino group, an N-phenyl-N-tolylamino group, an N,N-ditolylamino group, an N-methyl-N-phenylamino group, an N,N-dianisoylamino group, an N-mesityl-N-phenylamino group, an N,N-dimesitylamino group, an N-phenyl-N-(4-tert-butylphenyl)amino group, and an N-phenyl-N-(4-trifluoromethylphenyl)amino group; aromatic hydrocarbon groups such as a phenyl group, a naphthyl group, a phenanthryl group, an anthryl group, a fluorenyl group, a biphenylenyl group, an acenaphthylenyl group, a chrysenyl group, a pyrenyl group, a triphenylenyl group, a picenyl group, a fluoranthenyl group, a perylenyl group, a naphthacenyl group, a biphenyl group, and a terphenyl group; heteroaromatic groups such as a thienyl group, a pyrrolyl group, a pyrazinyl group, a pyridyl group, an indolyl group, a quinolyl group, an isoquinolyl group, a naphthyridinyl group, an acridinyl group, a phenanthrolinyl group, a carbazolyl group, a benzo[a]carbazolyl group, a benzo[b]carbazolyl group, a benzo[c]carbazolyl group, a phenazinyl group, a phenoxazinyl group, a phenothiazinyl group, a benzothiophenyl group, a dibenzothiophenyl group, a benzofuranyl group, a dibenzofuranyl group, an oxazolyl group, and an oxadiazolyl group; a cyano group; and a trifluoromethyl group.

Next, an even more preferred aspect of the host according to the present invention is described.

In the heterocycle-containing compound represented by the general formula [6], the heterocycle formed of W, the ring B₁, and the ring B₂, and Z and the ring B₁ are each preferably any one of heterocycles represented in the following group A1.

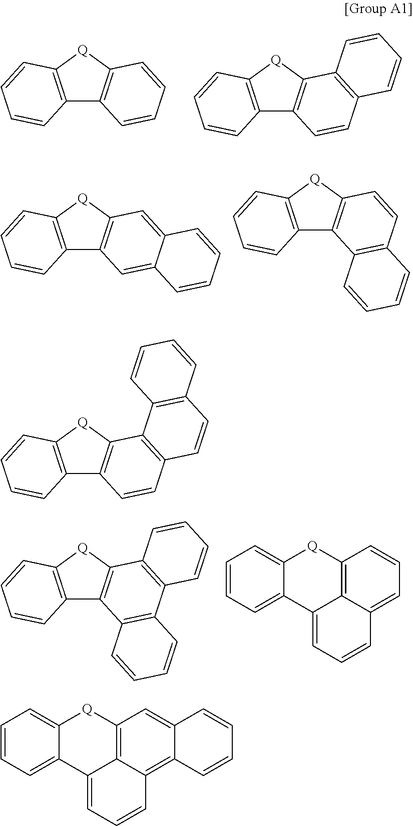

(In the general formula, Q represents a nitrogen atom.)

In addition, in the heterocycle-containing compound represented by the general formula [7], the heterocycle formed of Z, the ring B₁, and the ring B₂ is preferably any one of heterocycles represented in the following group A2.

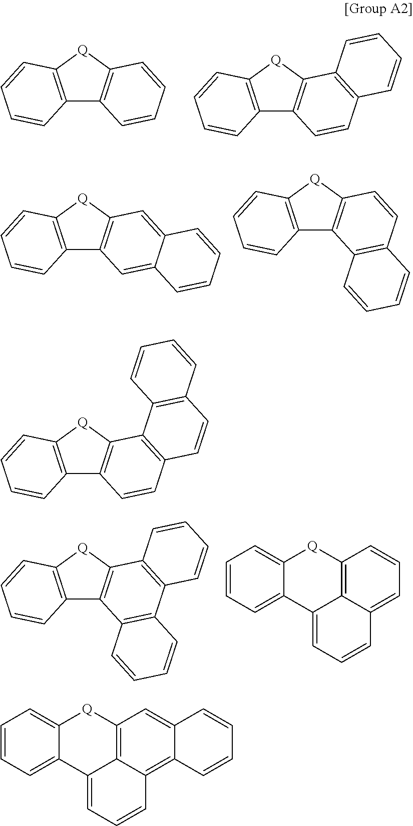

(In the formula, Q represents an oxygen atom or a sulfur atom.)

Further, the extensive studies carried out by the inventor of the present invention show that any one of compounds represented by the following general formulae [8] to [13] is particularly preferred as the host for the iridium complex represented by the general formula [1].

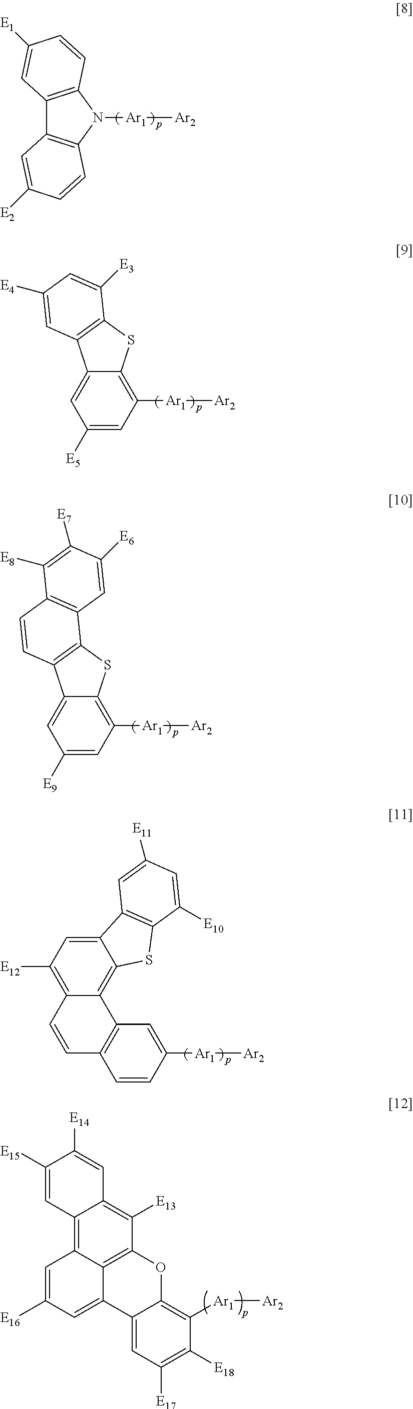

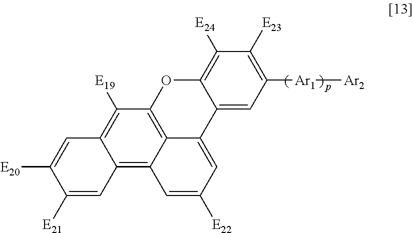

In the formula [8], E₁ and E₂ each represent a hydrogen atom, an alkyl group, or a substituted or unsubstituted aromatic hydrocarbon group. Specific examples of the alkyl group and aromatic hydrocarbon group represented by E₁, and the substituent that the aromatic hydrocarbon group may further have are the same as the specific examples of Y₁ in the general formula [6]. An alkyl group having 1 or more and 10 or less carbon atoms, a phenyl group, a naphthyl group, a fluorenyl group, a biphenyl group, or a terphenyl group is preferred, and an alkyl group having 1 or more and 6 or less carbon atoms typified by a methyl group or a tert-butyl group, or a phenyl group is more preferred. In addition, specific examples of the alkyl group and aromatic hydrocarbon group represented by E₂, and the substituent that the aromatic hydrocarbon group may further have are the same as the specific examples of Y₂ in the general formula [6]. An alkyl group having 1 or more and 10 or less carbon atoms, a phenyl group, a naphthyl group, a fluorenyl group, a biphenyl group, or a terphenyl group is preferred, and an alkyl group having 1 or more and 6 or less carbon atoms typified by a methyl group or a tert-butyl group, or a phenyl group is more preferred.

In the formula [9], E₃ to E₅ each represent a hydrogen atom, an alkyl group, or a substituted or unsubstituted aromatic hydrocarbon group. Specific examples of the alkyl group and aromatic hydrocarbon group represented by E₃ or E₄, and the substituent that the aromatic hydrocarbon group may further have are the same as the specific examples of Y₁ in the general formula [7]. An alkyl group having 1 or more and 10 or less carbon atoms, a phenyl group, a naphthyl group, a fluorenyl group, a biphenyl group, or a terphenyl group is preferred, and an alkyl group having 1 or more and 6 or less carbon atoms typified by a methyl group or a tert-butyl group, or a phenyl group is more preferred. In addition, specific examples of the alkyl group and aromatic hydrocarbon group represented by Eₛ, and the substituent that the aromatic hydrocarbon group may further have are the same as the specific examples of Y₂ in the general formula [7]. An alkyl group having 1 or more and 10 or less carbon atoms, a phenyl group, a naphthyl group, a fluorenyl group, a biphenyl group, or a terphenyl group is preferred, and an alkyl group having 1 or more and 6 or less carbon atoms typified by a methyl group or a tert-butyl group, or a phenyl group is more preferred.

In the formula [10], E₆ to E₉ each represent a hydrogen atom, an alkyl group, or a substituted or unsubstituted aromatic hydrocarbon group. Specific examples of the alkyl group and aromatic hydrocarbon group represented by any one of E₆ to E₈, and the substituent that the aromatic hydrocarbon group may further have are the same as the specific examples of Y₁ in the general formula [7]. An alkyl group having 1 or more and 10 or less carbon atoms, a phenyl group, a naphthyl group, a fluorenyl group, a biphenyl group, or a terphenyl group is preferred, and an alkyl group having 1 or more and 6 or less carbon atoms typified by a methyl group or a tert-butyl group, or a phenyl group is more preferred. In addition, specific examples of the alkyl group and aromatic hydrocarbon group represented by E₉, and the substituent that the aromatic hydrocarbon group may further have are the same as the specific examples of Y₂ in the general formula [7]. An alkyl group having 1 or more and 10 or less carbon atoms, a phenyl group, a naphthyl group, a fluorenyl group, a biphenyl group, or a terphenyl group is preferred, and an alkyl group having 1 or more and 6 or less carbon atoms typified by a methyl group or a tert-butyl group, or a phenyl group is more preferred.

In the formula [11], E₁₀ to E₁₂ each represent a hydrogen atom, an alkyl group, or a substituted or unsubstituted aromatic hydrocarbon group. Specific examples of the alkyl group and aromatic hydrocarbon group represented by E₁₀ or E₁₁, and the substituent that the aromatic hydrocarbon group may further have are the same as the specific examples of Y₁ in the general formula [7]. An alkyl group having 1 or more and 10 or less carbon atoms, a phenyl group, a naphthyl group, a fluorenyl group, a biphenyl group, or a terphenyl group is preferred, and an alkyl group having 1 or more and 6 or less carbon atoms typified by a methyl group or a tert-butyl group, or a phenyl group is more preferred. In addition, specific examples of the alkyl group and aromatic hydrocarbon group represented by E₁₂, and the substituent that the aromatic hydrocarbon group may further have are the same as the specific examples of Y₂ in the general formula [7]. An alkyl group having 1 or more and 10 or less carbon atoms, a phenyl group, a naphthyl group, a fluorenyl group, a biphenyl group, or a terphenyl group is preferred, and an alkyl group having 1 or more and 6 or less carbon atoms typified by a methyl group or a tert-butyl group, or a phenyl group is more preferred.

In the formula [12], E₁₃ to E₁₈ each represent a hydrogen atom, an alkyl group, or a substituted or unsubstituted aromatic hydrocarbon group. Specific examples of the alkyl group and aromatic hydrocarbon group represented by any one of E₁₃ to E₁₆, and the substituent that the aromatic hydrocarbon group may further have are the same as the specific examples of Y₁ in the general formula [7]. An alkyl group having 1 or more and 10 or less carbon atoms, a phenyl group, a naphthyl group, a fluorenyl group, a biphenyl group, or a terphenyl group is preferred, and an alkyl group having 1 or more and 6 or less carbon atoms typified by a methyl group or a tert-butyl group, or a phenyl group is more preferred. In addition, specific examples of the alkyl group and aromatic hydrocarbon group represented by E₁₇ or E₁₈, and the substituent that the aromatic hydrocarbon group may further have are the same as the specific examples of Y₂ in the general formula [7]. An alkyl group having 1 or more and 10 or less carbon atoms, a phenyl group, a naphthyl group, a fluorenyl group, a biphenyl group, or a terphenyl group is preferred, and an alkyl group having 1 or more and 6 or less carbon atoms typified by a methyl group or a tert-butyl group, or a phenyl group is more preferred.

In the formula [13], E₁₉ to E₂₄ each represent a hydrogen atom, an alkyl group, or a substituted or unsubstituted aromatic hydrocarbon group. Specific examples of the alkyl group and aromatic hydrocarbon group represented by any one of E₁₉ to E₂₂, and the substituent that the aromatic hydrocarbon group may further have are the same as the specific examples of Y₁ in the general formula [7]. An alkyl group having 1 or more and 10 or less carbon atoms, a phenyl group, a naphthyl group, a fluorenyl group, a biphenyl group, or a terphenyl group is preferred, and an alkyl group having 1 or more and 6 or less carbon atoms typified by a methyl group or a tert-butyl group, or a phenyl group is more preferred. In addition, specific examples of the alkyl group and aromatic hydrocarbon group represented by E₂₃ or E₂₄, and the substituent that the aromatic hydrocarbon group may further have are the same as the specific examples of Y₂ in the general formula [7]. An alkyl group having 1 or more and 10 or less carbon atoms, a phenyl group, a naphthyl group, a fluorenyl group, a biphenyl group, or a terphenyl group is preferred, and an alkyl group having 1 or more and 6 or less carbon atoms typified by a methyl group or a tert-butyl group, or a phenyl group is more preferred.

In the formulae [8] to [13], E₁ to E₂₄ each preferably represent a hydrogen atom. When all of E₁ to E₂₄ each represent a hydrogen atom, the molecular weight reduces, though the reduction is in a trade-off relationship with the chemical stability.

In the formulae [8] to [13], Ar₁ represents a substituted or unsubstituted divalent aromatic hydrocarbon group. It is to be noted that specific examples of Ar₁ are the same as the specific examples of Ar₁ in the formula [7].

In the formulae [8] to [13], Ar₂ represents a substituted or unsubstituted monovalent aromatic hydrocarbon group. It is to be noted that specific examples of Ar₂ are the same as the specific examples of Ar₂ in the formula [7].

In the formulae [8] to [13], p represents an integer of 0 to 4. p preferably represents 1. When p represents 2 or more, multiple Ar₁'s may be identical to or different from each other.

A first possible reason why the compounds represented by the formulae [8] to [13] are preferred as described above is as follows: in the case of a five-membered ring compound, a thiophene derivative is more stable than a furan derivative is, and in the case of a six-membered ring compound, a xanthene derivative is more stable than a thioxanthene derivative is. A second possible reason is that the presence of a substituent at a site having high chemical reactivity in an (aromatic) heterocyclic skeleton (each of ortho and para positions with respect to an oxygen atom or a sulfur atom) improves chemical stability.

In addition, a compound to be used as a constituent material for the organic light-emitting device of the present invention is desirably purified in advance. Sublimation purification is preferred as a method of purifying the compound. This is because the sublimation purification exhibits a large purifying effect in an improvement in purity of an organic compound. In general, in the sublimation purification, heating at higher temperature is needed as the molecular weight of an organic compound to be purified increases, and at that time, its thermal decomposition or the like is liable to occur owing to the high temperature. Therefore, the organic compound to be used as a constituent material for the organic light-emitting device preferably has a molecular weight of 1,500 or less so that the sublimation purification can be performed without excessive heating. Meanwhile, when the molecular weight is constant, a compound containing a smaller n-conjugated plane in its molecular skeleton is more advantageous for the sublimation purification because an intermolecular interaction becomes relatively small. In contrast, a compound containing a large n-conjugated plane in its molecular skeleton is disadvantageous for the sublimation purification because the intermolecular interaction is (relatively) large.

On the other hand, when the molecular weight of the heterocycle-containing compound as the host is excessively small, a deposition rate during its vacuum vapor deposition becomes unstable. Therefore, in consideration of a balance between the molecular weight and the size of the n-conjugated plane described in the foregoing, p in each of the heterocycle-containing compounds represented by the general formulae [8] to [13] preferably represents 1. Further, all of E₁ to E₂₂ each more preferably represent a hydrogen atom because the molecular weight reduces, though the reduction is in a trade-off relationship with the chemical stability.

(4) Actions and Effects Exhibited by Host and Guest

In the organic light-emitting device of the present invention, the organic compound layer (such as the emission layer) includes the iridium complex represented by the general formula [1] and the heterocycle-containing compound (preferably the heterocycle-containing compound represented by the general formula [6] or [7]).

The iridium complex represented by the general formula [1] is an organometallic complex in which at least one arylnaphtho[2,1-f]isoquinoline ligand coordinates to an iridium metal, i.e., an niq-based Ir complex. The niq-based Ir complex is a phosphorescent material having a high emission quantum yield and capable of emitting red light. Here, the term “red light emission” refers to such light emission that an emission peak wavelength is 580 nm or more and 650 nm or less, i.e., the lowest triplet excited level (T₁) falls within the range of 1.9 eV or more to 2.1 eV or less. In addition, the organic light-emitting device obtained by incorporating the niq-based Ir complex as the guest into the emission layer has extremely high emission efficiency.

By the way, an improvement in driving durability lifetime of the organic light-emitting device has the same meaning as an improvement in driving durability lifetime through a reduction in luminance degradation. Here, it has been known that the following measures have only to be taken on the emission layer for the improvement in driving durability lifetime through the reduction in luminance degradation:

(I) an improvement in carrier balance in the emission layer;

(II) the extension of a light-emitting region (carrier recombination region); and

(III) an improvement in structural stability of a host molecule in the emission layer.

That is, three factors considered to be factors for the luminance degradation, i.e., (i) carrier accumulation that may occur at an interface between the emission layer and a carrier-transporting layer, (ii) local light emission that leads to the degradation of the light-emitting material, and (iii) the degradation of the host are suppressed. Thus, the lifetime of the organic light-emitting device can be lengthened.

In addition, the inventors of the present invention have paid attention to the lifetime-lengthening guidelines, and have considered that the driving durability lifetime of the organic light-emitting device using the niq-based Ir complex can be additionally improved (a longer lifetime can be achieved) from the viewpoints of the material characteristics of the host in the emission layer. Specifically, the inventors of the present invention have considered that the lifetime of the organic light-emitting device can be additionally lengthened by incorporating the heterocycle-containing compound as well as the niq-based Ir complex into the organic compound layer (particularly the emission layer).

In consideration of a combination with the niq-based Ir complex to be incorporated as the guest into the organic compound layer (particularly the emission layer), when the host to be incorporated into the emission layer has a moderate hole-transporting property, it is considered that large effects are exhibited on the measure (I) (the improvement in carrier balance) and the measure (II) (the extension of the light-emitting region).

Then, as a result of their extensive studies, the inventors of the present invention have found that a compound having a heterocycle containing nitrogen, oxygen, or sulfur in its molecular structure, the compound being a material having moderate hole-transporting property, is suitable as a host for an emission layer to be used in combination with the niq-based Ir complex. The compound can have moderate hole-transporting property probably because a hole is moderately trapped by the nitrogen, oxygen, or sulfur atom on the heterocycle.

In addition, the heterocycle-containing compound that can be used (as the host) in the present invention, which is not particularly limited, is more preferably a compound free of any bond having low bond stability in its molecular structure. When a compound having a bond having low bond stability, i.e., an unstable bond having a small bond energy in its molecular structure is incorporated as the host into the emission layer constituting the organic light-emitting device, the structural degradation of the compound is liable to occur at the time of the driving of the device. In addition, there is a high risk that the compound adversely affects the durability lifetime of the light-emitting device.

When Exemplified Compound X-135 is taken as an example, the bond having low bond stability means a bond (nitrogen-carbon bond) that bonds a carbazole ring and a phenylene group. Shown below is comparison between calculated values for the bonding energies of Exemplified Compounds X-135 and H-308. It is to be noted that the calculation was performed by employing an approach “b3-lyp/def2-SV(P)”.

Bond Energy (Calculated Value)

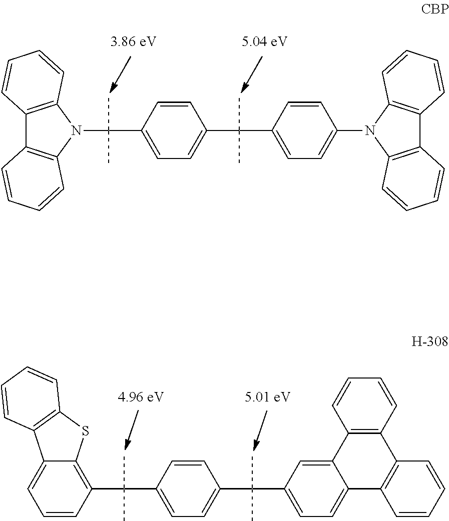

As can be seen from the results, when a bond between the heterocycle and aryl group of the heterocycle-containing compound as a constituent material for the organic light-emitting device of the present invention is a carbon-carbon bond, its bond energy is as large as about 5 eV and hence its bond stability is high. Accordingly, the incorporation of the heterocycle-containing compound, which is a constituent material for the organic light-emitting device of the present invention, as the host into the organic compound layer (e.g., the emission layer) can suppress the degradation of the material at the time of the driving of the device because the structural stability of the material is high. In other words, it is found that a large effect is exhibited on the measure (III) (an improvement in structural stability of a host molecule).

By the way, the heterocycle-containing compound and an analogue thereof are each used as a host for a green phosphorescent iridium complex as a guest in PTL 2 or the like. Meanwhile, the inventors of the present invention have found that the heterocycle-containing compound is suitable as a host for the red phosphorescent organometallic complex as the guest. This is because the S₁ energy value and T₁ energy value of the heterocycle-containing compound are suitable as the host for the red phosphorescent layer.

That is, the T₁ energy of the host is preferably 2.1 eV or more in order that the quenching of a T₁ exciton may be prevented. In addition, the S₁ energy of the host is desirably as low as possible in order that an increase in driving voltage may be prevented by good carrier injection, and the energy is preferably 3.0 eV or less. In other words, a ΔS-T value as a difference between the S₁ energy and the T₁ energy is preferably as small as possible. In view of the foregoing, it is suitable to incorporate the heterocycle-containing compound as the host into the red phosphorescent layer.

Accordingly, the organic light-emitting device obtained by incorporating the iridium complex represented by the general formula [1] and capable of emitting red light as the guest and the heterocycle-containing compound as the host has high emission efficiently and a long lifetime.

Next, a more preferred aspect of the host is described.

Compounds (such as pyridine, quinoline, and azafluorene) obtained by substituting sp² carbon atoms of benzene, naphthalene, and a fused polycyclic compound with nitrogen atoms are each available as the heterocycle-containing compound. Each of the highest occupied molecular orbital (HOMO) levels and lowest unoccupied molecular orbital (LUMO) levels of those compounds is known to reduce. Therefore, the use of a compound having the skeleton of each of the compounds obtained by substituting the sp² carbon atoms of benzene, naphthalene, and the fused polycyclic compound with nitrogen atoms as the host raises the difficulty with which a hole is injected into the emission layer while the use facilitates the injection of an electron into the layer. Accordingly, the kinds of applicable charge-transporting layers and guests are limited.

(5) Specific Examples of Iridium Complex

Specific examples of the iridium complex serving as the guest are shown below.

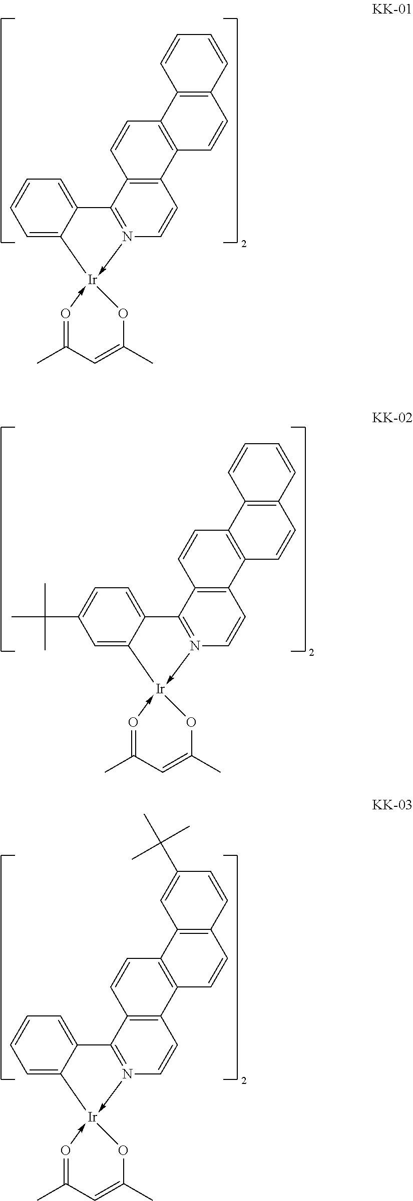

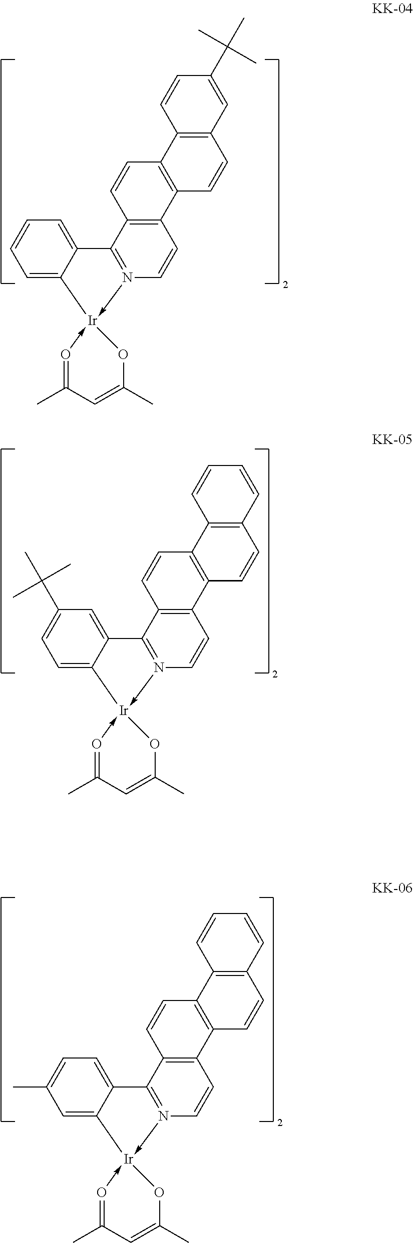

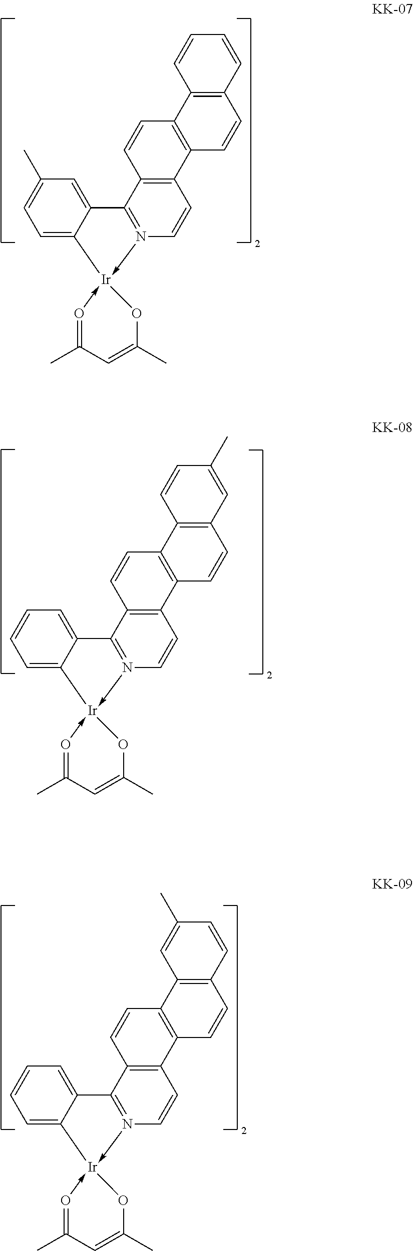

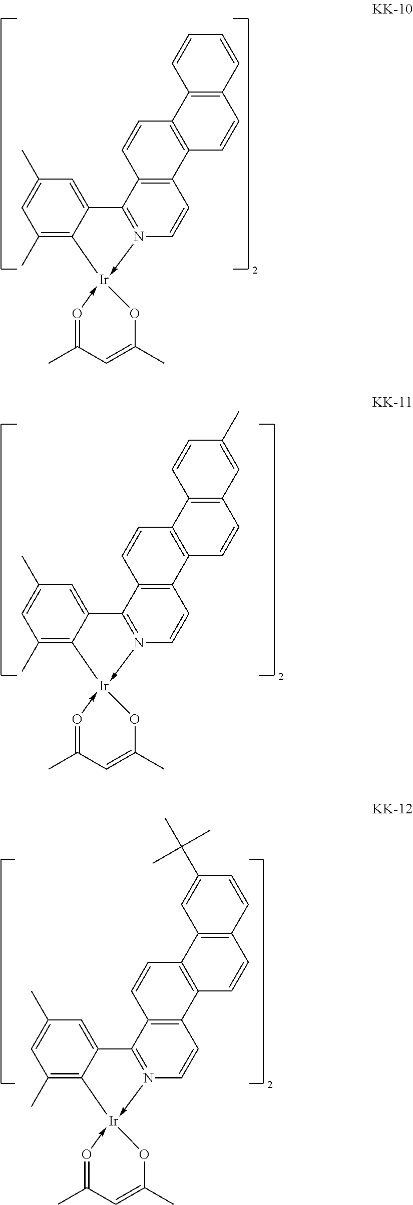

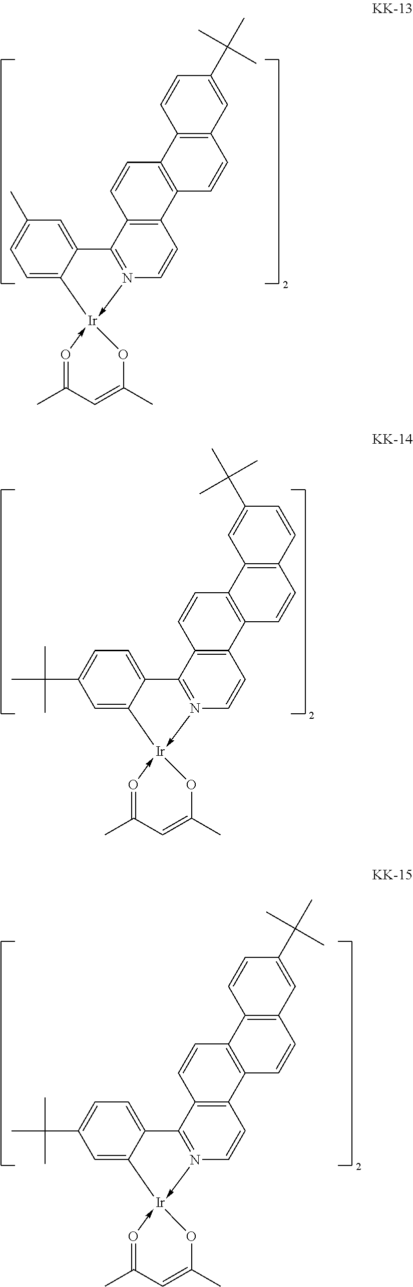

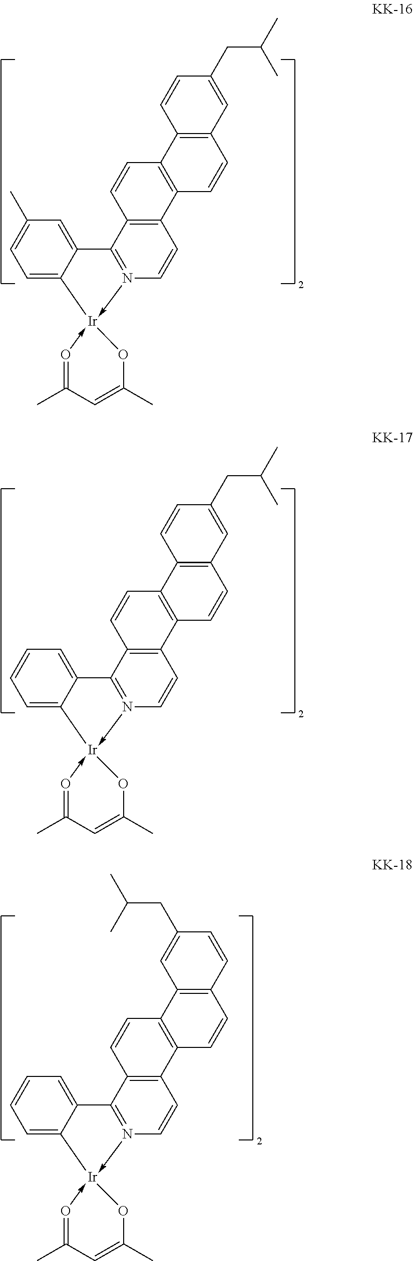

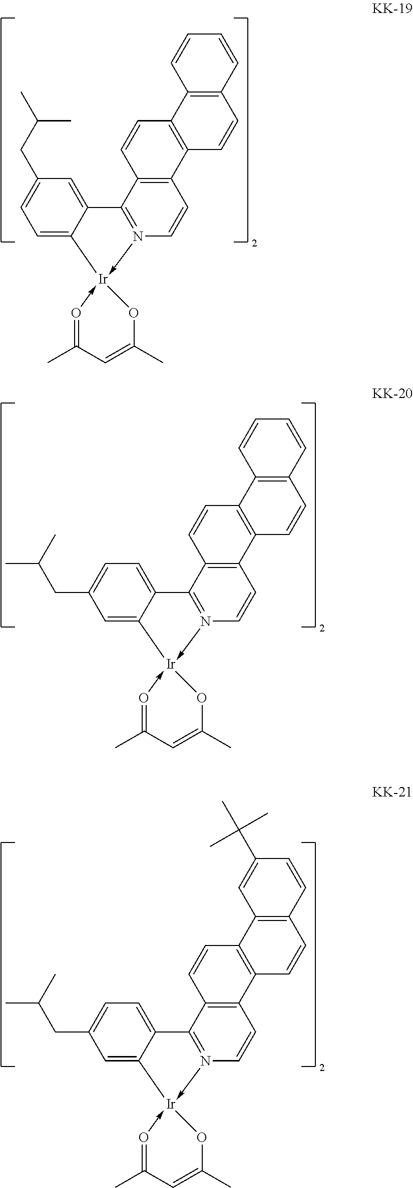

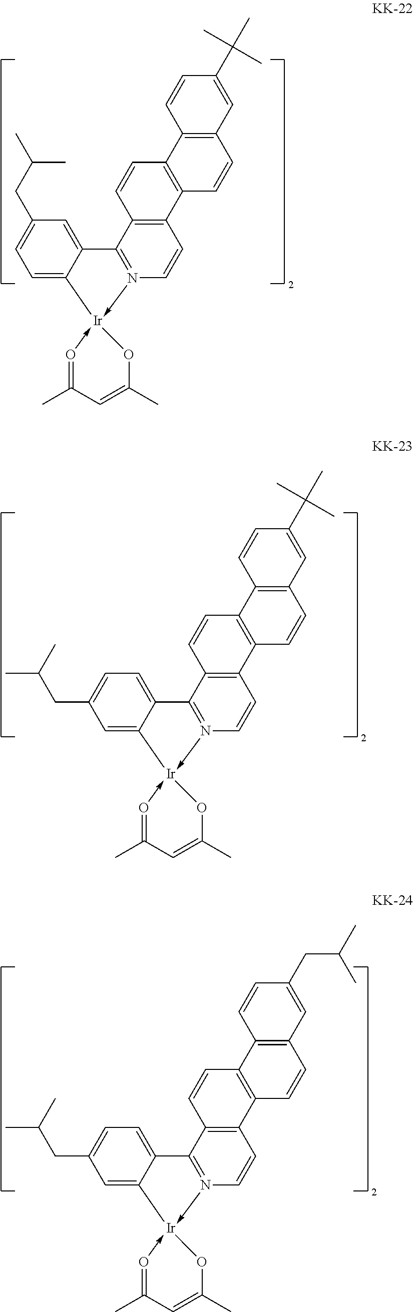

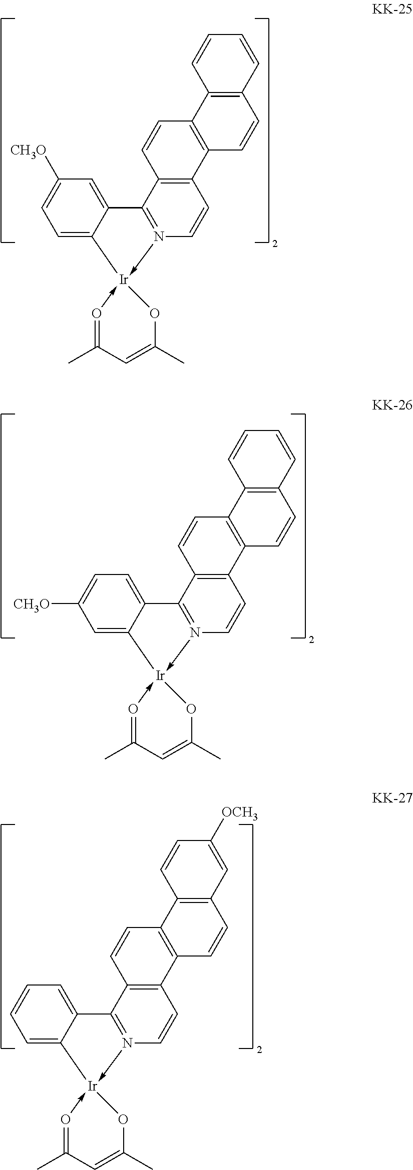

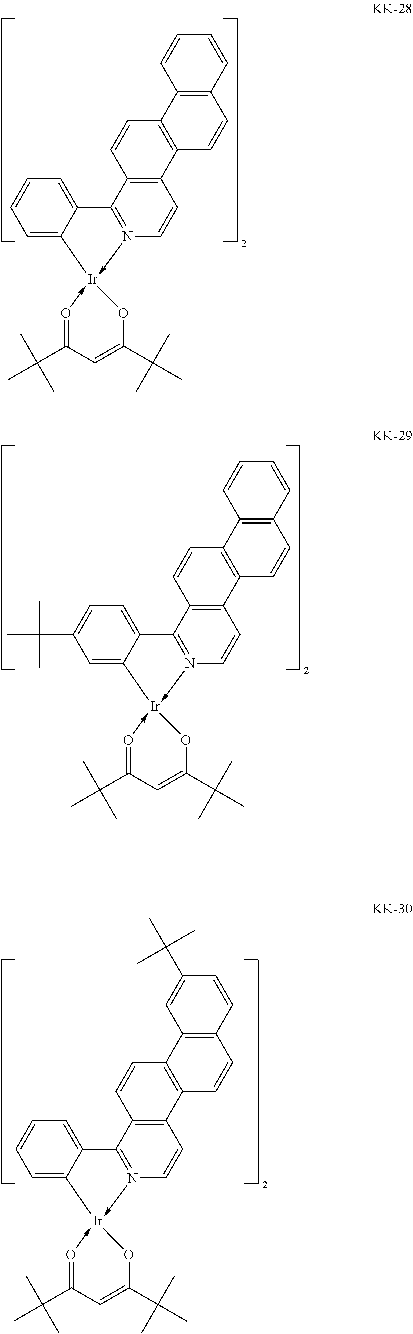

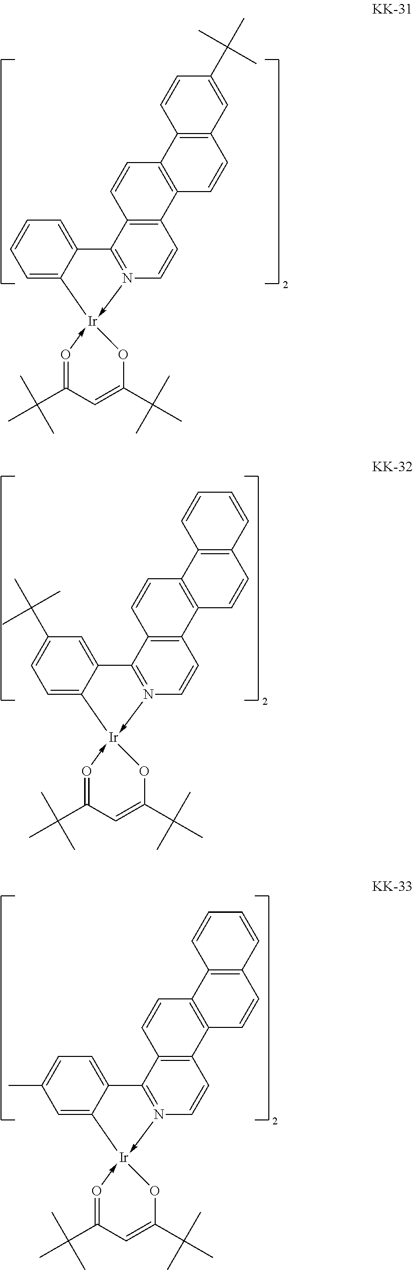

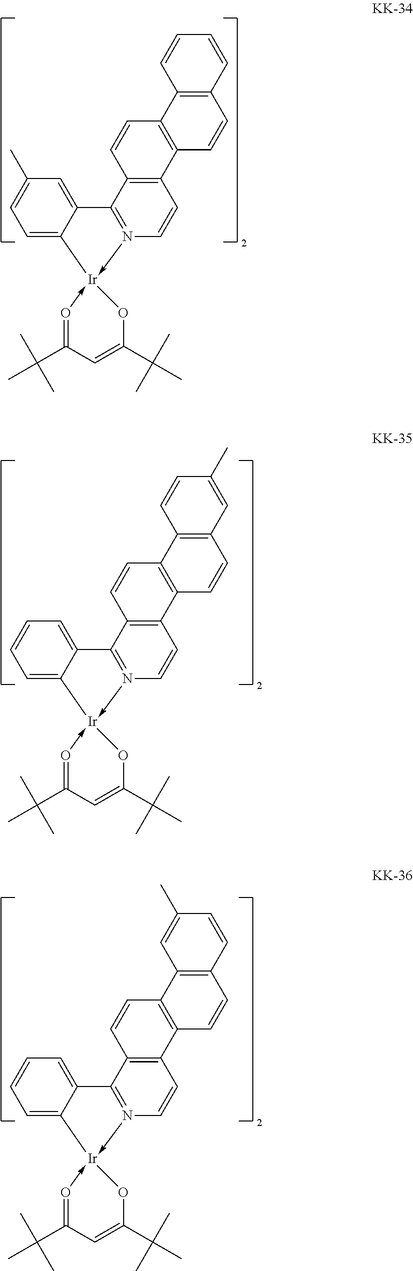

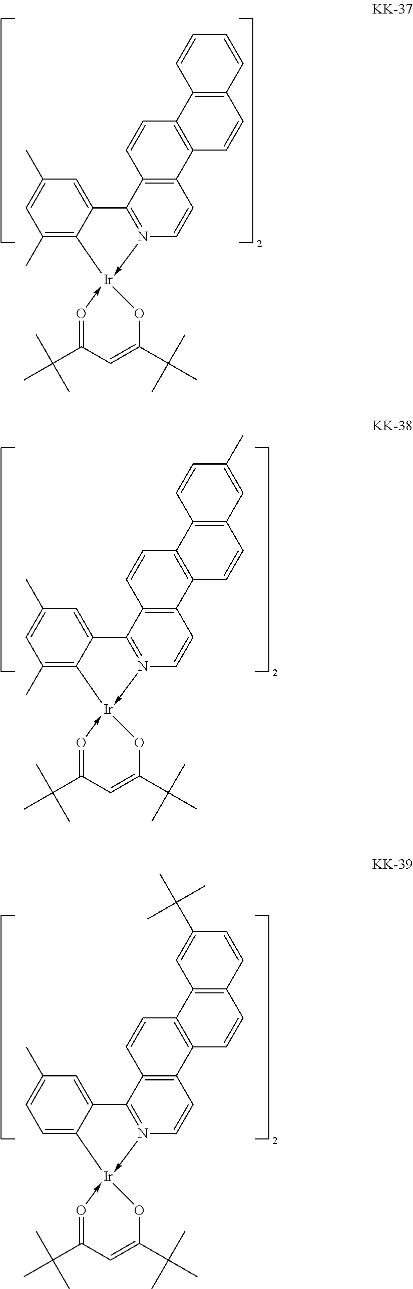

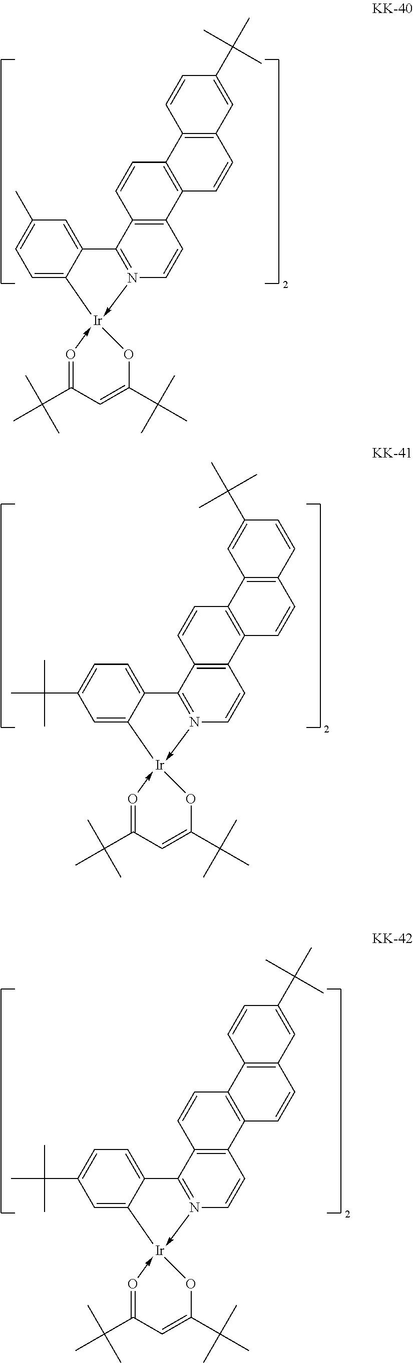

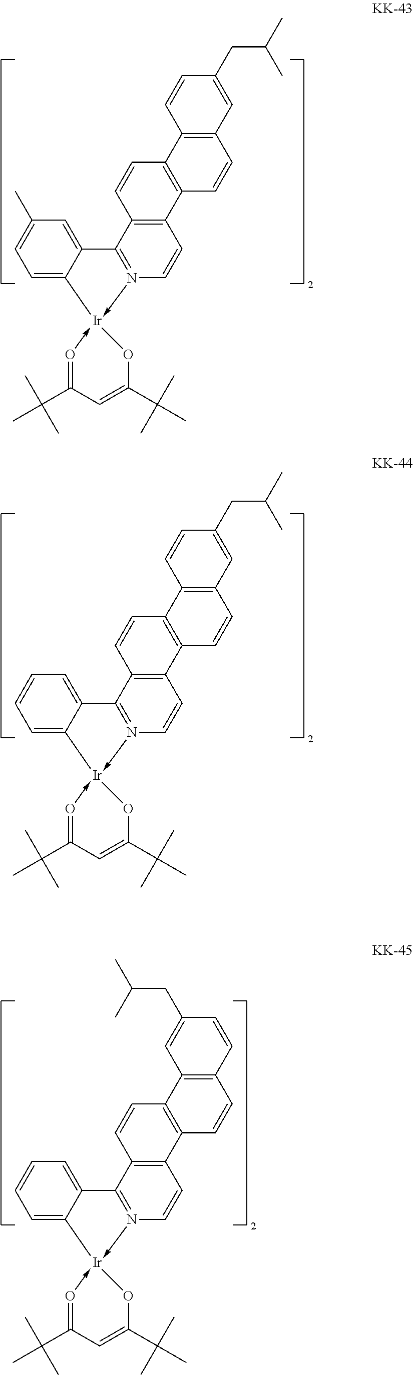

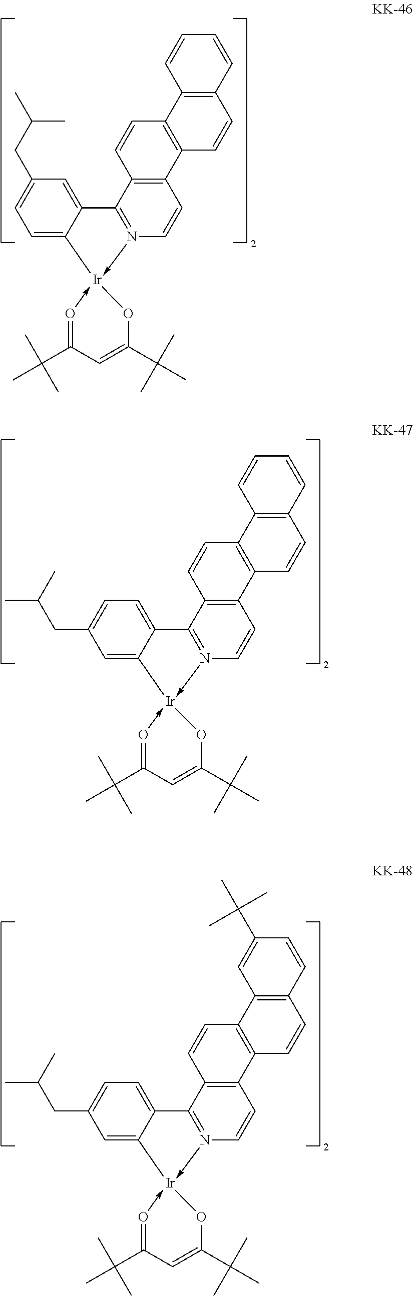

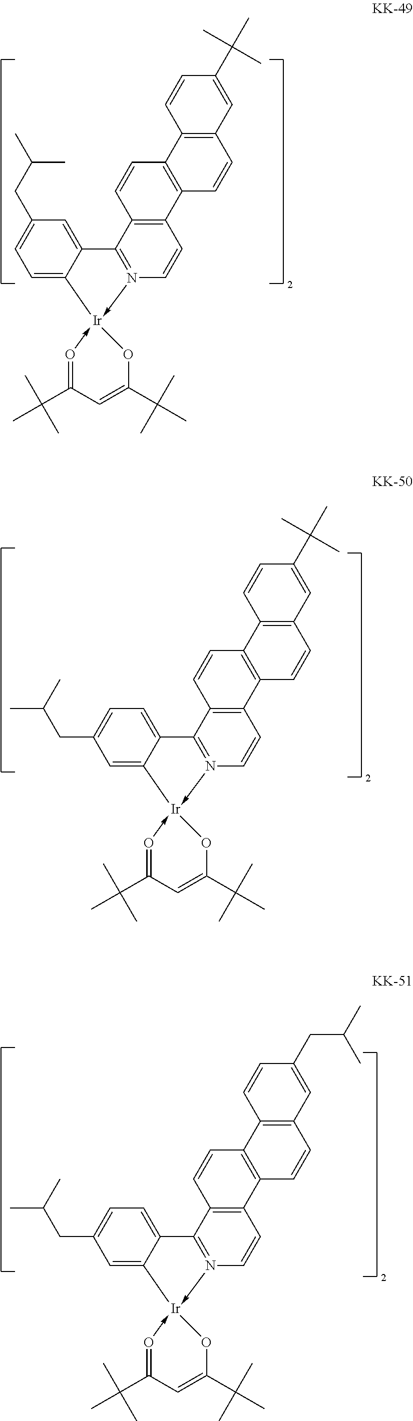

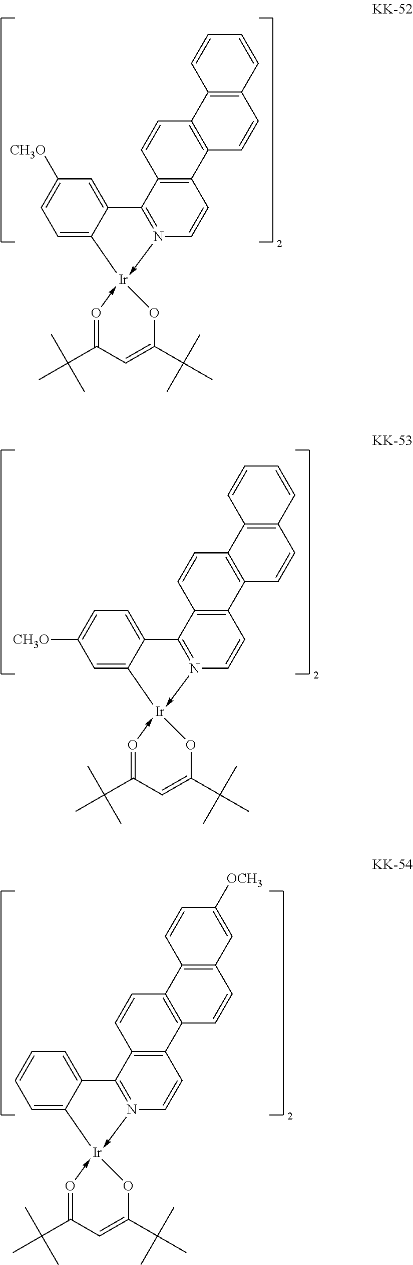

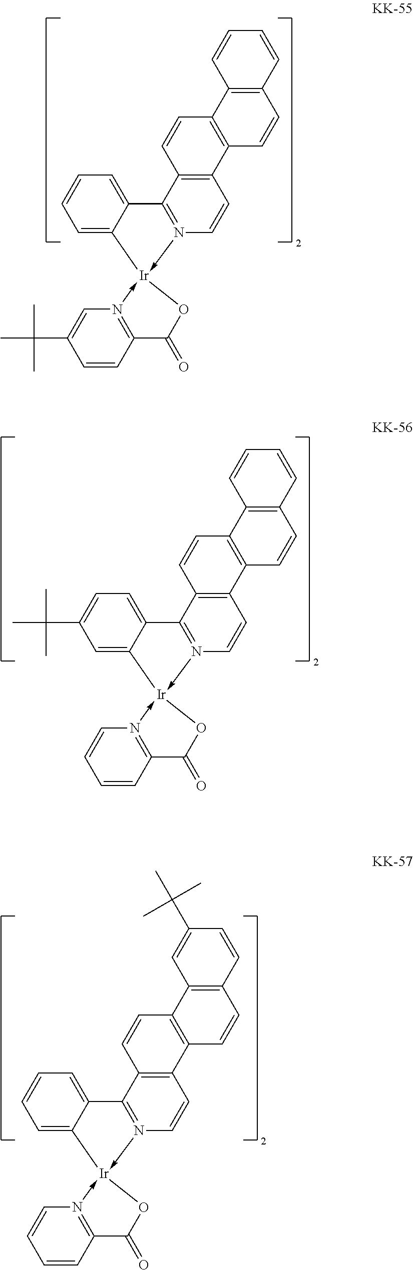

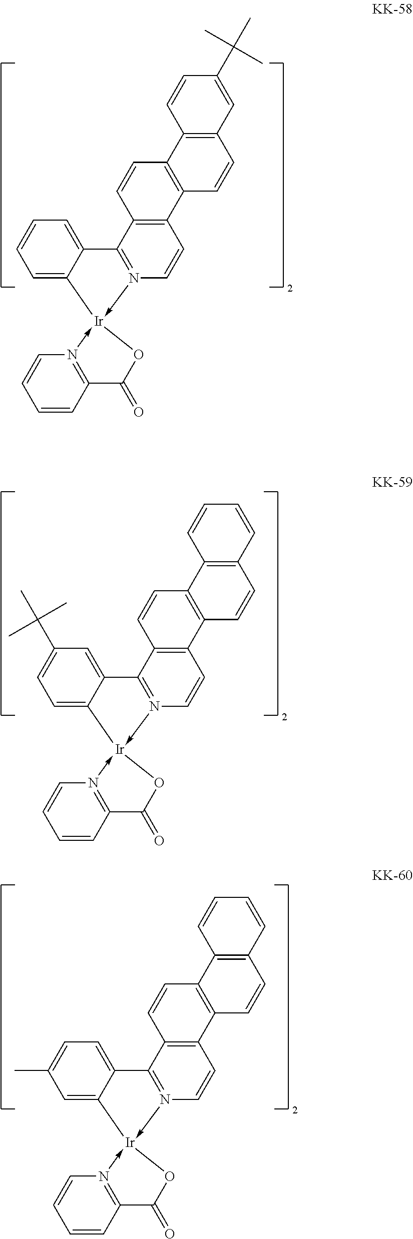

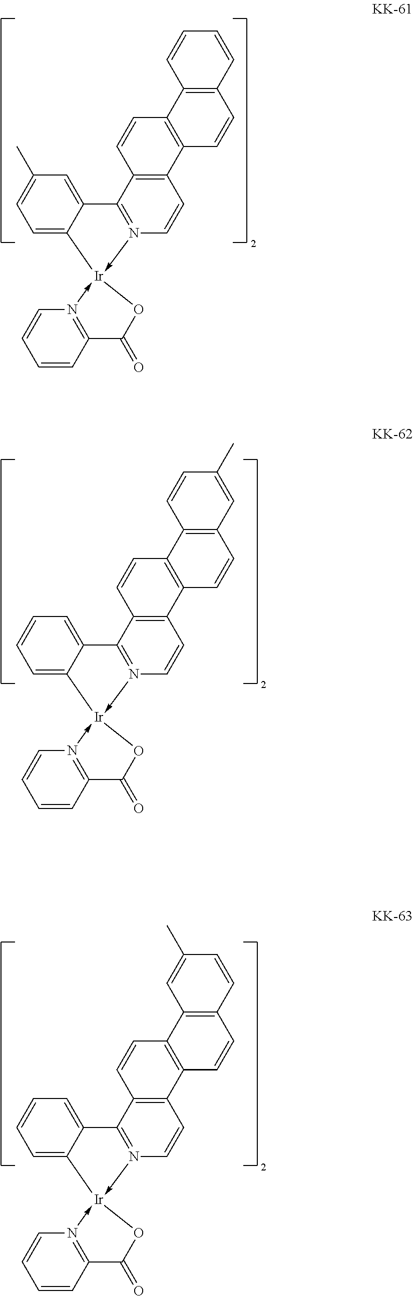

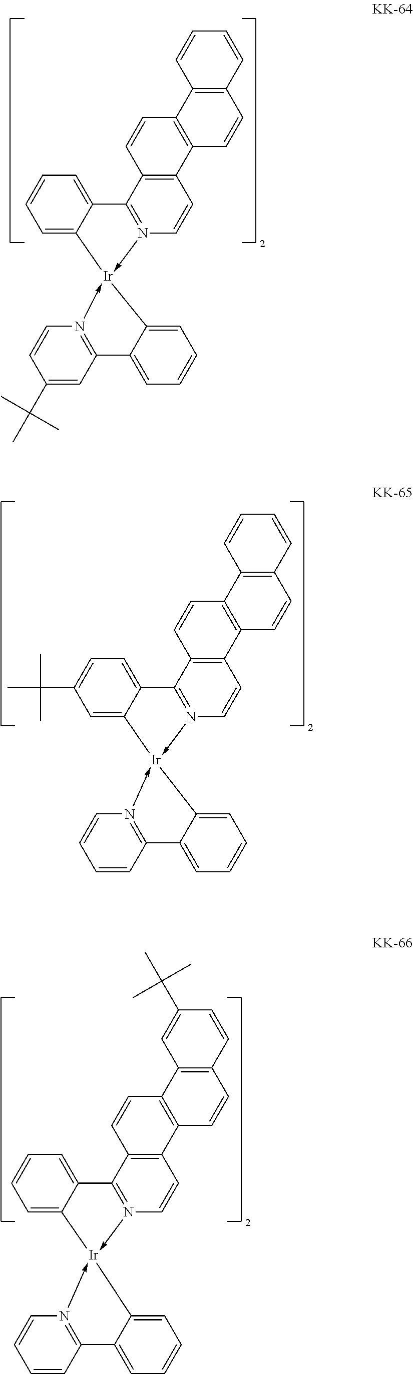

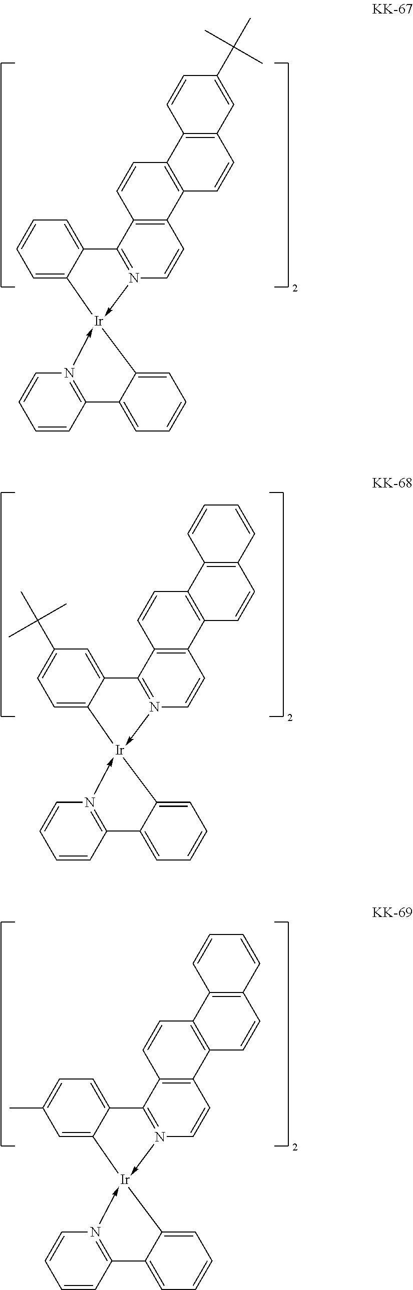

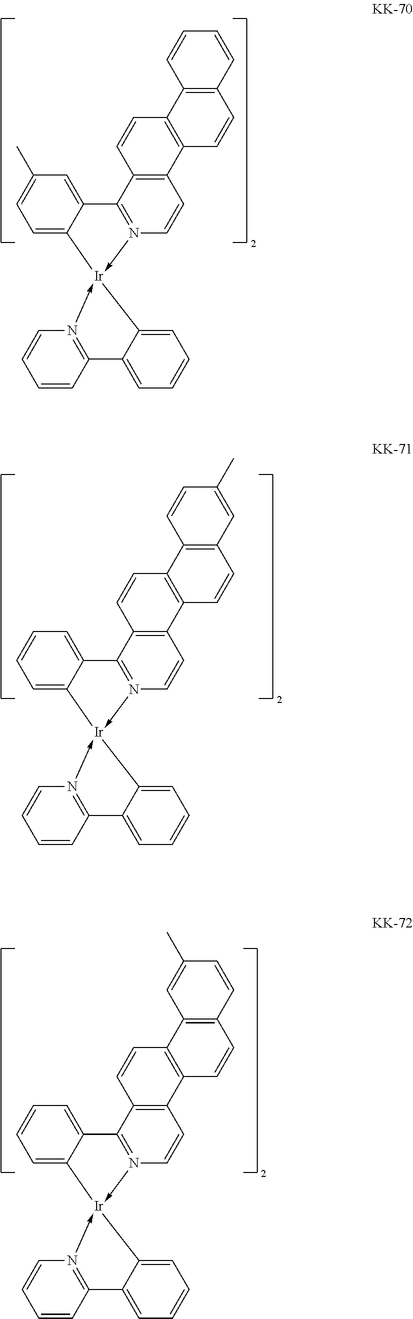

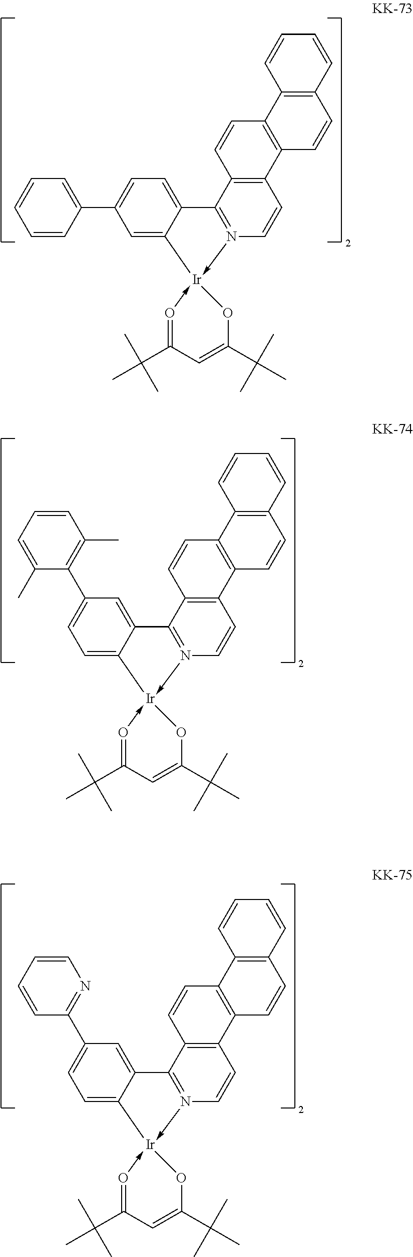

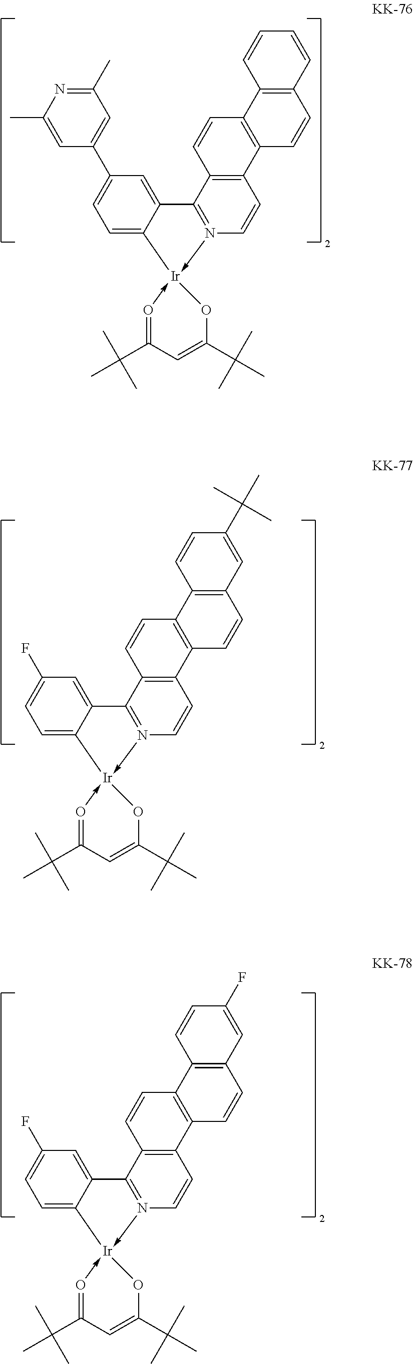

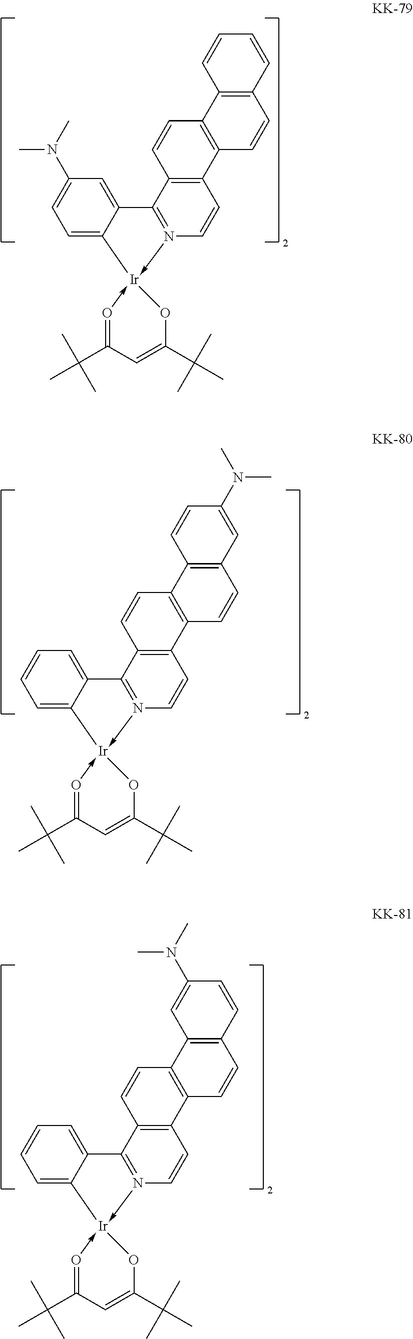

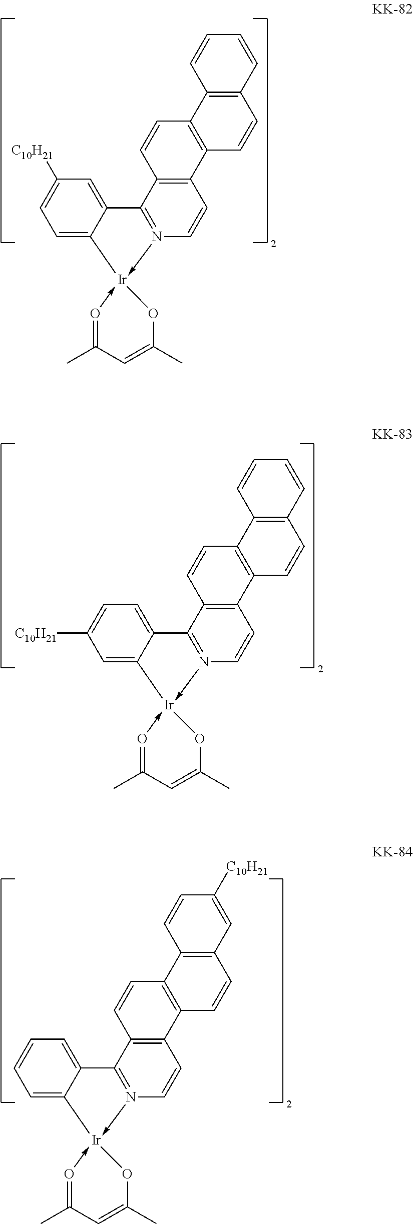

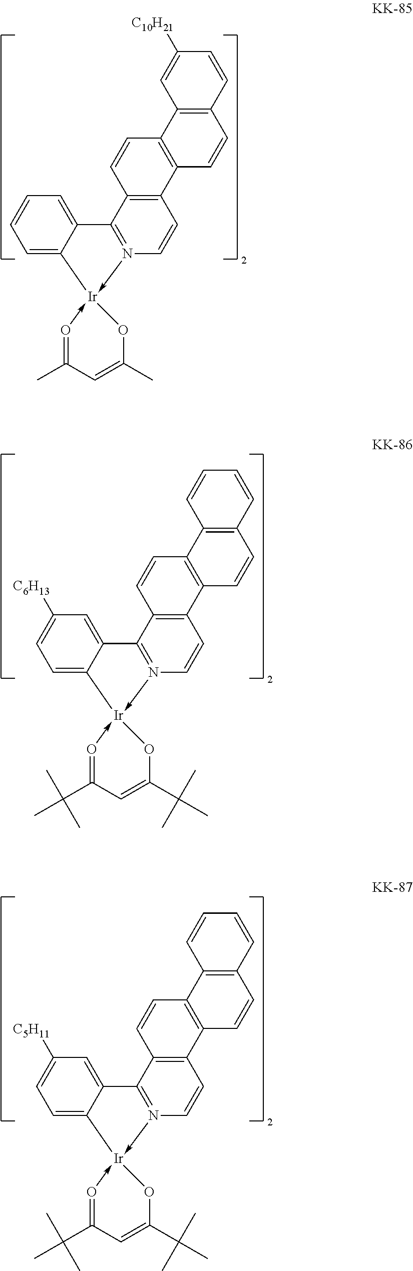

The iridium complexes in a group 1 to which Exemplified Compounds KK-01 to KK-27 correspond are each an iridium complex in which Ir(L′)ₙ is represented by the formula [3], and at least one of R₂₅ and R₂₇ represents a methyl group out of the iridium complexes each represented by the general formula [1].

Those iridium complexes in the group 1 are each a complex having an extremely high emission quantum yield, and hence the use of the complex as a guest molecule for the emission layer provides an organic light-emitting device having high emission efficiency. Further, the iridium complexes in the group 1 are each an iridium complex formed of two ligands of 1-phenylnaphtho[2,1-f]isoquinoline derivatives and one diketone-based bidentate ligand called acetylacetone. Accordingly, the complex can be easily subjected to the sublimation purification because of its relatively small molecular weight.

The iridium complexes in a group 2 to which Exemplified Compounds KK-28 to KK-54 correspond are each an iridium complex in which Ir(L′)ₙ is represented by the formula [3], and at least one of R₂₅ and R₂₇ represents a tert-butyl group out of the iridium complexes represented by the formula [1].

Those iridium complexes in the group 2 are each a complex having an extremely high emission quantum yield and hence the incorporation of the complex as the guest into the emission layer provides an organic light-emitting device having high emission efficiency. Further, the iridium complexes in the group 2 are each an iridium complex formed of two ligands of 1-phenylnaphtho[2,1-f]isoquinoline derivatives and one diketone-based bidentate ligand called dipivaloylmethane. Accordingly, the complex can be easily subjected to the sublimation purification because its molecular weight is relatively small and dipivaloylmethane serves as a steric hindrance group. Further, the complex can be easily handled at the time of its synthesis or purification because of its high solubility.

The iridium complexes in a group 3 to which Exemplified Compounds KK-55 to KK-63 correspond are each an iridium complex in which Ir(L′)ₙ is represented by the formula [4] out of the iridium complexes represented by the formula [1].

Those iridium complexes in the group 3 are each a complex having one picolinic acid derivative as a ligand and having a shorter emission peak wavelength than that in the case where the complex has a diketone-based bidentate ligand.

The iridium complexes in a group 4 to which Exemplified Compounds KK-64 to KK-72 correspond are each an iridium complex in which Ir(L′)ₙ is represented by the formula [5] out of the iridium complexes represented by the formula [1].

Each of those iridium complexes in the group 4 has one phenylpyridine derivative as a nonluminous ligand and provides red light emission derived from a 1-phenylnaphtho[2,1-f]isoquinoline ligand. Accordingly, the complex can be more easily subjected to the sublimation purification than a homoleptic iridium complex using 1-phenylnaphtho[2,1-f]isoquinoline as a ligand can be because of its smaller molecular weight. In addition, the complex can provide an organic light-emitting device having a lifetime as long as that provided by the homoleptic iridium complex.

The iridium complexes in a group 5 to which Exemplified Compounds KK-73 to KK-76 correspond are each an iridium complex in which Ir(L′)ₙ is represented by the formula [3] out of the iridium complexes represented by the formula [1].

Those iridium complexes in the group 5 are each a complex having an extremely high emission quantum yield and hence the incorporation of the complex as the guest into the emission layer provides an organic light-emitting device having high emission efficiency.

In addition, the iridium complexes in the group 5 are each an iridium complex obtained by introducing a substituted or unsubstituted aryl group such as a phenyl group, or a substituted or unsubstituted heteroaromatic group into a ligand formed of a 1-phenylnaphtho[2,1-f]isoquinoline derivative. Accordingly, the complex can be easily subjected to the sublimation purification because the aryl group or the heteroaromatic group functions as a substituent that induces steric hindrance.

The iridium complexes in a group 6 to which Exemplified Compounds KK-77 and KK-78 correspond are each an iridium complex in which Ir(L′)ₙ is represented by the formula [3] out of the iridium complexes represented by the formula [1].

Those iridium complexes in the group 6 are each a complex having an extremely high emission quantum yield and hence the incorporation of the complex as the guest into the emission layer provides an organic light-emitting device having high emission efficiency. Further, the iridium complexes in the group 6 are each an iridium complex in which a ligand is substituted with a fluorine atom. Accordingly, the complex can be easily subjected to the sublimation purification because of the steric hindrance group of an alkyl group and the occurrence of repulsion between the luminous ligands. In addition, even when the complex is doped at a concentration as high as 5 wt % or more with respect to a matrix, light emission showing no reduction in emission efficiency can be obtained.

The iridium complexes in a group 7 to which Exemplified Compounds KK-79 to KK-81 correspond are each an iridium complex in which Ir(L′)ₙ is represented by the formula [3] out of the iridium complexes represented by the formula [1].

Those iridium complexes in the group 7 are each a complex having an extremely high emission quantum yield and hence the use of the complex as the guest for the emission layer provides an organic light-emitting device having high emission efficiency. Further, the iridium complexes in the group 7 are each an iridium complex in which a ligand has a substituted amino group. Accordingly, the HOMO level of the compound is shallow (close to a vacuum level) and its combination with a host (host molecule) having a shallow HOMO level can reduce a charge barrier, and hence low-voltage driving of the device is realized. In addition, the complex can be easily subjected to the sublimation purification because the substituted amino group also functions as a steric hindrance group.

The iridium complexes in a group 8 to which Exemplified Compounds KK-82 to KK-87 correspond are each an iridium complex in which Ir(L′)ₙ is represented by the formula [3] out of the iridium complexes represented by the formula [1].

Those iridium complexes in the group 8 are each a complex having an extremely high emission quantum yield and hence the use of the complex as the guest (for the emission layer) provides an organic light-emitting device having high emission efficiency. Further, the iridium complexes in the group 8 are each an iridium complex having a long-chain alkyl group as a substituent. Accordingly, the solubility of the complex is so high that the complex can be easily formed into a film by application such as a wet method.

(5) Specific Examples of Heterocycle-Containing Compound

Specific structural formulae of the heterocycle-containing compound serving as the host are exemplified below.

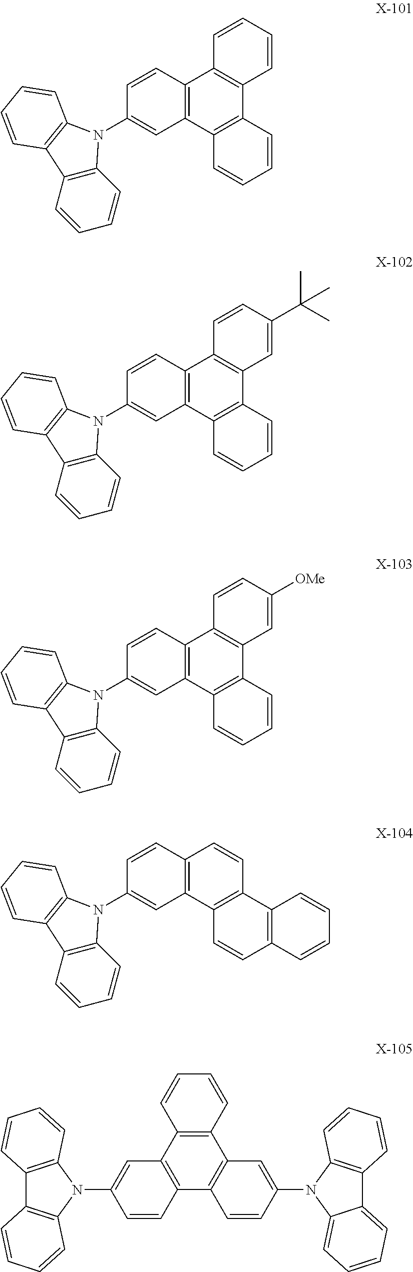 [SMILES: c1ccc2c(c1)c1ccccc1c1cc(-n3c4ccccc4c4ccccc43)ccc21 ; CC(C)(C)c1ccc2c3ccc(-n4c5ccccc5c5ccccc54)cc3c3ccccc3c2c1 ; COc1ccc2c3ccc(-n4c5ccccc5c5ccccc54)cc3c3ccccc3c2c1 ; c1ccc2c(c1)ccc1c3cc(-n4c5ccccc5c5ccccc54)ccc3ccc21 ; c1ccc2c(c1)c1cc(-n3c4ccccc4c4ccccc43)ccc1c1ccc(-n3c4ccccc4c4ccccc43)cc21]

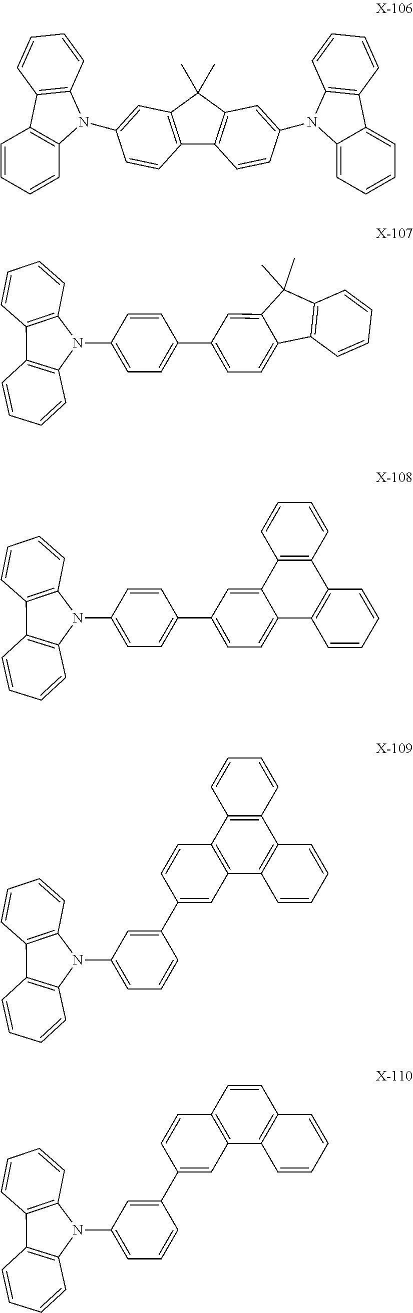 [SMILES: CC1(C)c2cc(-n3c4ccccc4c4ccccc43)ccc2-c2ccc(-n3c4ccccc4c4ccccc43)cc21 ; CC1(C)c2ccccc2-c2ccc(-c3ccc(-n4c5ccccc5c5ccccc54)cc3)cc21 ; c1ccc2c(c1)c1ccccc1c1cc(-c3ccc(-n4c5ccccc5c5ccccc54)cc3)ccc21 ; c1cc(-c2ccc3c4ccccc4c4ccccc4c3c2)cc(-n2c3ccccc3c3ccccc32)c1 ; c1cc(-c2ccc3ccc4ccccc4c3c2)cc(-n2c3ccccc3c3ccccc32)c1]

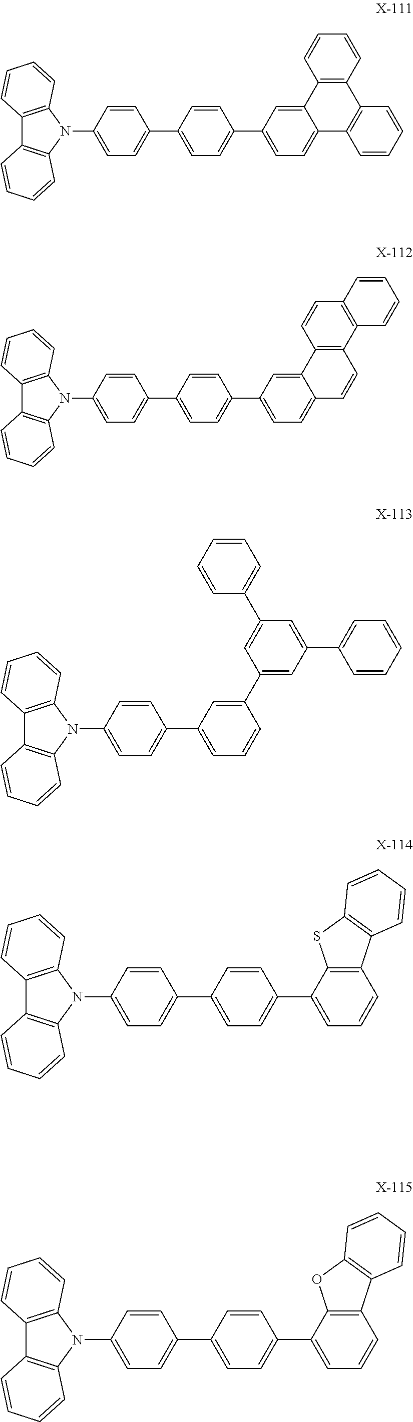 [SMILES: c1ccc2c(c1)c1ccccc1c1cc(-c3ccc(-c4ccc(-n5c6ccccc6c6ccccc65)cc4)cc3)ccc21 ; c1ccc2c(c1)ccc1c3cc(-c4ccc(-c5ccc(-n6c7ccccc7c7ccccc76)cc5)cc4)ccc3ccc21 ; c1ccc(-c2cc(-c3ccccc3)cc(-c3cccc(-c4ccc(-n5c6ccccc6c6ccccc65)cc4)c3)c2)cc1 ; c1ccc2c(c1)sc1c(-c3ccc(-c4ccc(-n5c6ccccc6c6ccccc65)cc4)cc3)cccc12 ; c1ccc2c(c1)oc1c(-c3ccc(-c4ccc(-n5c6ccccc6c6ccccc65)cc4)cc3)cccc12]

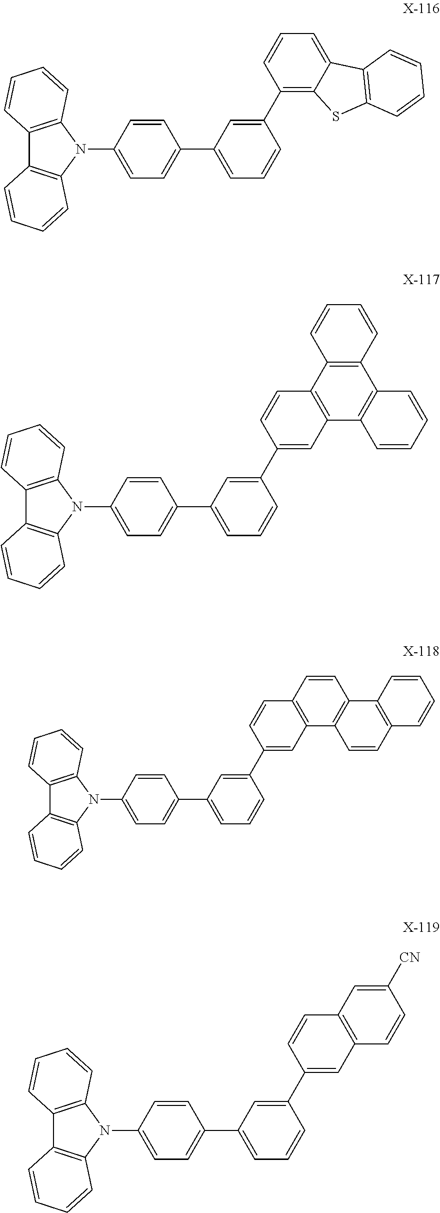 [SMILES: c1cc(-c2ccc(-n3c4ccccc4c4ccccc43)cc2)cc(-c2cccc3c2sc2ccccc23)c1 ; c1cc(-c2ccc(-n3c4ccccc4c4ccccc43)cc2)cc(-c2ccc3c4ccccc4c4ccccc4c3c2)c1 ; c1cc(-c2ccc(-n3c4ccccc4c4ccccc43)cc2)cc(-c2ccc3ccc4c5ccccc5ccc4c3c2)c1 ; N#Cc1ccc2cc(-c3cccc(-c4ccc(-n5c6ccccc6c6ccccc65)cc4)c3)ccc2c1]

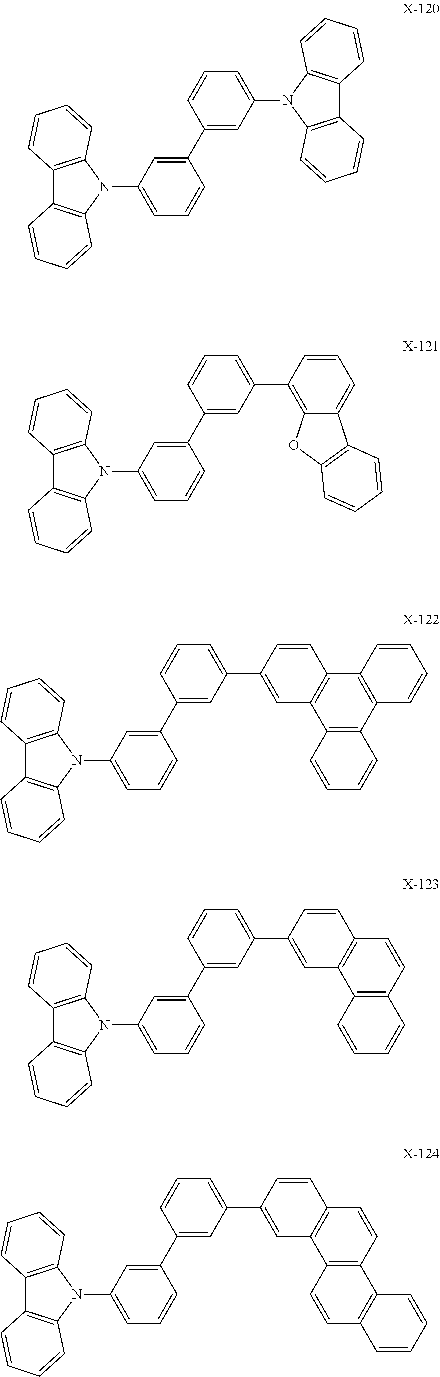 [SMILES: c1cc(-c2cccc(-n3c4ccccc4c4ccccc43)c2)cc(-n2c3ccccc3c3ccccc32)c1 ; c1cc(-c2cccc(-n3c4ccccc4c4ccccc43)c2)cc(-c2cccc3c2oc2ccccc23)c1 ; c1cc(-c2cccc(-n3c4ccccc4c4ccccc43)c2)cc(-c2ccc3c4ccccc4c4ccccc4c3c2)c1 ; c1cc(-c2cccc(-n3c4ccccc4c4ccccc43)c2)cc(-c2ccc3ccc4ccccc4c3c2)c1 ; c1cc(-c2cccc(-n3c4ccccc4c4ccccc43)c2)cc(-c2ccc3ccc4c5ccccc5ccc4c3c2)c1]

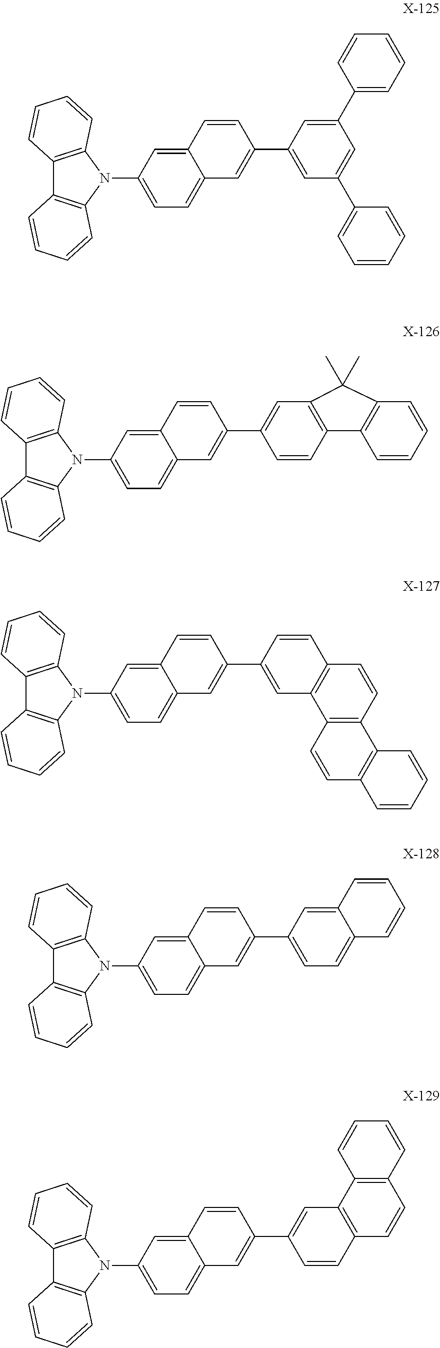 [SMILES: c1ccc(-c2cc(-c3ccccc3)cc(-c3ccc4cc(-n5c6ccccc6c6ccccc65)ccc4c3)c2)cc1 ; CC1(C)c2ccccc2-c2ccc(-c3ccc4cc(-n5c6ccccc6c6ccccc65)ccc4c3)cc21 ; c1ccc2c(c1)ccc1c3cc(-c4ccc5cc(-n6c7ccccc7c7ccccc76)ccc5c4)ccc3ccc21 ; c1ccc2cc(-c3ccc4cc(-n5c6ccccc6c6ccccc65)ccc4c3)ccc2c1 ; c1ccc2c(c1)ccc1ccc(-c3ccc4cc(-n5c6ccccc6c6ccccc65)ccc4c3)cc12]

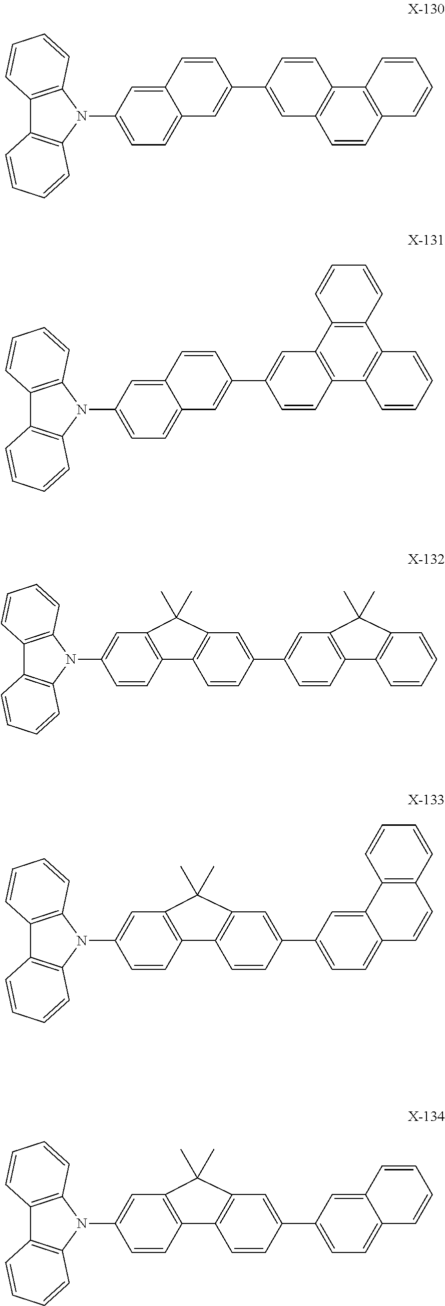 [SMILES: c1ccc2c(c1)ccc1cc(-c3ccc4cc(-n5c6ccccc6c6ccccc65)ccc4c3)ccc12 ; c1ccc2c(c1)c1ccccc1c1cc(-c3ccc4cc(-n5c6ccccc6c6ccccc65)ccc4c3)ccc21 ; CC1(C)c2ccccc2-c2ccc(-c3ccc4c(c3)C(C)(C)c3cc(-n5c6ccccc6c6ccccc65)ccc3-4)cc21 ; CC1(C)c2cc(-c3ccc4ccc5ccccc5c4c3)ccc2-c2ccc(-n3c4ccccc4c4ccccc43)cc21 ; CC1(C)c2cc(-c3ccc4ccccc4c3)ccc2-c2ccc(-n3c4ccccc4c4ccccc43)cc21]

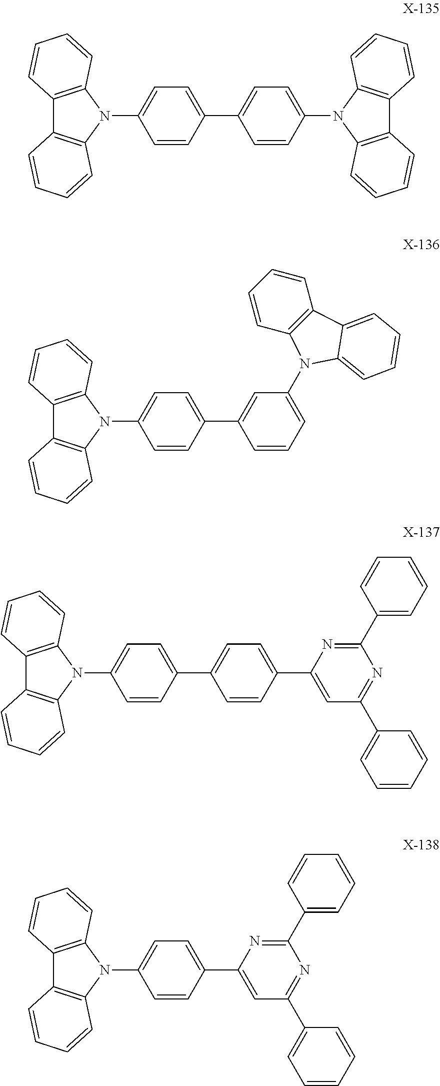 [SMILES: c1ccc2c(c1)c1ccccc1n2-c1ccc(-c2ccc(-n3c4ccccc4c4ccccc43)cc2)cc1 ; c1cc(-c2ccc(-n3c4ccccc4c4ccccc43)cc2)cc(-n2c3ccccc3c3ccccc32)c1 ; c1ccc(-c2cc(-c3ccc(-c4ccc(-n5c6ccccc6c6ccccc65)cc4)cc3)nc(-c3ccccc3)n2)cc1 ; c1ccc(-c2cc(-c3ccc(-n4c5ccccc5c5ccccc54)cc3)nc(-c3ccccc3)n2)cc1]

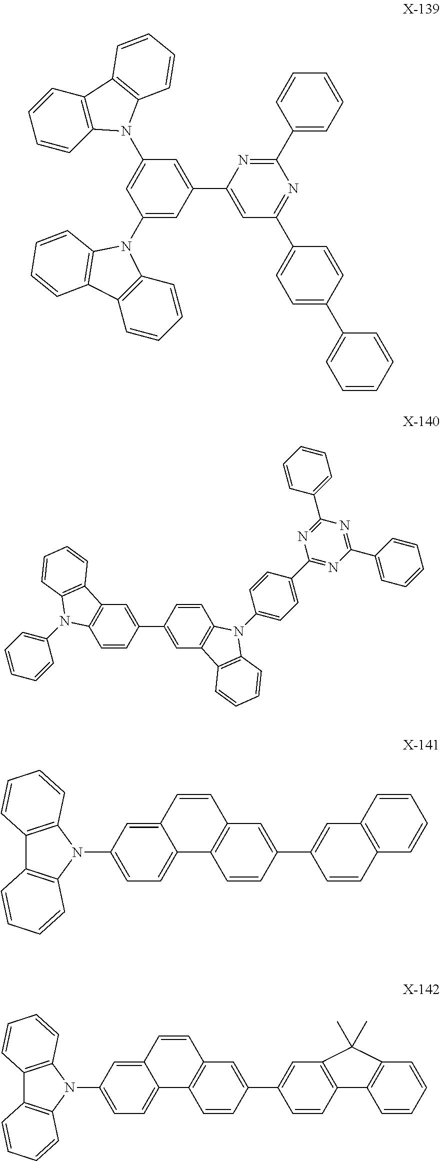 [SMILES: c1ccc(-c2ccc(-c3cc(-c4cc(-n5c6ccccc6c6ccccc65)cc(-n5c6ccccc6c6ccccc65)c4)nc(-c4ccccc4)n3)cc2)cc1 ; c1ccc(-c2nc(-c3ccccc3)nc(-c3ccc(-n4c5ccccc5c5cc(-c6ccc7c(c6)c6ccccc6n7-c6ccccc6)ccc54)cc3)n2)cc1 ; c1ccc2cc(-c3ccc4c(ccc5cc(-n6c7ccccc7c7ccccc76)ccc54)c3)ccc2c1 ; CC1(C)c2ccccc2-c2ccc(-c3ccc4c(ccc5cc(-n6c7ccccc7c7ccccc76)ccc54)c3)cc21]

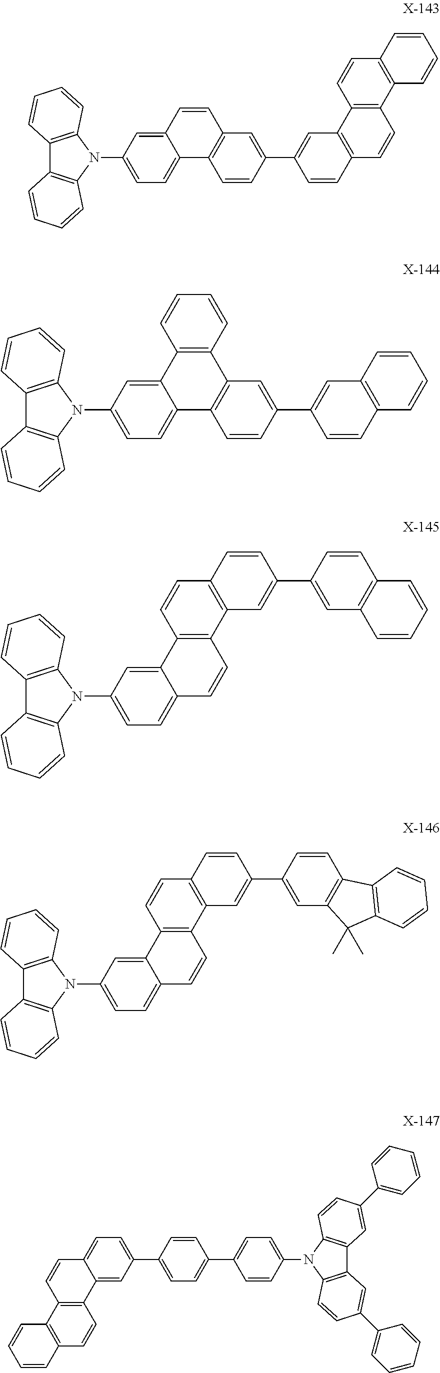 [SMILES: c1ccc2c(c1)ccc1c3cc(-c4ccc5c(ccc6cc(-n7c8ccccc8c8ccccc87)ccc65)c4)ccc3ccc21 ; c1ccc2cc(-c3ccc4c5ccc(-n6c7ccccc7c7ccccc76)cc5c5ccccc5c4c3)ccc2c1 ; c1ccc2cc(-c3ccc4ccc5c6cc(-n7c8ccccc8c8ccccc87)ccc6ccc5c4c3)ccc2c1 ; CC1(C)c2ccccc2-c2ccc(-c3ccc4ccc5c6cc(-n7c8ccccc8c8ccccc87)ccc6ccc5c4c3)cc21 ; c1ccc(-c2ccc3c(c2)c2cc(-c4ccccc4)ccc2n3-c2ccc(-c3ccc(-c4ccc5ccc6c7ccccc7ccc6c5c4)cc3)cc2)cc1]

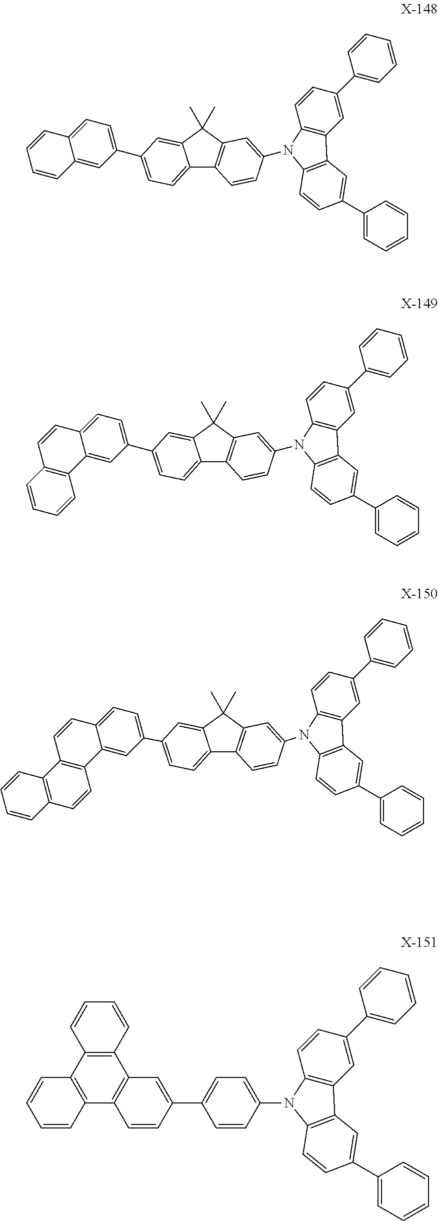 [SMILES: CC1(C)c2cc(-c3ccc4ccccc4c3)ccc2-c2ccc(-n3c4ccc(-c5ccccc5)cc4c4cc(-c5ccccc5)ccc43)cc21 ; CC1(C)c2cc(-c3ccc4ccc5ccccc5c4c3)ccc2-c2ccc(-n3c4ccc(-c5ccccc5)cc4c4cc(-c5ccccc5)ccc43)cc21 ; CC1(C)c2cc(-c3ccc4ccc5c6ccccc6ccc5c4c3)ccc2-c2ccc(-n3c4ccc(-c5ccccc5)cc4c4cc(-c5ccccc5)ccc43)cc21 ; c1ccc(-c2ccc3c(c2)c2cc(-c4ccccc4)ccc2n3-c2ccc(-c3ccc4c5ccccc5c5ccccc5c4c3)cc2)cc1]

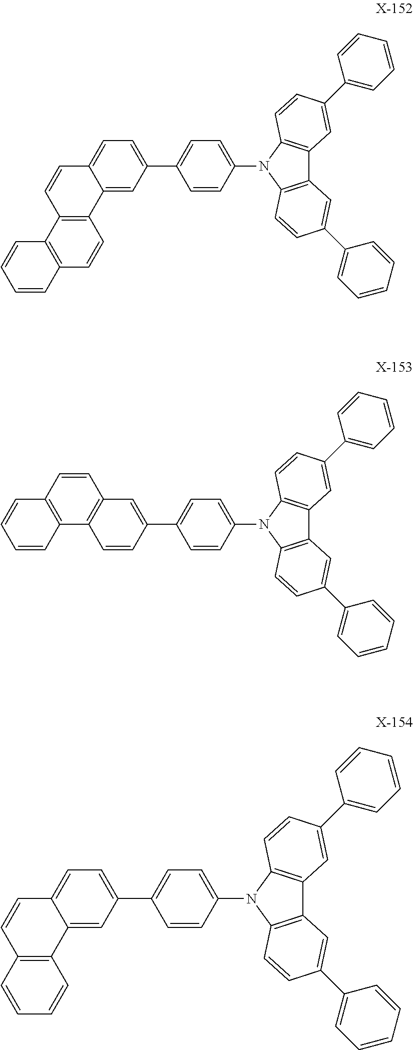 [SMILES: c1ccc(-c2ccc3c(c2)c2cc(-c4ccccc4)ccc2n3-c2ccc(-c3ccc4ccc5c6ccccc6ccc5c4c3)cc2)cc1 ; c1ccc(-c2ccc3c(c2)c2cc(-c4ccccc4)ccc2n3-c2ccc(-c3ccc4c(ccc5ccccc54)c3)cc2)cc1 ; c1ccc(-c2ccc3c(c2)c2cc(-c4ccccc4)ccc2n3-c2ccc(-c3ccc4ccc5ccccc5c4c3)cc2)cc1]

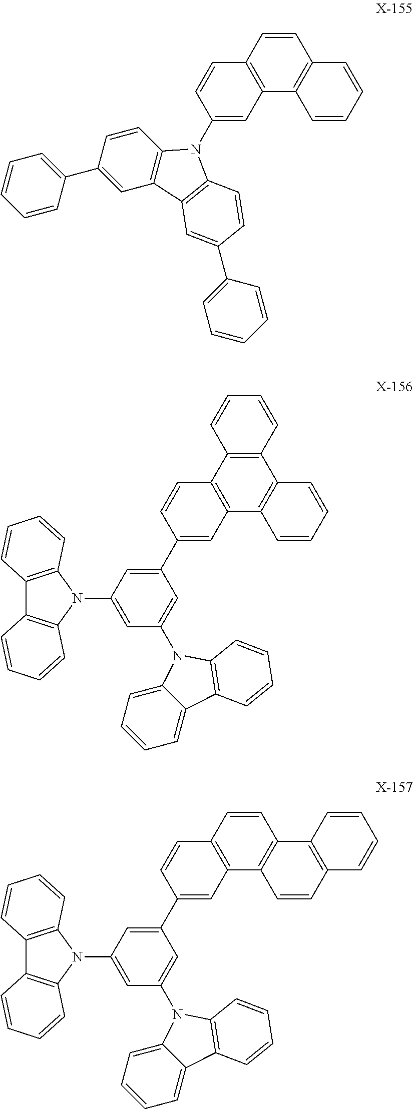 [SMILES: c1ccc(-c2ccc3c(c2)c2cc(-c4ccccc4)ccc2n3-c2ccc3ccc4ccccc4c3c2)cc1 ; c1ccc2c(c1)c1ccccc1c1cc(-c3cc(-n4c5ccccc5c5ccccc54)cc(-n4c5ccccc5c5ccccc54)c3)ccc21 ; c1ccc2c(c1)ccc1c3cc(-c4cc(-n5c6ccccc6c6ccccc65)cc(-n5c6ccccc6c6ccccc65)c4)ccc3ccc21]

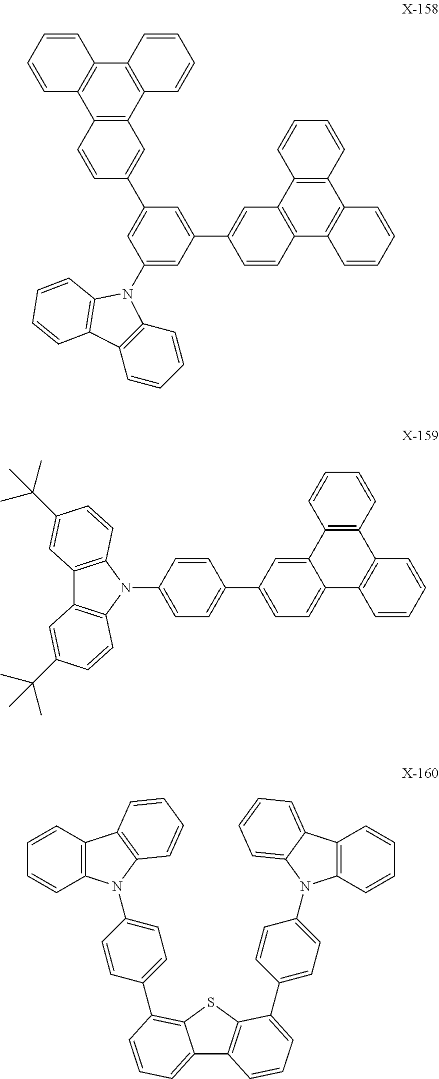 [SMILES: c1ccc2c(c1)c1ccccc1c1cc(-c3cc(-c4ccc5c6ccccc6c6ccccc6c5c4)cc(-n4c5ccccc5c5ccccc54)c3)ccc21 ; CC(C)(C)c1ccc2c(c1)c1cc(C(C)(C)C)ccc1n2-c1ccc(-c2ccc3c4ccccc4c4ccccc4c3c2)cc1 ; c1cc(-c2ccc(-n3c4ccccc4c4ccccc43)cc2)c2sc3c(-c4ccc(-n5c6ccccc6c6ccccc65)cc4)cccc3c2c1]

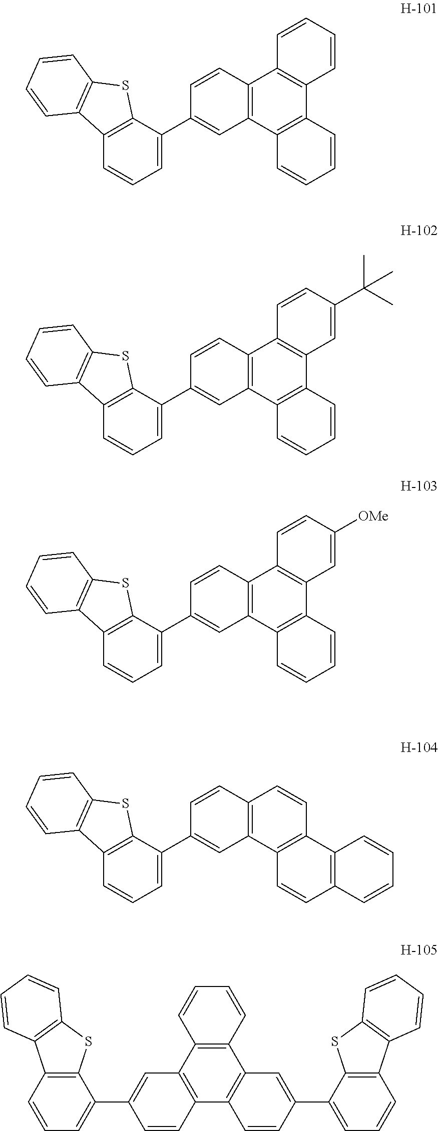 [SMILES: c1ccc2c(c1)sc1c(-c3ccc4c5ccccc5c5ccccc5c4c3)cccc12 ; CC(C)(C)c1ccc2c3ccc(-c4cccc5c4sc4ccccc45)cc3c3ccccc3c2c1 ; COc1ccc2c3ccc(-c4cccc5c4sc4ccccc45)cc3c3ccccc3c2c1 ; c1ccc2c(c1)ccc1c3cc(-c4cccc5c4sc4ccccc45)ccc3ccc21 ; c1ccc2c(c1)sc1c(-c3ccc4c5ccc(-c6cccc7c6sc6ccccc67)cc5c5ccccc5c4c3)cccc12]

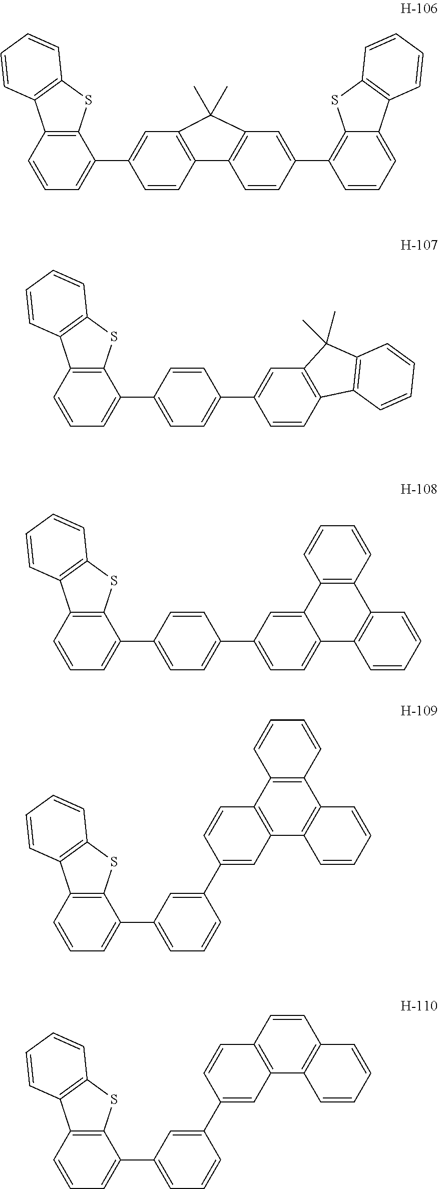 [SMILES: CC1(C)c2cc(-c3cccc4c3sc3ccccc34)ccc2-c2ccc(-c3cccc4c3sc3ccccc34)cc21 ; CC1(C)c2ccccc2-c2ccc(-c3ccc(-c4cccc5c4sc4ccccc45)cc3)cc21 ; c1ccc2c(c1)sc1c(-c3ccc(-c4ccc5c6ccccc6c6ccccc6c5c4)cc3)cccc12 ; c1cc(-c2ccc3c4ccccc4c4ccccc4c3c2)cc(-c2cccc3c2sc2ccccc23)c1 ; c1cc(-c2ccc3ccc4ccccc4c3c2)cc(-c2cccc3c2sc2ccccc23)c1]

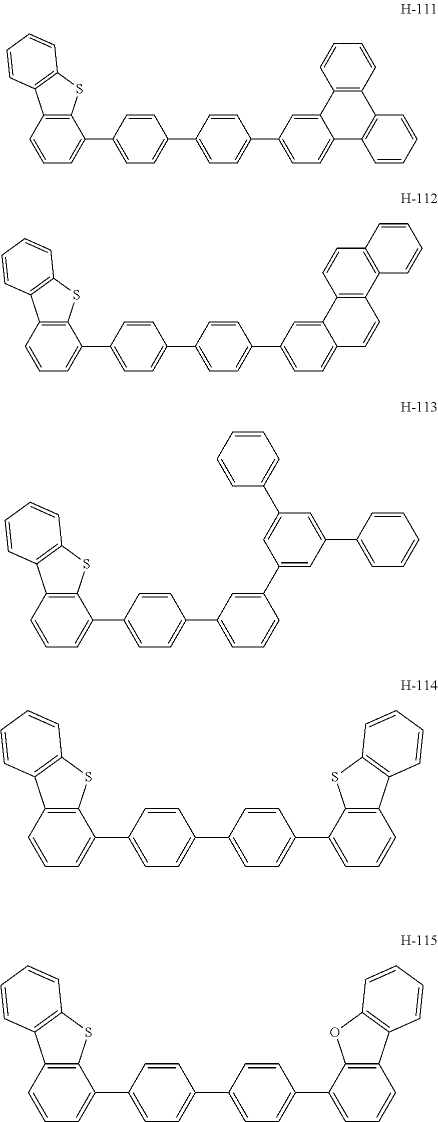 [SMILES: c1ccc2c(c1)sc1c(-c3ccc(-c4ccc(-c5ccc6c7ccccc7c7ccccc7c6c5)cc4)cc3)cccc12 ; c1ccc2c(c1)ccc1c3cc(-c4ccc(-c5ccc(-c6cccc7c6sc6ccccc67)cc5)cc4)ccc3ccc21 ; c1ccc(-c2cc(-c3ccccc3)cc(-c3cccc(-c4ccc(-c5cccc6c5sc5ccccc56)cc4)c3)c2)cc1 ; c1ccc2c(c1)sc1c(-c3ccc(-c4ccc(-c5cccc6c5sc5ccccc56)cc4)cc3)cccc12 ; c1ccc2c(c1)oc1c(-c3ccc(-c4ccc(-c5cccc6c5sc5ccccc56)cc4)cc3)cccc12]

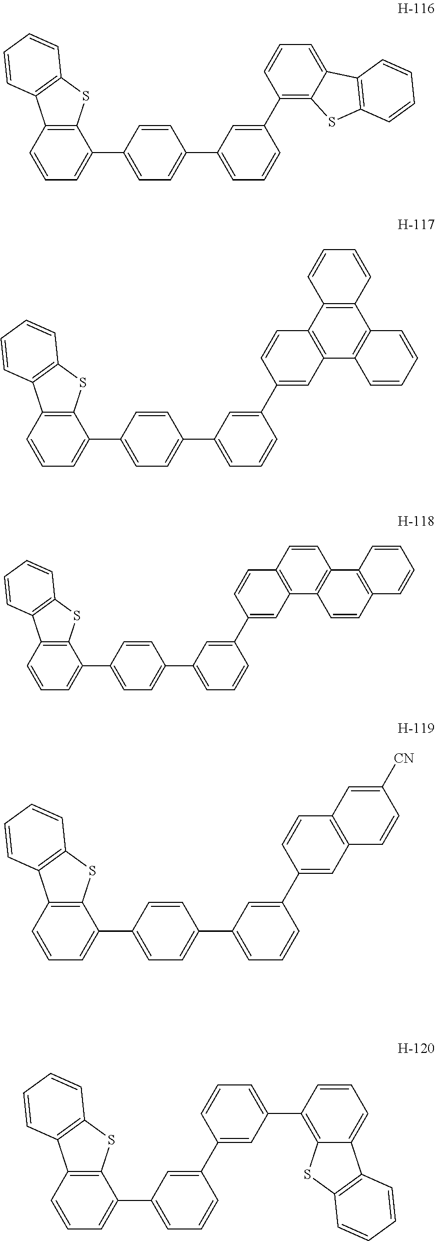 [SMILES: c1cc(-c2ccc(-c3cccc4c3sc3ccccc34)cc2)cc(-c2cccc3c2sc2ccccc23)c1 ; c1cc(-c2ccc(-c3cccc4c3sc3ccccc34)cc2)cc(-c2ccc3c4ccccc4c4ccccc4c3c2)c1 ; c1cc(-c2ccc(-c3cccc4c3sc3ccccc34)cc2)cc(-c2ccc3ccc4c5ccccc5ccc4c3c2)c1 ; N#Cc1ccc2cc(-c3cccc(-c4ccc(-c5cccc6c5sc5ccccc56)cc4)c3)ccc2c1 ; c1cc(-c2cccc(-c3cccc4c3sc3ccccc34)c2)cc(-c2cccc3c2sc2ccccc23)c1]

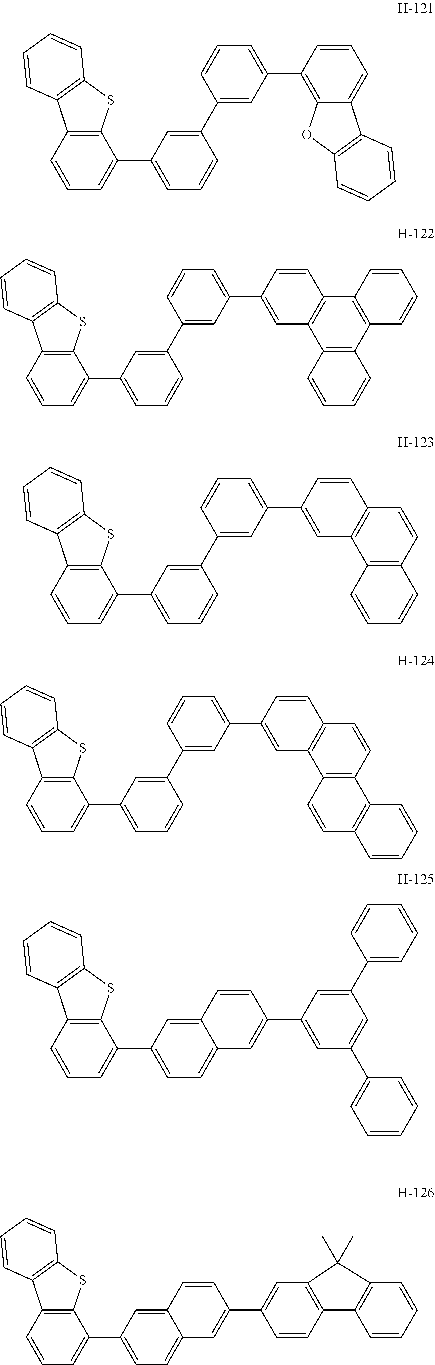 [SMILES: c1cc(-c2cccc(-c3cccc4c3sc3ccccc34)c2)cc(-c2cccc3c2oc2ccccc23)c1 ; c1cc(-c2cccc(-c3cccc4c3sc3ccccc34)c2)cc(-c2ccc3c4ccccc4c4ccccc4c3c2)c1 ; c1cc(-c2cccc(-c3cccc4c3sc3ccccc34)c2)cc(-c2ccc3ccc4ccccc4c3c2)c1 ; c1cc(-c2cccc(-c3cccc4c3sc3ccccc34)c2)cc(-c2ccc3ccc4c5ccccc5ccc4c3c2)c1 ; c1ccc(-c2cc(-c3ccccc3)cc(-c3ccc4cc(-c5cccc6c5sc5ccccc56)ccc4c3)c2)cc1 ; CC1(C)c2ccccc2-c2ccc(-c3ccc4cc(-c5cccc6c5sc5ccccc56)ccc4c3)cc21]

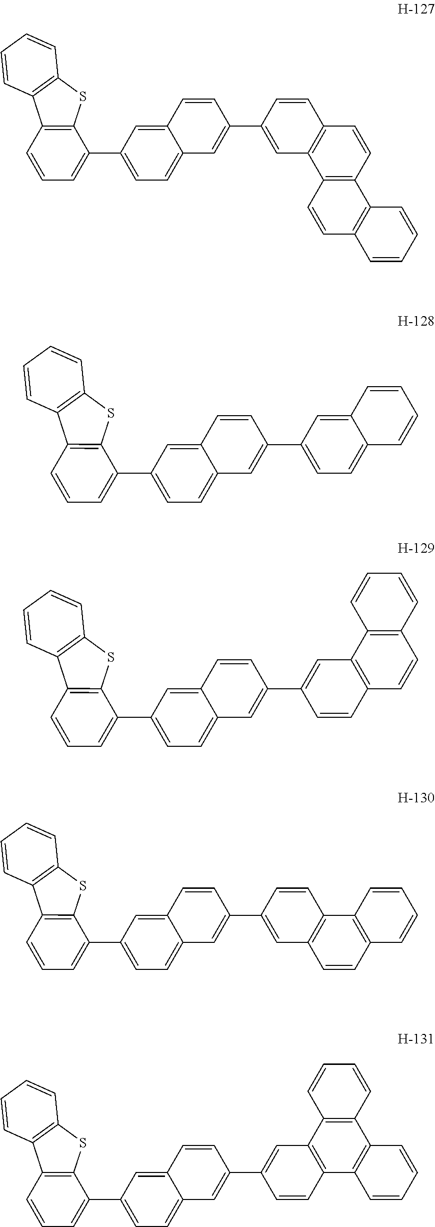 [SMILES: c1ccc2c(c1)ccc1c3cc(-c4ccc5cc(-c6cccc7c6sc6ccccc67)ccc5c4)ccc3ccc21 ; c1ccc2cc(-c3ccc4cc(-c5cccc6c5sc5ccccc56)ccc4c3)ccc2c1 ; c1ccc2c(c1)ccc1ccc(-c3ccc4cc(-c5cccc6c5sc5ccccc56)ccc4c3)cc12 ; c1ccc2c(c1)ccc1cc(-c3ccc4cc(-c5cccc6c5sc5ccccc56)ccc4c3)ccc12 ; c1ccc2c(c1)sc1c(-c3ccc4cc(-c5ccc6c7ccccc7c7ccccc7c6c5)ccc4c3)cccc12]

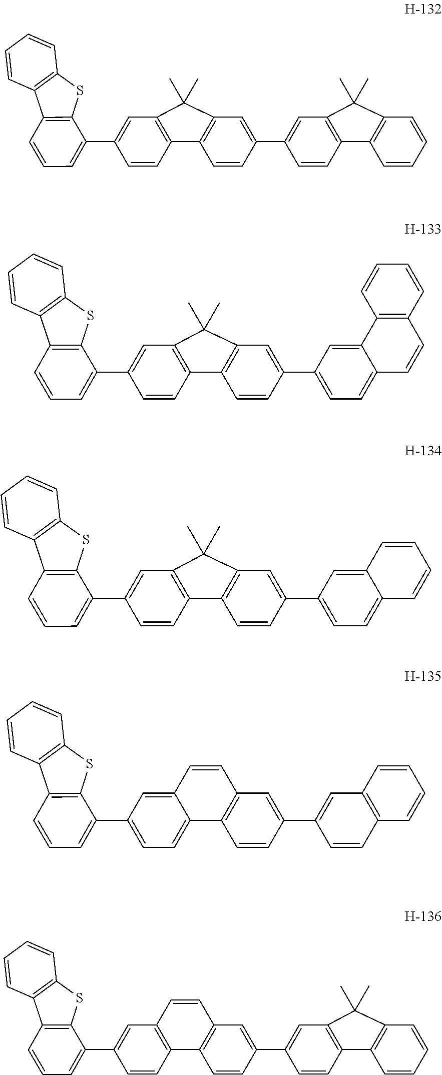 [SMILES: CC1(C)c2ccccc2-c2ccc(-c3ccc4c(c3)C(C)(C)c3cc(-c5cccc6c5sc5ccccc56)ccc3-4)cc21 ; CC1(C)c2cc(-c3ccc4ccc5ccccc5c4c3)ccc2-c2ccc(-c3cccc4c3sc3ccccc34)cc21 ; CC1(C)c2cc(-c3ccc4ccccc4c3)ccc2-c2ccc(-c3cccc4c3sc3ccccc34)cc21 ; c1ccc2cc(-c3ccc4c(ccc5cc(-c6cccc7c6sc6ccccc67)ccc54)c3)ccc2c1 ; CC1(C)c2ccccc2-c2ccc(-c3ccc4c(ccc5cc(-c6cccc7c6sc6ccccc67)ccc54)c3)cc21]

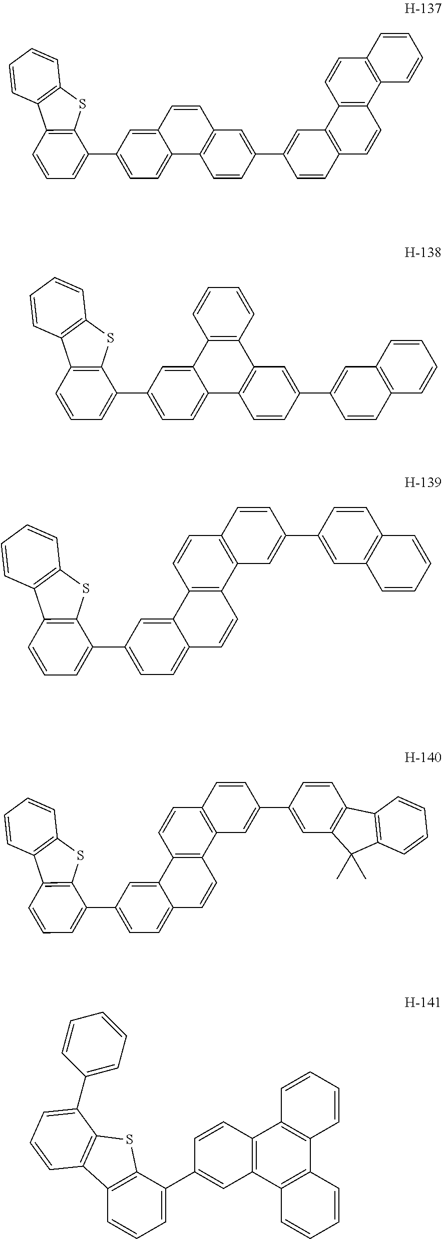 [SMILES: c1ccc2c(c1)ccc1c3cc(-c4ccc5c(ccc6cc(-c7cccc8c7sc7ccccc78)ccc65)c4)ccc3ccc21 ; c1ccc2cc(-c3ccc4c5ccc(-c6cccc7c6sc6ccccc67)cc5c5ccccc5c4c3)ccc2c1 ; c1ccc2cc(-c3ccc4ccc5c6cc(-c7cccc8c7sc7ccccc78)ccc6ccc5c4c3)ccc2c1 ; CC1(C)c2ccccc2-c2ccc(-c3ccc4ccc5c6cc(-c7cccc8c7sc7ccccc78)ccc6ccc5c4c3)cc21 ; c1ccc(-c2cccc3c2sc2c(-c4ccc5c6ccccc6c6ccccc6c5c4)cccc23)cc1]

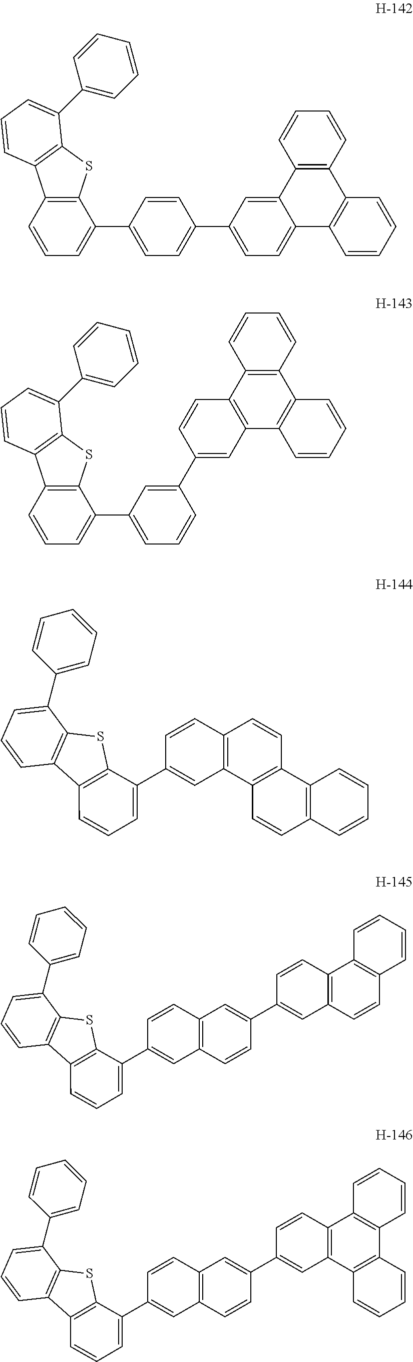 [SMILES: c1ccc(-c2cccc3c2sc2c(-c4ccc(-c5ccc6c7ccccc7c7ccccc7c6c5)cc4)cccc23)cc1 ; c1ccc(-c2cccc3c2sc2c(-c4cccc(-c5ccc6c7ccccc7c7ccccc7c6c5)c4)cccc23)cc1 ; c1ccc(-c2cccc3c2sc2c(-c4ccc5ccc6c7ccccc7ccc6c5c4)cccc23)cc1 ; c1ccc(-c2cccc3c2sc2c(-c4ccc5cc(-c6ccc7c(ccc8ccccc87)c6)ccc5c4)cccc23)cc1 ; c1ccc(-c2cccc3c2sc2c(-c4ccc5cc(-c6ccc7c8ccccc8c8ccccc8c7c6)ccc5c4)cccc23)cc1]

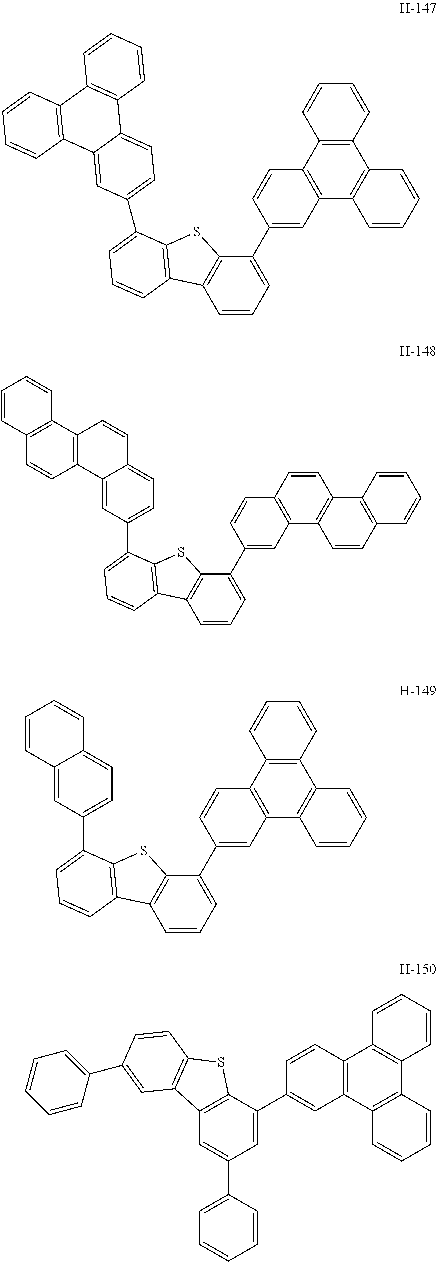 [SMILES: c1cc(-c2ccc3c4ccccc4c4ccccc4c3c2)c2sc3c(-c4ccc5c6ccccc6c6ccccc6c5c4)cccc3c2c1 ; c1ccc2c(c1)ccc1c3cc(-c4cccc5c4sc4c(-c6ccc7ccc8c9ccccc9ccc8c7c6)cccc45)ccc3ccc21 ; c1ccc2cc(-c3cccc4c3sc3c(-c5ccc6c7ccccc7c7ccccc7c6c5)cccc34)ccc2c1 ; c1ccc(-c2ccc3sc4c(-c5ccc6c7ccccc7c7ccccc7c6c5)cc(-c5ccccc5)cc4c3c2)cc1]

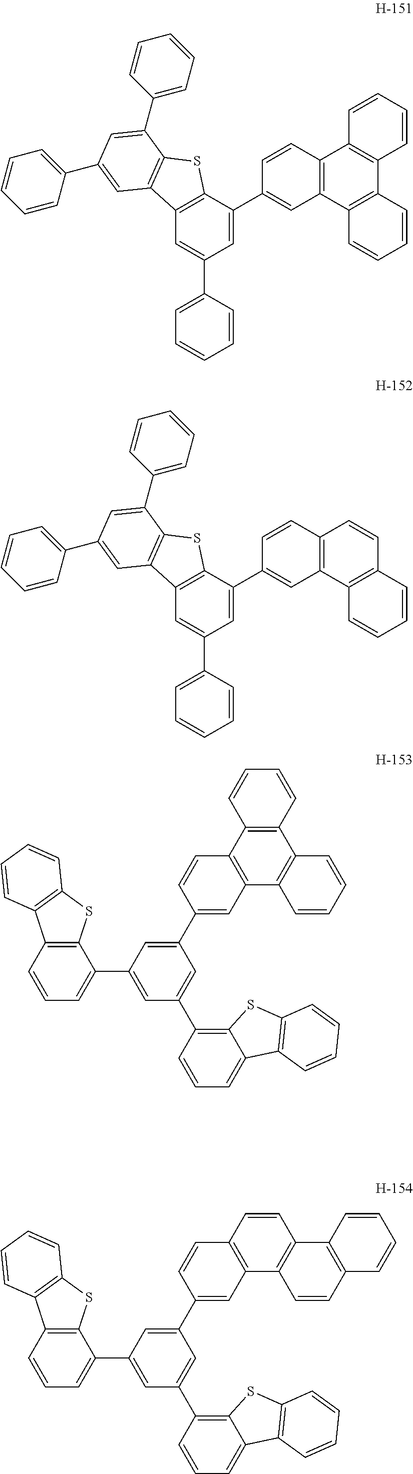 [SMILES: c1ccc(-c2cc(-c3ccccc3)c3sc4c(-c5ccc6c7ccccc7c7ccccc7c6c5)cc(-c5ccccc5)cc4c3c2)cc1 ; c1ccc(-c2cc(-c3ccccc3)c3sc4c(-c5ccc6ccc7ccccc7c6c5)cc(-c5ccccc5)cc4c3c2)cc1 ; c1ccc2c(c1)sc1c(-c3cc(-c4ccc5c6ccccc6c6ccccc6c5c4)cc(-c4cccc5c4sc4ccccc45)c3)cccc12 ; c1ccc2c(c1)ccc1c3cc(-c4cc(-c5cccc6c5sc5ccccc56)cc(-c5cccc6c5sc5ccccc56)c4)ccc3ccc21]

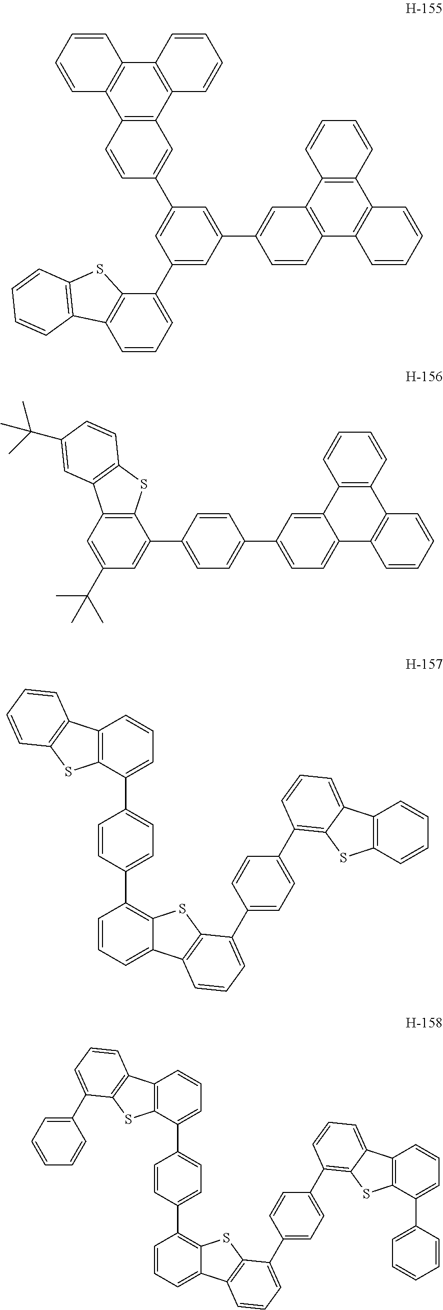 [SMILES: c1ccc2c(c1)sc1c(-c3cc(-c4ccc5c6ccccc6c6ccccc6c5c4)cc(-c4ccc5c6ccccc6c6ccccc6c5c4)c3)cccc12 ; CC(C)(C)c1ccc2sc3c(-c4ccc(-c5ccc6c7ccccc7c7ccccc7c6c5)cc4)cc(C(C)(C)C)cc3c2c1 ; c1ccc2c(c1)sc1c(-c3ccc(-c4cccc5c4sc4c(-c6ccc(-c7cccc8c7sc7ccccc78)cc6)cccc45)cc3)cccc12 ; c1ccc(-c2cccc3c2sc2c(-c4ccc(-c5cccc6c5sc5c(-c7ccc(-c8cccc9c8sc8c(-c%10ccccc%10)cccc89)cc7)cccc56)cc4)cccc23)cc1]

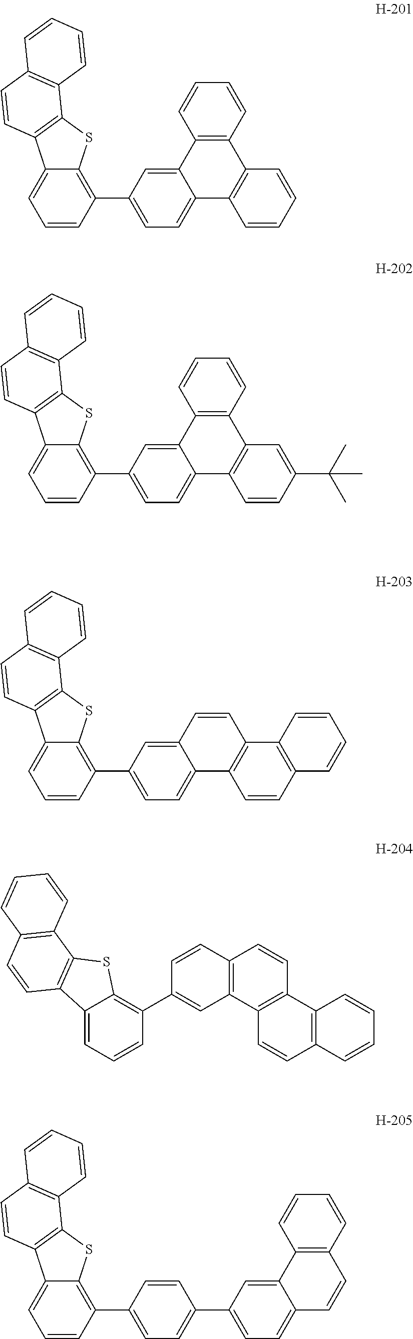 [SMILES: c1ccc2c(c1)ccc1c3cccc(-c4ccc5c6ccccc6c6ccccc6c5c4)c3sc21 ; CC(C)(C)c1ccc2c3ccc(-c4cccc5c4sc4c6ccccc6ccc54)cc3c3ccccc3c2c1 ; c1ccc2c(c1)ccc1c3ccc(-c4cccc5c4sc4c6ccccc6ccc54)cc3ccc21 ; c1ccc2c(c1)ccc1c3cc(-c4cccc5c4sc4c6ccccc6ccc54)ccc3ccc21 ; c1ccc2c(c1)ccc1ccc(-c3ccc(-c4cccc5c4sc4c6ccccc6ccc54)cc3)cc12]

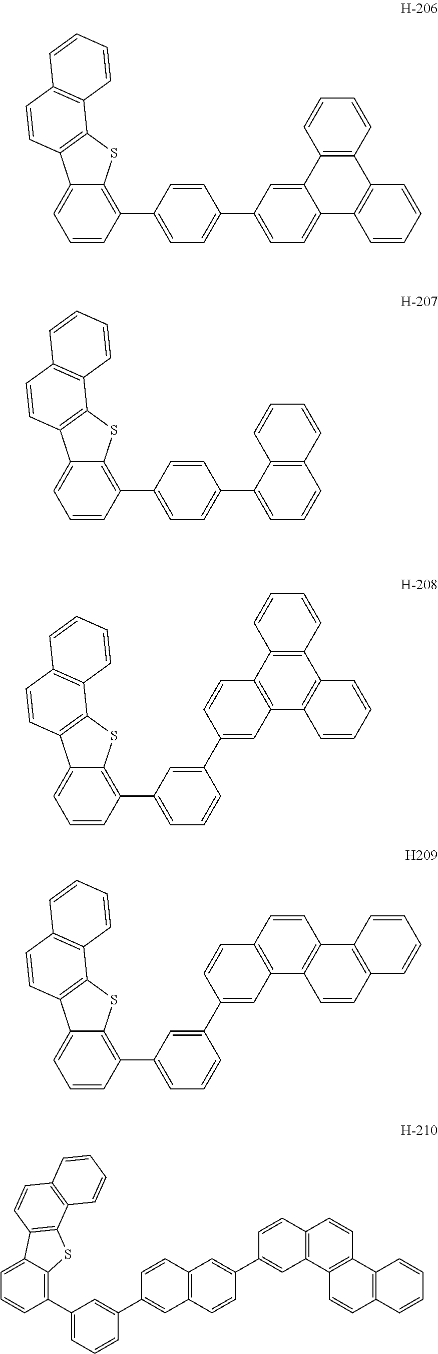 [SMILES: c1ccc2c(c1)ccc1c3cccc(-c4ccc(-c5ccc6c7ccccc7c7ccccc7c6c5)cc4)c3sc21 ; c1ccc2c(-c3ccc(-c4cccc5c4sc4c6ccccc6ccc54)cc3)cccc2c1 ; c1cc(-c2ccc3c4ccccc4c4ccccc4c3c2)cc(-c2cccc3c2sc2c4ccccc4ccc32)c1 ; c1cc(-c2ccc3ccc4c5ccccc5ccc4c3c2)cc(-c2cccc3c2sc2c4ccccc4ccc32)c1 ; c1cc(-c2ccc3cc(-c4ccc5ccc6c7ccccc7ccc6c5c4)ccc3c2)cc(-c2cccc3c2sc2c4ccccc4ccc32)c1]

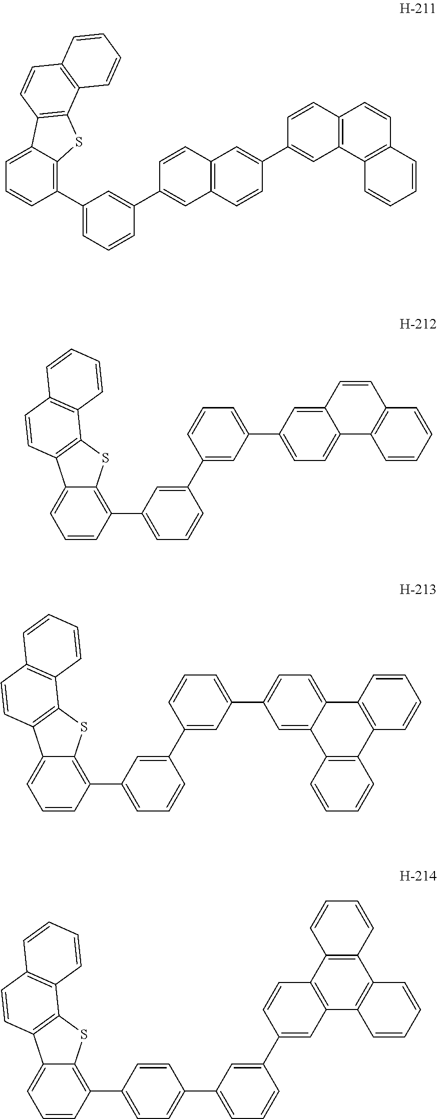 [SMILES: c1cc(-c2ccc3cc(-c4ccc5ccc6ccccc6c5c4)ccc3c2)cc(-c2cccc3c2sc2c4ccccc4ccc32)c1 ; c1cc(-c2cccc(-c3cccc4c3sc3c5ccccc5ccc43)c2)cc(-c2ccc3c(ccc4ccccc43)c2)c1 ; c1cc(-c2cccc(-c3cccc4c3sc3c5ccccc5ccc43)c2)cc(-c2ccc3c4ccccc4c4ccccc4c3c2)c1 ; c1cc(-c2ccc(-c3cccc4c3sc3c5ccccc5ccc43)cc2)cc(-c2ccc3c4ccccc4c4ccccc4c3c2)c1]

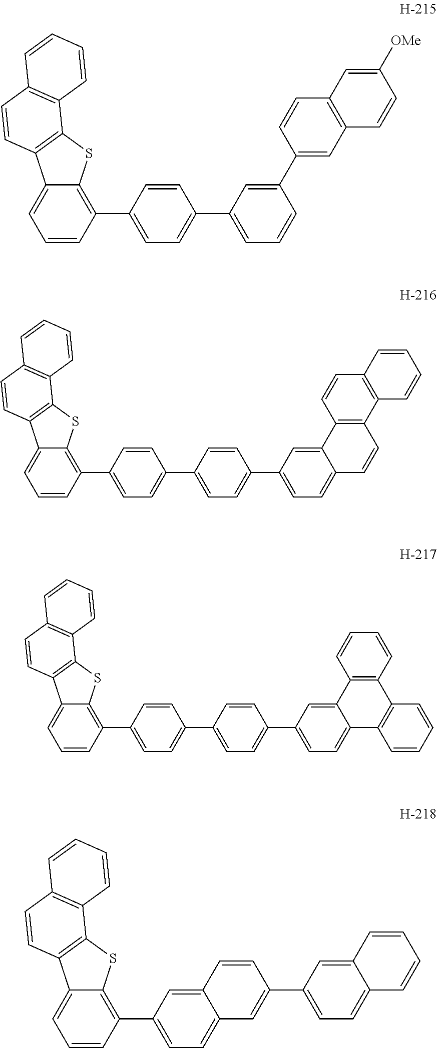 [SMILES: COc1ccc2cc(-c3cccc(-c4ccc(-c5cccc6c5sc5c7ccccc7ccc65)cc4)c3)ccc2c1 ; c1ccc2c(c1)ccc1c3cc(-c4ccc(-c5ccc(-c6cccc7c6sc6c8ccccc8ccc76)cc5)cc4)ccc3ccc21 ; c1ccc2c(c1)ccc1c3cccc(-c4ccc(-c5ccc(-c6ccc7c8ccccc8c8ccccc8c7c6)cc5)cc4)c3sc21 ; c1ccc2cc(-c3ccc4cc(-c5cccc6c5sc5c7ccccc7ccc65)ccc4c3)ccc2c1]

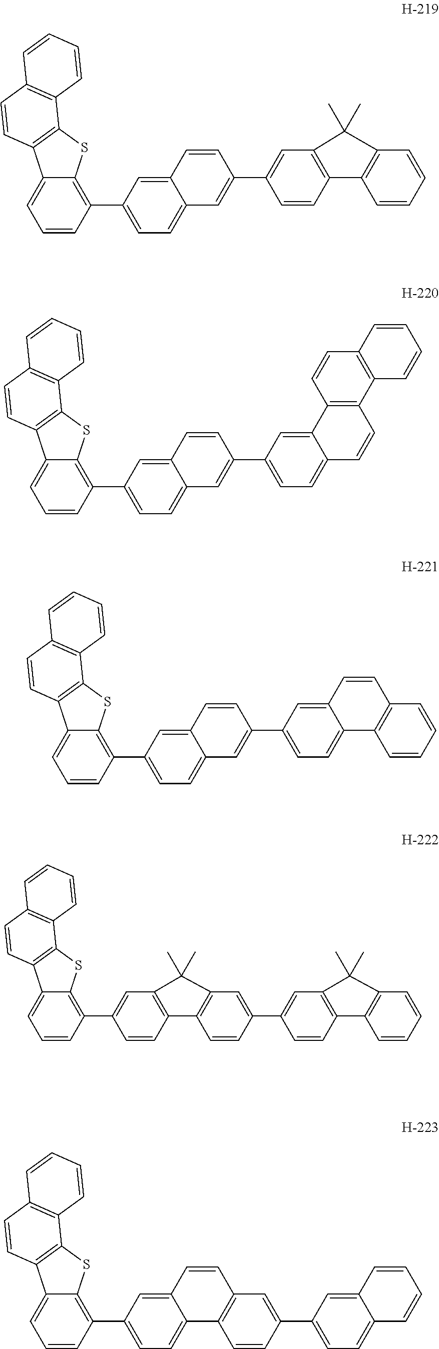 [SMILES: CC1(C)c2ccccc2-c2ccc(-c3ccc4cc(-c5cccc6c5sc5c7ccccc7ccc65)ccc4c3)cc21 ; c1ccc2c(c1)ccc1c3cc(-c4ccc5cc(-c6cccc7c6sc6c8ccccc8ccc76)ccc5c4)ccc3ccc21 ; c1ccc2c(c1)ccc1cc(-c3ccc4cc(-c5cccc6c5sc5c7ccccc7ccc65)ccc4c3)ccc12 ; CC1(C)c2ccccc2-c2ccc(-c3ccc4c(c3)C(C)(C)c3cc(-c5cccc6c5sc5c7ccccc7ccc65)ccc3-4)cc21 ; c1ccc2cc(-c3ccc4c(ccc5cc(-c6cccc7c6sc6c8ccccc8ccc76)ccc54)c3)ccc2c1]

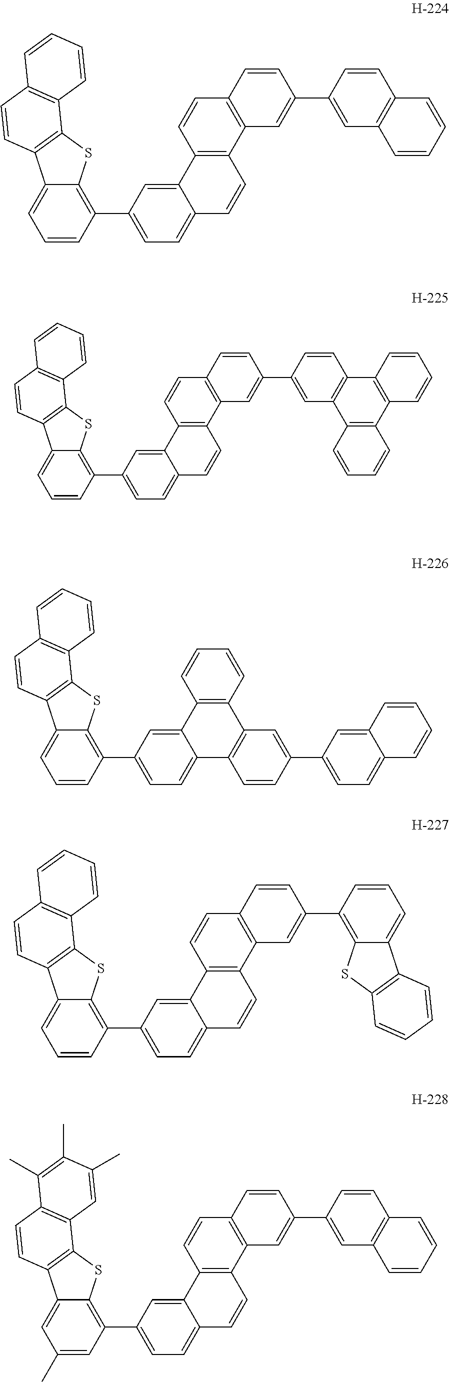 [SMILES: c1ccc2cc(-c3ccc4ccc5c6cc(-c7cccc8c7sc7c9ccccc9ccc87)ccc6ccc5c4c3)ccc2c1 ; c1ccc2c(c1)ccc1c3cccc(-c4ccc5ccc6c7cc(-c8ccc9c%10ccccc%10c%10ccccc%10c9c8)ccc7ccc6c5c4)c3sc21 ; c1ccc2cc(-c3ccc4c5ccc(-c6cccc7c6sc6c8ccccc8ccc76)cc5c5ccccc5c4c3)ccc2c1 ; c1ccc2c(c1)ccc1c3cccc(-c4ccc5ccc6c7cc(-c8cccc9c8sc8ccccc89)ccc7ccc6c5c4)c3sc21 ; Cc1cc(-c2ccc3ccc4c5cc(-c6ccc7ccccc7c6)ccc5ccc4c3c2)c2sc3c4cc(C)c(C)c(C)c4ccc3c2c1]

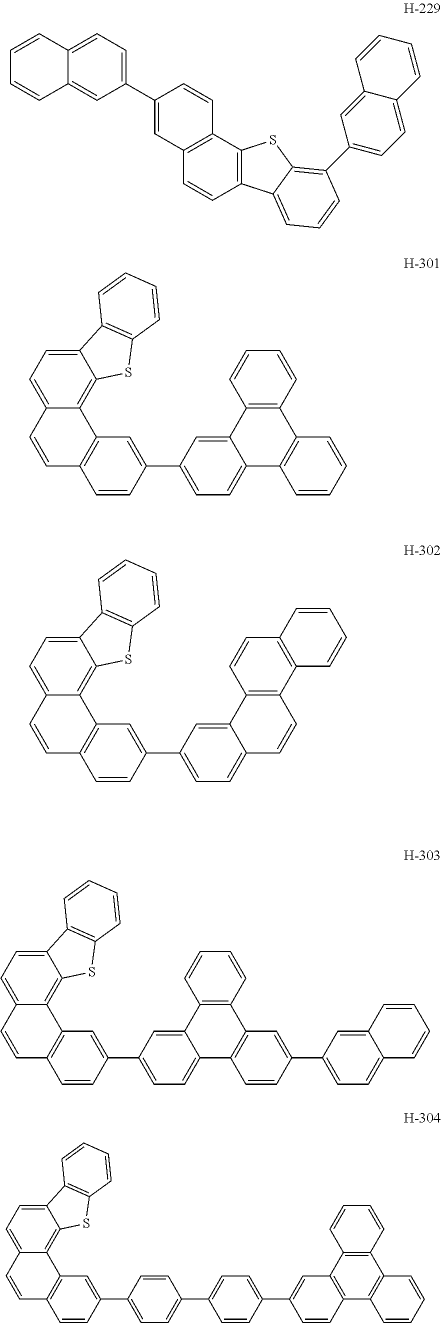 [SMILES: c1ccc2cc(-c3ccc4c(ccc5c6cccc(-c7ccc8ccccc8c7)c6sc45)c3)ccc2c1 ; c1ccc2c(c1)sc1c2ccc2ccc3ccc(-c4ccc5c6ccccc6c6ccccc6c5c4)cc3c21 ; c1ccc2c(c1)ccc1c3cc(-c4ccc5ccc6ccc7c8ccccc8sc7c6c5c4)ccc3ccc21 ; c1ccc2cc(-c3ccc4c5ccc(-c6ccc7ccc8ccc9c%10ccccc%10sc9c8c7c6)cc5c5ccccc5c4c3)ccc2c1 ; c1ccc2c(c1)sc1c2ccc2ccc3ccc(-c4ccc(-c5ccc(-c6ccc7c8ccccc8c8ccccc8c7c6)cc5)cc4)cc3c21]

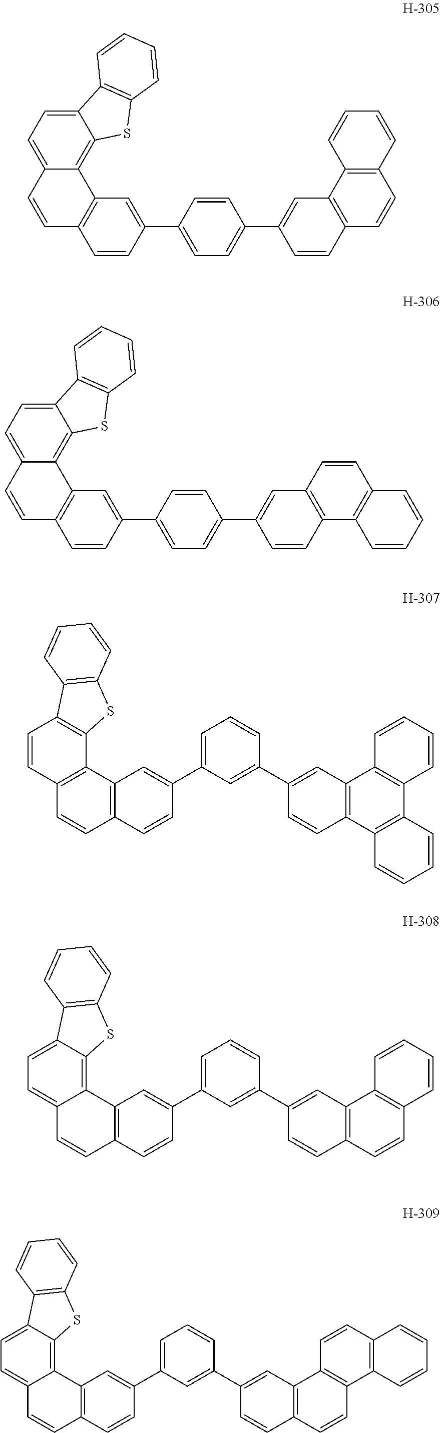 [SMILES: c1ccc2c(c1)ccc1ccc(-c3ccc(-c4ccc5ccc6ccc7c8ccccc8sc7c6c5c4)cc3)cc12 ; c1ccc2c(c1)ccc1cc(-c3ccc(-c4ccc5ccc6ccc7c8ccccc8sc7c6c5c4)cc3)ccc12 ; c1cc(-c2ccc3c4ccccc4c4ccccc4c3c2)cc(-c2ccc3ccc4ccc5c6ccccc6sc5c4c3c2)c1 ; c1cc(-c2ccc3ccc4ccccc4c3c2)cc(-c2ccc3ccc4ccc5c6ccccc6sc5c4c3c2)c1 ; c1cc(-c2ccc3ccc4c5ccccc5ccc4c3c2)cc(-c2ccc3ccc4ccc5c6ccccc6sc5c4c3c2)c1]

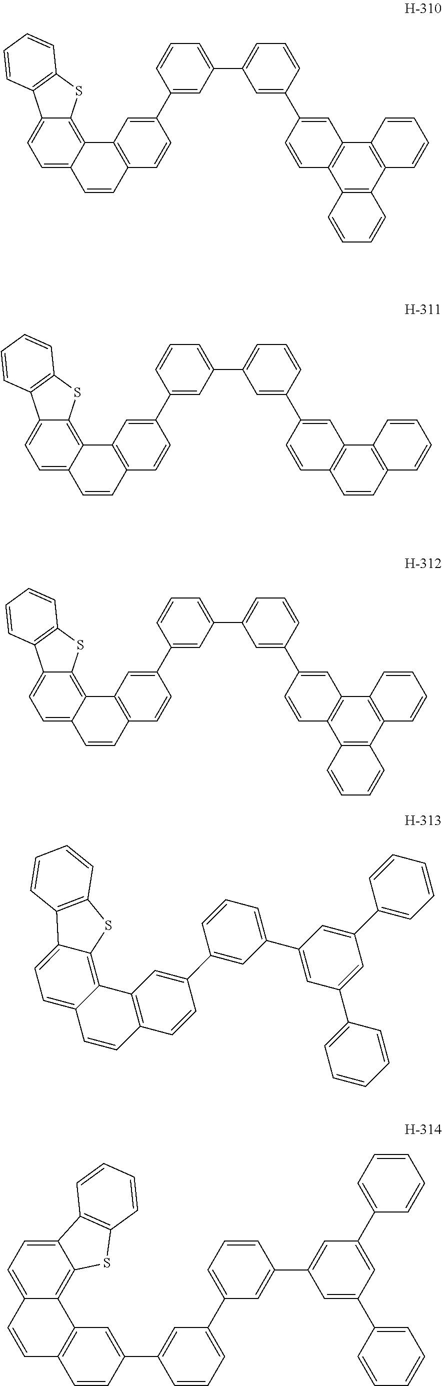 [SMILES: c1cc(-c2cccc(-c3ccc4ccc5ccc6c7ccccc7sc6c5c4c3)c2)cc(-c2ccc3c4ccccc4c4ccccc4c3c2)c1 ; c1cc(-c2cccc(-c3ccc4ccc5ccc6c7ccccc7sc6c5c4c3)c2)cc(-c2ccc3ccc4ccccc4c3c2)c1 ; c1cc(-c2cccc(-c3ccc4ccc5ccc6c7ccccc7sc6c5c4c3)c2)cc(-c2ccc3c4ccccc4c4ccccc4c3c2)c1 ; c1ccc(-c2cc(-c3ccccc3)cc(-c3cccc(-c4ccc5ccc6ccc7c8ccccc8sc7c6c5c4)c3)c2)cc1 ; c1ccc(-c2cc(-c3ccccc3)cc(-c3cccc(-c4cccc(-c5ccc6ccc7ccc8c9ccccc9sc8c7c6c5)c4)c3)c2)cc1]

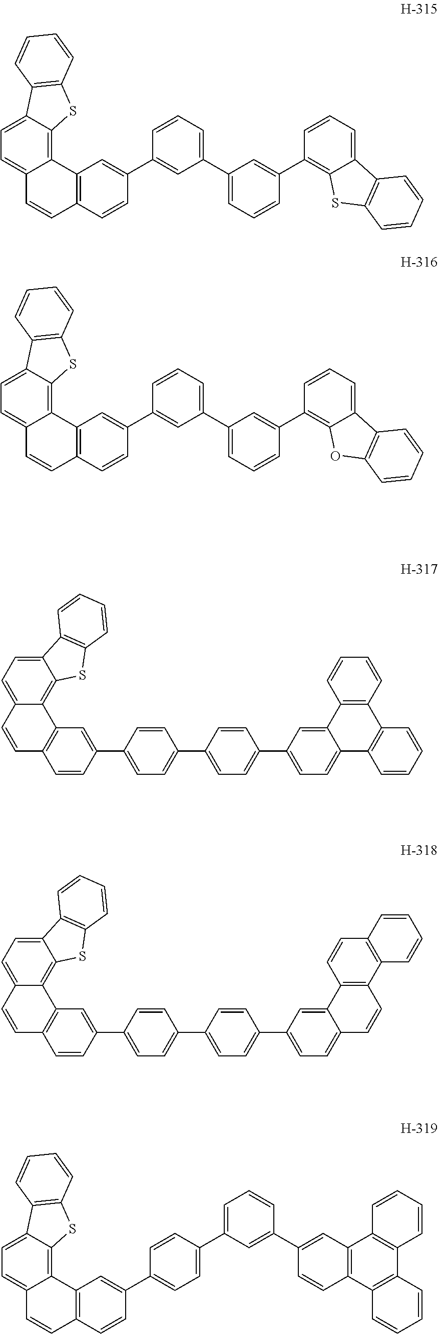 [SMILES: c1cc(-c2cccc(-c3cccc4c3sc3ccccc34)c2)cc(-c2ccc3ccc4ccc5c6ccccc6sc5c4c3c2)c1 ; c1cc(-c2cccc(-c3cccc4c3oc3ccccc34)c2)cc(-c2ccc3ccc4ccc5c6ccccc6sc5c4c3c2)c1 ; c1ccc2c(c1)sc1c2ccc2ccc3ccc(-c4ccc(-c5ccc(-c6ccc7c8ccccc8c8ccccc8c7c6)cc5)cc4)cc3c21 ; c1ccc2c(c1)ccc1c3cc(-c4ccc(-c5ccc(-c6ccc7ccc8ccc9c%10ccccc%10sc9c8c7c6)cc5)cc4)ccc3ccc21 ; c1cc(-c2ccc(-c3ccc4ccc5ccc6c7ccccc7sc6c5c4c3)cc2)cc(-c2ccc3c4ccccc4c4ccccc4c3c2)c1]

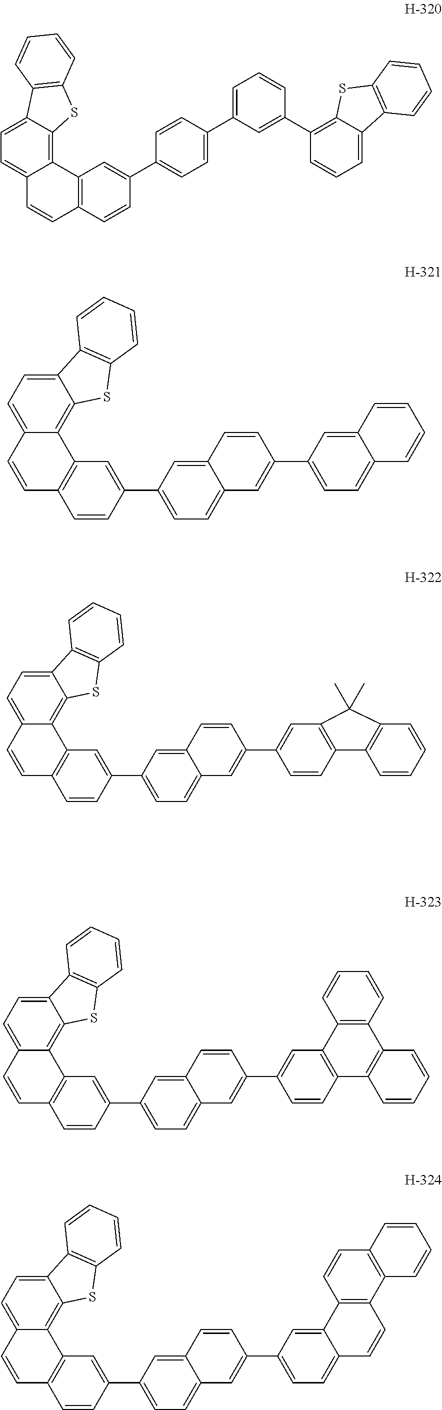 [SMILES: c1cc(-c2ccc(-c3ccc4ccc5ccc6c7ccccc7sc6c5c4c3)cc2)cc(-c2cccc3c2sc2ccccc23)c1 ; c1ccc2cc(-c3ccc4cc(-c5ccc6ccc7ccc8c9ccccc9sc8c7c6c5)ccc4c3)ccc2c1 ; CC1(C)c2ccccc2-c2ccc(-c3ccc4cc(-c5ccc6ccc7ccc8c9ccccc9sc8c7c6c5)ccc4c3)cc21 ; c1ccc2c(c1)sc1c2ccc2ccc3ccc(-c4ccc5cc(-c6ccc7c8ccccc8c8ccccc8c7c6)ccc5c4)cc3c21 ; c1ccc2c(c1)ccc1c3cc(-c4ccc5cc(-c6ccc7ccc8ccc9c%10ccccc%10sc9c8c7c6)ccc5c4)ccc3ccc21]

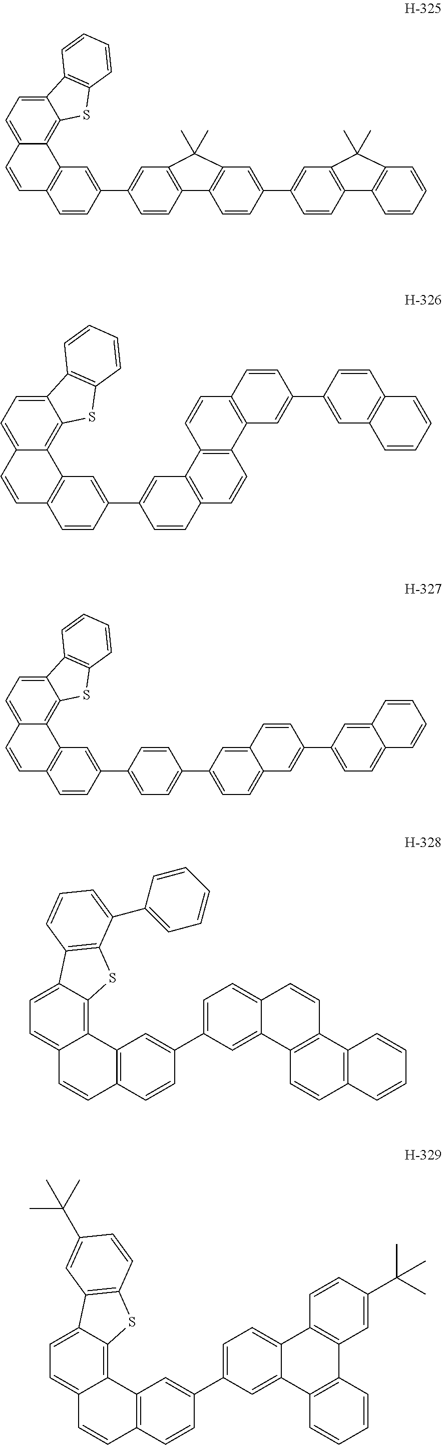 [SMILES: CC1(C)c2ccccc2-c2ccc(-c3ccc4c(c3)C(C)(C)c3cc(-c5ccc6ccc7ccc8c9ccccc9sc8c7c6c5)ccc3-4)cc21 ; c1ccc2cc(-c3ccc4ccc5c6cc(-c7ccc8ccc9ccc%10c%11ccccc%11sc%10c9c8c7)ccc6ccc5c4c3)ccc2c1 ; c1ccc2cc(-c3ccc4cc(-c5ccc(-c6ccc7ccc8ccc9c%10ccccc%10sc9c8c7c6)cc5)ccc4c3)ccc2c1 ; c1ccc(-c2cccc3c2sc2c3ccc3ccc4ccc(-c5ccc6ccc7c8ccccc8ccc7c6c5)cc4c32)cc1 ; CC(C)(C)c1ccc2sc3c(ccc4ccc5ccc(-c6ccc7c8ccc(C(C)(C)C)cc8c8ccccc8c7c6)cc5c43)c2c1]

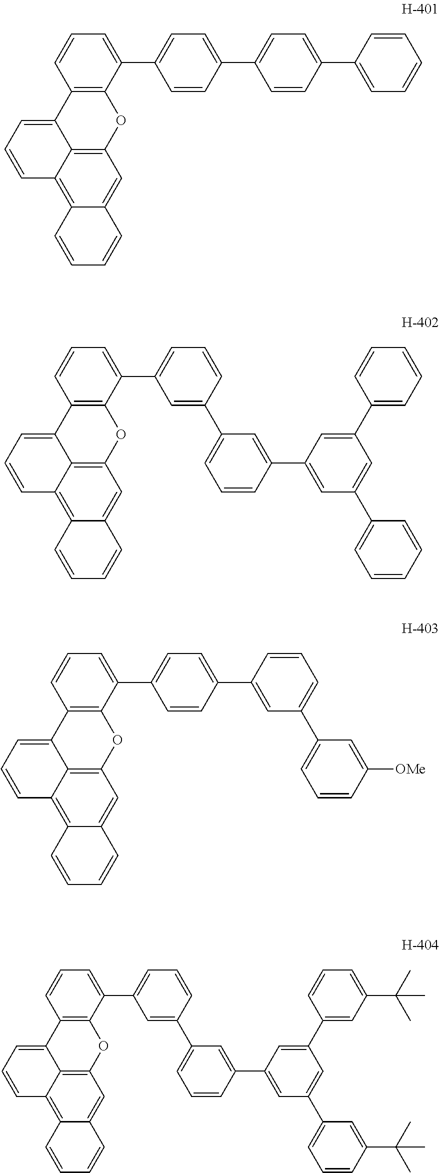 [SMILES: c1ccc(-c2ccc(-c3ccc(-c4cccc5c4Oc4cc6ccccc6c6cccc-5c46)cc3)cc2)cc1 ; c1ccc(-c2cc(-c3ccccc3)cc(-c3cccc(-c4cccc(-c5cccc6c5Oc5cc7ccccc7c7cccc-6c57)c4)c3)c2)cc1 ; COc1cccc(-c2cccc(-c3ccc(-c4cccc5c4Oc4cc6ccccc6c6cccc-5c46)cc3)c2)c1 ; CC(C)(C)c1cccc(-c2cc(-c3cccc(-c4cccc(-c5cccc6c5Oc5cc7ccccc7c7cccc-6c57)c4)c3)cc(-c3cccc(C(C)(C)C)c3)c2)c1]

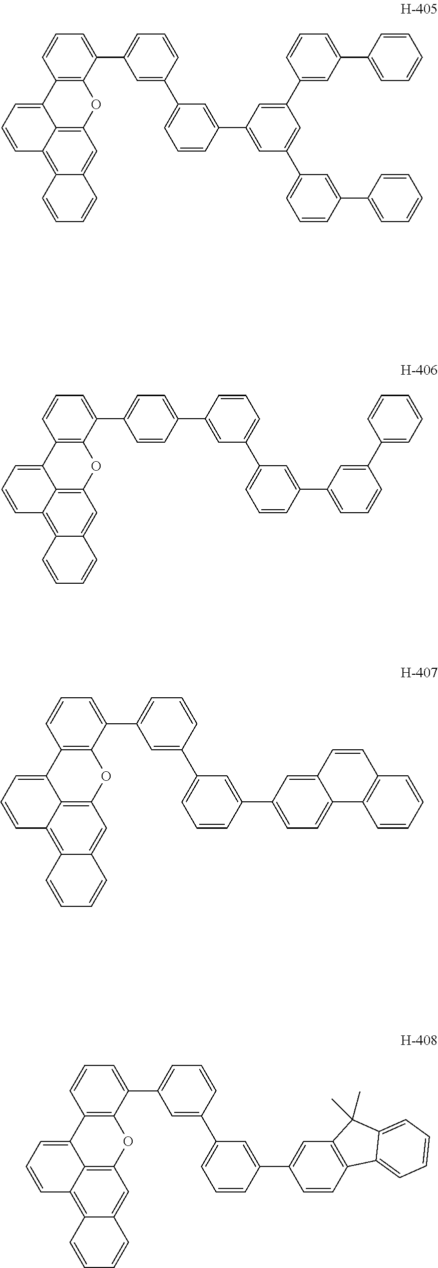 [SMILES: c1ccc(-c2cccc(-c3cc(-c4cccc(-c5ccccc5)c4)cc(-c4cccc(-c5cccc(-c6cccc7c6Oc6cc8ccccc8c8cccc-7c68)c5)c4)c3)c2)cc1 ; c1ccc(-c2cccc(-c3cccc(-c4cccc(-c5ccc(-c6cccc7c6Oc6cc8ccccc8c8cccc-7c68)cc5)c4)c3)c2)cc1 ; c1cc(-c2cccc(-c3cccc4c3Oc3cc5ccccc5c5cccc-4c35)c2)cc(-c2ccc3c(ccc4ccccc43)c2)c1 ; CC1(C)c2ccccc2-c2ccc(-c3cccc(-c4cccc(-c5cccc6c5Oc5cc7ccccc7c7cccc-6c57)c4)c3)cc21]

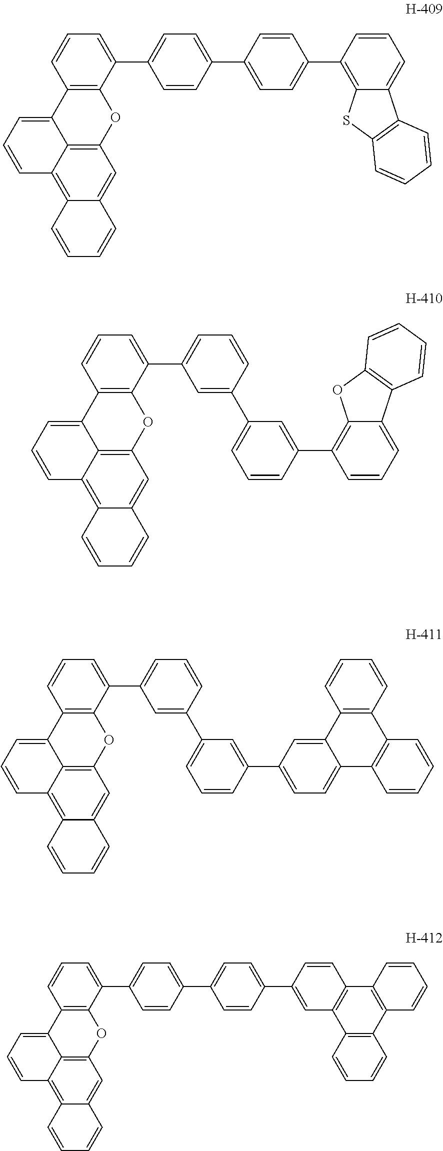 [SMILES: c1cc(-c2ccc(-c3ccc(-c4cccc5c4sc4ccccc45)cc3)cc2)c2c(c1)-c1cccc3c1c(cc1ccccc13)O2 ; c1cc(-c2cccc(-c3cccc4c3oc3ccccc34)c2)cc(-c2cccc3c2Oc2cc4ccccc4c4cccc-3c24)c1 ; c1cc(-c2cccc(-c3cccc4c3Oc3cc5ccccc5c5cccc-4c35)c2)cc(-c2ccc3c4ccccc4c4ccccc4c3c2)c1 ; c1cc(-c2ccc(-c3ccc(-c4ccc5c6ccccc6c6ccccc6c5c4)cc3)cc2)c2c(c1)-c1cccc3c1c(cc1ccccc13)O2]

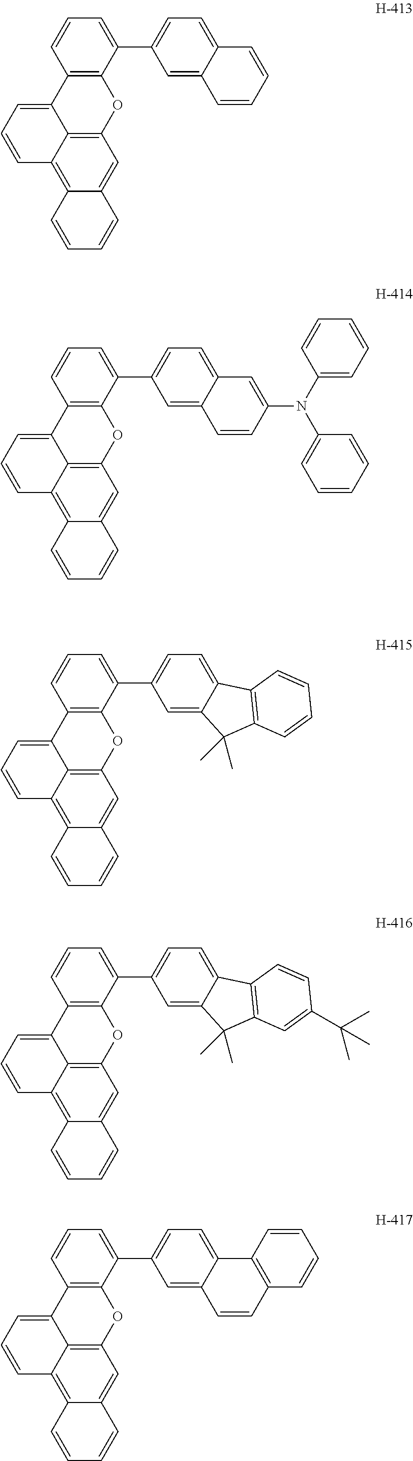 [SMILES: c1cc(-c2ccc3ccccc3c2)c2c(c1)-c1cccc3c1c(cc1ccccc13)O2 ; c1ccc(N(c2ccccc2)c2ccc3cc(-c4cccc5c4Oc4cc6ccccc6c6cccc-5c46)ccc3c2)cc1 ; CC1(C)c2ccccc2-c2ccc(-c3cccc4c3Oc3cc5ccccc5c5cccc-4c35)cc21 ; CC(C)(C)c1ccc2c(c1)C(C)(C)c1cc(-c3cccc4c3Oc3cc5ccccc5c5cccc-4c35)ccc1-2 ; c1cc(-c2ccc3c(ccc4ccccc43)c2)c2c(c1)-c1cccc3c1c(cc1ccccc13)O2]

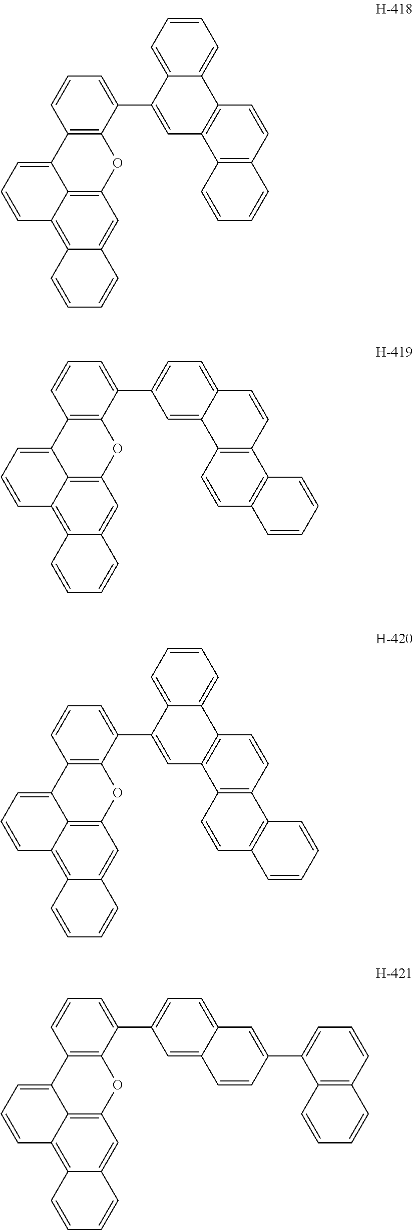 [SMILES: c1cc(-c2cc3c4ccccc4ccc3c3ccccc23)c2c(c1)-c1cccc3c1c(cc1ccccc13)O2 ; c1cc(-c2ccc3ccc4c5ccccc5ccc4c3c2)c2c(c1)-c1cccc3c1c(cc1ccccc13)O2 ; c1cc(-c2cc3c(ccc4c5ccccc5ccc43)c3ccccc23)c2c(c1)-c1cccc3c1c(cc1ccccc13)O2 ; c1cc(-c2ccc3cc(-c4cccc5ccccc45)ccc3c2)c2c(c1)-c1cccc3c1c(cc1ccccc13)O2]

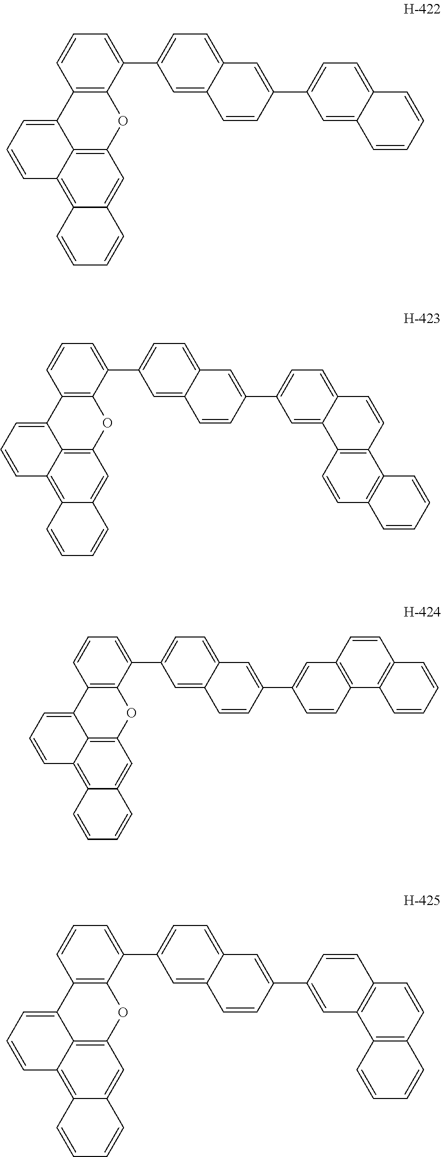 [SMILES: c1cc(-c2ccc3cc(-c4ccc5ccccc5c4)ccc3c2)c2c(c1)-c1cccc3c1c(cc1ccccc13)O2 ; c1cc(-c2ccc3cc(-c4ccc5ccc6c7ccccc7ccc6c5c4)ccc3c2)c2c(c1)-c1cccc3c1c(cc1ccccc13)O2 ; c1cc(-c2ccc3cc(-c4ccc5c(ccc6ccccc65)c4)ccc3c2)c2c(c1)-c1cccc3c1c(cc1ccccc13)O2 ; c1cc(-c2ccc3cc(-c4ccc5ccc6ccccc6c5c4)ccc3c2)c2c(c1)-c1cccc3c1c(cc1ccccc13)O2]

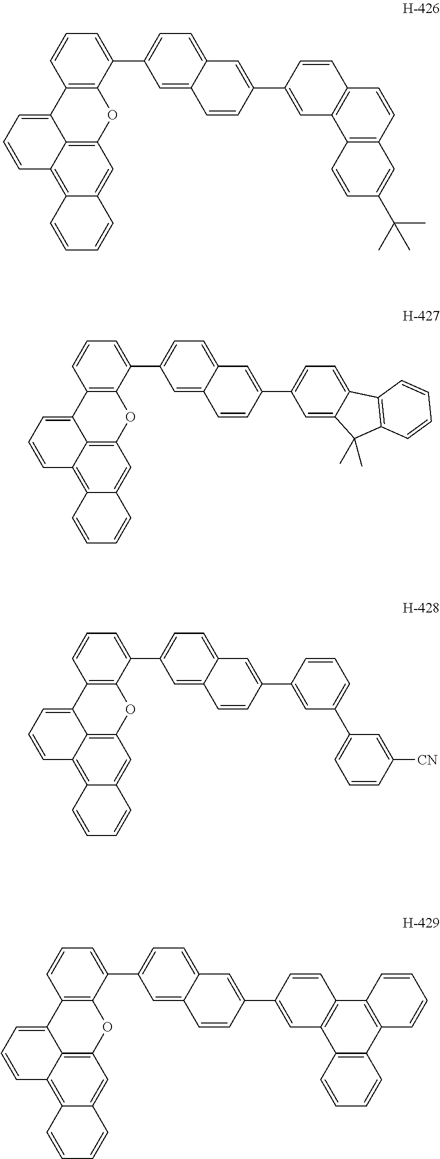 [SMILES: CC(C)(C)c1ccc2c(ccc3ccc(-c4ccc5cc(-c6cccc7c6Oc6cc8ccccc8c8cccc-7c68)ccc5c4)cc32)c1 ; CC1(C)c2ccccc2-c2ccc(-c3ccc4cc(-c5cccc6c5Oc5cc7ccccc7c7cccc-6c57)ccc4c3)cc21 ; N#Cc1cccc(-c2cccc(-c3ccc4cc(-c5cccc6c5Oc5cc7ccccc7c7cccc-6c57)ccc4c3)c2)c1 ; c1cc(-c2ccc3cc(-c4ccc5c6ccccc6c6ccccc6c5c4)ccc3c2)c2c(c1)-c1cccc3c1c(cc1ccccc13)O2]

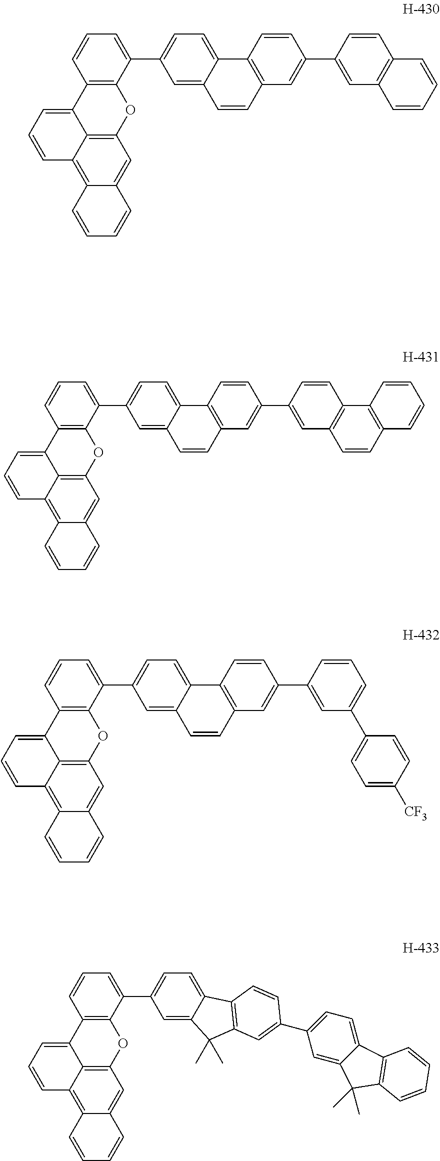 [SMILES: c1cc(-c2ccc3c(ccc4cc(-c5ccc6ccccc6c5)ccc43)c2)c2c(c1)-c1cccc3c1c(cc1ccccc13)O2 ; c1cc(-c2ccc3c(ccc4cc(-c5ccc6c(ccc7ccccc76)c5)ccc43)c2)c2c(c1)-c1cccc3c1c(cc1ccccc13)O2 ; FC(F)(F)c1ccc(-c2cccc(-c3ccc4c(ccc5cc(-c6cccc7c6Oc6cc8ccccc8c8cccc-7c68)ccc54)c3)c2)cc1 ; CC1(C)c2ccccc2-c2ccc(-c3ccc4c(c3)C(C)(C)c3cc(-c5cccc6c5Oc5cc7ccccc7c7cccc-6c57)ccc3-4)cc21]

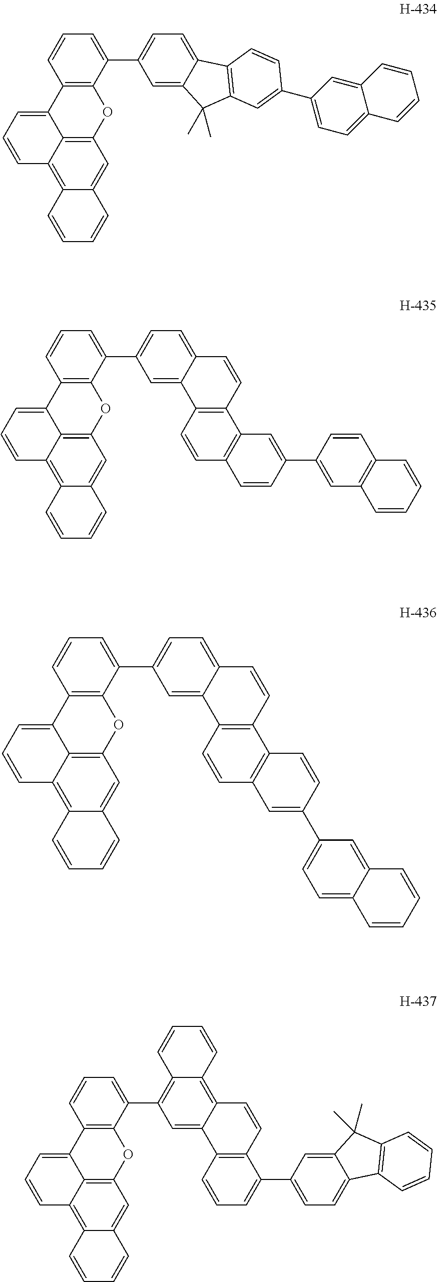 [SMILES: CC1(C)c2cc(-c3ccc4ccccc4c3)ccc2-c2ccc(-c3cccc4c3Oc3cc5ccccc5c5cccc-4c35)cc21 ; c1cc(-c2ccc3ccc4c5cc(-c6ccc7ccccc7c6)ccc5ccc4c3c2)c2c(c1)-c1cccc3c1c(cc1ccccc13)O2 ; c1cc(-c2ccc3ccc4c5ccc(-c6ccc7ccccc7c6)cc5ccc4c3c2)c2c(c1)-c1cccc3c1c(cc1ccccc13)O2 ; CC1(C)c2ccccc2-c2ccc(-c3cccc4c3ccc3c5ccccc5c(-c5cccc6c5Oc5cc7ccccc7c7cccc-6c57)cc43)cc21]

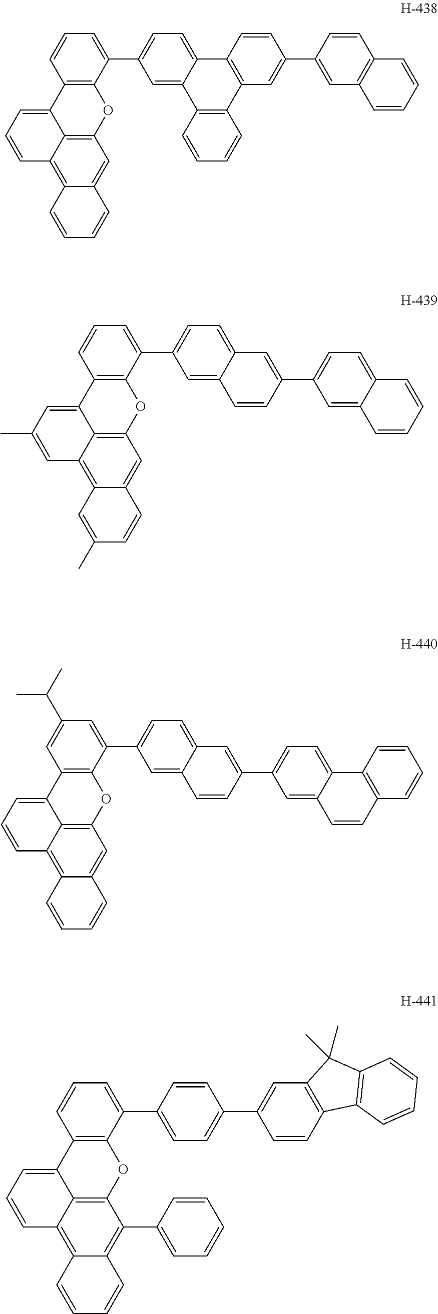 [SMILES: c1cc(-c2ccc3c4ccc(-c5ccc6ccccc6c5)cc4c4ccccc4c3c2)c2c(c1)-c1cccc3c1c(cc1ccccc13)O2 ; Cc1ccc2cc3c4c(cc(C)cc4c2c1)-c1cccc(-c2ccc4cc(-c5ccc6ccccc6c5)ccc4c2)c1O3 ; CC(C)c1cc(-c2ccc3cc(-c4ccc5c(ccc6ccccc65)c4)ccc3c2)c2c(c1)-c1cccc3c1c(cc1ccccc13)O2 ; CC1(C)c2ccccc2-c2ccc(-c3ccc(-c4cccc5c4Oc4c(-c6ccccc6)c6ccccc6c6cccc-5c46)cc3)cc21]

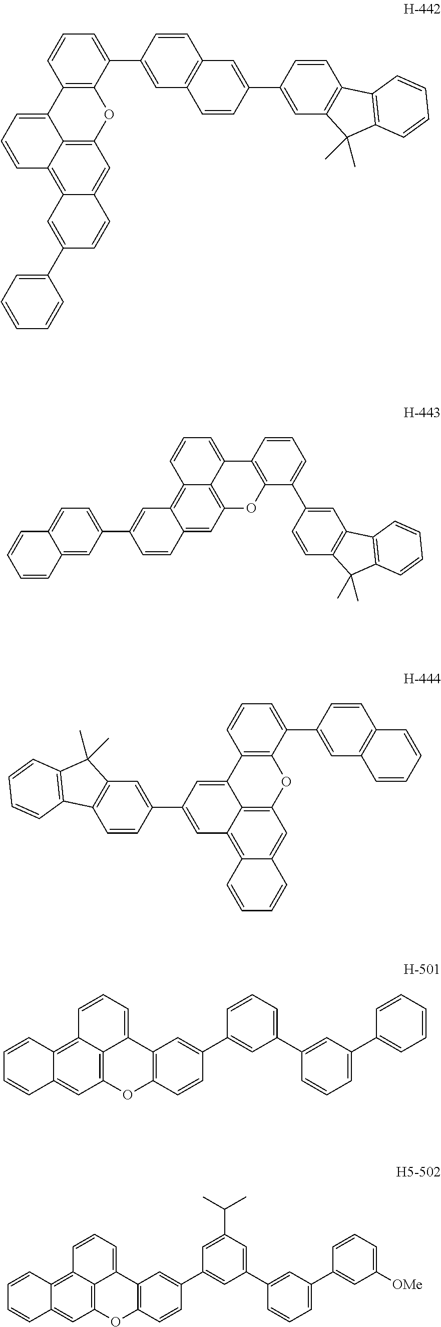 [SMILES: CC1(C)c2ccccc2-c2ccc(-c3ccc4cc(-c5cccc6c5Oc5cc7ccc(-c8ccccc8)cc7c7cccc-6c57)ccc4c3)cc21 ; CC1(C)c2ccccc2-c2cc(-c3cccc4c3Oc3cc5ccc(-c6ccc7ccccc7c6)cc5c5cccc-4c35)ccc21 ; CC1(C)c2ccccc2-c2ccc(-c3cc4c5c(cc6ccccc6c5c3)Oc3c(-c5ccc6ccccc6c5)cccc3-4)cc21 ; c1ccc(-c2cccc(-c3cccc(-c4ccc5c(c4)-c4cccc6c4c(cc4ccccc46)O5)c3)c2)cc1 ; COc1cccc(-c2cccc(-c3cc(-c4ccc5c(c4)-c4cccc6c4c(cc4ccccc46)O5)cc(C(C)C)c3)c2)c1]

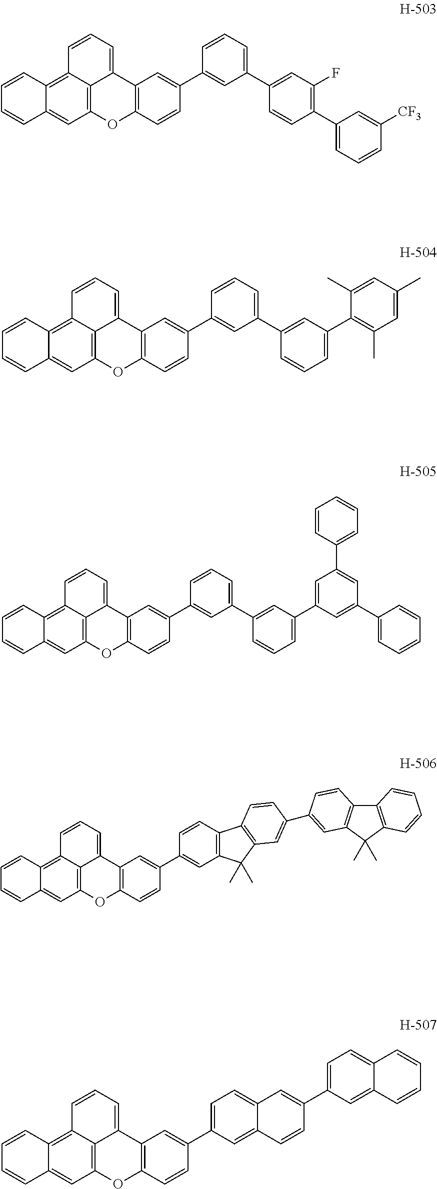 [SMILES: Fc1cc(-c2cccc(-c3ccc4c(c3)-c3cccc5c3c(cc3ccccc35)O4)c2)ccc1-c1cccc(C(F)(F)F)c1 ; Cc1cc(C)c(-c2cccc(-c3cccc(-c4ccc5c(c4)-c4cccc6c4c(cc4ccccc46)O5)c3)c2)c(C)c1 ; c1ccc(-c2cc(-c3ccccc3)cc(-c3cccc(-c4cccc(-c5ccc6c(c5)-c5cccc7c5c(cc5ccccc57)O6)c4)c3)c2)cc1 ; CC1(C)c2ccccc2-c2ccc(-c3ccc4c(c3)C(C)(C)c3cc(-c5ccc6c(c5)-c5cccc7c5c(cc5ccccc57)O6)ccc3-4)cc21 ; c1ccc2cc(-c3ccc4cc(-c5ccc6c(c5)-c5cccc7c5c(cc5ccccc57)O6)ccc4c3)ccc2c1]

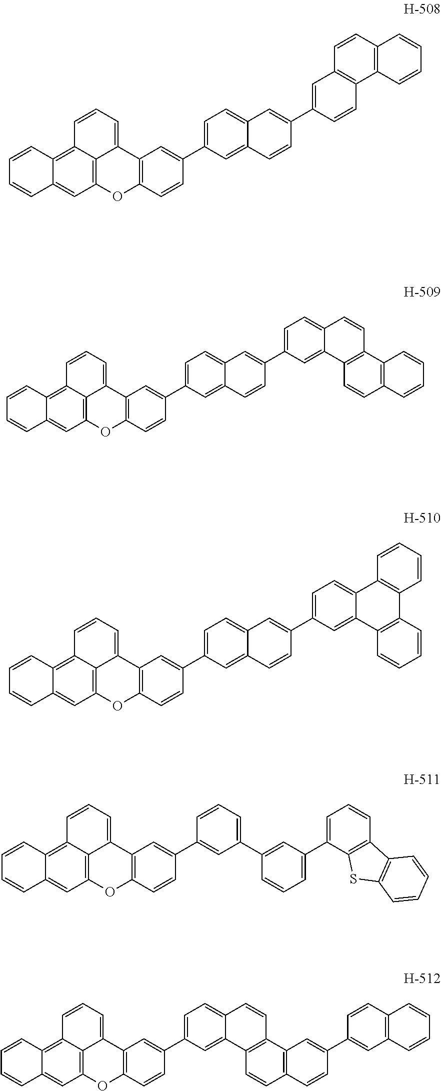 [SMILES: c1ccc2c(c1)ccc1cc(-c3ccc4cc(-c5ccc6c(c5)-c5cccc7c5c(cc5ccccc57)O6)ccc4c3)ccc12 ; c1ccc2c(c1)cc1c3c(cccc32)-c2cc(-c3ccc4cc(-c5ccc6ccc7c8ccccc8ccc7c6c5)ccc4c3)ccc2O1 ; c1ccc2c(c1)cc1c3c(cccc32)-c2cc(-c3ccc4cc(-c5ccc6c7ccccc7c7ccccc7c6c5)ccc4c3)ccc2O1 ; c1cc(-c2cccc(-c3cccc4c3sc3ccccc34)c2)cc(-c2ccc3c(c2)-c2cccc4c2c(cc2ccccc24)O3)c1 ; c1ccc2cc(-c3ccc4ccc5c6cc(-c7ccc8c(c7)-c7cccc9c7c(cc7ccccc79)O8)ccc6ccc5c4c3)ccc2c1]

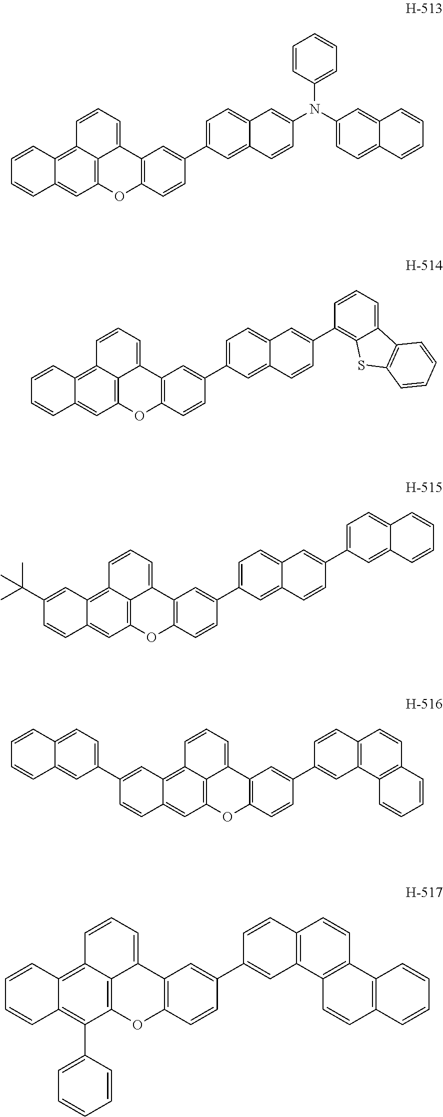 [SMILES: c1ccc(N(c2ccc3ccccc3c2)c2ccc3cc(-c4ccc5c(c4)-c4cccc6c4c(cc4ccccc46)O5)ccc3c2)cc1 ; c1ccc2c(c1)cc1c3c(cccc32)-c2cc(-c3ccc4cc(-c5cccc6c5sc5ccccc56)ccc4c3)ccc2O1 ; CC(C)(C)c1ccc2cc3c4c(cccc4c2c1)-c1cc(-c2ccc4cc(-c5ccc6ccccc6c5)ccc4c2)ccc1O3 ; c1ccc2cc(-c3ccc4cc5c6c(cccc6c4c3)-c3cc(-c4ccc6ccc7ccccc7c6c4)ccc3O5)ccc2c1 ; c1ccc(-c2c3c4c(cccc4c4ccccc24)-c2cc(-c4ccc5ccc6c7ccccc7ccc6c5c4)ccc2O3)cc1]

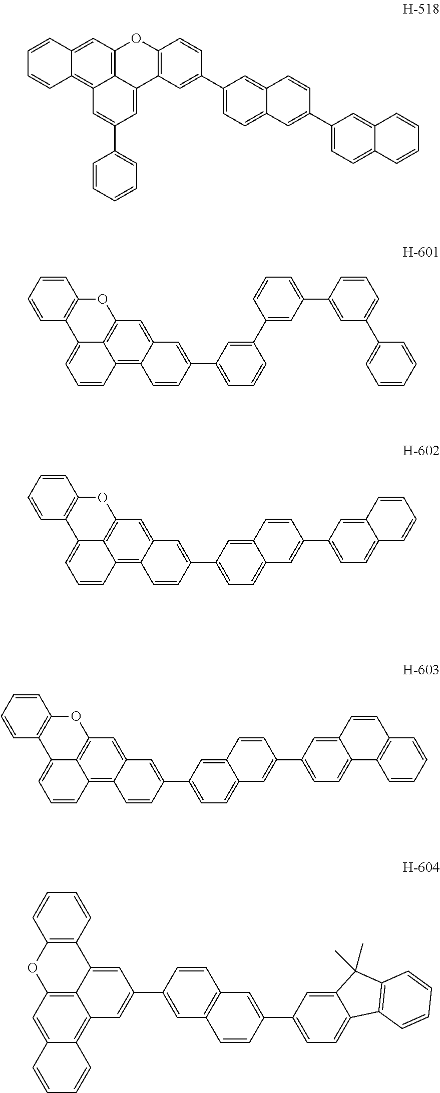 [SMILES: c1ccc(-c2cc3c4c(cc5ccccc5c4c2)Oc2ccc(-c4ccc5cc(-c6ccc7ccccc7c6)ccc5c4)cc2-3)cc1 ; c1ccc(-c2cccc(-c3cccc(-c4cccc(-c5ccc6c(c5)cc5c7c(cccc76)-c6ccccc6O5)c4)c3)c2)cc1 ; c1ccc2c(c1)Oc1cc3cc(-c4ccc5cc(-c6ccc7ccccc7c6)ccc5c4)ccc3c3cccc-2c13 ; c1ccc2c(c1)Oc1cc3cc(-c4ccc5cc(-c6ccc7c(ccc8ccccc87)c6)ccc5c4)ccc3c3cccc-2c13 ; CC1(C)c2ccccc2-c2ccc(-c3ccc4cc(-c5cc6c7c(cc8ccccc8c7c5)Oc5ccccc5-6)ccc4c3)cc21]

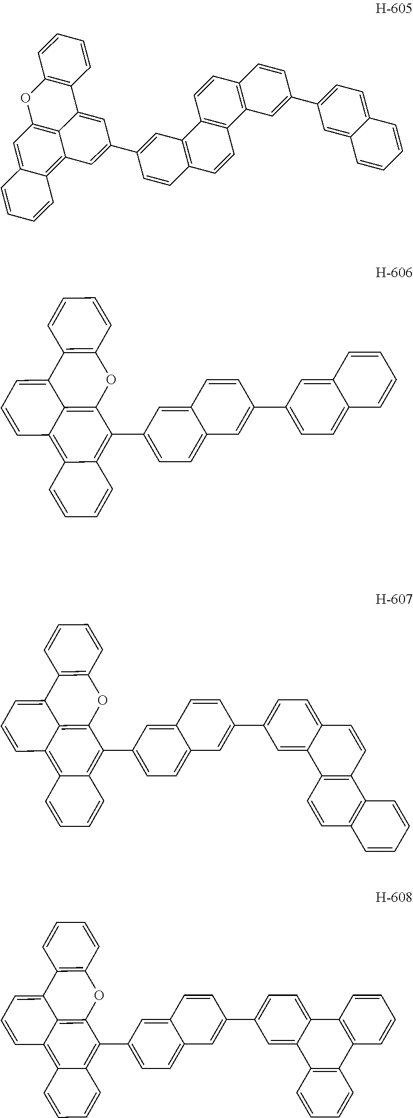 [SMILES: c1ccc2c(c1)Oc1cc3ccccc3c3cc(-c4ccc5ccc6c7cc(-c8ccc9ccccc9c8)ccc7ccc6c5c4)cc-2c13 ; c1ccc2c(c1)Oc1c(-c3ccc4cc(-c5ccc6ccccc6c5)ccc4c3)c3ccccc3c3cccc-2c13 ; c1ccc2c(c1)Oc1c(-c3ccc4cc(-c5ccc6ccc7c8ccccc8ccc7c6c5)ccc4c3)c3ccccc3c3cccc-2c13 ; c1ccc2c(c1)Oc1c(-c3ccc4cc(-c5ccc6c7ccccc7c7ccccc7c6c5)ccc4c3)c3ccccc3c3cccc-2c13]

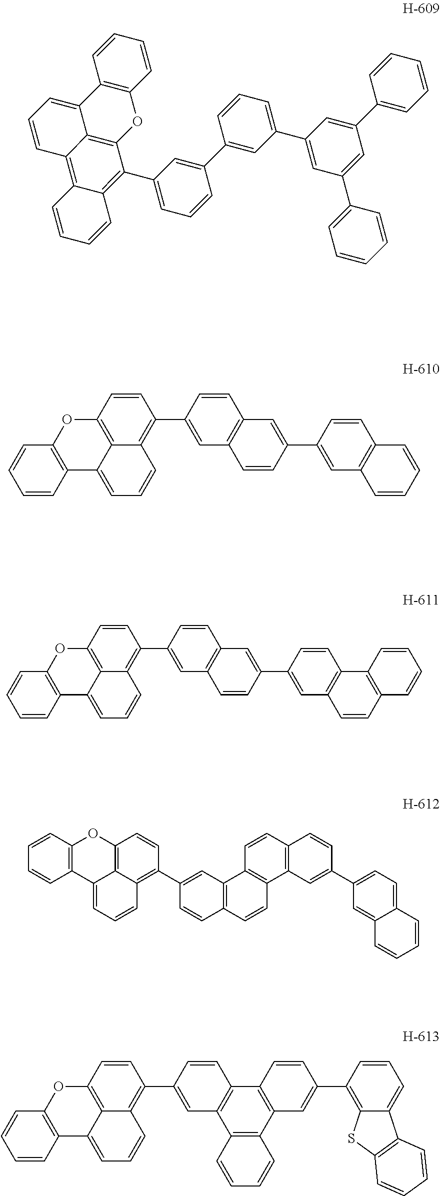 [SMILES: c1ccc(-c2cc(-c3ccccc3)cc(-c3cccc(-c4cccc(-c5c6c7c(cccc7c7ccccc57)-c5ccccc5O6)c4)c3)c2)cc1 ; c1ccc2c(c1)Oc1ccc(-c3ccc4cc(-c5ccc6ccccc6c5)ccc4c3)c3cccc-2c13 ; c1ccc2c(c1)Oc1ccc(-c3ccc4cc(-c5ccc6c(ccc7ccccc76)c5)ccc4c3)c3cccc-2c13 ; c1ccc2c(c1)Oc1ccc(-c3ccc4ccc5c6cc(-c7ccc8ccccc8c7)ccc6ccc5c4c3)c3cccc-2c13 ; c1ccc2c(c1)Oc1ccc(-c3ccc4c5ccc(-c6cccc7c6sc6ccccc67)cc5c5ccccc5c4c3)c3cccc-2c13]

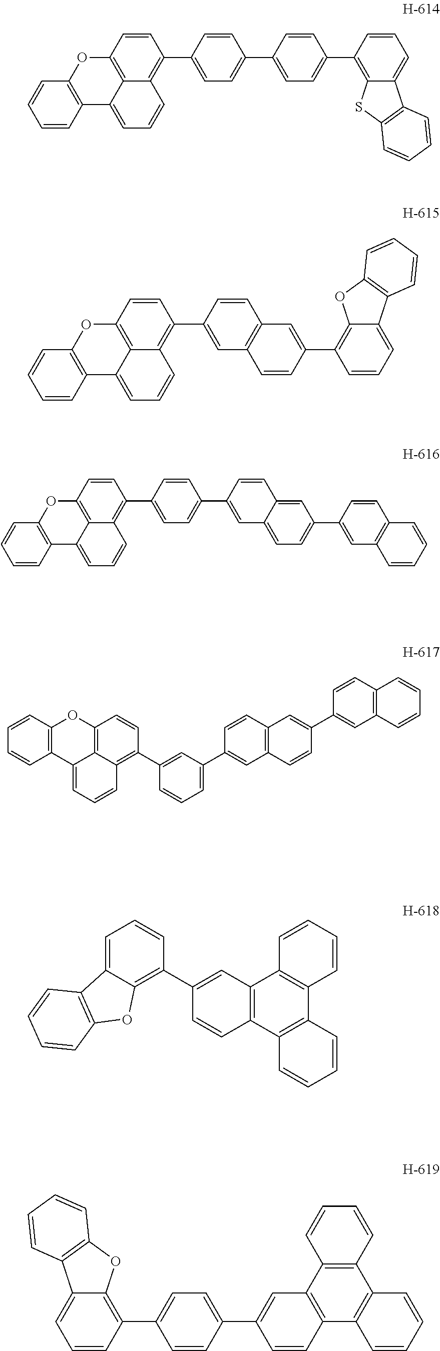 [SMILES: c1ccc2c(c1)Oc1ccc(-c3ccc(-c4ccc(-c5cccc6c5sc5ccccc56)cc4)cc3)c3cccc-2c13 ; c1ccc2c(c1)Oc1ccc(-c3ccc4cc(-c5cccc6c5oc5ccccc56)ccc4c3)c3cccc-2c13 ; c1ccc2c(c1)Oc1ccc(-c3ccc(-c4ccc5cc(-c6ccc7ccccc7c6)ccc5c4)cc3)c3cccc-2c13 ; c1cc(-c2ccc3cc(-c4ccc5ccccc5c4)ccc3c2)cc(-c2ccc3c4c(cccc24)-c2ccccc2O3)c1 ; c1ccc2c(c1)oc1c(-c3ccc4c5ccccc5c5ccccc5c4c3)cccc12 ; c1ccc2c(c1)oc1c(-c3ccc(-c4ccc5c6ccccc6c6ccccc6c5c4)cc3)cccc12]

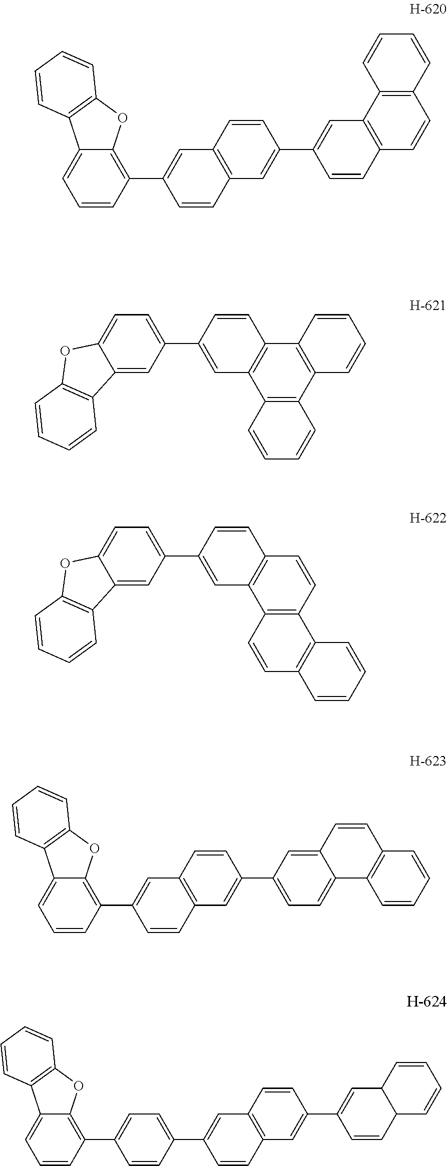 [SMILES: c1ccc2c(c1)ccc1ccc(-c3ccc4cc(-c5cccc6c5oc5ccccc56)ccc4c3)cc12 ; c1ccc2c(c1)oc1ccc(-c3ccc4c5ccccc5c5ccccc5c4c3)cc12 ; c1ccc2c(c1)ccc1c3cc(-c4ccc5oc6ccccc6c5c4)ccc3ccc21 ; c1ccc2c(c1)ccc1cc(-c3ccc4cc(-c5cccc6c5oc5ccccc56)ccc4c3)ccc12 ; C1=CC2C=CC(c3ccc4cc(-c5ccc(-c6cccc7c6oc6ccccc67)cc5)ccc4c3)=CC2C=C1]

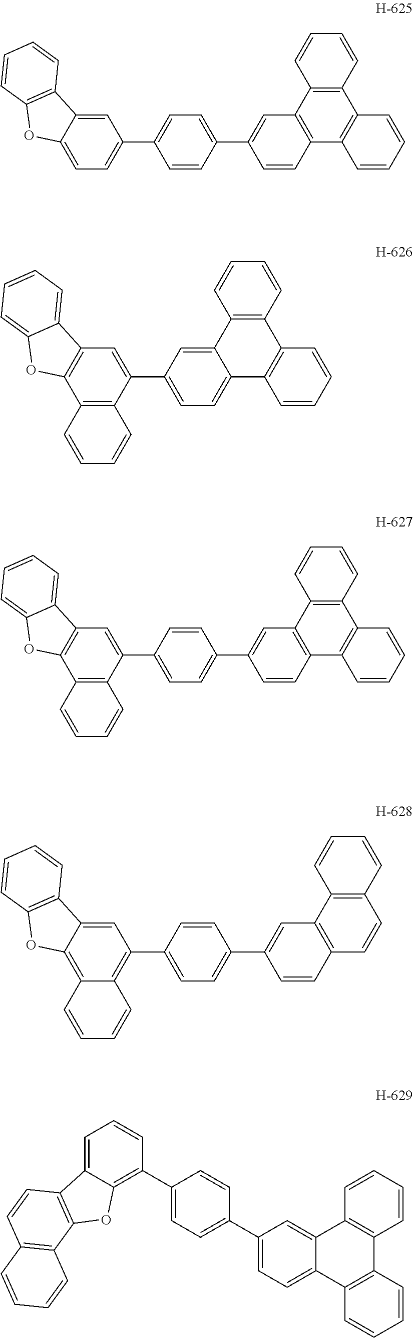 [SMILES: c1ccc2c(c1)oc1ccc(-c3ccc(-c4ccc5c6ccccc6c6ccccc6c5c4)cc3)cc12 ; c1ccc2c(c1)oc1c3ccccc3c(-c3ccc4c5ccccc5c5ccccc5c4c3)cc21 ; c1ccc2c(c1)oc1c3ccccc3c(-c3ccc(-c4ccc5c6ccccc6c6ccccc6c5c4)cc3)cc21 ; c1ccc2c(c1)ccc1ccc(-c3ccc(-c4cc5c6ccccc6oc5c5ccccc45)cc3)cc12 ; c1ccc2c(c1)ccc1c3cccc(-c4ccc(-c5ccc6c7ccccc7c7ccccc7c6c5)cc4)c3oc21]

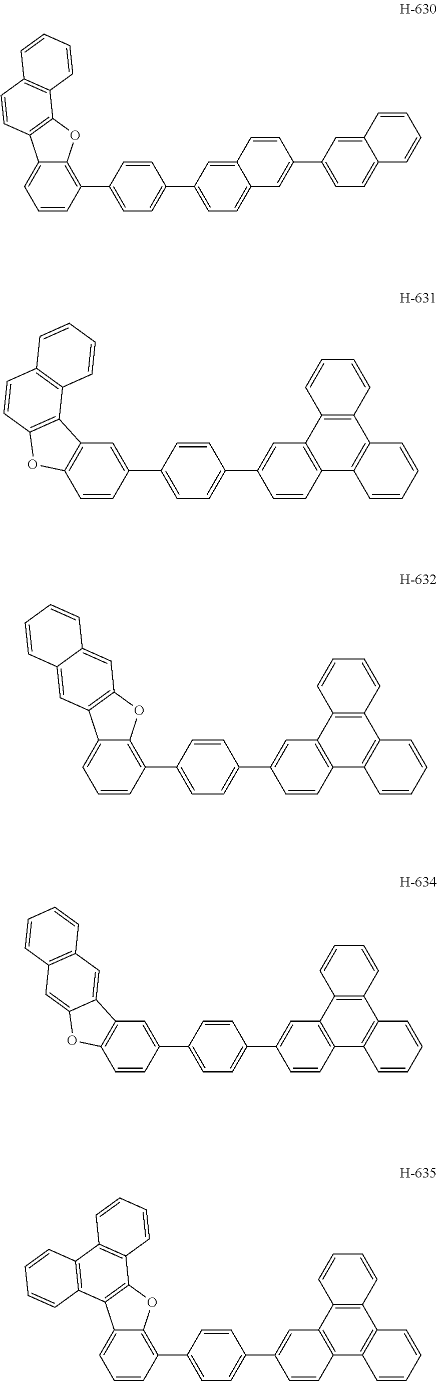 [SMILES: c1ccc2cc(-c3ccc4cc(-c5ccc(-c6cccc7c6oc6c8ccccc8ccc76)cc5)ccc4c3)ccc2c1 ; c1ccc2c(c1)ccc1oc3ccc(-c4ccc(-c5ccc6c7ccccc7c7ccccc7c6c5)cc4)cc3c12 ; c1ccc2cc3c(cc2c1)oc1c(-c2ccc(-c4ccc5c6ccccc6c6ccccc6c5c4)cc2)cccc13 ; c1ccc2cc3c(cc2c1)oc1ccc(-c2ccc(-c4ccc5c6ccccc6c6ccccc6c5c4)cc2)cc13 ; c1ccc2c(c1)c1ccccc1c1cc(-c3ccc(-c4cccc5c4oc4c6ccccc6c6ccccc6c54)cc3)ccc21]

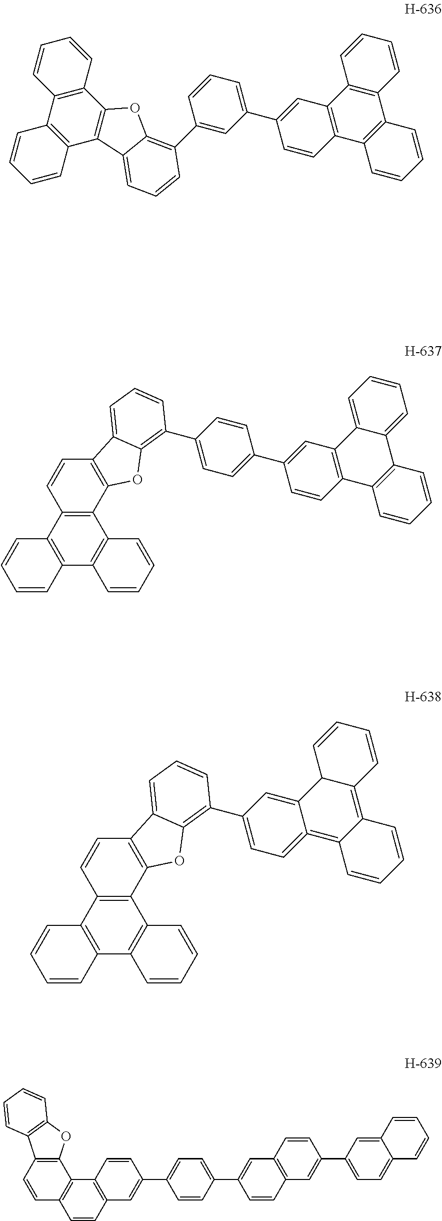

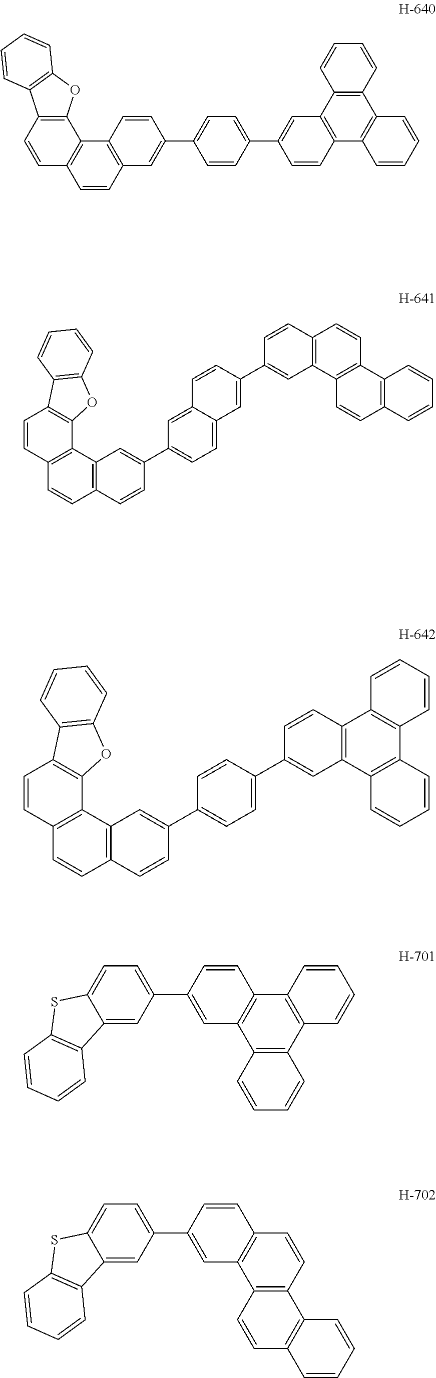 [SMILES: c1ccc2c(c1)oc1c2ccc2ccc3cc(-c4ccc(-c5ccc6c7ccccc7c7ccccc7c6c5)cc4)ccc3c21 ; c1ccc2c(c1)ccc1c3cc(-c4ccc5cc(-c6ccc7ccc8ccc9c%10ccccc%10oc9c8c7c6)ccc5c4)ccc3ccc21 ; c1ccc2c(c1)oc1c2ccc2ccc3ccc(-c4ccc(-c5ccc6c7ccccc7c7ccccc7c6c5)cc4)cc3c21 ; c1ccc2c(c1)sc1ccc(-c3ccc4c5ccccc5c5ccccc5c4c3)cc12 ; c1ccc2c(c1)ccc1c3cc(-c4ccc5sc6ccccc6c5c4)ccc3ccc21]

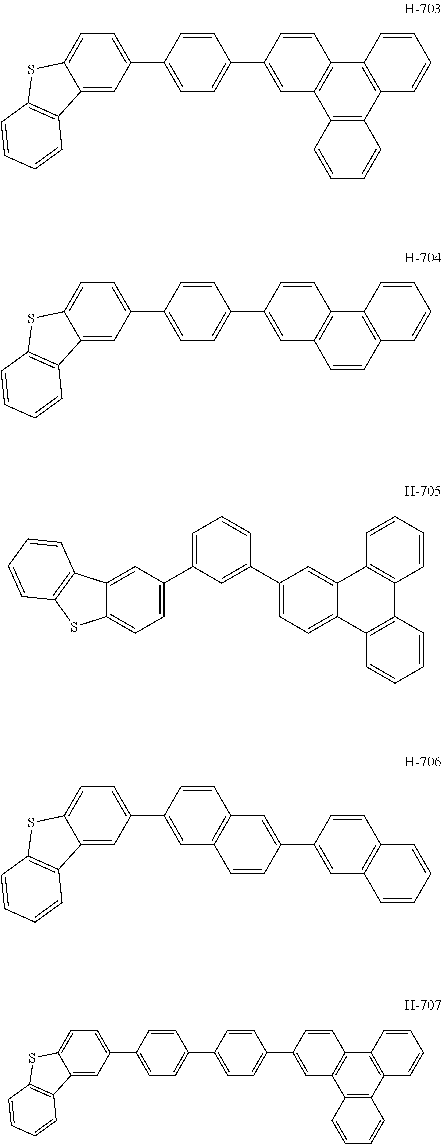 [SMILES: c1ccc2c(c1)sc1ccc(-c3ccc(-c4ccc5c6ccccc6c6ccccc6c5c4)cc3)cc12 ; c1ccc2c(c1)ccc1cc(-c3ccc(-c4ccc5sc6ccccc6c5c4)cc3)ccc12 ; c1cc(-c2ccc3sc4ccccc4c3c2)cc(-c2ccc3c4ccccc4c4ccccc4c3c2)c1 ; c1ccc2cc(-c3ccc4cc(-c5ccc6sc7ccccc7c6c5)ccc4c3)ccc2c1 ; c1ccc2c(c1)sc1ccc(-c3ccc(-c4ccc(-c5ccc6c7ccccc7c7ccccc7c6c5)cc4)cc3)cc12]

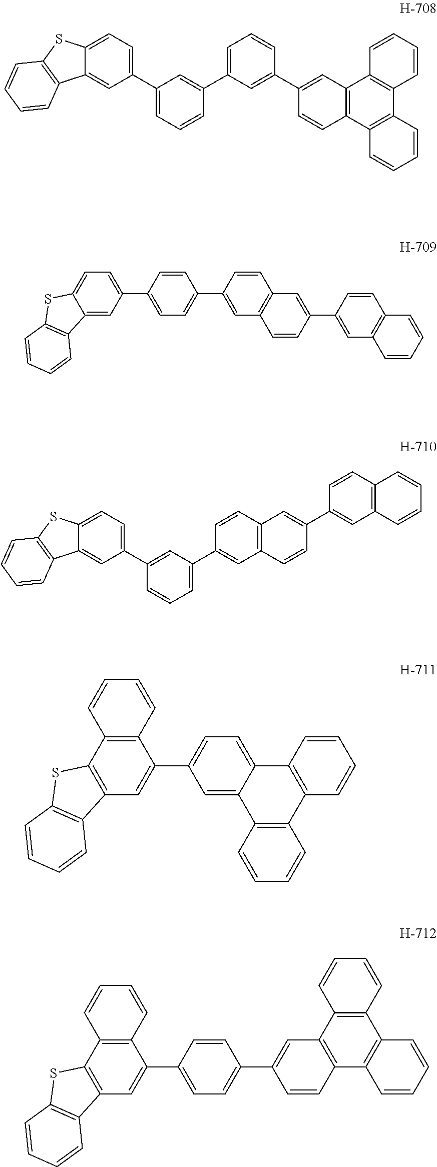 [SMILES: c1cc(-c2cccc(-c3ccc4c5ccccc5c5ccccc5c4c3)c2)cc(-c2ccc3sc4ccccc4c3c2)c1 ; c1ccc2cc(-c3ccc4cc(-c5ccc(-c6ccc7sc8ccccc8c7c6)cc5)ccc4c3)ccc2c1 ; c1cc(-c2ccc3cc(-c4ccc5ccccc5c4)ccc3c2)cc(-c2ccc3sc4ccccc4c3c2)c1 ; c1ccc2c(c1)sc1c3ccccc3c(-c3ccc4c5ccccc5c5ccccc5c4c3)cc21 ; c1ccc2c(c1)sc1c3ccccc3c(-c3ccc(-c4ccc5c6ccccc6c6ccccc6c5c4)cc3)cc21]

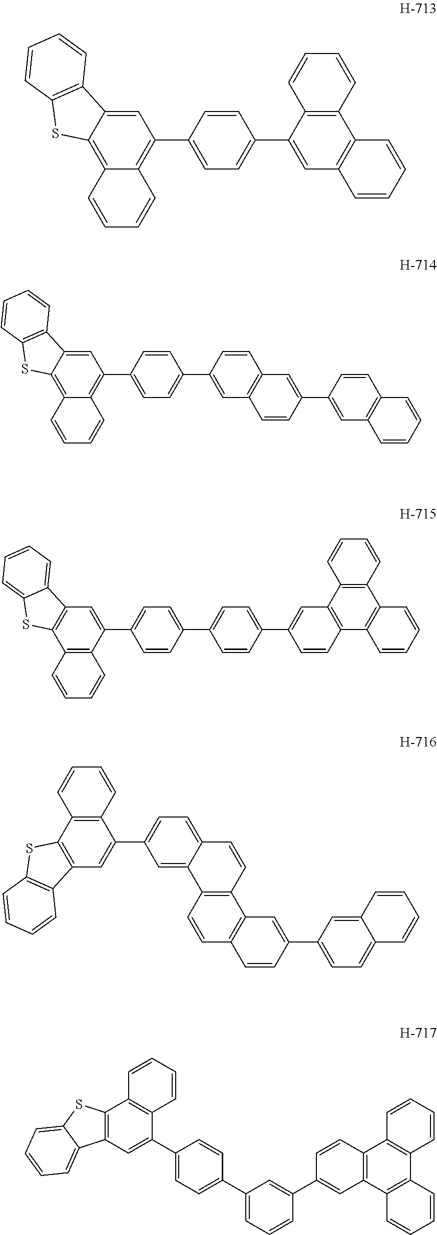 [SMILES: c1ccc2c(c1)cc(-c1ccc(-c3cc4c5ccccc5sc4c4ccccc34)cc1)c1ccccc12 ; c1ccc2cc(-c3ccc4cc(-c5ccc(-c6cc7c8ccccc8sc7c7ccccc67)cc5)ccc4c3)ccc2c1 ; c1ccc2c(c1)sc1c3ccccc3c(-c3ccc(-c4ccc(-c5ccc6c7ccccc7c7ccccc7c6c5)cc4)cc3)cc21 ; c1ccc2cc(-c3ccc4ccc5c6cc(-c7cc8c9ccccc9sc8c8ccccc78)ccc6ccc5c4c3)ccc2c1 ; c1cc(-c2ccc(-c3cc4c5ccccc5sc4c4ccccc34)cc2)cc(-c2ccc3c4ccccc4c4ccccc4c3c2)c1]

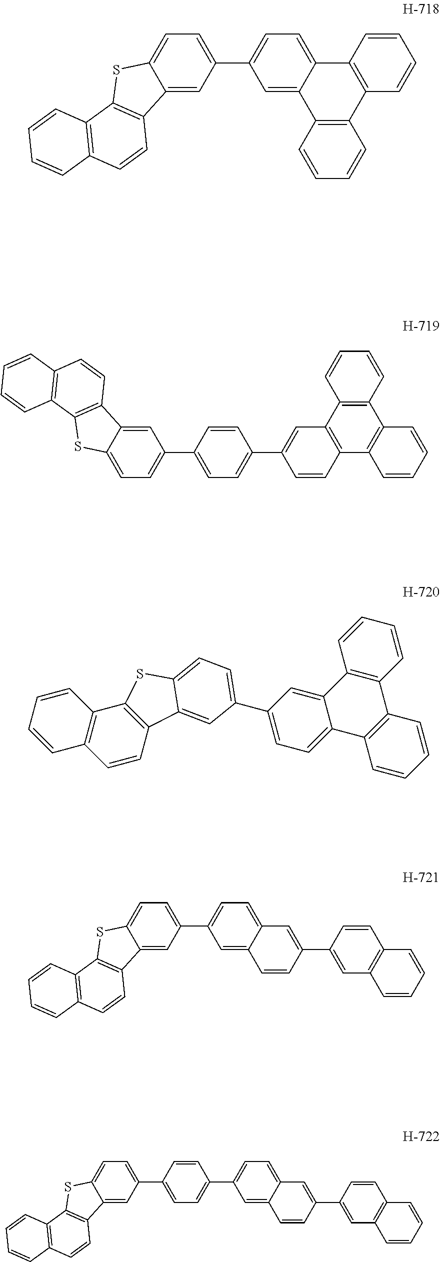 [SMILES: c1ccc2c(c1)ccc1c3cc(-c4ccc5c6ccccc6c6ccccc6c5c4)ccc3sc21 ; c1ccc2c(c1)ccc1c3cc(-c4ccc(-c5ccc6c7ccccc7c7ccccc7c6c5)cc4)ccc3sc21 ; c1ccc2c(c1)ccc1c3cc(-c4ccc5c6ccccc6c6ccccc6c5c4)ccc3sc21 ; c1ccc2cc(-c3ccc4cc(-c5ccc6sc7c8ccccc8ccc7c6c5)ccc4c3)ccc2c1 ; c1ccc2cc(-c3ccc4cc(-c5ccc(-c6ccc7sc8c9ccccc9ccc8c7c6)cc5)ccc4c3)ccc2c1]

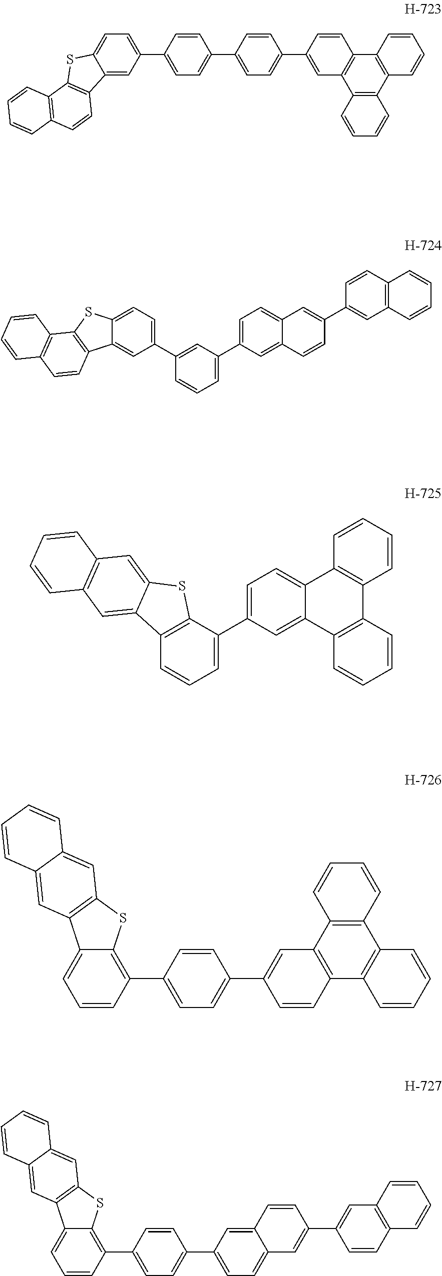 [SMILES: c1ccc2c(c1)ccc1c3cc(-c4ccc(-c5ccc(-c6ccc7c8ccccc8c8ccccc8c7c6)cc5)cc4)ccc3sc21 ; c1cc(-c2ccc3cc(-c4ccc5ccccc5c4)ccc3c2)cc(-c2ccc3sc4c5ccccc5ccc4c3c2)c1 ; c1ccc2cc3c(cc2c1)sc1c(-c2ccc4c5ccccc5c5ccccc5c4c2)cccc13 ; c1ccc2cc3c(cc2c1)sc1c(-c2ccc(-c4ccc5c6ccccc6c6ccccc6c5c4)cc2)cccc13 ; c1ccc2cc(-c3ccc4cc(-c5ccc(-c6cccc7c6sc6cc8ccccc8cc67)cc5)ccc4c3)ccc2c1]

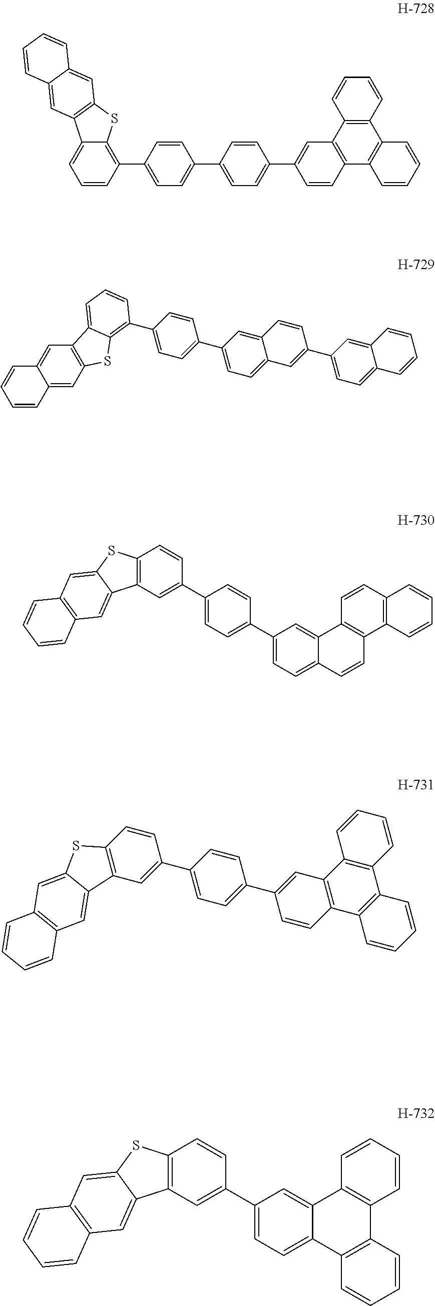 [SMILES: c1ccc2cc3c(cc2c1)sc1c(-c2ccc(-c4ccc(-c5ccc6c7ccccc7c7ccccc7c6c5)cc4)cc2)cccc13 ; c1ccc2cc(-c3ccc4cc(-c5ccc(-c6cccc7c6sc6cc8ccccc8cc67)cc5)ccc4c3)ccc2c1 ; c1ccc2cc3c(cc2c1)sc1ccc(-c2ccc(-c4ccc5ccc6c7ccccc7ccc6c5c4)cc2)cc13 ; c1ccc2cc3c(cc2c1)sc1ccc(-c2ccc(-c4ccc5c6ccccc6c6ccccc6c5c4)cc2)cc13 ; c1ccc2cc3c(cc2c1)sc1ccc(-c2ccc4c5ccccc5c5ccccc5c4c2)cc13]

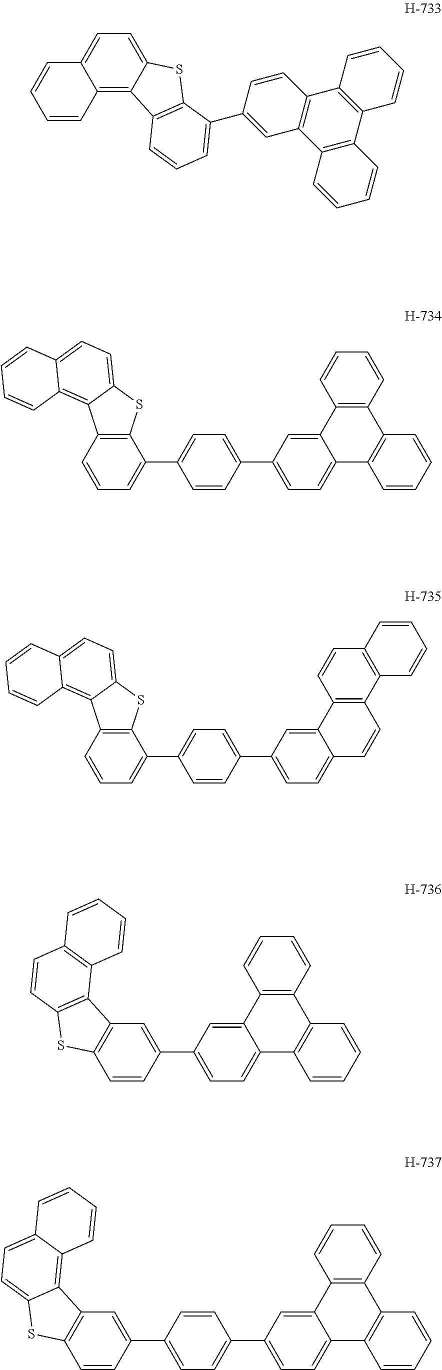 [SMILES: c1ccc2c(c1)ccc1sc3c(-c4ccc5c6ccccc6c6ccccc6c5c4)cccc3c12 ; c1ccc2c(c1)ccc1sc3c(-c4ccc(-c5ccc6c7ccccc7c7ccccc7c6c5)cc4)cccc3c12 ; c1ccc2c(c1)ccc1c3cc(-c4ccc(-c5cccc6c5sc5ccc7ccccc7c56)cc4)ccc3ccc21 ; c1ccc2c(c1)ccc1sc3ccc(-c4ccc5c6ccccc6c6ccccc6c5c4)cc3c12 ; c1ccc2c(c1)ccc1sc3ccc(-c4ccc(-c5ccc6c7ccccc7c7ccccc7c6c5)cc4)cc3c12]

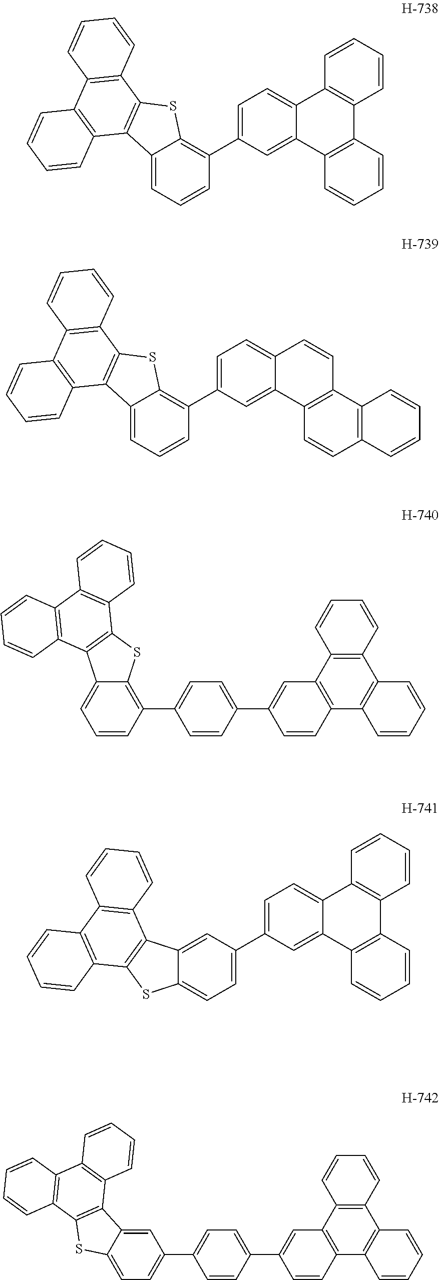 [SMILES: c1ccc2c(c1)c1ccccc1c1cc(-c3cccc4c3sc3c5ccccc5c5ccccc5c43)ccc21 ; c1ccc2c(c1)ccc1c3cc(-c4cccc5c4sc4c6ccccc6c6ccccc6c54)ccc3ccc21 ; c1ccc2c(c1)c1ccccc1c1cc(-c3ccc(-c4cccc5c4sc4c6ccccc6c6ccccc6c54)cc3)ccc21 ; c1ccc2c(c1)c1ccccc1c1cc(-c3ccc4sc5c6ccccc6c6ccccc6c5c4c3)ccc21 ; c1ccc2c(c1)c1ccccc1c1cc(-c3ccc(-c4ccc5sc6c7ccccc7c7ccccc7c6c5c4)cc3)ccc21]

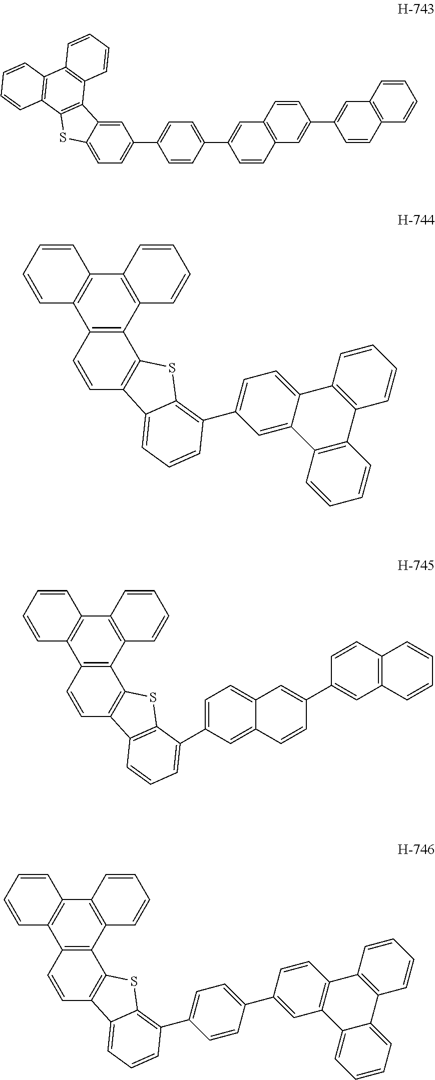 [SMILES: c1ccc2cc(-c3ccc4cc(-c5ccc(-c6ccc7sc8c9ccccc9c9ccccc9c8c7c6)cc5)ccc4c3)ccc2c1 ; c1cc(-c2ccc3c4ccccc4c4ccccc4c3c2)c2sc3c(ccc4c5ccccc5c5ccccc5c43)c2c1 ; c1ccc2cc(-c3ccc4cc(-c5cccc6c5sc5c6ccc6c7ccccc7c7ccccc7c65)ccc4c3)ccc2c1 ; c1cc(-c2ccc(-c3ccc4c5ccccc5c5ccccc5c4c3)cc2)c2sc3c(ccc4c5ccccc5c5ccccc5c43)c2c1]

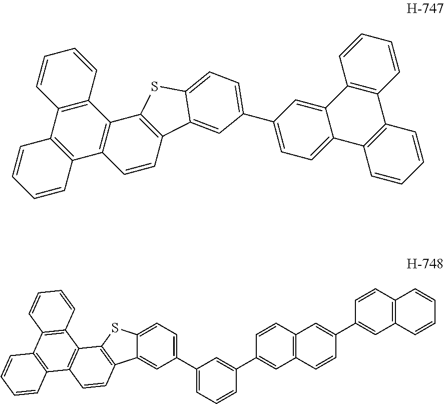 [SMILES: c1ccc2c(c1)c1ccccc1c1cc(-c3ccc4sc5c(ccc6c7ccccc7c7ccccc7c65)c4c3)ccc21 ; c1cc(-c2ccc3cc(-c4ccc5ccccc5c4)ccc3c2)cc(-c2ccc3sc4c(ccc5c6ccccc6c6ccccc6c54)c3c2)c1]

Of the exemplified compounds, the heterocycle-containing compounds represented by X-101 to X-140 are each a carbazole compound represented by the general formula [8]. Those heterocycle-containing compounds in the group 1 each have a moderately low hole mobility and high structural stability because the advantage of carbazole has been brought into play. Therefore, the incorporation of any one of those heterocycle-containing compounds in the group 1 as the host into the emission layer optimizes a carrier balance between the host and guest (iridium complex represented by the general formula [1]) in the emission layer. Therefore, an organic light-emitting device having high emission efficiency and a long lifetime is obtained.

Of the exemplified compounds, the heterocycle-containing compounds represented by H-101 to H-158 are each a dibenzothiophene compound represented by the general formula [9]. Those heterocycle-containing compounds in the group 2 each have a moderately low hole mobility and high structural stability because the advantage of dibenzothiophene has been brought into play. Therefore, as in the heterocycle-containing compounds in the group 1, the incorporation of any one of those heterocycle-containing compounds in the group 2 as the host into the emission layer optimizes the carrier balance between the host and guest (iridium complex represented by the general formula [1]) in the emission layer. Therefore, an organic light-emitting device having high emission efficiency and a long lifetime is obtained.

Of the exemplified compounds, the heterocycle-containing compounds represented by H-201 to H-229 are each a benzonaphthothiophene compound represented by the general formula [10]. As in the heterocycle-containing compounds in the group 1 and the group 2, those heterocycle-containing compounds in the group 3 can each also optimize the carrier balance between the host and guest (iridium complex represented by the general formula [1]) in the emission layer. Therefore, an organic light-emitting device having high emission efficiency and a long lifetime is obtained. In addition, the S₁ energy (HOMO-LUMO energy gap) of each heterocycle-containing compound in the group 3 is smaller than that of each heterocycle-containing compound in the group 2 because the n conjugation of benzonaphthothiophene is larger than that of dibenzothiophene. Therefore, the incorporation of the compound as the host into the emission layer can reduce the driving voltage of the light-emitting device because the introduction reduces a carrier injection barrier from the carrier-transporting layer.

Of the exemplified compounds, the heterocycle-containing compounds represented by H-301 to H-329 are each a benzophenanthrothiophene compound represented by the general formula [11]. As in the heterocycle-containing compounds in the group 1 and the group 3, those heterocycle-containing compounds in the group 4 can each also optimize the carrier balance between the host and guest (iridium complex represented by the general formula [1]) in the emission layer. Therefore, an organic light-emitting device having high emission efficiency and a long lifetime is obtained. In addition, the n conjugation of benzophenanthrothiophene is larger than those of benzonaphthothiophene and dibenzothiophene. Therefore, for the same reason as described above, the driving voltage of the light-emitting device can be reduced more.

Of the exemplified compounds, the heterocycle-containing compounds represented by H-401 to H-444 are each a dibenzoxanthene compound represented by the general formula [12]. Those heterocycle-containing compounds in the group 5 each have a moderately low hole mobility, high structural stability, and a relatively shallow HOMO level because the advantage of dibenzoxanthene has been brought into play. As in the heterocycle-containing compounds in the group 1 to the group 4, the incorporation of any one of those heterocycle-containing compounds in the group 5 as the host into the emission layer can also optimize the carrier balance between the host and guest (iridium complex represented by the general formula [1]) in the emission layer. Therefore, an organic light-emitting device having high emission efficiency and a long lifetime is obtained.

Of the exemplified compounds, the heterocycle-containing compounds represented by H-501 to H-518 are each a dibenzoxanthene compound represented by the general formula [13]. As in the heterocycle-containing compounds in the group 5, the incorporation of any one of those heterocycle-containing compounds in the group 6 as the host into the emission layer can also optimize the carrier balance between the host and guest (iridium complex represented by the general formula [1]) in the emission layer. Therefore, an organic light-emitting device having high emission efficiency and a long lifetime is obtained.

Of the exemplified compounds, the heterocycle-containing compounds represented by H-601 to H-642 are each a compound having an oxygen-containing heterocycle in which Z represents an oxygen atom out of the heterocycle-containing compounds each represented by the general formula [7]. In this regard, the compounds in the group (group 7) are each an oxygen-containing heterocycle-containing compound except the dibenzoxanthene compounds represented by the general formulae [12] and [13]. Those heterocycle-containing compounds in the group 7 are each a compound having high structural stability as in the heterocycle-containing compounds in the group 1 to the group 6, and are each a compound having a relatively shallow HOMO level because the electron-donating property of the oxygen atom comes into play. As in the heterocycle-containing compounds in the group 1 to the group 6, the incorporation of any one of those heterocycle-containing compounds in the group 7 as the host into the emission layer can also optimize the carrier balance between the host and guest (iridium complex represented by the general formula [1]) in the emission layer. Therefore, an organic light-emitting device having high emission efficiency and a long lifetime is obtained.

Of the exemplified compounds, the heterocycle-containing compounds represented by H-701 to H-748 are each a compound in which Z in the formula [7] represents a sulfur atom, and that does not correspond to the benzo-fused thiophene compounds represented by the general formulae [9] to [11] out of the heterocycle-containing compounds each represented by the general formula [7]. As in the heterocycle-containing compounds in the group 1 to the group 6, those heterocycle-containing compounds in the group 8 are each a compound having high structural stability. In addition, the compounds are each a compound having a relatively small S₁ energy because the compound contains the sulfur atom in a molecule thereof. As in the heterocycle-containing compounds in the group 1 to the group 7, the incorporation of any one of those heterocycle-containing compounds in the group 8 as the host into the emission layer can also optimize the carrier balance between the host and guest (iridium complex represented by the general formula [1]) in the emission layer. Therefore, an organic light-emitting device having high emission efficiency and a long lifetime is obtained. In addition, the incorporation of any one of the heterocycle-containing compounds in the group 8 as the host into the emission layer can reduce the driving voltage.

(6) Other Materials

As described above, in the organic light-emitting device of the present invention, the organic compound layer includes at least the iridium complex represented by the general formula [1] as the guest and the heterocycle-containing compound as the host. However, in the present invention, conventionally known low-molecular weight and high-molecular weight materials can each be used as required in addition to these compounds. More specifically, a hole-injectable/transportable material, a host, a light emission assist material, an electron-injectable/transportable material, or the like can be used together with the iridium complex and the heterocycle-containing compound.

Examples of those materials are listed below.

The hole-injectable/transportable material is preferably a material having a high hole mobility so that the injection of a hole from the anode may be facilitated and the injected hole can be transported to the emission layer. In addition, the material is preferably a material having a high glass transition point for preventing the degradation of film quality such as crystallization in the organic light-emitting device. Examples of the low-molecular weight and high-molecular weight materials each having hole-injecting/transporting performance include a triarylamine derivative, an arylcarbazole derivative, a phenylenediamine derivative, a stilbene derivative, a phthalocyanine derivative, a porphyrin derivative, poly(vinyl carbazole), poly(thiophene), and other conductive polymers. Further, the hole-injectable/transportable material is suitably used for the electron blocking layer as well.

Specific examples of a compound to be used as the hole-injectable/transportable material are shown below. However, the compound is of course not limited thereto.

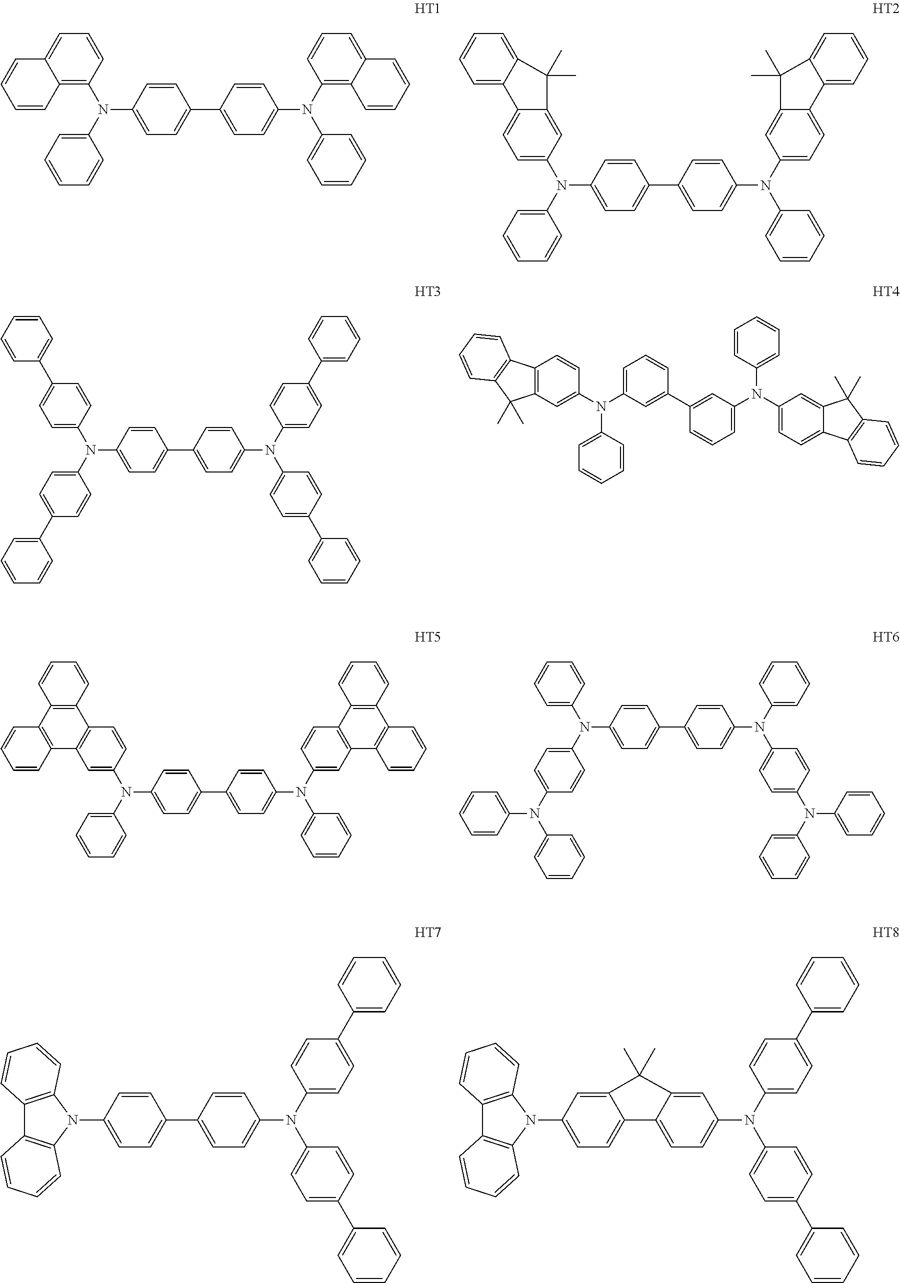

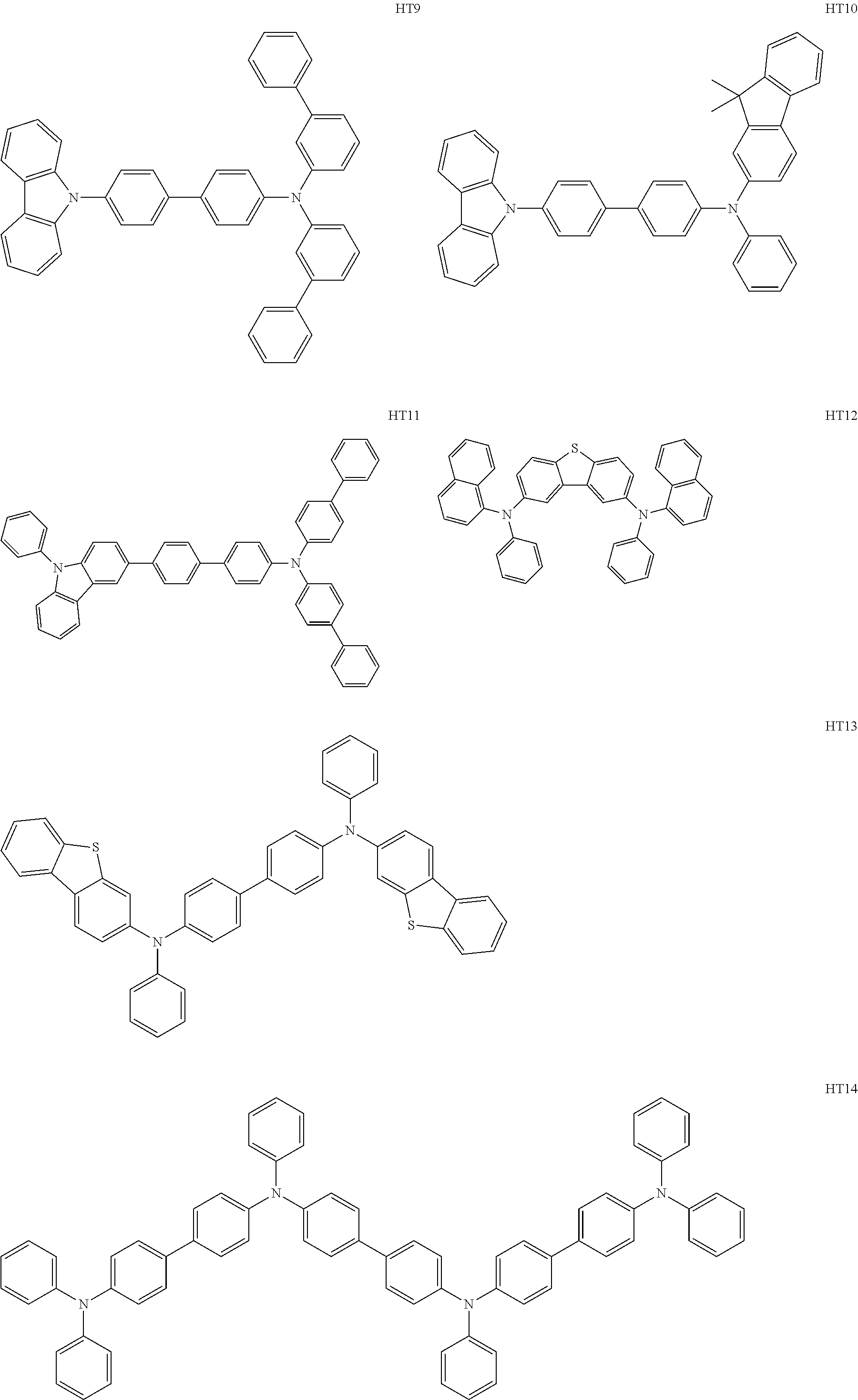

Examples of the light-emitting material mainly involved in a light-emitting function include: condensed ring compounds (such as a fluorene derivative, a naphthalene derivative, a pyrene derivative, a perylene derivative, a tetracene derivative, an anthracene derivative, and rubrene); a quinacridone derivative; a coumarin derivative; a stilbene derivative; an organic aluminum complex such as tris(8-quinolinolato)aluminum; a platinum complex; a rhenium complex; a copper complex; a europium complex; a ruthenium complex; and polymer derivatives such as a poly(phenylene vinylene) derivative, a poly(fluorene) derivative, and a poly(phenylene) derivative in addition to the iridium complex represented by the general formula [1] or a derivative thereof.

Specific examples of a compound to be used as the light-emitting material are shown below. However, the compound is of course not limited thereto.

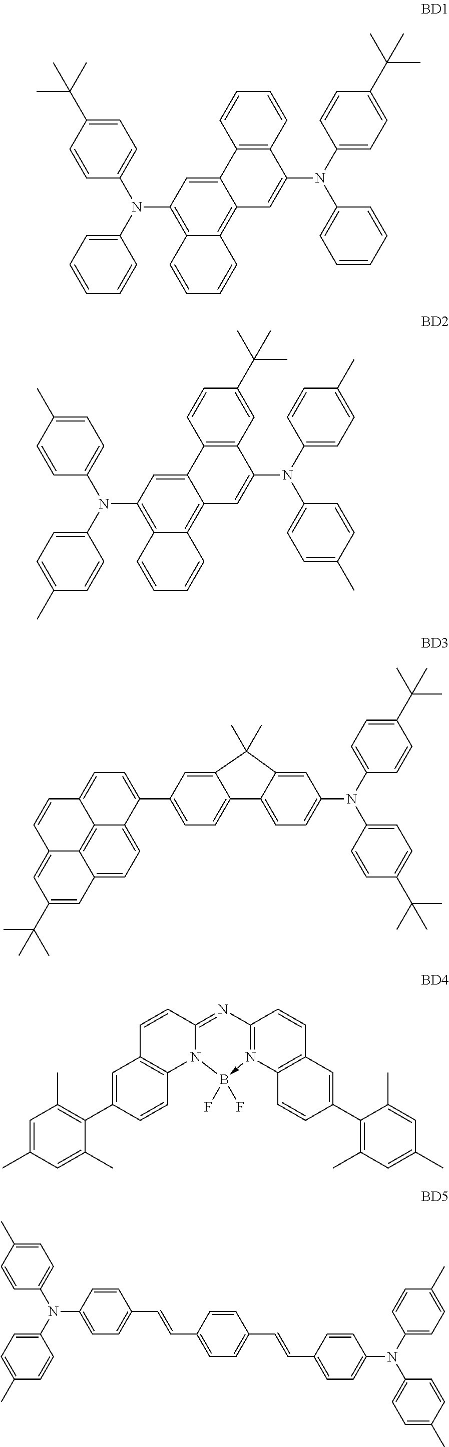

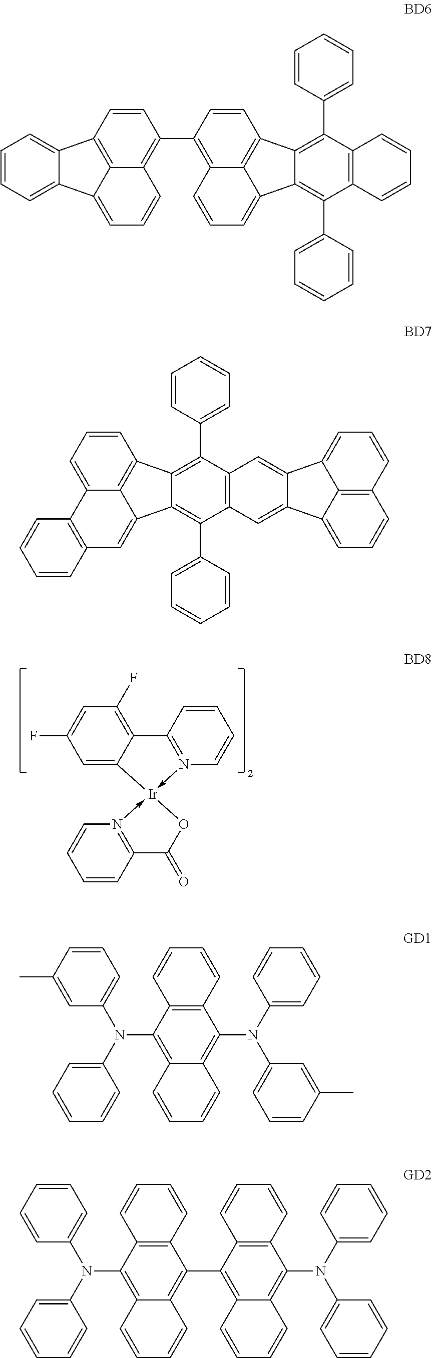

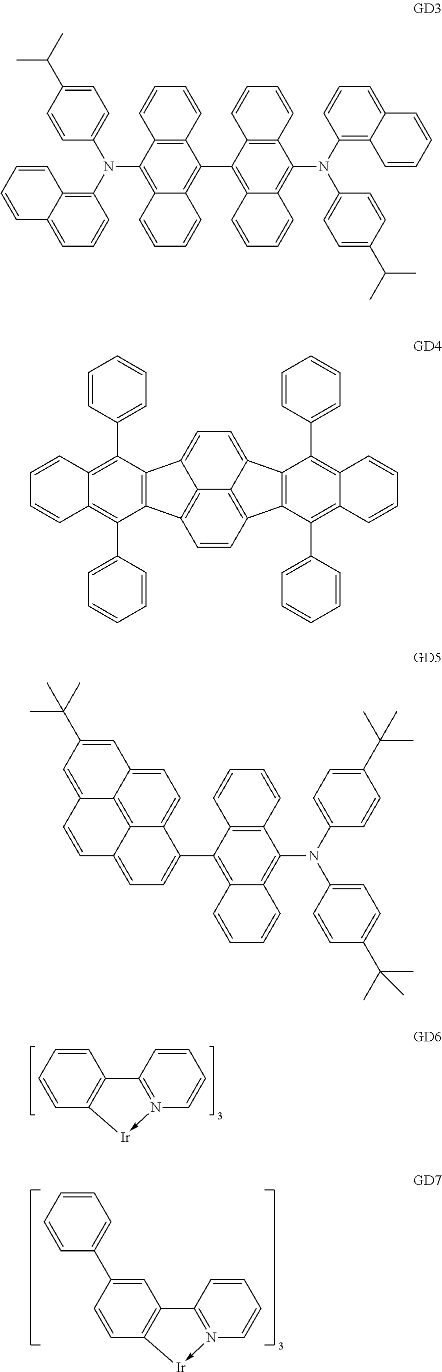

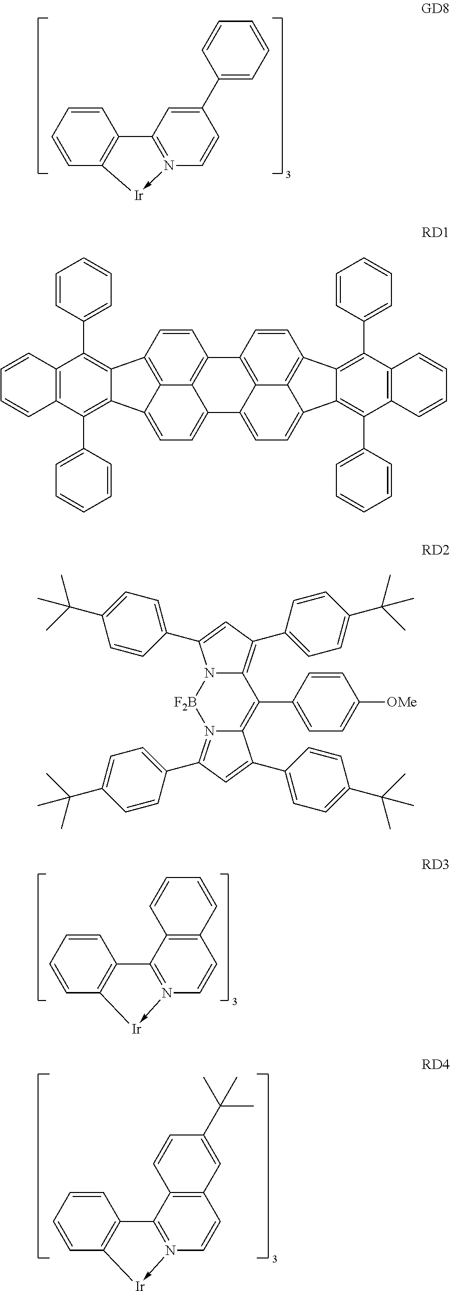

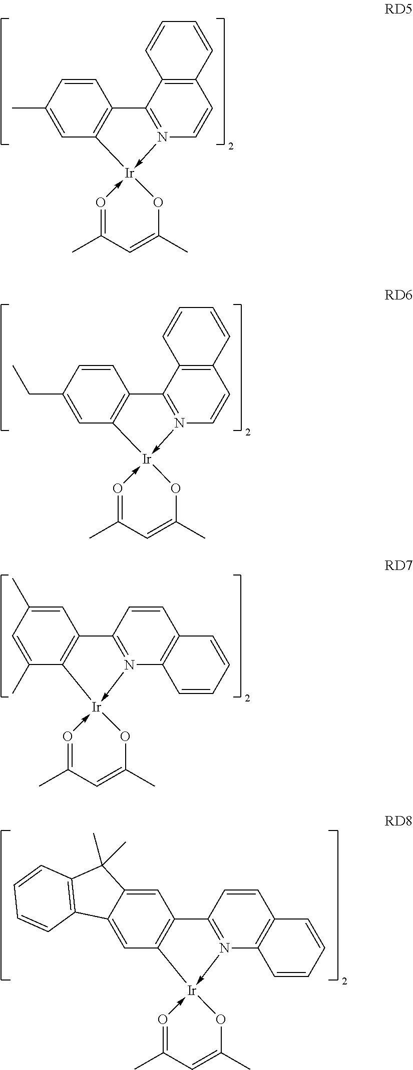

Examples of the host or assist material to be incorporated into the emission layer include: an aromatic hydrocarbon compound or a derivative thereof; a carbazole derivative; a dibenzofuran derivative; a dibenzothiophene derivative; an organic aluminum complex such as tris(8-quinolinolato)aluminum; and an organic beryllium complex in addition to the heterocycle-containing compound.

Specific examples of a compound to be used as the host or assist material to be incorporated into the emission layer are shown below. However, the compound is of course not limited thereto.

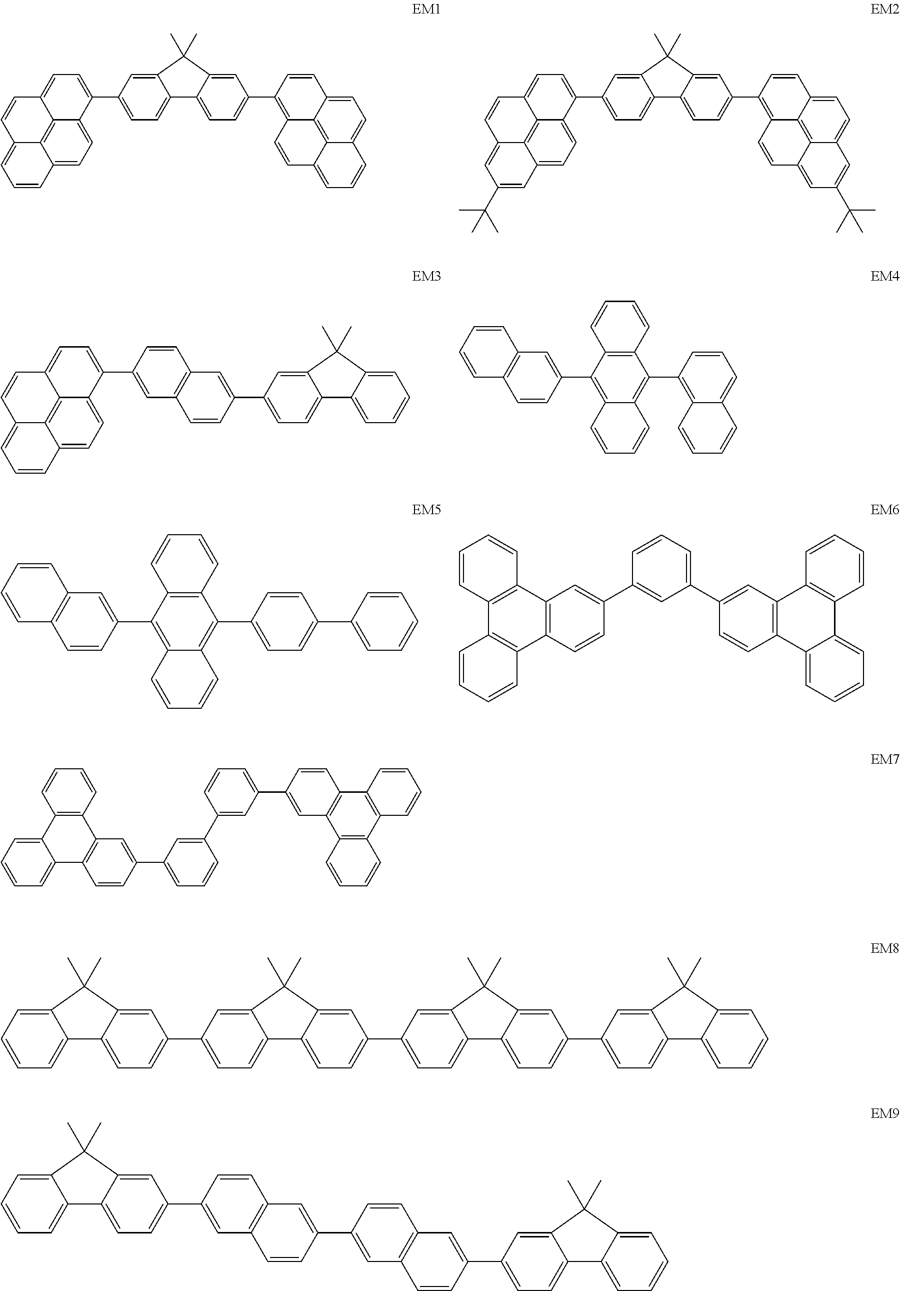

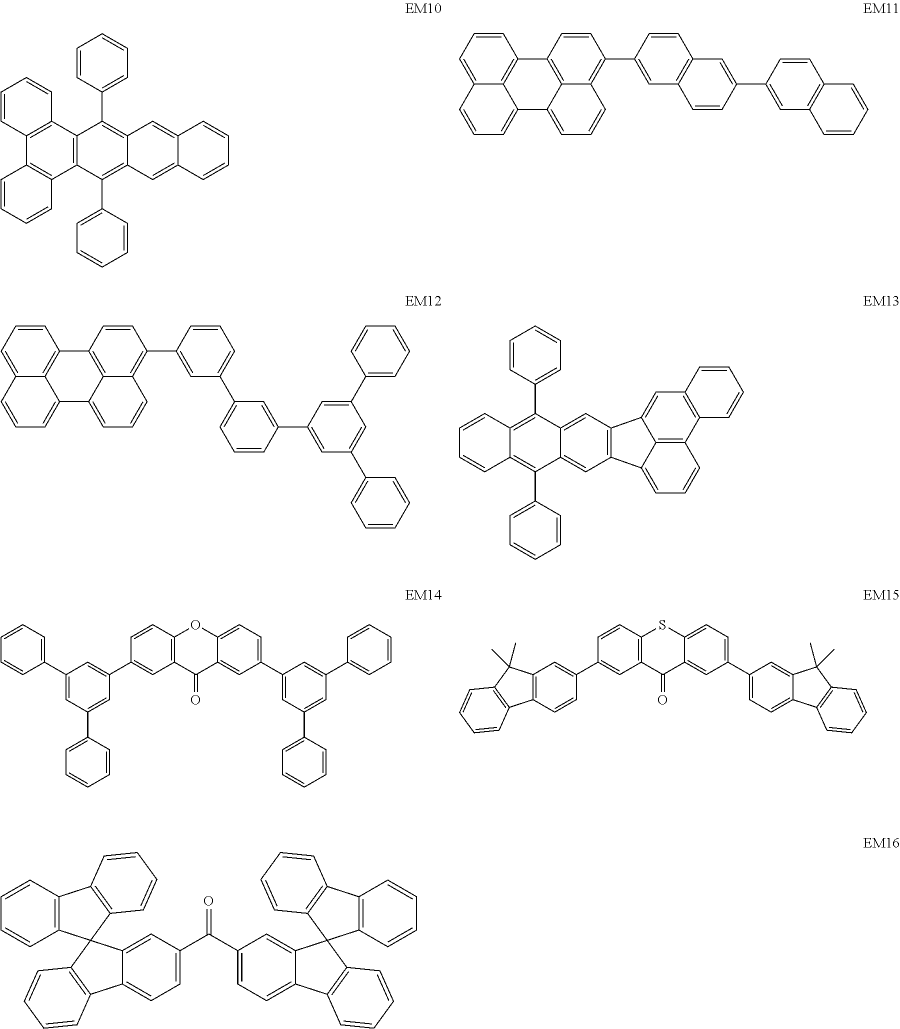

The electron-injectable/transportable material can be arbitrarily selected from materials that allow electrons to be easily injected from the cathode and can transport the injected electrons to the emission layer in consideration of, for example, the balance with the hole mobility of the hole-transportable material. Examples of the material having electron-injecting performance and electron-transporting performance include an oxadiazole derivative, an oxazole derivative, a pyrazine derivative, a triazole derivative, a triazine derivative, a quinoline derivative, a quinoxaline derivative, a phenanthroline derivative, and an organic aluminum complex. Further, the electron-injectable/transportable material is suitably used for the hole blocking layer as well.

Specific examples of a compound to be used as the electron-injectable/transportable material are shown below. However, the compound is of course not limited thereto.

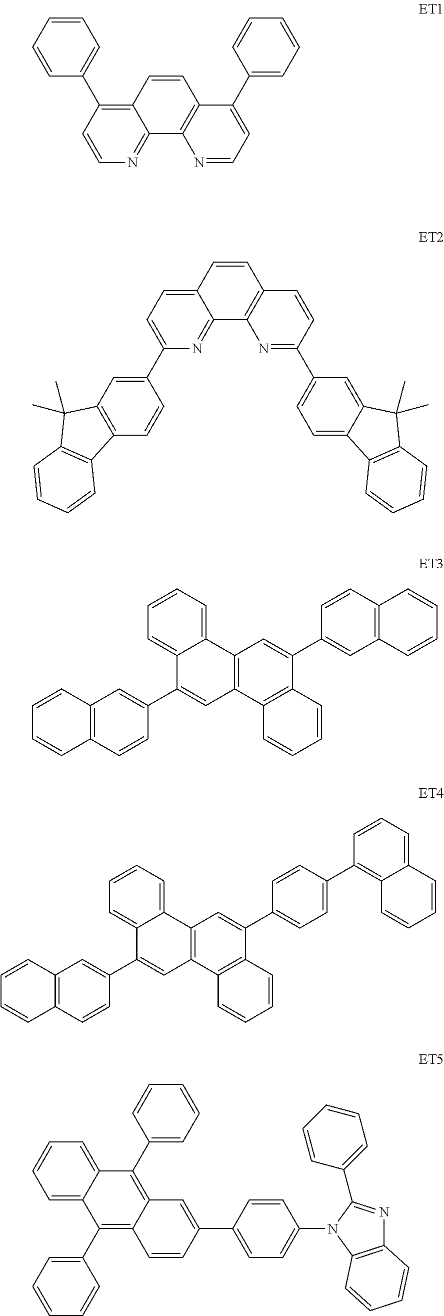

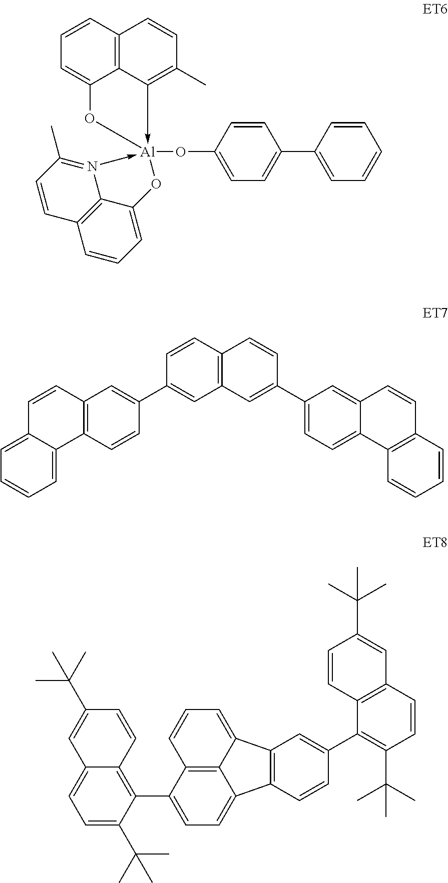

In addition, a mixture obtained by mixing the electron-injectable/transportable material and an alkali metal or alkaline earth metal compound may be used as the electron-injectable/transportable material. Examples of the metal compound to be mixed with the electron-injectable/transportable material include LiF, KF, Cs₂CO₃, and CsF.

A constituent material for the anode desirably has as large a work function as possible. For example, there may be used: metal simple substances such as gold, platinum, silver, copper, nickel, palladium, cobalt, selenium, vanadium, and tungsten or alloys obtained by combining those metal simple substances; and metal oxides such as tin oxide, zinc oxide, indium oxide, indium tin oxide (ITO), indium zinc oxide, gallium zinc oxide, and indium gallium zinc oxide. In addition, there may be used conductive polymers such as polyaniline, polypyrrole, and polythiophene. Of those, a transparent oxide semiconductor (e.g., indium tin oxide (ITO), indium zinc oxide, or indium gallium zinc oxide) is suitable as an electrode material because of its high mobility.

One kind of those electrode substances may be used alone, or two or more kinds thereof may be used in combination. In addition, the anode may be of a single-layer construction or may be of a multilayer construction.

On the other hand, a constituent material for the cathode desirably has as small a work function as possible. Examples thereof include: alkali metals such as lithium; alkaline earth metals such as calcium; and metal simple substances such as aluminum, titanium, manganese, silver, lead, and chromium. Alternatively, alloys obtained by combining those metal simple substances can be used. For example, a magnesium-silver alloy, an aluminum-lithium alloy, or an aluminum-magnesium alloy can be used. A metal oxide such as indium tin oxide (ITO) can also be utilized. One kind of those electrode substances may be used alone, or two or more kinds thereof may be used in combination. In addition, the cathode may be of a single-layer construction or may be of a multilayer construction.

The organic compound layer (such as the hole injection layer, the hole transport layer, the electron blocking layer, the emission layer, the hole blocking layer, the electron transport layer, or the electron injection layer) for forming the organic light-emitting device of the present invention is formed by the following method.

A dry process such as a vacuum vapor deposition method, an ionized vapor deposition method, sputtering, or a plasma process can be used for the formation of the organic compound layer for forming the organic light-emitting device of the present invention. In addition, a wet process involving dissolving the constituent materials in an appropriate solvent and forming a layer by a known application method (such as a spin coating method, a dipping method, a casting method, an LB method, or an ink jet method) can be used instead of the dry process.

Here, when the layer is formed by the vacuum vapor deposition method, the solution application method, or the like, the layer hardly undergoes crystallization or the like, and is excellent in stability over time. In addition, when the layer is formed by the application method, the film can be formed by using the constituent materials in combination with an appropriate binder resin.

Examples of the binder resin include, but not limited to, a polyvinyl carbazole resin, a polycarbonate resin, a polyester resin, an ABS resin, an acrylic resin, a polyimide resin, a phenol resin, an epoxy resin, a silicone resin, and a urea resin.

In addition, one kind of those binder resins may be used alone as a homopolymer or a copolymer, or two or more kinds thereof may be used as a mixture. Further, a known additive such as a plasticizer, an antioxidant, or a UV absorber may be used in combination as required.

(7) Application of Organic Light-Emitting Device of the Present Invention

The organic light-emitting device of the present invention can be used as a constituent member for a display apparatus or lighting apparatus. In addition, the device finds use in applications such as an exposure light source for an image-forming apparatus of an electrophotographic system, a backlight for a liquid crystal display apparatus, and a light-emitting apparatus including a white light source and a color filter. Examples of the color filter include filters that transmit light beams having three colors, i.e., red, green, and blue colors.

A display apparatus of the present invention includes the organic light-emitting device of the present invention in its display portion. It is to be noted that the display portion includes multiple pixels.

In addition, the pixels each have the organic light-emitting device of the present invention and a transistor as an example of an active device (switching device) or amplifying device for controlling emission luminance, and the anode or cathode of the organic light-emitting device and the drain electrode or source electrode of the transistor are electrically connected to each other. Here, the display apparatus can be used as an image display apparatus for a PC or the like. The transistor is, for example, a TFT device and the TFT device is, for example, a device formed of a transparent oxide semiconductor, and is provided on, for example, the insulating surface of a substrate.

The display apparatus may be an information processing apparatus that includes an image input portion for inputting image information from, for example, an area CCD, a linear CCD, or a memory card, and displays an input image on its display portion.

In addition, the display portion of an imaging apparatus or inkjet printer may have a touch panel function. The drive system of the touch panel function is not particularly limited.

In addition, the display apparatus may be used in the display portion of a multifunction printer.

A lighting apparatus is an apparatus for lighting, for example, the inside of a room. The lighting apparatus may emit light having any one of the following colors: a white color (having a color temperature of 4,200 K), a daylight color (having a color temperature of 5,000 K), and colors ranging from blue to red colors.

A lighting apparatus of the present invention includes the organic light-emitting device of the present invention and an inverter circuit connected to the organic light-emitting device. It is to be noted that the lighting apparatus may further include a color filter.

An image-forming apparatus of the present invention is an image-forming apparatus including: a photosensitive member; charging unit for charging the surface of the photosensitive member; exposing unit for exposing the photosensitive member to form an electrostatic latent image; and a developing unit for developing the electrostatic latent image formed on the surface of the photosensitive member. Here, the exposing unit to be provided in the image-forming apparatus includes the organic light-emitting device of the present invention.

In addition, the organic light-emitting device of the present invention can be used as a constituent member for an exposing apparatus for exposing a photosensitive member. An exposing apparatus including a plurality of the organic light-emitting devices of the present invention is, for example, an exposing apparatus in which the organic light-emitting devices of the present invention are placed to form a line along a predetermined direction.

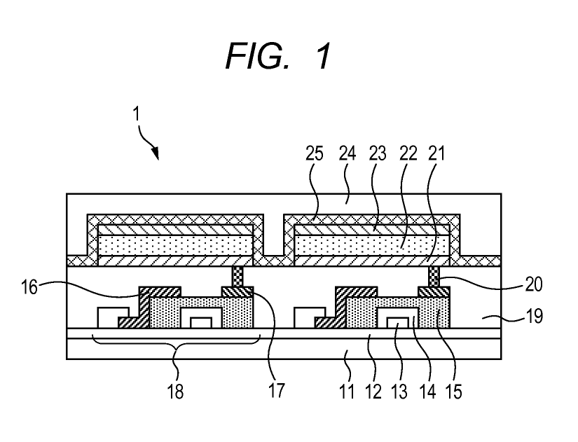

Next, the display apparatus of the present invention is described with reference to the drawing. FIG. 1 is a schematic sectional view illustrating an example of a display apparatus including an organic light-emitting device and a TFT device connected to the organic light-emitting device. It is to be noted that the organic light-emitting device of the present invention is used as the organic light-emitting device constituting a display apparatus 1 of FIG. 1.

The display apparatus 1 of FIG. 1 includes a substrate 11 made of glass or the like and a moisture-proof film 12 for protecting a TFT device or organic compound layer, the film being provided on the substrate. In addition, a metal gate electrode 13 is represented by reference numeral 13, a gate insulating film 14 is represented by reference numeral 14, and a semiconductor layer is represented by reference numeral 15.

A TFT device 18 includes the semiconductor layer 15, a drain electrode 16, and a source electrode 17. An insulating film 19 is provided on the TFT device 18. An anode 21 constituting the organic light-emitting device and the source electrode 17 are connected to each other through a contact hole 20.

It is to be noted that a system for the electrical connection between the electrode (anode or cathode) in the organic light-emitting device and the electrode (source electrode or drain electrode) in the TFT is not limited to the aspect illustrated in FIG. 1. In other words, one of the anode and the cathode, and one of the source electrode and drain electrode of the TFT device have only to be electrically connected to each other.

Although multiple organic compound layers are illustrated like one layer in the display apparatus 1 of FIG. 1, an organic compound layer 22 may be multiple layers. A first protective layer 24 and second protective layer 25 for suppressing the degradation of the organic light-emitting device are provided on a cathode 23.

When the display apparatus 1 of FIG. 1 is a display apparatus that emits white light, an emission layer in the organic compound layer 22 in FIG. 1 may be a layer obtained by mixing a red light-emitting material, a green light-emitting material, and a blue light-emitting material. In addition, the layer may be a stacked emission layer obtained by stacking a layer formed of the red light-emitting material, a layer formed of the green light-emitting material, and a layer formed of the blue light-emitting material. Further, alternatively, the following aspect is permitted: the layer formed of the red light-emitting material, the layer formed of the green light-emitting material, and the layer formed of the blue light-emitting material are, for example, arranged side by side to form domains in one emission layer.

Although the transistor is used as the switching device in the display apparatus 1 of FIG. 1, an MIM device may be used instead of the transistor as the switching device.

In addition, the transistor to be used in the display apparatus 1 of FIG. 1 is not limited to a transistor using a monocrystalline silicon wafer and may be a thin-film transistor including an active layer on the insulating surface of a substrate. A thin-film transistor using monocrystalline silicon as the active layer, a thin-film transistor using non-monocrystalline silicon such as amorphous silicon or microcrystalline silicon as the active layer, or a thin-film transistor using a non-monocrystalline oxide semiconductor such as an indium zinc oxide or an indium gallium zinc oxide as the active layer is also permitted. It is to be noted that the thin-film transistor is also called a TFT device.

The transistor in the display apparatus 1 of FIG. 1 may be formed in a substrate such as an Si substrate. Here, the phrase “formed in a substrate” means that the transistor is produced by processing the substrate itself such as an Si substrate. In other words, the presence of the transistor in the substrate can be regarded as follows: the substrate and the transistor are integrally formed.

Whether the transistor is provided in the substrate is selected depending on definition. In the case of, for example, a definition of about a QVGA per inch, the organic light-emitting device is preferably provided in the Si substrate.

As described above, the driving of the display apparatus using the organic light-emitting device of the present invention enables display that has good image quality and is stable over a long time period.

### EXAMPLES

### Synthesis Example 1

### Synthesis of Exemplified Compound KK-01

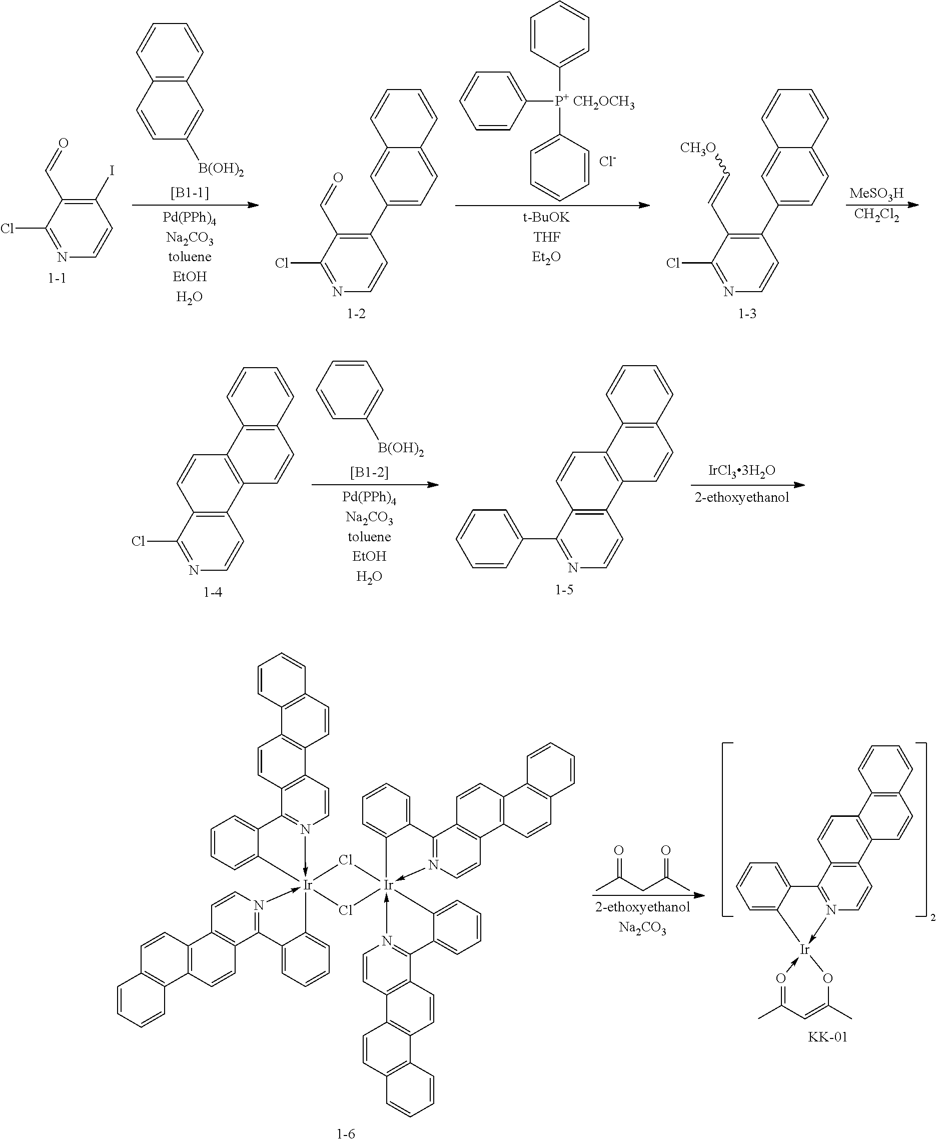

(1) Synthesis of Compound 1-2

The following reagents and solvents were loaded into a reaction vessel.

Compound [1-1]: 6.0 g (22.4 mmol)

Compound [B1-1]: 3.47 g (20.2 mmol)

Toluene: 160 ml

Ethanol: 80 ml

Aqueous solution of sodium carbonate (2 N): 80 ml

Next, 1.30 g (1.12 mmol) of tetrakis(triphenylphosphine)palladium(0) were added while the reaction solution was stirred at room temperature under a nitrogen atmosphere. Next, the temperature of the reaction solution was increased to 60° C. and then the reaction solution was stirred at the temperature (60° C.) for 7 hours. After the completion of the reaction, water was charged into the resultant, and then the organic layer was extracted with toluene and dried with anhydrous sodium sulfate. After that, the solvent was removed by distillation under reduced pressure. Next, the residue was purified by column chromatography (gel for chromatography: BW300 (manufactured by FUJI SILYSIA CHEMICAL LTD.), eluent: chloroform) and then washed with methanol to provide 4.0 g of Compound 1-2 (yield: 74%).

(2) Synthesis of Compound 1-3

The following reagents and solvent were loaded in a reaction vessel.

(Methoxymethyl)triphenylphosphonium chloride: 5.76 g (16.8 mmol)

Potassium tert-butoxide (1 M solution in THF): 16.8 ml (16.8 mmol)

Dry ether: 30 ml.

Next, those loaded into the reaction vessel were stirred at room temperature for 30 minutes to be suspended. Next, a THF solution obtained by dissolving Compound [1-2] (1.8 g, 6.72 mmol) in 45 ml of dry THF was dropped to the suspension, and then the mixture was stirred for 10 hours while its temperature was kept at room temperature. After the completion of the reaction, water was charged into the resultant, and then the organic layer was extracted with toluene and dried with anhydrous sodium sulfate. After that, the solvent was removed by distillation under reduced pressure. Next, the residue was purified by column chromatography (gel for chromatography: BW300 (manufactured by FUJI SILYSIA CHEMICAL LTD.), eluent: chloroform), and was then recrystallized with a mixed solvent of toluene and ethanol to provide 780 mg of Compound 1-3 (yield: 39%).

(3) Synthesis of Compound 1-4

4 Milliliters of methanesulfonic acid were dropped to a solution obtained by dissolving Compound 1-3 (2.0 g, 6.76 mmol) in 40 ml of dry dichloromethane, and then the mixture was stirred at room temperature for 18 hours. After the completion of the reaction, water was charged into the resultant, and then the organic layer was extracted with chloroform and dried with anhydrous sodium sulfate. After that, the solvent was removed by distillation under reduced pressure. Next, the residue was purified by column chromatography (gel for chromatography: BW300 (manufactured by FUJI SILYSIA CHEMICAL LTD.), eluent: chloroform), and was then recrystallized with a mixed solvent of toluene and ethanol three times. Next, the resultant crystal was washed with methanol to provide 485 mg of Compound 1-4 (yield: 27%).

(4) Synthesis of Compound 1-5

The following reagents and solvents were loaded into a reaction vessel.

Compound [1-4]: 0.485 g (1.84 mmol)

Compound [B1-2]: 0.269 g (2.21 mmol)

Toluene: 40 ml

Ethanol: 20 ml

Aqueous solution of sodium carbonate (2 N): 20 ml

Next, 106 mg (0.092 mmol) of tetrakis(triphenylphosphine)palladium(0) were added while the reaction solution was stirred at room temperature under a nitrogen atmosphere. Next, the temperature of the reaction solution was increased to 85° C. and then the reaction solution was stirred for 7 hours. After the completion of the reaction, water was charged into the resultant, and then the organic layer was extracted with toluene and dried with anhydrous sodium sulfate. After that, the solvent was removed by distillation under reduced pressure. Next, the residue was purified by column chromatography (gel for chromatography: BW300 (manufactured by FUJI SILYSIA CHEMICAL LTD.), eluent: hot toluene) and then recrystallized with toluene to provide 365 mg of Compound 1-5 (yield: 65%).

The structure of the compound was confirmed by ¹H-NMR measurement (400 MHz, CDCl₃).

σ (ppm): 8.88-8.84 (d, 1H), 8.78-8.76 (d, 1H), 8.75-8.71 (t, 2H), 8.54-8.53 (d, 1H), 8.25-8.22 (d, 1H), 8.10-8.08 (d, 1H), 8.05-8.03 (d, 1H), 7.78-7.69 (m, 4H), 7.60-7.51 (m, 3H).

(5) Synthesis of Compound 1-6

300 Milligrams (0.982 mmol) of Compound 1-5 and 157 mg (0.447 mmol) of iridium(III) chloride hydrate were dissolved in 12 ml of 2-ethoxyethanol and 3 ml of water, and then the temperature of the mixture was increased to 100° C. in a nitrogen atmosphere, followed by stirring for 7 hours. After the completion of the reaction, water was charged into the resultant, and then the precipitated solid was collected by filtration and washed with water, ethanol, and toluene. After drying, 300 mg of Compound 1-6 were obtained (yield: 73%).

(6) Synthesis of Exemplified Compound KK-01

The following reagents and solvent were loaded into a reaction vessel.

Compound 1-6: 200 mg (0.12 mmol)

Acetylacetone: 2.0 g (20.2 mmol)

Sodium carbonate: 500 mg (4.72 mmol)

2-Ethoxyethanol: 5 ml

Next, the temperature of the reaction solution was increased to 95° C. in a nitrogen atmosphere, and then the reaction solution was stirred at the temperature (95° C.) for 7 hours. After the completion of the reaction, water was charged into the resultant, and the precipitated solid was collected by filtration, and was then washed with water and ethanol. After drying, the residue was purified by column chromatography (gel for chromatography: BW200 (manufactured by FUJI SILYSIA CHEMICAL LTD.), eluent: hot chlorobenzene), and after that, 190 mg of Exemplified Compound KK-01 were obtained (yield: 88%). Subsequently, sublimation purification was performed under the conditions of 1×10⁻⁴ Pa and 390° C. to provide 5 mg of a sublimated product of Exemplified Compound KK-01.

The structure of the compound was confirmed by ¹H-NMR measurement (400 MHz, CDCl₃).

σ (ppm): 9.14-9.11 (d, 2H), 8.92-8.90 (d, 2H), 8.86-8.84 (d, 2H), 8.73-8.69 (m, 4H), 8.41-8.39 (d, 2H), 8.29-8.27 (d, 2H), 8.13-8.11 (d, 2H), 8.08-8.06 (d, 2H), 7.82-7.79 (t, 2H), 7.76-7.72 (t, 2H), 6.97-6.93 (t, 2H), 6.71-6.67 (t, 2H), 6.46-6.44 (d, 2H), 5.26 (s, 1H), 1.81 (s, 3H).

Matrix assisted ionization time-of-flight mass spectrometry (MALDI-TOF MS) confirmed that the compound had an M⁺ of 900.22. In addition, the emission spectrum of a 1×10⁻⁵ mol/l solution of the resultant compound in toluene at room temperature was measured with an F-4500 manufactured by Hitachi, Ltd. at an excitation wavelength of 480 nm. As a result, its maximum emission wavelength was found to be 613 nm. In addition, the absolute quantum yield of the compound at room temperature in a solution state was measured with an absolute PL quantum yield measurement system (C9920-02) manufactured by Hamamatsu Photonics K.K. As a result, the absolute quantum yield was found to be 0.9 (relative value when the absolute quantum yield of Ir(pbiq)₃ was defined as 1.0).

### Synthesis Examples 2

### Synthesis of Exemplified Compound KK-03

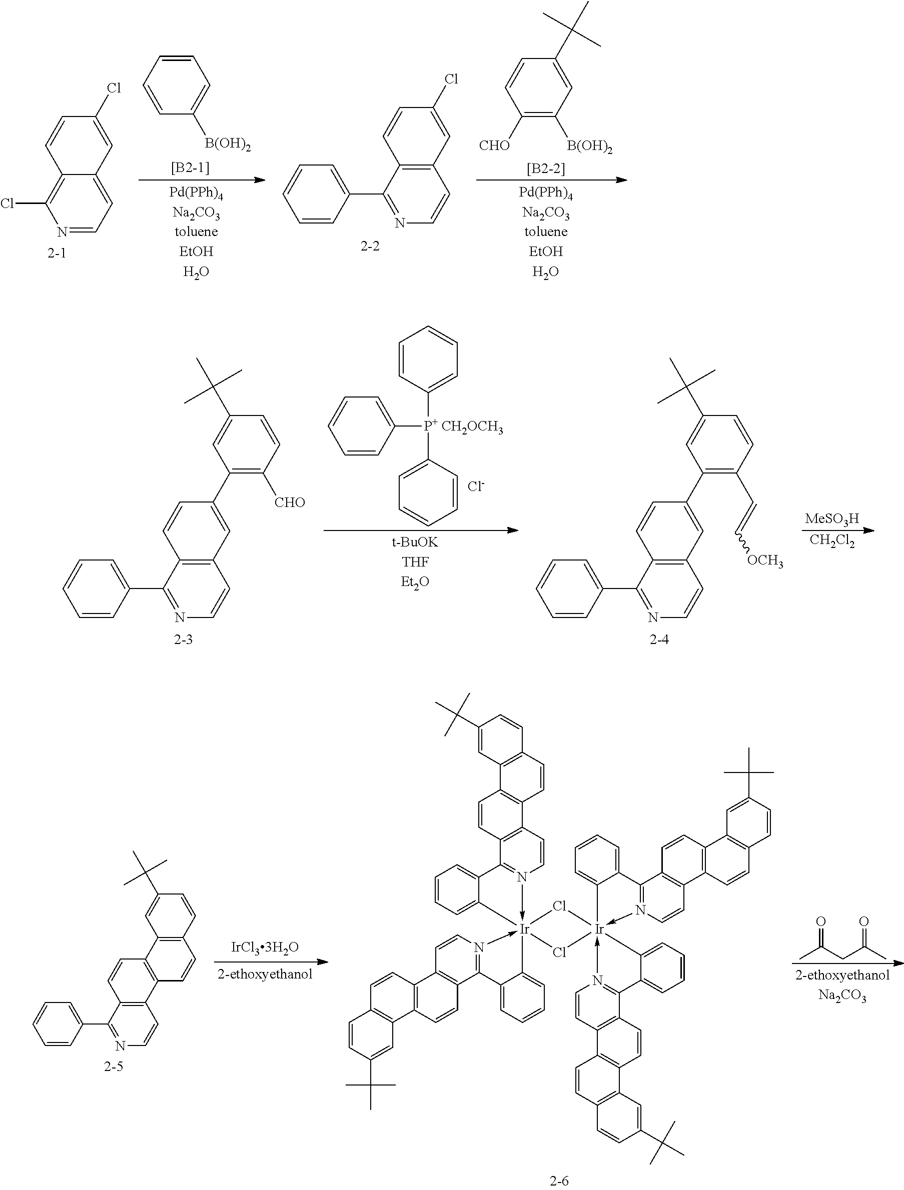

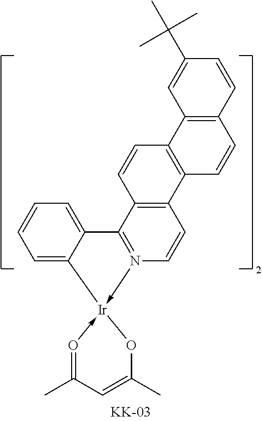

(1) Synthesis of Compound 2-2

The following reagents and solvents were loaded into a reaction vessel.

Compound [2-1]: 8.0 g (40.4 mmol)

Compound [B2-1]: 5.91 g (48.5 mmol)

Toluene: 200 ml

Ethanol: 100 ml

Aqueous solution of sodium carbonate (2 N): 100 ml

Next, 2.33 g (2.02 mmol) of tetrakis(triphenylphosphine)palladium(0) were added while the reaction solution was stirred at room temperature under a nitrogen atmosphere. Next, the temperature of the reaction solution was increased to 60° C. and then the reaction solution was stirred at the temperature (60° C.) for 7 hours. After the completion of the reaction, water was charged into the resultant, and then the organic layer was extracted with toluene and dried with anhydrous sodium sulfate. After that, the solvent was removed by distillation under reduced pressure. Next, the residue was purified by column chromatography (gel for chromatography: BW300 (manufactured by FUJI SILYSIA CHEMICAL LTD.), eluent: ethyl acetate/heptane=1/2) and then washed with methanol to provide 5.89 g of Compound 2-2 (yield: 61%).

(2) Synthesis of Compound B2-2

8.64 Milliliters (68 mmol) of N,N,N′-trimethylethylenediamine were dissolved in 160 ml of dry THF in a reaction vessel. After that, the reaction solution was stirred at −40° C. for 30 minutes. 40 Milliliters (64 mmol) of n-butyllithium (1.6 M solution in hexane) were dropped to the reaction solution, and then the reaction solution was stirred for 30 minutes while its temperature was maintained at −40° C. Next, 10 ml (60 mmol) of 4-tert-butylbenzaldehyde were dropped to the reaction solution, and then the reaction solution was stirred for 30 minutes while its temperature was maintained at −40° C. Next, 112 ml (180 mmol) of n-butyllithium (1.6 M solution in hexane) were dropped to the reaction solution, and then the reaction solution was stirred for 30 minutes while its temperature was maintained at −40° C. Next, the reaction solution was stirred for 10 hours while its temperature was slowly increased to room temperature. Next, the reaction solution was cooled to −40° C. again. After that, 40 ml (360 mmol) of trimethyl borate were dropped to the reaction solution, and then the reaction solution was stirred for 30 minutes while its temperature was maintained at −40° C. Next, the reaction solution was stirred for 20 hours while its temperature was slowly increased to room temperature. Next, the reaction solution was poured into 400 ml of 2 N hydrochloric acid, and then the mixture was stirred at room temperature for 30 minutes. Next, water was charged into the resultant, and then the organic layer was extracted with chloroform and dried with anhydrous sodium sulfate. After that, the solvent was removed by distillation under reduced pressure. Next, the residue was purified by column chromatography (gel for chromatography: BW300 (manufactured by FUJI SILYSIA CHEMICAL LTD.), eluent: ethyl acetate/heptane=1/2), and was then washed with heptane to provide 2.45 g of Compound B2-2 (yield: 20%).

(3) Synthesis of Compound 2-3

The following reagents and solvents were loaded into a reaction vessel.

Compound 2-2: 2.0 g (8.34 mmol)

Compound [B2-2]: 1.89 g (9.18 mmol)

Bis(dibenzylideneacetone)palladium(0): 0.24 g (0.417 mmol)

2-Dicyclohexylphosphino-2′,6′-dimethoxybiphenyl: 0.34 g (0.834 mmol)

Potassium phosphate: 3.54 g (16.7 mmol)

Dry toluene: 350 ml

Water: 1 ml

Next, the temperature of the reaction solution was increased to 130° C. and then the reaction solution was stirred at the temperature (130° C.) for 6 hours. After the completion of the reaction, water was charged into the resultant, and then the organic layer was extracted with toluene and dried with anhydrous sodium sulfate. After that, the solvent was removed by distillation under reduced pressure. Next, the residue was purified by column chromatography (gel for chromatography: BW300 (manufactured by FUJI SILYSIA CHEMICAL LTD.), eluent: ethyl acetate/heptane=1/2) to provide 1.98 g of Compound 2-3 (yield: 65%).

(4) Synthesis of Compound 2-4

The following reagents and solvent were loaded into a reaction vessel.

(Methoxymethyl)triphenylphosphonium chloride: 4.64 g (13.5 mmol)

Potassium tert-butoxide (1 M solution in THF): 13.5 ml (13.5 mmol)

Dry ether: 25 ml

Next, those loaded into the reaction vessel were stirred at room temperature for 30 minutes to be suspended. Next, a solution obtained by dissolving Compound [2-3] (1.98 g, 5.42 mmol) in 50 ml of dry THF was dropped to the suspension, and then the mixture was stirred for 16 hours while its temperature was kept at room temperature. After the completion of the reaction, water was charged into the resultant, and then the organic layer was extracted with toluene and dried with anhydrous sodium sulfate. After that, the solvent was removed by distillation under reduced pressure. Next, the residue was purified by column chromatography (gel for chromatography: BW300 (manufactured by FUJI SILYSIA CHEMICAL LTD.), eluent: ethyl acetate/heptane=1/2) to provide 2.0 g of Compound 2-4 (yield: 94%).

(5) Synthesis of Compound 2-5

4 Milliliters of methanesulfonic acid were dropped to a solution obtained by dissolving Compound 2-4 (2.0 g, 5.08 mmol) in 40 ml of dry dichloromethane in a reaction vessel, and then the mixture was stirred at room temperature for 18 hours. After the completion of the reaction, water was charged into the resultant, and then the organic layer was extracted with chloroform and dried with anhydrous sodium sulfate. After that, the solvent was removed by distillation under reduced pressure. Next, the residue was purified by column chromatography (gel for chromatography: BW300 (manufactured by FUJI SILYSIA CHEMICAL LTD.), eluent: ethyl acetate/heptane=1/2) to provide 1.5 g of Compound 2-5 (yield: 82%).

The structure of the compound was confirmed by ¹H-NMR measurement (400 MHz, CDCl₃).

σ (ppm): 8.85-8.83 (d, 1H), 8.79-8.77 (d, 1H), 8.74 (s, 1H), 8.68-8.66 (d, 1H), 8.54-8.52 (d, 1H), 8.06-8.04 (d, 1H), 7.99-7.97 (d, 1H), 7.81-7.76 (m, 3H), 7.60-7.51 (m, 3H), 1.52 (s, 9H).

(6) Synthesis of Compound 2-6

The following reagents and solvents were loaded into a reaction vessel.

Compound 2-5: 650 mg (1.80 mmol)

Iridium(III) chloride hydrate: 288 mg (0.817 mmol)

2-Ethoxyethanol: 20 ml

Water: 5 ml

Next, the temperature of the reaction solution was increased to 100° C. in a nitrogen atmosphere, and then the reaction solution was stirred at the temperature (100° C.) for 8 hours. After the completion of the reaction, water was charged into the resultant, and the precipitated solid was collected by filtration, and was then washed with water and ethanol, followed by drying. Thus, 620 mg of Compound 2-6 were obtained (yield: 73%).

(7) Synthesis of Exemplified Compound KK-03

The following reagents and solvent were loaded into a reaction vessel.

Compound 2-6: 300 mg (0.16 mmol)

Acetylacetone: 2.0 g (20.2 mmol)

Sodium carbonate: 600 mg (5.66 mmol)

2-Ethoxyethanol: 7 ml

In a nitrogen atmosphere, the temperature of the mixture was increased to 95° C. and then the mixture was stirred for 8 hours. After the reaction, water was charged into the resultant, and then the precipitated solid was collected by filtration and washed with water and ethanol. After drying, the resultant solid (residue) was purified by column chromatography (gel for chromatography: BW200 (manufactured by FUJI SILYSIA CHEMICAL LTD.), eluent: chloroform) to provide 180 mg of Exemplified Compound KK-03 (yield: 56%). Subsequently, sublimation purification was performed under the conditions of 1×10⁻⁴ Pa and 375° C. to provide 4 mg of Exemplified Compound KK-03 as a sublimated product.

The structure of the compound was confirmed by ¹H-NMR measurement (400 MHz, CDCl₃).

σ (ppm): 9.13-9.11 (d, 2H), 8.96-8.94 (d, 2H), 8.81 (s, 2H), 8.72-8.70 (d, 2H), 8.66-8.64 (d, 2H), 8.40-8.38 (d, 2H), 8.29-8.27 (d, 2H), 8.09-8.07 (d, 2H), 8.02-8.00 (d, 2H), 7.84-7.82 (d, 2H), 6.96-6.92 (t, 2H), 6.71-6.68 (t, 2H), 6.47-6.45 (d, 2H), 5.26 (s, 1H), 1.81 (s, 3H), 1.56 (s, 9H).

Matrix assisted ionization time-of-flight mass spectrometry (MALDI-TOF MS) confirmed that the compound had an M⁺ of 1012.32. In addition, the emission spectrum of a 1×10⁻⁵ mol/l solution of the resultant compound in toluene at room temperature was measured with an F-4500 manufactured by Hitachi, Ltd. at an excitation wavelength of 480 nm. As a result, its maximum emission wavelength was found to be 613 nm. In addition, the absolute quantum yield of the compound at room temperature in a solution state was measured with an absolute PL quantum yield-measuring apparatus (C9920-02) manufactured by Hamamatsu Photonics K.K. As a result, the absolute quantum yield was found to be 1.0 (relative value when the absolute quantum yield of Ir(pbiq)₃ was defined as 1.0).

### Synthesis Examples 3

### Synthesis of Exemplified Compound KK-02

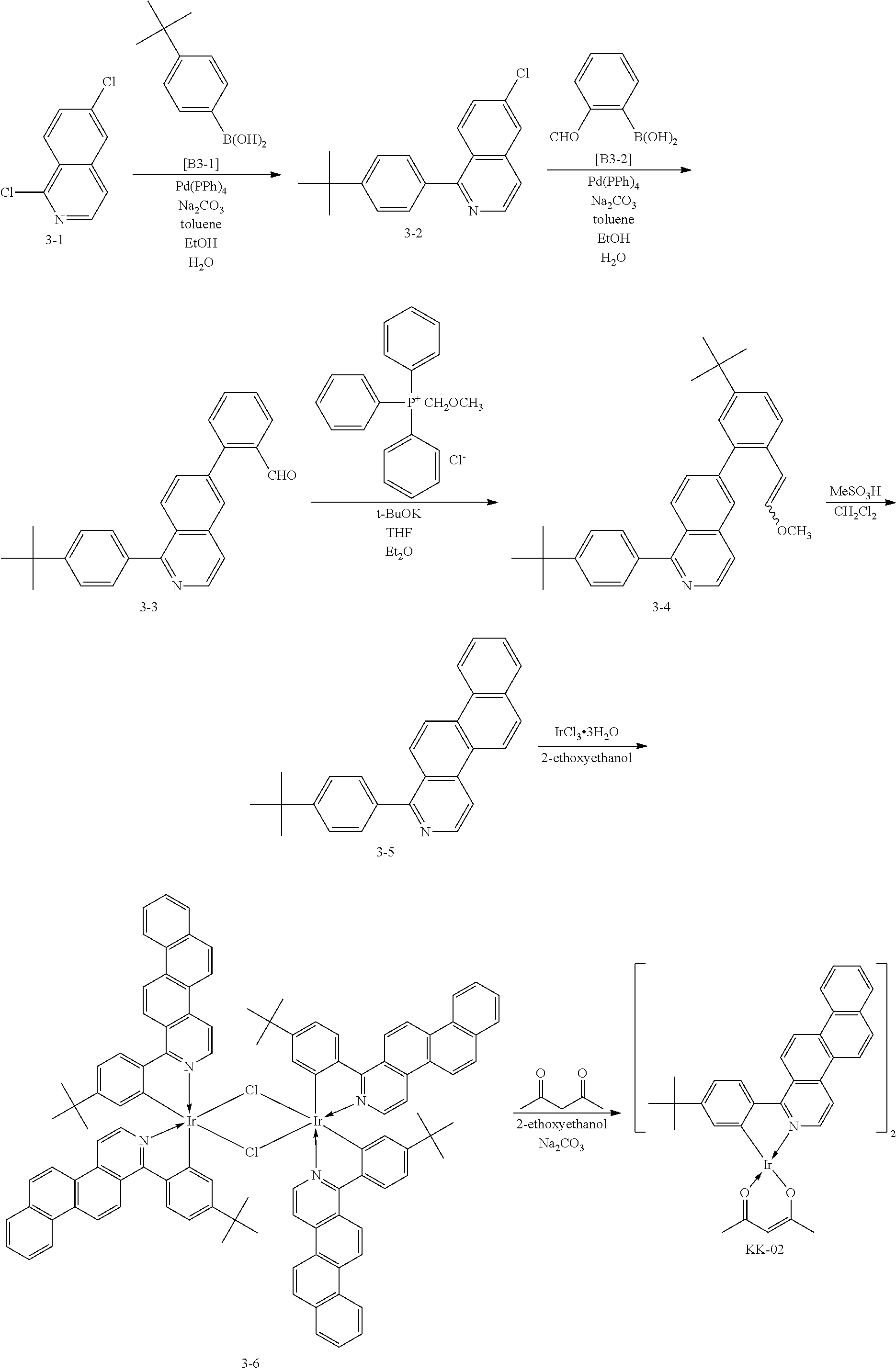

(1) Synthesis of Compound 3-2

The following reagents and solvents were loaded into a reaction vessel.

Compound [3-1]: 4.0 g (20.2 mmol)

Compound [B3-1]: 3.96 g (22.2 mmol)

Toluene: 100 ml

Ethanol: 50 ml

Aqueous solution of sodium carbonate (2 N): 50 ml

Next, 1.17 g (1.01 mmol) of tetrakis(triphenylphosphine)palladium(0) were added while the reaction solution was stirred at room temperature under a nitrogen atmosphere. Next, the temperature of the reaction solution was increased to 60° C. and then the reaction solution was stirred for 6 hours. After the completion of the reaction, water was charged into the resultant, and then the organic layer was extracted with toluene and dried with anhydrous sodium sulfate. After that, the solvent was removed by distillation under reduced pressure. Next, the residue was roughly purified by column chromatography (gel for chromatography: BW300 (manufactured by FUJI SILYSIA CHEMICAL LTD.), eluent: ethyl acetate/heptane=1/3) and then washed with methanol to provide 5.98 g of Compound 3-2 as a crude product (yield: 100%).

(2) Synthesis of Compound 3-3

The following reagents and solvents were loaded into a reaction vessel.

Compound 3-2 (crude product): 5.98 g (20.2 mmol)

Compound [B3-2]: 3.63 g (24.2 mmol)

Bis(dibenzylideneacetone)palladium(0): 0.58 g (1.01 mmol)

2-Dicyclohexylphosphino-2′,6′-dimethoxybiphenyl: 0.88 g (2.13 mmol)

Potassium phosphate: 8.58 g (40.4 mmol)

Dry toluene: 300 ml

Water: 1 ml

Next, the temperature of the reaction solution was increased to 130° C. and then the reaction solution was stirred at the temperature (130° C.) for 5 hours. After the completion of the reaction, water was charged into the resultant, and then the organic layer was extracted with toluene and dried with anhydrous sodium sulfate. After that, the solvent was removed by distillation under reduced pressure. Next, the residue was purified by column chromatography (gel for chromatography: BW300 (manufactured by FUJI SILYSIA CHEMICAL LTD.), eluent: ethyl acetate/heptane=1/3) to provide 5.0 g of Compound 3-3 (yield: 68%).

(3) Synthesis of Compound 3-4

The following reagents and solvent were loaded into a reaction vessel.

(Methoxymethyl)triphenylphosphonium chloride: 11.7 g (34.2 mmol)

Potassium tert-butoxide (1 M solution in THF): 34.2 ml (34.2 mmol)

Dry ether: 60 ml

Next, the contents in the reaction vessel were stirred at room temperature for 30 minutes to be suspended. Next, a THF solution obtained by dissolving Compound [3-3] (5.0 g, 13.7 mmol) in 120 ml of dry THF was dropped to the suspension, and then the mixture was stirred for 16 hours while its temperature was kept at room temperature. After the completion of the reaction, water was charged into the resultant, and then the organic layer was extracted with toluene and dried with anhydrous sodium sulfate. After that, the solvent was removed by distillation under reduced pressure. Next, the residue was purified by column chromatography (gel for chromatography: BW300 (manufactured by FUJI SILYSIA CHEMICAL LTD.), eluent: ethyl acetate/heptane=1/2) to provide 5.15 g of Compound 3-4 (yield: 96%).

(4) Synthesis of Compound 3-5

4 Milliliters of methanesulfonic acid and 30 ml of dry dichloromethane were charged into a reaction vessel, and then the mixture was stirred at room temperature for 5 minutes. Next, a solution obtained by dissolving Compound 3-4 (2.1 g, 2.96 mmol) in 20 ml of dry dichloromethane was dropped to the mixture, and then the whole was stirred for 17 hours while its temperature was kept at room temperature. After the completion of the reaction, water was charged into the resultant, and then the organic layer was extracted with chloroform and dried with anhydrous sodium sulfate. After that, the solvent was removed by distillation under reduced pressure. Next, the residue was purified by column chromatography (gel for chromatography: BW300 (manufactured by FUJI SILYSIA CHEMICAL LTD.), eluent: chloroform) to provide 1.07 g of Compound 3-5 (yield: 55%).

The structure of the compound was confirmed by ¹H-NMR measurement (400 MHz, CDCl₃).

σ (ppm): 8.84-8.83 (d, 1H), 8.79-8.77 (d, 1H), 8.75-8.71 (m, 2H), 8.52-8.51 (d, 1H), 8.32-8.30 (d, 1H), 8.09-8.07 (d, 1H), 8.05-8.03 (d, 1H), 7.75-7.69 (m, 4H), 7.60-7.58 (m, 2H), 1.43 (s, 9H).

(5) Synthesis of Compound 3-6

The following reagents and solvent were loaded into a reaction vessel.

Compound 3-5: 650 mg (1.80 mmol)

Iridium(III) chloride hydrate: 288 mg (0.817 mmol)

2-Ethoxyethanol: 20 ml

Water: 5 ml

Next, the temperature of the reaction solution was increased to 100° C. in a nitrogen atmosphere, and then the reaction solution was stirred at the temperature (100° C.) for 8 hours. After the completion of the reaction, water was charged into the resultant, and the precipitated solid was collected by filtration, and was then washed with water and ethanol. Next, the washed solid was dried to provide 710 mg of Compound 3-6 (yield: 83%).

(6) Synthesis of Exemplified Compound KK-02

The following reagents and solvent were loaded into a reaction vessel.

Compound 3-6: 350 mg (0.18 mmol)

Acetylacetone: 2.0 g (20.2 mmol)

Sodium carbonate: 650 mg (6.13 mmol)

2-Ethoxyethanol: 8 ml

Next, the temperature of the reaction solution was increased to 95° C., and then the reaction solution was stirred at the temperature (95° C.) for 8 hours. After the completion of the reaction, water was charged into the resultant, and the precipitated solid was collected by filtration, and was then washed with water and ethanol. After drying, the residue was purified by column chromatography (gel for chromatography: BW200 (manufactured by FUJI SILYSIA CHEMICAL LTD.), eluent: hot chlorobenzene) to provide 140 mg of Exemplified Compound KK-02 (yield: 67%). Subsequently, sublimation purification was performed under the conditions of 1×10⁻⁴ Pa and 335° C. to provide 4 mg of Exemplified Compound KK-02 as a sublimated product.

Matrix assisted ionization time-of-flight mass spectrometry (MALDI-TOF MS) confirmed that the compound had an M⁺ of 1012.87. In addition, the emission spectrum of a 1×10⁻⁵ mol/l solution of the resultant compound in toluene at room temperature was measured with an F-4500 manufactured by Hitachi, Ltd. at an excitation wavelength of 480 nm. As a result, its maximum emission wavelength was found to be 614 nm. In addition, the absolute quantum yield of the compound at room temperature in a solution state was measured with an absolute PL quantum yield-measuring apparatus (C9920-02) manufactured by Hamamatsu Photonics K.K. As a result, the absolute quantum yield was found to be 0.9 (relative value when the absolute quantum yield of Ir(pbiq)₃ was defined as 1.0).

### Synthesis Example 4

### Synthesis of Exemplified Compound KK-04

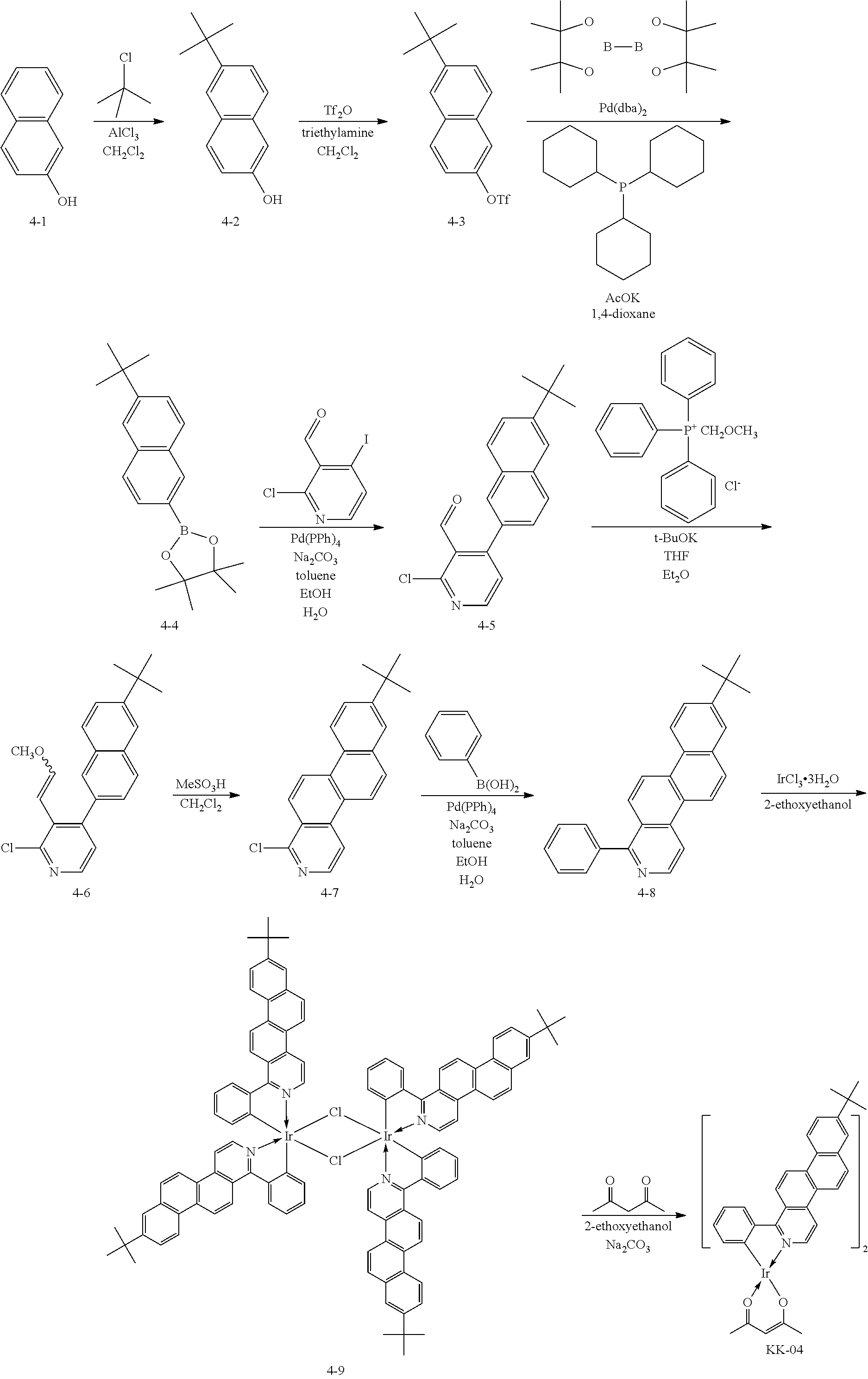

(1) Synthesis of Compound 4-2

The following reagents and solvent were loaded into a reaction vessel whose system was in a nitrogen atmosphere.

2-Naphthol: 34.9 g (242 mmol)

2-Chloro-2-methylpropane: 47.3 g (510 mmol)

Aluminum chloride: 2.45 g (18.4 mmol)

Dry dichloromethane: 150 ml

Next, the temperature of the reaction solution was increased to 40° C. and then the reaction solution was stirred at the temperature (40° C.) for 6 hours. After the completion of the reaction, the resultant was cooled to room temperature and then the solvent was removed by distillation under reduced pressure. Next, 300 ml of a 5% aqueous solution of sodium hydroxide were added to the residue. The mixture was stirred at 80° C. for 2 hours and then filtered. Next, a crystal collected by the filtration was dissolved in 500 ml of chloroform and then 50 ml of hydrochloric acid were dropped to the solution, followed by stirring at room temperature for 1 hour. Next, water was charged into the resultant, and then the organic layer was extracted with chloroform and dried with anhydrous sodium sulfate. After that, the solvent was removed by distillation under reduced pressure. Next, the residue was purified by column chromatography (gel for chromatography: BW300 (manufactured by FUJI SILYSIA CHEMICAL LTD.), eluent: ethyl acetate/chloroform=1/1) to provide 5.9 g of Compound 4-2 (yield: 12%).

(2) Synthesis of Compound 4-3

The following reagents and solvent were loaded into a reaction vessel whose system was in a nitrogen atmosphere.

Compound 4-2: 5.7 g (28.5 mmol)

Triethylamine: 82 ml (58.7 mmol)

Dry dichloromethane: 100 ml

Next, the reaction solution was cooled to 0° C. and then the reaction solution was stirred at the temperature (0° C.) for 30 minutes. Next, 5.7 ml (33.6 mmol) of trifluoromethane anhydride were slowly dropped to the reaction solution, and then the reaction solution was stirred for 2 hours while its temperature was maintained at 0° C. After the completion of the reaction, 150 ml of hydrochloric acid were added to the resultant, and then the organic layer was extracted with chloroform and dried with anhydrous sodium sulfate. After that, the solvent was removed by distillation under reduced pressure. Next, the residue was purified by column chromatography (gel for chromatography: BW300 (manufactured by FUJI SILYSIA CHEMICAL LTD.), eluent: heptane/chloroform=2/1) to provide 8.6 g of Compound 4-3 (yield: 90%).

(3) Synthesis of Compound 4-4

The following reagents and solvent were loaded into a reaction vessel.

Compound 4-3: 10.0 g (30.1 mmol)

Bis(pinacolato)diboron: 11.5 g (45.1 mmol)

Bis(dibenzylideneacetone)palladium(0): 0.87 g (1.50 mmol)

Tricyclohexylphosphine: 0.84 g (3.01 mmol)

Potassium acetate: 8.86 g (90.3 mmol)

1,4-Dioxane: 200 ml

Next, the temperature of the reaction solution was increased to 100° C. and then the reaction solution was stirred at the temperature (100° C.) for 4 hours. After the completion of the reaction, water was charged into the resultant, and then the organic layer was extracted with toluene and dried with anhydrous sodium sulfate. After that, the solvent was removed by distillation under reduced pressure. Next, the residue was purified by column chromatography (gel for chromatography: BW300 (manufactured by FUJI SILYSIA CHEMICAL LTD.), eluent: toluene/heptane=2/1) to provide 7.33 g of Compound 4-4 (yield: 78%).

(4) Synthesis of Compound 4-5

The following reagents and solvents were loaded into a reaction vessel.

Compound 1-1: 3.83 g (14.3 mmol)

Compound 4-4: 4.0 g (12.9 mmol)

Toluene: 200 ml

Ethanol: 100 ml

Aqueous solution of sodium carbonate (2 N): 100 ml

Next, 0.83 g (0.72 mmol) of tetrakis(triphenylphosphine)palladium(0) was added while the reaction solution was stirred under a nitrogen atmosphere at room temperature. Next, the temperature of the reaction solution was increased to 60° C. and then the reaction solution was stirred at the temperature (60° C.) for 7 hours. After the completion of the reaction, water was charged into the resultant, and then the organic layer was extracted with toluene and dried with anhydrous sodium sulfate. After that, the solvent was removed by distillation under reduced pressure. Next, the residue was purified by column chromatography (gel for chromatography: BW300 (manufactured by FUJI SILYSIA CHEMICAL LTD.), eluent: ethyl acetate/heptane=1/2) and then washed with methanol to provide 1.6 g of Compound 4-5 (yield: 38%).

(5) Synthesis of Compound 4-6

The following reagents and solvent were loaded into a reaction vessel.

(Methoxymethyl)triphenylphosphonium chloride: 4.23 g (12.4 mmol)

Potassium tert-butoxide (1 M solution in THF): 12.4 ml (12.4 mmol)

Dry ether: 25 ml

Next, the contents in the reaction vessel were stirred at room temperature for 30 minutes to be suspended. Next, a THF solution obtained by dissolving Compound 4-5 (1.6 g, 4.94 mmol) in 40 ml of dry THF was dropped to the suspension, and then the mixture was stirred for 10 hours while its temperature was kept at room temperature. After the completion of the reaction, water was charged into the resultant, and then the organic layer was extracted with toluene and dried with anhydrous sodium sulfate. After that, the solvent was removed by distillation under reduced pressure. Next, the residue was purified by column chromatography (gel for chromatography: BW300 (manufactured by FUJI SILYSIA CHEMICAL LTD.), eluent: ethyl acetate/heptane=1/3) to provide 1.5 g of Compound 4-6 (yield: 86%).

(6) Synthesis of Compound 4-7

4 Milliliters of methanesulfonic acid and 20 ml of dry dichloromethane were charged into a reaction vessel, and then the mixture was stirred at room temperature for 5 minutes. Next, a solution obtained by dissolving Compound 4-6 (1.5 g, 4.69 mmol) in 20 ml of dry dichloromethane was dropped to the mixture, and then the whole was stirred for 17 hours while its temperature was kept at room temperature. After the completion of the reaction, water was charged into the resultant, and then the organic layer was extracted with chloroform and dried with anhydrous sodium sulfate. After that, the solvent was removed by distillation under reduced pressure. Next, the residue was purified by column chromatography (gel for chromatography: BW300 (manufactured by FUJI SILYSIA CHEMICAL LTD.), eluent: chloroform) and then recrystallized with toluene twice to provide 600 mg of Compound 4-7 (yield: 40%).

(7) Synthesis of Compound 4-8

The following reagents and solvents were loaded into a reaction vessel.

Compound 4-7: 600 mg (1.88 mmol)

Compound B2-1: 274 mg (2.25 mmol)

Toluene: 60 ml

Ethanol: 30 ml

Aqueous solution of sodium carbonate (2 N): 30 ml

Next, 108 mg (0.094 mmol) of tetrakis(triphenylphosphine)palladium(0) were added while the reaction solution was stirred at room temperature under a nitrogen atmosphere. Next, the temperature of the reaction solution was increased to 85° C. and then the reaction solution was stirred at the temperature (85° C.) for 7 hours. After the completion of the reaction, water was charged into the resultant, and then the organic layer was extracted with toluene and dried with anhydrous sodium sulfate. After that, the solvent was removed by distillation under reduced pressure. Next, the residue was purified by column chromatography (gel for chromatography: BW300 (manufactured by FUJI SILYSIA CHEMICAL LTD.), eluent: chloroform) and then washed with methanol to provide 540 mg of Compound 4-8 (yield: 80%).

The structure of the compound was confirmed by ¹H-NMR measurement (400 MHz, CDCl₃).

σ (ppm): 8.84-8.83 (d, 1H), 8.72-8.68 (m, 3H), 8.53-8.52 (d, 1H), 8.22-8.20 (d, 1H), 8.08-8.05 (d, 1H), 7.98 (s, 1H), 7.84-7.82 (d, 1H), 7.78-7.76 (m, 2H), 7.60-7.52 (m, 3H), 1.49 (s, 9H).

(8) Synthesis of Compound 4-9

The following reagents and solvent were loaded into a reaction vessel.

Compound 4-8: 500 mg (1.38 mmol)

Iridium(III) chloride hydrate: 222 mg (0.63 mmol)

2-Ethoxyethanol: 20 ml

Water: 5 ml

Next, the temperature of the reaction solution was increased to 100° C. in a nitrogen atmosphere, and then the reaction solution was stirred at the temperature (100° C.) for 7 hours. After the completion of the reaction, water was charged into the resultant, and the precipitated solid was collected by filtration. Next, the solid collected by the filtration was washed with water and ethanol, followed by drying. Thus, 550 mg of Compound 4-9 were obtained (yield: 84%).

(9) Synthesis of Exemplified Compound KK-04

The following reagents and solvent were loaded into a reaction vessel.

Compound 4-8: 250 mg (0.13 mmol)

Acetylacetone: 2.0 g (20.2 mmol)

Sodium carbonate: 500 mg (4.72 mmol)

2-Ethoxyethanol: 5 ml

Next, the temperature of the reaction solution was increased to 95° C. in a nitrogen atmosphere, and then the reaction solution was stirred at the temperature (95° C.) for 7 hours. After the completion of the reaction, water was charged into the resultant, and the precipitated solid was collected by filtration, and was then washed with water and ethanol. After drying, the residue was purified by column chromatography (gel for chromatography: BW200 (manufactured by FUJI SILYSIA CHEMICAL LTD.), eluent: chloroform) to provide 160 mg of Exemplified Compound KK-04 (yield: 60%). Subsequently, sublimation purification was performed under the conditions of 1×10⁻⁴ Pa and 390° C. to provide 10 mg of Exemplified Compound KK-04 as a sublimated product.

The structure of the compound was confirmed by ¹H-NMR measurement (400 MHz, CDCl₃).

σ (ppm): 9.11-9.09 (d, 2H), 8.88-8.86 (d, 2H), 8.78-8.76 (d, 2H), 8.71-8.70 (d, 2H), 8.68-8.66 (d, 2H), 8.39-8.37 (d, 2H), 8.29-8.27 (d, 2H), 8.10-8.08 (d, 2H), 8.00 (s, 2H), 7.89-7.87 (d, 2H), 6.96-6.93 (t, 2H), 6.71-6.67 (t, 2H), 6.47-6.45 (d, 2H), 5.26 (s, 1H), 1.81 (s, 3H), 1.52 (s, 9H).

In addition, matrix assisted ionization time-of-flight mass spectrometry (MALDI-TOF MS) confirmed that the compound had an M⁺ of 1012.29. In addition, the emission spectrum of a 1×10⁻⁵ mol/l solution of the resultant compound in toluene at room temperature was measured with an F-4500 manufactured by Hitachi, Ltd. at an excitation wavelength of 480 nm. As a result, its maximum emission wavelength was found to be 612 nm. In addition, the absolute quantum yield of the compound at room temperature in a solution state was measured with an absolute PL quantum yield-measuring apparatus (C9920-02) manufactured by Hamamatsu Photonics K.K. As a result, the absolute quantum yield was found to be 1.0 (relative value when the absolute quantum yield of Ir(pbiq)₃ was defined as 1.0).

### Synthesis Example 5

### Synthesis of Exemplified Compound KK-28

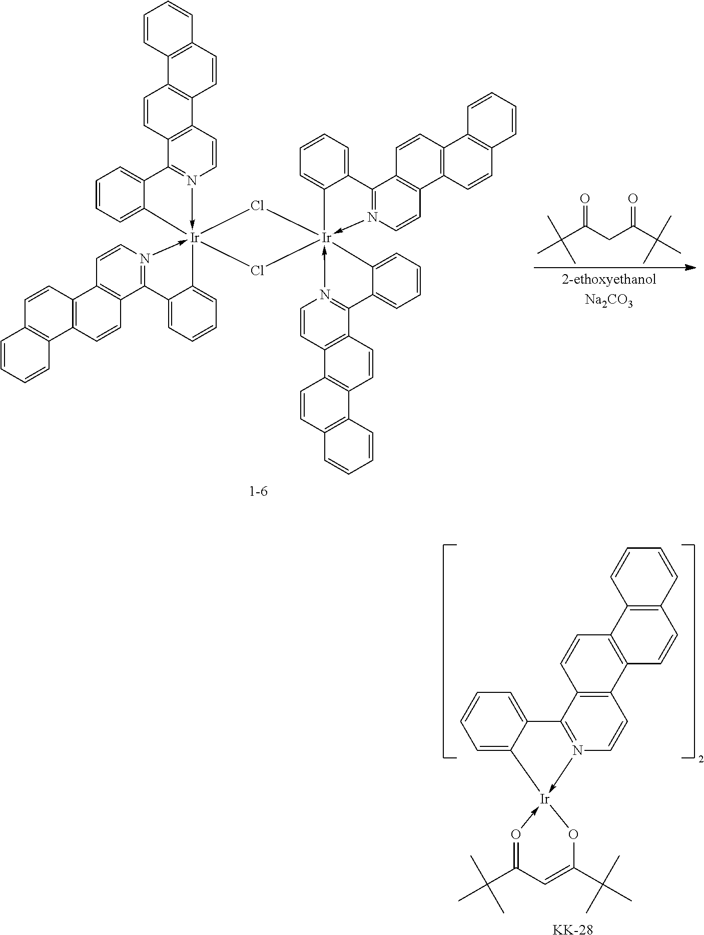

The following reagents and solvent were loaded into a reaction vessel.

Compound 1-6: 100 mg (0.060 mmol)

Dipivaloylmethane: 3.0 g (16.3 mmol)

Sodium carbonate: 200 mg (1.89 mmol)

2-Ethoxyethanol: 5 ml

Next, the temperature of the reaction solution was increased to 95° C. in a nitrogen atmosphere, and then the reaction solution was stirred at the temperature (95° C.) for 7 hours. After the completion of the reaction, water was charged into the resultant, and the precipitated solid was collected by filtration, and was then washed with water and ethanol. After drying, the residue was purified by column chromatography (gel for chromatography: BW200 (manufactured by FUJI SILYSIA CHEMICAL LTD.), eluent: chloroform) to provide 56 mg of Exemplified Compound KK-01 (yield: 48%). Subsequently, sublimation purification was performed under the conditions of 1×10⁻⁴ Pa and 385° C. to provide 7 mg of Exemplified Compound KK-28 as a sublimated product.

The structure of the compound was confirmed by ¹H-NMR measurement (400 MHz, CDCl₃).

σ (ppm): 9.16-9.14 (d, 2H), 8.91-8.88 (d, 2H), 8.86-8.84 (d, 2H), 8.71-8.69 (d, 2H), 8.61-8.60 (d, 2H), 8.32-8.28 (m, 4H), 8.11-8.09 (d, 2H), 8.07-8.05 (d, 2H), 7.82-7.78 (t, 2H), 7.75-7.71 (t, 2H), 6.98-6.95 (t, 2H), 6.71-6.68 (t, 2H), 6.60-6.59 (d, 2H), 5.46 (s, 1H), 0.85 (s, 18H).

Matrix assisted ionization time-of-flight mass spectrometry (MALDI-TOF MS) confirmed that the compound had an M⁺ of 984.35. In addition, the emission spectrum of a 1×10⁻⁵ mol/l solution of the resultant compound in toluene at room temperature was measured with an F-4500 manufactured by Hitachi, Ltd. at an excitation wavelength of 480 nm. As a result, its maximum emission wavelength was found to be 616 nm. In addition, the absolute quantum yield of the compound at room temperature in a solution state was measured with an absolute PL quantum yield-measuring apparatus (C9920-02) manufactured by Hamamatsu Photonics K.K. As a result, the absolute quantum yield was found to be 1.0 (relative value when the absolute quantum yield of Ir(pbiq)₃ was defined as 1.0).

### Synthesis Example 6

### Synthesis of Exemplified Compound KK-31

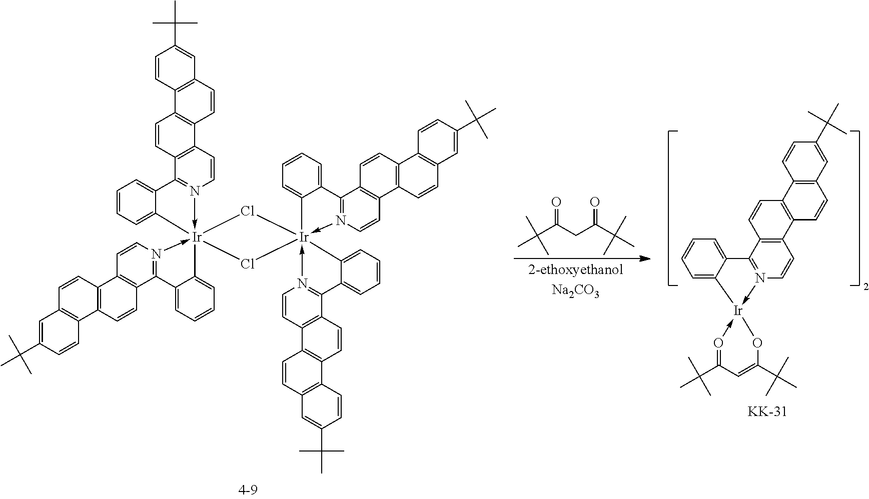

The following reagents and solvent were loaded into a reaction vessel.

Compound 4-9: 250 mg (0.13 mmol)

Dipivaloylmethane: 3.0 g (16.3 mmol)

Sodium carbonate: 500 mg (1.89 mmol)

2-Ethoxyethanol: 12 ml

Next, the temperature of the reaction solution was increased to 95° C. in a nitrogen atmosphere, and then the reaction solution was stirred at the temperature (95° C.) for 7 hours. After the completion of the reaction, water was charged into the resultant, and the precipitated solid was collected by filtration, and was then washed with water and ethanol. After drying, the residue was purified by column chromatography (gel for chromatography: BW200 (manufactured by FUJI SILYSIA CHEMICAL LTD.), eluent: chloroform) to provide 175 mg of Exemplified Compound KK-31 (yield: 61%). Subsequently, sublimation purification was performed under the conditions of 1×10⁻⁴ Pa and 390° C. to provide 15 mg of Exemplified Compound KK-31 as a sublimated product.

The structure of the compound was confirmed by ¹H-NMR measurement (400 MHz, CDCl₃).

σ (ppm): 9.13-9.11 (d, 2H), 8.87-8.84 (d, 2H), 8.78-8.76 (d, 2H), 8.68-8.65 (d, 2H), 8.60-8.58 (d, 2H), 8.30-8.28 (m, 4H), 8.08-8.06 (d, 2H), 7.99 (s, 2H), 7.89-7.86 (d, 2H), 6.97-6.94 (t, 2H), 6.71-6.67 (t, 2H), 6.61-6.59 (d, 2H), 5.45 (s, 1H), 1.51 (s, 18H), 0.84 (s, 18H).

Matrix assisted ionization time-of-flight mass spectrometry (MALDI-TOF MS) confirmed that the compound had an M⁺ of 1096.53. In addition, the emission spectrum of a 1×10⁻⁵ mol/l solution of the resultant compound in toluene at room temperature was measured with an F-4500 manufactured by Hitachi, Ltd. at an excitation wavelength of 480 nm. As a result, its maximum emission wavelength was found to be 614 nm. In addition, the absolute quantum yield of the compound at room temperature in a solution state was measured with an absolute PL quantum yield-measuring apparatus (C9920-02) manufactured by Hamamatsu Photonics K.K. As a result, the absolute quantum yield was found to be 1.0 (relative value when the absolute quantum yield of Ir(pbiq)₃ was defined as 1.0).

### Synthesis Example 7

### Synthesis of Exemplified Compound KK-29

Exemplified Compound KK-29 was obtained by the same method as that of Synthesis Example 3 with the exception that in the section (6) of Synthesis Example 3, dipivaloylmethane was used instead of acetylacetone. Matrix assisted ionization time-of-flight mass spectrometry (MALDI-TOF MS) confirmed that the compound had an M⁺ of 1096.10.

### Synthesis Example 8

### Synthesis of Exemplified Compound KK-30

Exemplified Compound KK-30 was obtained by the same method as that of Synthesis Example 2 with the exception that in the section (7) of Example 2, dipivaloylmethane was used instead of acetylacetone. Matrix assisted ionization time-of-flight mass spectrometry (MALDI-TOF MS) confirmed that the compound had an M⁺ of 1096.85.

### Synthesis Example 9

### Synthesis of Exemplified Compound KK-35

Exemplified Compound KK-35 was obtained by the same method as that of Synthesis Example 1 with the exception that in the section (6) of Example 1, Compound B1-A shown below was used instead of Compound B1-1 and dipivaloylmethane was used instead of acetylacetone.

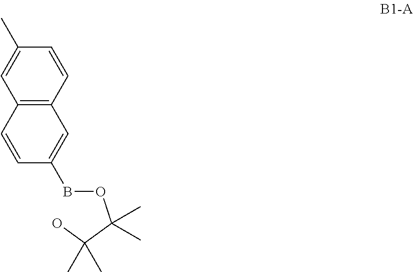

Matrix assisted ionization time-of-flight mass spectrometry (MALDI-TOF MS) confirmed that the compound had an M⁺ of 1012.55.

### Synthesis Example 10

### Synthesis of Exemplified Compound KK-36

Exemplified Compound KK-36 was obtained by the same method as that of Synthesis Example 2 with the exception that in the section (7) of Synthesis Example 2, Compound B2-A shown below was used instead of Compound B2-1 and dipivaloylmethane was used instead of acetylacetone.

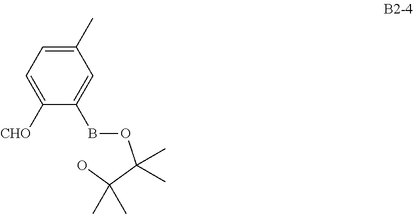

Matrix assisted ionization time-of-flight mass spectrometry (MALDI-TOF MS) confirmed that the compound had an M⁺ of 1012.49.

### Synthesis Examples 11 to 15

### Synthesis of Exemplified Compounds X-106, X-131, X-135, X-137, and X-145

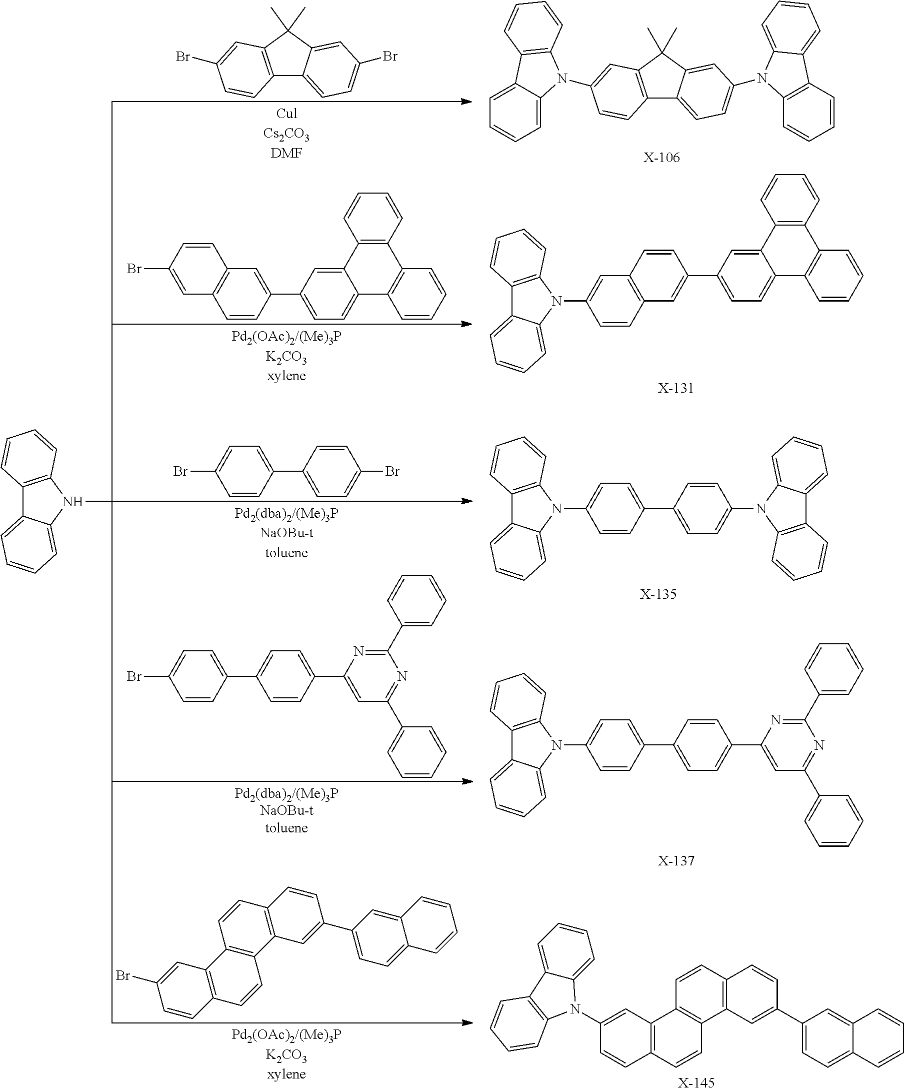

Exemplified Compounds X-106, X-131, X-135, X-137, and X-145 were each synthesized according to the above-mentioned synthesis scheme with 9H-carbazole as a starting raw material by employing a cross-coupling reaction involving using a Pd catalyst. The structures of the resultant compounds (Exemplified Compound X-106, X-131, X-135, X-137, and X-145) were confirmed by MALDI-TOF-MS. Table 1 shows the results.

### Synthesis Examples 16 to 18

### Synthesis of Exemplified Compounds H-108, H-131, and H-139

Exemplified Compounds H-108, H-131, and H-139 were each synthesized according to the following synthesis scheme with 4-dibenzothiopheneboronic acid as a starting raw material by employing a cross-coupling reaction involving using a Pd catalyst.

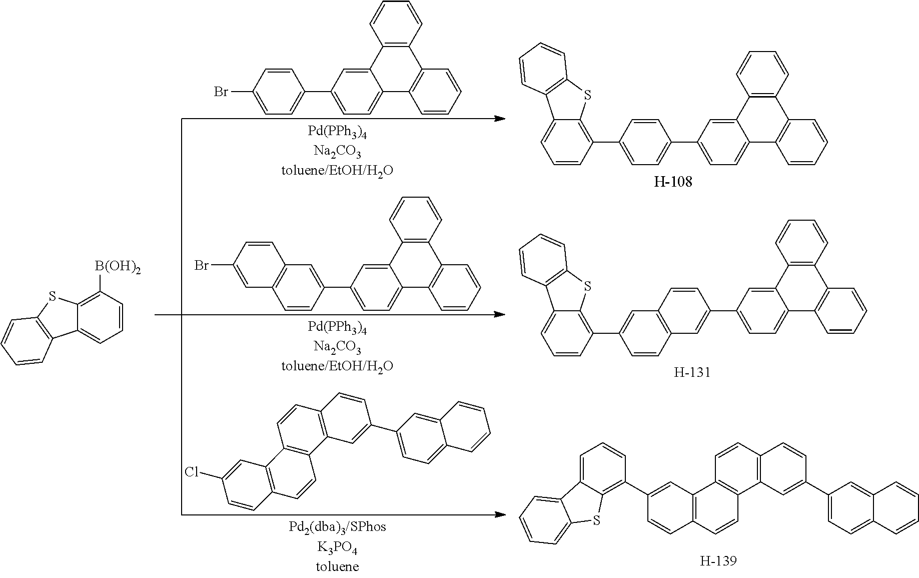

The resultant compounds (Exemplified Compounds H-108, H-131, and H-139) were identified by MALDI-TOF-MS. Table 2 shows the results.

### Synthesis Examples 19 and 20

### Synthesis of Exemplified Compounds H-206 and H-210

Exemplified Compounds H-206 and H-210 were each synthesized according to the following synthesis scheme by synthesizing benzo[b]naphtho[2,1-d]thiophene-10-boronic acid and then performing a cross-coupling reaction involving using a Pd catalyst.

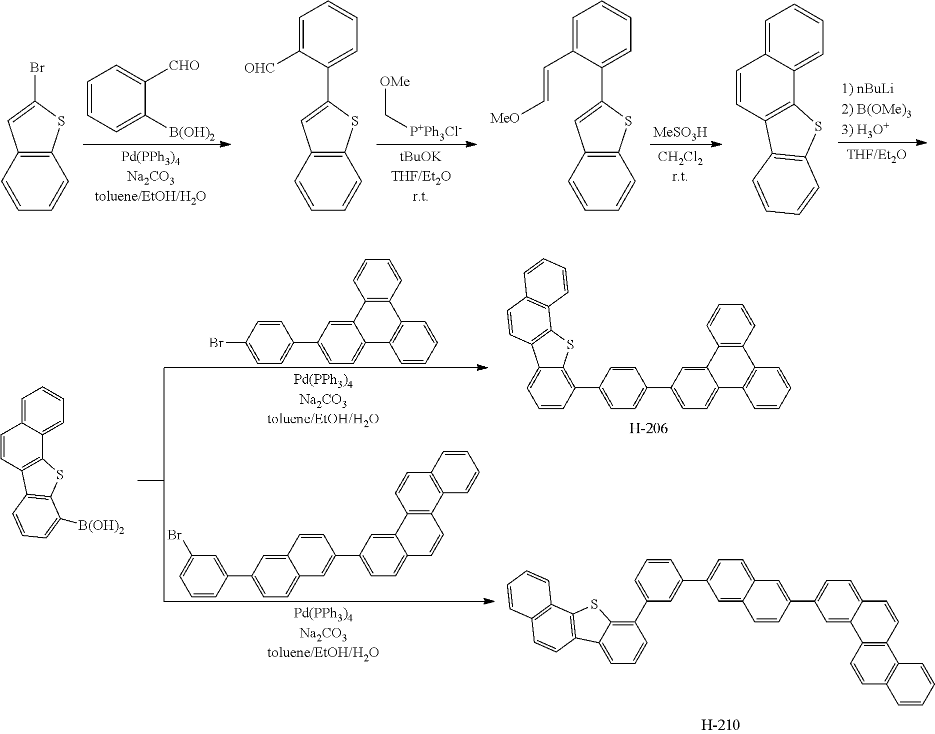

The resultant compounds (Exemplified Compounds H-206 and H-210) were identified by MALDI-TOF-MS. Table 2 shows the results.

### Synthesis Examples 21 and 22

### Synthesis of Exemplified Compounds H-317 and H-322

Exemplified Compounds H-317 and H-322 were each synthesized according to the following synthesis scheme by synthesizing 2-chlorobenzo[b]phenanthro[3,4-d]thiophene and then performing a cross-coupling reaction involving using a Pd catalyst.

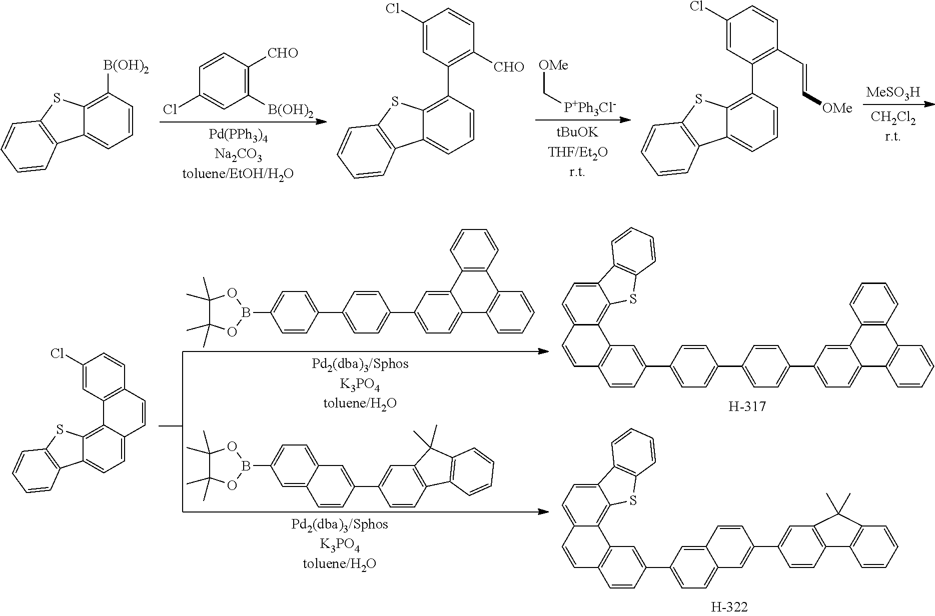

The resultant compounds (Exemplified Compounds H-317 and H-322) were identified by MALDI-TOF-MS. Table 2 shows the results.

### Synthesis Examples 23 to 25

### Synthesis of Exemplified Compounds H-401, H-422, and H-424

Dibenzo[b,mn]xanthene-7-boronic acid was synthesized according to the following synthesis scheme. Subsequently, Exemplified Compounds H-401, H-422, and H-424 were each synthesized by performing a cross-coupling reaction involving using a Pd catalyst.

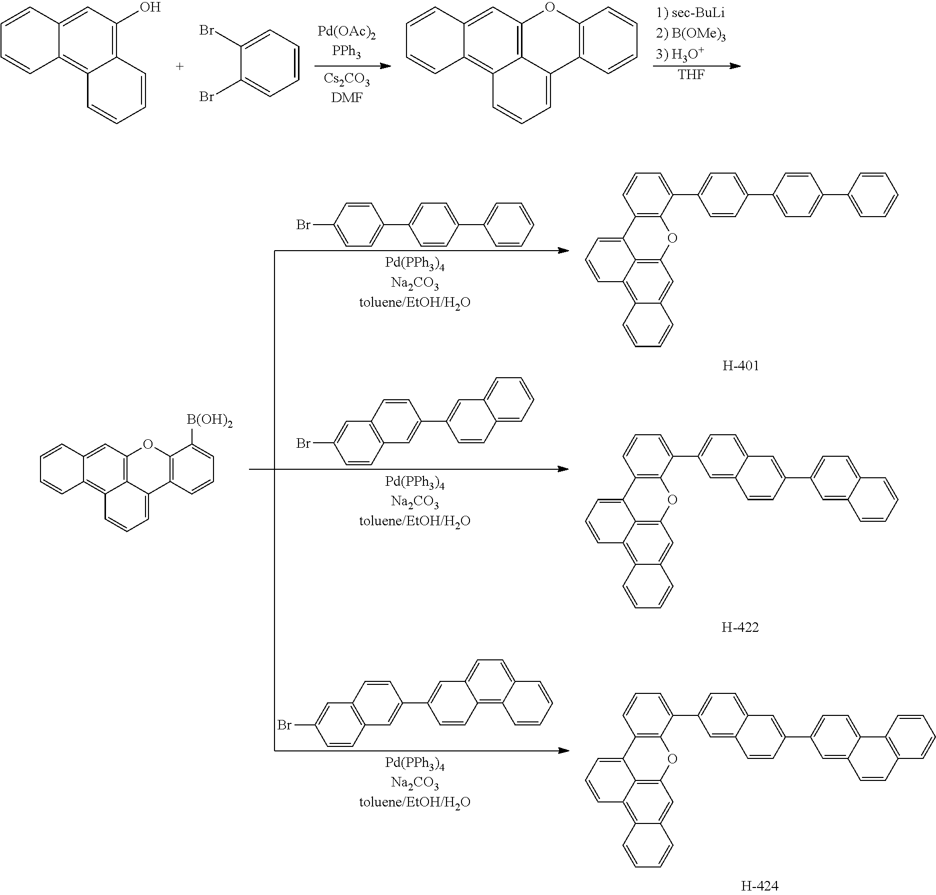

The resultant compounds (Exemplified Compounds H-401, H-422, and H-424) were identified by MALDI-TOF-MS. Table 2 shows the results.

### Synthesis Example 26

### Synthesis of Exemplified Compound H-439

Exemplified Compound H-439 was synthesized by the same method as that of Synthesis Example 27 with the exception that in Synthesis Example 27, the starting raw material was changed from 9-hydroxyphenanthrene to 3,6-dimethylphenanthrene-9-ol. The resultant compound (Exemplified Compound H-439) was identified by MALDI-TOF-MS. Table 2 shows the result.

### Synthesis Examples 27 to 29

### Synthesis of Exemplified Compounds H-507, H-508, and H-509

Exemplified Compounds H-507, H-508, and H-509 were each synthesized according to the following synthesis scheme by synthesizing 5-chlorodibenzo[b,mn]xanthene and then performing a cross-coupling reaction involving using a Pd catalyst.

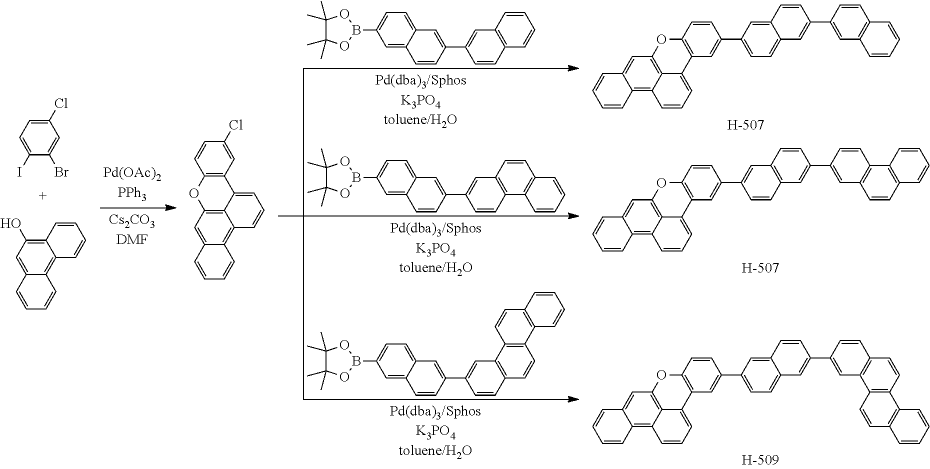

The resultant compounds (Exemplified Compounds H-507, H-508, and H-509) were identified by MALDI-TOF-MS. Table 2 shows the results.

### Synthesis Example 30

### Synthesis of Exemplified Compound H-629

Exemplified Compound H-629 was synthesized by the same method as that of Synthesis Example 22 with the exception that in Synthesis Example 22, the starting raw material was changed from 2-bromobenzo[b]thiophene to 2-bromobenzofuran.

The resultant compound (Exemplified Compound H-629) was identified by MALDI-TOF-MS. Table 2 shows the result.

### Synthesis Example 31

### Synthesis of Exemplified Compound H-712

Exemplified Compound H-712 was synthesized according to the following synthesis scheme.

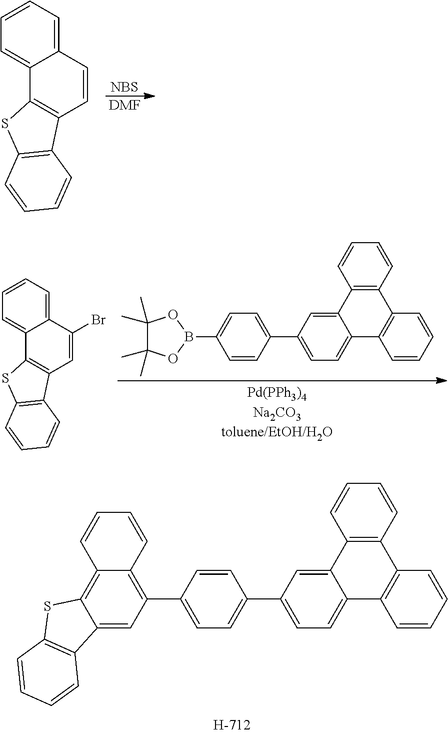

Specifically, 5-bromobenzo[b]naphtho[2,1-d]thiophene was synthesized from benzo[b]naphtho[2,1-d]thiophene obtained as a compound in Synthesis Examples 22 and 23. Subsequently, Exemplified Compound H-712 was synthesized by performing a cross-coupling reaction involving using a Pd catalyst.

The resultant compound (Exemplified Compound H-712) was identified by MALDI-TOF-MS. Table 2 shows the result.

TABLE 1

| | | MS | MS |
|---|---|---|---|
| | Exemplified | (calculated | (measured |
| | Compound | value) | value) |
| Synthesis | X-106 | 524.65 | 524.12 |
| Example 11 | | | |
| Synthesis | X-131 | 519.63 | 519.23 |
| Example 12 | | | |
| Synthesis | X-135 | 484.59 | 484.71 |
| Example 13 | | | |
| Synthesis | X-137 | 549.66 | 549.83 |
| Example 14 | | | |
| Synthesis | X-145 | 519.63 | 519.69 |
| Example 15 | | | |
| Synthesis | H-108 | 486.14 | 486.33 |
| Example 16 | | | |
| Synthesis | H-131 | 536.16 | 536.31 |
| Example 17 | | | |
| Synthesis | H-139 | 536.16 | 536.28 |
| Example 18 | | | |
| Synthesis | H-206 | 536.16 | 536.35 |
| Example 19 | | | |
| Synthesis | H-210 | 662.21 | 662.39 |
| Example 20 | | | |
| Synthesis | H-317 | 662.21 | 662.42 |
| Example 21 | | | |
| Synthesis | H-322 | 602.21 | 602.41 |
| Example 22 | | | |
| Synthesis | H-401 | 496.18 | 496.38 |
| Example 23 | | | |
| Synthesis | H-422 | 520.18 | 520.35 |
| Example 24 | | | |
| Synthesis | H-424 | 570.20 | 570.43 |
| Example 25 | | | |
| Synthesis | H-439 | 548.21 | 548.40 |
| Example 26 | | | |
| Synthesis | H-507 | 520.18 | 520.35 |
| Example 27 | | | |
| Synthesis | H-508 | 570.20 | 570.42 |
| Example 28 | | | |
| Synthesis | H-509 | 620.21 | 620.35 |
| Example 29 | | | |
| Synthesis | H-629 | 520.18 | 520.36 |
| Example 30 | | | |
| Synthesis | H-712 | 536.16 | 536.33 |
| Example 31 | | | |

### Example 1

In this example, an organic light-emitting device having a construction in which “an anode/a hole transport layer/an electron blocking layer/an emission layer/a hole blocking layer/an electron transport layer/a cathode” were formed on a substrate in the stated order was produced by the following method.

First, ITO was formed into a film on a glass substrate and then subjected to desired patterning processing to form an ITO electrode (anode). At this time, the thickness of the ITO electrode was set to 100 nm. The substrate on which the ITO electrode had been thus formed was used as an ITO substrate in the following steps.

An organic light-emitting device was obtained by continuously forming, on the ITO substrate, organic compound layers and electrode layers shown in Table 3 below. It is to be noted that at this time, the electrode area of the opposing electrode (metal electrode layers, cathode) was set to 3 mm².

TABLE 2

| | | Thick- |
|---|---|---|
| | | ness |
| | Material | (nm) |
| Hole transport layer: HTL | HT-2 | 40 |
| Electron blocking layer: EBL | HT-7 | 10 |
| Emission layer | X-106 (host) | 30 |
| Host: HOST | KK-01 (guest) | |
| Guest: GUEST | (X-106:KK-01 = 96:4 | |
| | (weight ratio)) | |
| Hole blocking layer: HBL | ET-3 | 10 |
| Electron transport layer: ETL | ET-2 | 50 |
| First metal electrode layer | LiF | 0.5 |
| Second metal electrode layer | Al | 100 |

The characteristics of the resultant device were measured and evaluated by measuring its current-voltage characteristics with a microammeter 4140B manufactured by Hewlett-Packard Company and measuring its emission luminance with a BM-7 manufactured by TOPCON CORPORATION. In this example, the light-emitting device had a maximum emission wavelength of 618 nm and chromaticity coordinates (x, y) of (0.67, 0.33).

As a result, emission efficiency in the case where the organic light-emitting device of this example was caused to emit light with its luminance set to 2,000 cd/m² was 23.6 cd/A. In addition, the luminance half lifetime of the organic light-emitting device of this example at a current value of 100 mA/cm² was 300 hours.

### Examples 2 to 26 and Comparative Examples 1 to 5

Organic light-emitting devices were each produced by the same method as that of Example 1 with the exception that in Example 1, the compounds used as the hole transport layer (HTL), the electron blocking layer (EBL), the emission layer host (HOST), the emission layer guest (GUEST), the hole blocking layer (HBL), and the electron transport layer (ETL) were appropriately changed to compounds shown in Table 4 below. The characteristics of the resultant devices were measured and evaluated in the same manner as in Example 1. Table 4 shows the results of the measurement.

TABLE 3

| | | | | | | | Emission | |
|---|---|---|---|---|---|---|---|---|
| | | | | | | | efficiency at | Half lifetime |
| | | | | | | | 2,000 cd/m² | at 100 mA/cm² |
| | HTL | EBL | HOST | GUEST | HBL | ETL | [cd/A] | [h] |
| Example 1 | HT2 | HT7 | X-106 | KK-01 | ET3 | ET2 | 23.6 | 300 |
| Example 2 | HT2 | HT7 | X-135 | KK-31 | ET3 | ET2 | 24.3 | 350 |
| Example 3 | HT1 | HT7 | X-137 | KK-03 | ET3 | ET2 | 22.1 | 290 |
| Example 4 | HT1 | HT7 | H-108 | KK-04 | ET3 | ET2 | 21.3 | 390 |
| Example 5 | HT1 | HT7 | H-131 | KK-28 | ET3 | ET2 | 23.3 | 680 |
| Example 6 | HT1 | HT7 | H-131 | KK-28 | ET4 | ET2 | 24.1 | 630 |
| Example 7 | HT1 | HT7 | H-131 | KK-36 | ET4 | ET1 | 22.2 | 550 |
| Example 8 | HT2 | HT7 | H-206 | KK-35 | ET3 | ET2 | 21.6 | 520 |
| Example 9 | HT2 | HT7 | H-210 | KK-31 | ET3 | ET2 | 24.6 | 480 |
| Example 10 | HT2 | HT7 | H-322 | KK-30 | ET4 | ET2 | 24.1 | 380 |
| Example 11 | HT2 | HT7 | H-322 | KK-03 | ET4 | ET1 | 21.9 | 500 |
| Example 12 | HT2 | HT7 | H-322 | KK-04 | ET3 | ET2 | 21.8 | 470 |
| Example 13 | HT1 | HT7 | H-401 | KK-31 | ET3 | ET2 | 23.4 | 660 |
| Example 14 | HT1 | HT7 | H-401 | KK-28 | ET4 | ET2 | 23.8 | 650 |
| Example 15 | HT2 | HT7 | H-422 | KK-31 | ET3 | ET2 | 24.1 | 620 |
| Example 16 | HT2 | HT7 | H-422 | KK-02 | ET3 | ET2 | 21.6 | 290 |
| Example 17 | HT2 | HT7 | H-424 | KK-31 | ET3 | ET2 | 23.5 | 640 |
| Example 18 | HT2 | HT7 | H-424 | KK-28 | ET3 | ET2 | 23.2 | 510 |
| Example 19 | HT2 | HT7 | H-424 | KK-04 | ET4 | ET2 | 22.6 | 710 |
| Example 20 | HT2 | HT7 | H-424 | KK-29 | ET7 | ET2 | 22.8 | 410 |
| Example 21 | HT2 | HT7 | H-507 | KK-03 | ET3 | ET2 | 21.8 | 340 |
| Example 22 | HT2 | HT7 | H-508 | KK-04 | ET3 | ET1 | 21.7 | 670 |
| Example 23 | HT1 | HT7 | H-509 | KK-30 | ET3 | ET2 | 23.3 | 620 |
| Example 24 | HT1 | HT7 | H-629 | KK-31 | ET3 | ET2 | 24.9 | 700 |
| Example 25 | HT2 | HT7 | H-712 | KK-31 | ET4 | ET2 | 24.6 | 730 |
| Example 26 | HT3 | HT7 | H-712 | KK-36 | ET4 | ET2 | 23.8 | 490 |
| Comparative | HT2 | HT7 | EM9 | KK-01 | ET4 | ET2 | 22.5 | 100 |
| Example 1 | | | | | | | | |
| Comparative | HT2 | HT7 | EM9 | KK-03 | ET3 | ET2 | 21.9 | 90 |
| Example 2 | | | | | | | | |
| Comparative | HT2 | HT7 | H-108 | RD5 | ET4 | ET2 | 13.2 | 370 |
| Example 3 | | | | | | | | |
| Comparative | HT2 | HT7 | H-131 | RD3 | ET4 | ET2 | 7.8 | 360 |
| Example 4 | | | | | | | | |
| Comparative | HT2 | HT8 | H-424 | RD5 | ET3 | ET2 | 13.3 | 410 |
| Example 5 | | | | | | | | |

The organic light-emitting devices of Comparative Examples 1 and 2 had shorter luminance half lifetimes than those of the organic light-emitting devices of Examples, though the former devices were each substantially comparable to the latter devices in emission efficiency. This is caused by the fact that the host in the emission layer is not the heterocycle-containing compound represented by the general formula [5]. Therefore, the heterocycle-containing compound represented by the general formula [5] used as a host for the emission layer in the organic light-emitting device of the present invention is a compound having high structural stability and moderate hole-transporting property. Accordingly, the organic light-emitting device of the present invention was found to have high emission efficiency and a long luminance half lifetime.

On the other hand, the light-emitting devices used in Comparative Examples 3 to 5 had lower emission efficiencies than those of the organic light-emitting devices of Examples, though the former devices were each substantially comparable to the latter devices in luminance half lifetime. This is caused by the fact that the guest in the emission layer is not the big-based Ir complex represented by the general formula [1]. Therefore, an organic light-emitting device improved in emission efficiency and luminance half lifetime is obtained only when the heterocycle-containing compound represented by the general formula [5] having a lifetime-lengthening effect and the big-based Ir complex represented by the general formula [1] having high emission efficiency are combined like the organic light-emitting devices of Examples.

### Example 27

In this example, an organic light-emitting device having a construction in which “an anode/a hole transport layer/an electron blocking layer/an emission layer/a hole blocking layer/an electron transport layer/a cathode” were formed on a substrate in the stated order was produced. It is to be noted that in this example, the emission layer contains an assist material.

First, organic compound layers and electrode layers shown in Table 5 below were continuously formed on an ITO substrate that had been produced by the same method as that of Example 1. It is to be noted that at this time, the electrode area of the opposing electrode (metal electrode layers, cathode) was set to 3 mm².

TABLE 4

| | | Thick- |
|---|---|---|
| | | ness |
| | Material | (nm) |
| Hole transport layer: HTL | HT-2 | 40 |
| Electron blocking layer: EBL | HT-7 | 10 |
| Emission layer | X-106 (host) | 30 |
| Host: HOST | HT-02 (assist) | |
| Assist: ASSIST | KK-01 (guest) | |
| Guest: GUEST | (X-106:HT-2:KK-01 = 80:15:5 | |
| | (weight ratio)) | |
| Hole blocking layer: HBL | ET-3 | 10 |
| Electron transport layer: ETL | ET-2 | 50 |
| First metal electrode layer | LiF | 0.5 |
| Second metal electrode layer | Al | 100 |

The characteristics of the resultant device were measured and evaluated in the same manner as in Example 1. Here, the organic light-emitting device of this example had a maximum emission wavelength of 621 nm and chromaticity coordinates (x, y) of (0.67, 0.33). In addition, the device had an emission efficiency at the time of its light emission at a luminance of 1,500 cd/m² of 24.1 cd/A and a luminance half lifetime at a current value of 100 mA/cm² of 270 hours.

### Examples 28 to 34 and Comparative Examples 6 and 7

Organic light-emitting devices were each produced by the same method as that of Example 27 with the exception that in Example 27, the compounds used as the hole transport layer (HTL), the electron blocking layer (EBL), the emission layer host (HOST), the emission layer assist (ASSIST), the emission layer guest (GUEST), the hole blocking layer (HBL), and the electron transport layer (ETL) were changed as shown in Table 6. The characteristics of the resultant devices were measured and evaluated in the same manner as in Example 27. Table 6 shows the results of the measurement.

TABLE 5

| | | | | | | | | Emission | Half |
|---|---|---|---|---|---|---|---|---|---|
| | | | | | | | | efficiency at | lifetime at |
| | | | | | | | | 1,500 cd/m² | 100 mA/cm² |
| | HTL | EBL | HOST | ASSIST | GUEST | HBL | ETL | [cd/A] | [h] |
| Example 27 | HT2 | HT7 | X-106 | HT2 | KK-04 | ET3 | ET1 | 24.1 | 270 |
| Example 28 | HT1 | HT8 | H-108 | GD6 | KK-31 | ET3 | ET2 | 24.9 | 600 |
| Example 29 | HT2 | HT7 | H-206 | HT2 | KK-28 | ET3 | ET2 | 24.3 | 450 |
| Example 30 | HT2 | HT11 | H-210 | GD6 | KK-01 | ET4 | ET1 | 23.8 | 520 |
| Example 31 | HT2 | HT7 | H-317 | GD6 | KK-31 | ET7 | ET2 | 24.7 | 400 |
| Example 32 | HT2 | HT7 | H-424 | GD6 | KK-36 | ET3 | ET2 | 23.9 | 480 |
| Example 33 | HT3 | HT8 | H-439 | HT1 | KK-04 | ET4 | ET2 | 23.1 | 460 |
| Example 34 | HT2 | HT7 | H-507 | HT2 | KK-31 | ET3 | ET2 | 22.9 | 560 |
| Comparative | HT2 | HT8 | EM9 | HT2 | KK-04 | ET3 | ET2 | 23.5 | 130 |
| Example 6 | | | | | | | | | |
| Comparative | HT1 | HT8 | H-108 | GD6 | RD5 | ET4 | ET2 | 14.0 | 560 |
| Example 7 | | | | | | | | | |

Examples 27 to 34 showed that even when part of the host in the emission layer was changed to the assist material, an organic light-emitting device having high emission efficiency and a long lifetime was obtained as in Examples 1 to 26.

On the other hand, the organic light-emitting device of Comparative Example 6 had a shorter luminance half lifetime than those of Examples even when the assist material was incorporated into the emission layer because the host in the emission layer was not the heterocycle-containing compound represented by the general formula [5].

In addition, the organic light-emitting device of Comparative Example 7 had a lower emission efficiency than those of Examples even when the assist material was incorporated into the emission layer because the guest in the emission layer was not the big-based Ir complex represented by the general formula [1].

The foregoing showed that even in the case where the assist material was incorporated into the emission layer, an organic light-emitting device having high emission efficiency and a long luminance half lifetime was obtained only when the heterocycle-containing compound represented by the general formula [5] and the biq-based Ir complex represented by the general formula [1] were combined.

### INDUSTRIAL APPLICABILITY

As described above, the organic light-emitting device according to the present invention is a light-emitting device using both an iridium complex, which has a naphtho[2,1-f]isoquinoline skeleton having high emission efficiency as a ligand, as an emission layer guest and a heterocycle-containing compound, which has a lifetime-lengthening effect and high structural stability, as an emission layer host in combination. Thus, an organic light-emitting device having high emission efficiency and a good lifetime characteristic can be provided.

As described above by way of the embodiments and Examples, the organic compound layer (in particular, emission layer) of the organic light-emitting device of the present invention contains an niq-based Ir complex having a high emission quantum yield and a high color purity of a red color, and a heterocyclic compound having high bond stability. Therefore, according to one embodiment of the present invention, it is possible to provide the organic light-emitting device having high efficiency and improved in driving durability.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2013-021049, filed Feb. 6, 2013, which is hereby incorporated by reference herein in its entirety.

1. An organic light-emitting device comprising:
a pair of electrodes; and
an organic compound layer placed between the pair of electrodes,
wherein the organic compound layer comprises an iridium complex represented by the following general formula [1] and a heterocycle-containing compound as a host:
Ir(L)ₘ(L′)ₙ [1]
in the formula [1], Ir represents iridium, L and L′ represent bidentate ligands different from each other, provided that L and L′ each represent a ligand containing at least one alkyl group, m represents 2, n represents 1, and a partial structure Ir(L)ₘ comprises a partial structure represented by the following general formula [2]: 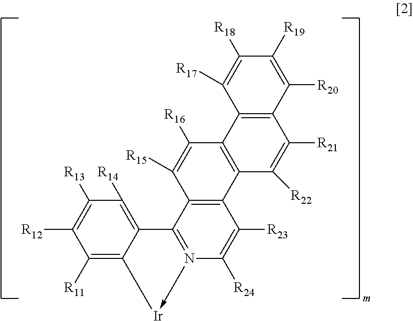
in the formula [2], R₁₁ to R₁₄ each represent a hydrogen atom, a fluorine atom, a substituted or unsubstituted alkyl group, an alkoxy group, a substituted amino group, a substituted or unsubstituted aryl group, or a substituted or unsubstituted heterocyclic group, and may be identical to or different from one another, and R₁₅ to R₂₄ each represent a hydrogen atom, a fluorine atom, a substituted or unsubstituted alkyl group, an alkoxy group, or a substituted amino group, and may be identical to or different from one another; and
a partial structure Ir(L′)ₙ comprises a partial structure containing a monovalent bidentate ligand.

2. The organic light-emitting device according to claim 1, wherein the partial structure Ir(L′)ₙ comprises a partial structure represented by any one of the following general formulae [3] to [5]: 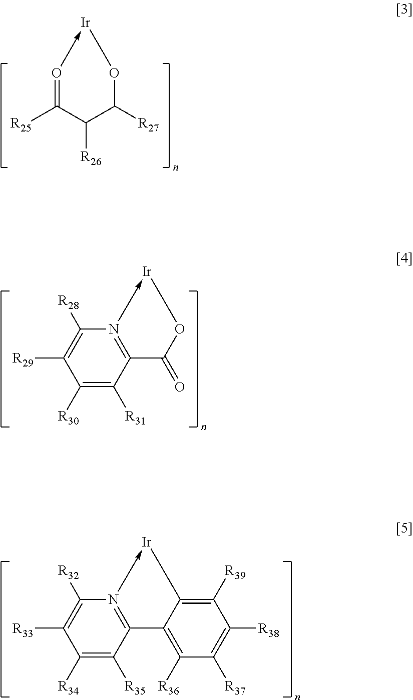
in the formulae [3] to [5], R₂₅ to R₃₉ each represent a hydrogen atom, an alkyl group, an alkoxy group, a substituted amino group, a substituted or unsubstituted aryl group, or a substituted or unsubstituted heterocyclic group, and may be identical to or different from one another.

3. The organic light-emitting device according to claim 2, wherein R₁₁ to R₂₄ in the general formula [2] each represent a substituent selected from a hydrogen atom, a fluorine atom, and an alkyl group having 1 to 10 carbon atoms;
R₂₅ to R₃₉ in the general formulae [3] to [5] each represent a substituent selected from a hydrogen atom and an alkyl group having 1 to 10 carbon atoms; and
at least one of the R₁₁ to R₃₉ represents an alkyl group having 1 to 10 carbon atoms.

4. The organic light-emitting device according to claim 2, wherein R₁₁ to R₂₄ in the general formula [2] each represent a substituent selected from a hydrogen atom, a fluorine atom, a methyl group, and a tert-butyl group;
R₂₅ to R₃₉ in the general formulae [3] to [5] each represent a substituent selected from a hydrogen atom, a methyl group, and a tert-butyl group; and
at least one of the R₁₁ to R₃₉ represents a structure that comprises a methyl group or a tert-butyl group.

5. The organic light-emitting device according to claim 2, wherein the partial structure Ir(L′)ₙ in the general formula [1] comprises a partial structure represented by the general formula [3].

6. The organic light-emitting device according to claim 1, wherein the heterocycle-containing compound comprises a compound represented by the following general formula [6] or [7]: 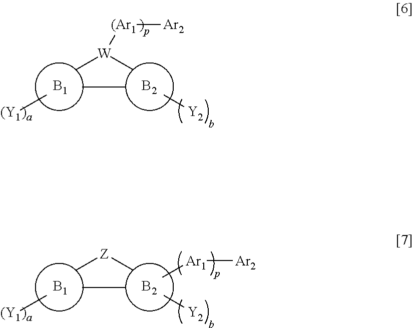
in the formula [6] and the formula [7], a ring B₁ and a ring B₂ each represent an aromatic ring selected from a benzene ring, a naphthalene ring, a phenanthrene ring, a triphenylene ring, and a chrysene ring, and the ring B₁ and the ring B₂ may each further have a substituent, Y₁ and Y₂ each represent an alkyl group, or a substituted or unsubstituted aryl group, a and b each represent an integer of 0 to 4, when a represents 2 or more, multiple Y₁'s may be identical to or different from each other, and when b represents 2 or more, multiple Y₂'s may be identical to or different from each other, Ar₁ represents a divalent aryl group that may have a substituent or a divalent heterocyclic group that may have a substituent, Ar₂ represents a monovalent aryl group that may have a substituent or a heterocyclic group that may have a substituent, and p represents an integer of 0 to 4, and when p represents 2 or more, multiple Ar₁'s may be identical to or different from each other,
in the formula [6], W represents a nitrogen atom, and
in the formula [7], Z represents an oxygen atom or a sulfur atom.

7. The organic light-emitting device according to claim 6, wherein a heterocycle formed of the W, the ring B₁, and the ring B₂ comprises any one of heterocycles represented in the following group A1; and
a heterocycle formed of the Z, the ring B₁, and the ring B₂ comprises any one of heterocycles represented in the following group A2: 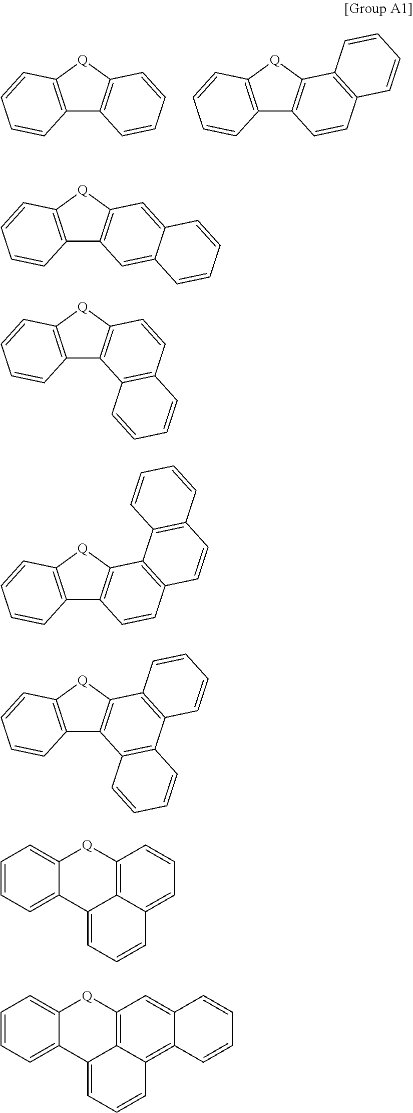
in the formulae, Q represents a nitrogen atom; 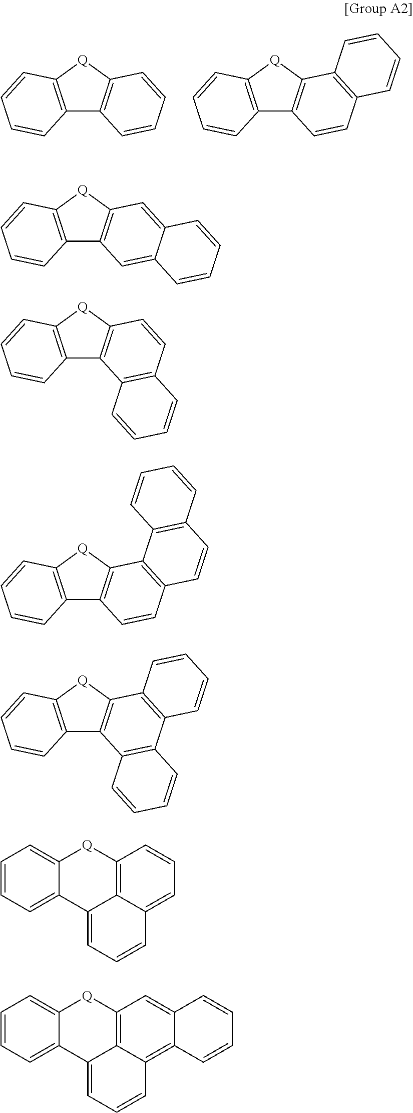
in the formulae, Q represents an oxygen atom or a sulfur atom.

8. The organic light-emitting device according to claim 6, wherein the heterocycle-containing compound represented by the formula [6] comprises a carbazole compound represented by the following general formula [8]: 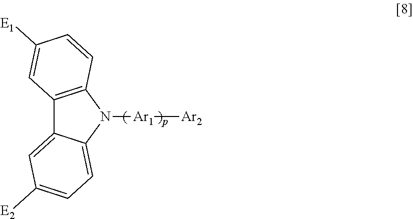
in the formula [8], E₁ and E₂ each represent a hydrogen atom, an alkyl group, or a substituted or unsubstituted aryl group.

9. The organic light-emitting device according to claim 6, wherein the heterocycle-containing compound represented by the formula [7] comprises a dibenzothiophene compound represented by the following general formula [9]: 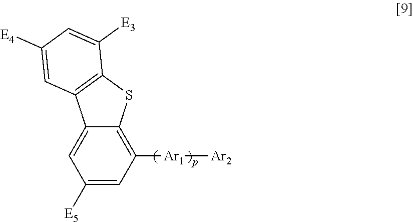
in the formula [9], E₃ to E₅ each represent a hydrogen atom, an alkyl group, or a substituted or unsubstituted aryl group.

10. The organic light-emitting device according to claim 6, wherein the heterocycle-containing compound represented by the formula [7] comprises a benzonaphtothiophene compound represented by the following general formula [10]: 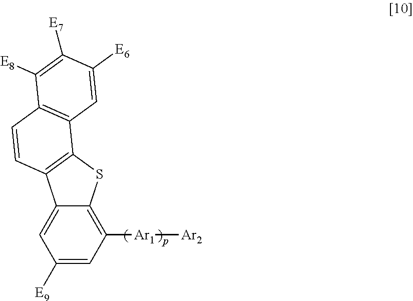
in the formula [10], E₆ to E₉ each represent a hydrogen atom, an alkyl group, or a substituted or unsubstituted aryl group.

11. The organic light-emitting device according to claim 6, wherein the heterocycle-containing compound represented by the formula [7] comprises a benzophenanthrothiophene compound represented by the following general formula [11]: 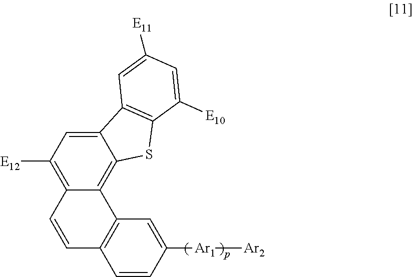
in the formula [11], E₁₀ to E₁₂ each represent a hydrogen atom, an alkyl group, or a substituted or unsubstituted aryl group.

12. The organic light-emitting device according to claim 6, wherein the heterocycle-containing compound represented by the formula [7] comprises a dibenzoxanthene compound represented by the following general formula [12]: 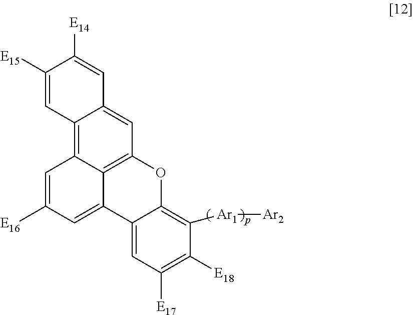
in the formula [12], E₁₃ to E₁₈ each represent a hydrogen atom, an alkyl group, or a substituted or unsubstituted aryl group.

13. The organic light-emitting device according to claim 6, wherein the heterocycle-containing compound represented by the formula [7] comprises a dibenzoxanthene compound represented by the following general formula [13]: 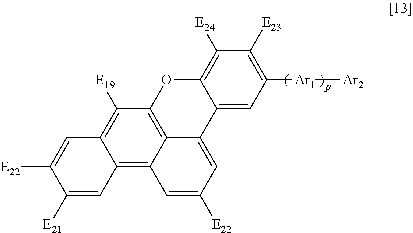
in the formula [13], E₁₉ to E₂₄ each represent a hydrogen atom, an alkyl group, or a substituted or unsubstituted aryl group.

14. The organic light-emitting device according to claim 8, wherein in the heterocycle-containing compounds represented by the formulae [8] to [13], all of the E₁ to E₂₄ each represent a hydrogen atom.

15. The organic light-emitting device according to claim 1, wherein the organic compound layer comprises an emission layer;
the guest in the emission layer comprises the iridium complex represented by the formula [1]; and
the host comprises a heterocycle-containing compound.

16. The organic light-emitting device according to claim 15, wherein the organic compound layer further includes an assist material different from the host and the guest.

17. The organic light-emitting device according to claim 16, wherein the assist material comprises an iridium complex.

18. The organic light-emitting device according to claim 1, wherein the organic light-emitting device emits red light.

19. A display apparatus comprising multiple pixels, wherein the pixels each include the organic light-emitting device according to claim 1 and an active device connected to the organic light-emitting device.

20. An information processing apparatus comprising:
a display portion for displaying an image; and
an input portion for inputting image information,
wherein the display portion comprises the display apparatus according to claim 19.

21. A lighting apparatus comprising:
the organic light-emitting device according to claim 1; and
an inverter circuit connected to the organic light-emitting device.

22. An image-forming apparatus comprising:
a photosensitive member;
charging unit for charging a surface of the photosensitive member;
exposing unit for exposing the photosensitive member to form an electrostatic latent image; and
developing unit for developing the electrostatic latent image formed on the surface of the photosensitive member,
wherein the exposing unit includes the organic light-emitting device according to claim 1.

23. An exposing apparatus for exposing a photosensitive member comprising a plurality of the organic light-emitting devices according to claim 1, wherein the organic light-emitting devices are placed to form a line.

24. The organic light-emitting device according to claim 9, wherein in the heterocycle-containing compounds represented by the formulae [8] to [13], all of the E₁ to E₂₄ each represent a hydrogen atom.

25. The organic light-emitting device according to claim 10, wherein in the heterocycle-containing compounds represented by the formulae [8] to [13], all of the E₁ to E₂₄ each represent a hydrogen atom.

26. The organic light-emitting device according to claim 11, wherein in the heterocycle-containing compounds represented by the formulae [8] to [13], all of the E₁ to E₂₄ each represent a hydrogen atom.

27. The organic light-emitting device according to claim 12, wherein in the heterocycle-containing compounds represented by the formulae [8] to [13], all of the E₁ to E₂₄ each represent a hydrogen atom.

28. The organic light-emitting device according to claim 13, wherein in the heterocycle-containing compounds represented by the formulae [8] to [13], all of the E₁ to E₂₄ each represent a hydrogen atom.
